(19) United States
(12) Patent Application Publication
(10) Pub. No.: US 2011/0189743 A1
(43) Pub. Date: Aug. 4, 2011

(54) MICROBIAL SYSTEMS FOR PRODUCING COMMODITY CHEMICALS

(75) Inventors: Yasuo Yoshikuni, Albany, CA (US); Asael Herman, Nes Ziona (IL); Adam J. Wargacki, Berkeley, CA (US)

(73) Assignee: BIO ARCHITECTURE LAB, INC., Berkeley, CA (US)

(21) Appl. No.: 12/899,419

(22) Filed: Oct. 6, 2010

Related U.S. Application Data
(60) Provisional application No. 61/249,205, filed on Oct. 6, 2009.

(51) Int. Cl. C12P 1/00; C12P 7/06; C12P 1/02; C12P 1/04

(52) U.S. Cl. 435/161; 435/171; 435/170; 435/41

(57) ABSTRACT

Provided are improved recombinant microorganisms, and methods of use thereof, for metabolizing biomolecules and producing commodity chemicals such as ethanol therefrom, and genetic constructs to achieve that end.

### CROSS-REFERENCE TO RELATED APPLICATIONS

This application claims the benefit under 35 U.S.C. §119(e) of U.S. Application No. 61/249,205, filed Oct. 6, 2009, which application is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 150097_{—}405_SEQUENCE_LISTING.txt. The text file is 433 KB, was created on Oct. 6, 2010, and is being submitted electronically via EFS-Web, concurrent with the filing of the specification.

### TECHNICAL FIELD

The present invention relates to improved recombinant microorganisms, and methods of use thereof, for metabolizing biomolecules and producing commodity chemicals therefrom.

### BACKGROUND OF THE INVENTION

Petroleum is facing declining global reserves and contributes to more than 30% of greenhouse gas emissions driving global warming. Annually 800 billion barrels of transportation fuel are consumed globally. Diesel and jet fuels account for greater than 50% of global transportation fuels.

Recently, significant legislation has been passed, requiring fuel producers to cap or reduce the carbon emissions from the production and use of transportation fuels. Fuel producers are seeking substantially similar, low carbon fuels that can be blended and distributed through existing infrastructure (e.g., refineries, pipelines, tankers).

Due to increasing petroleum costs and reliance on petrochemical feedstocks, the chemical industry is also looking for ways to improve margin and price stability, while reducing its environmental footprint. The chemical industry is striving to develop greener products that are more energy, water, and CO₂ efficient than current products. Fuels produced from biological sources, such as biomass, represent one aspect of process.

Many present methods for converting biomass into biofuels focus on the use of lignocellulolic biomass. However, there are many problems associated with using this process. Large-scale cultivation of lignocellulolic biomass requires a substantial amount of cultivated land, which can be only achieved by replacing food crop production with energy crop production, deforestation, and by recultivating currently uncultivated land. Other problems include a decrease in water availability and quality as well as an increase in the use of pesticides and fertilizers.

The degradation of lignocellulolic biomass using most biological systems is a very difficult challenge due to its substantial mechanistic strength and the complex chemical components. Approximately thirty different enzymes are required to fully convert lignocellulose to monosaccharides. The main available alternate to this complex approach requires a substantial amount of heat, pressure, and strong acids.

The art therefore requires an economic and technically simple process for converting biomass into hydrocarbons for use as biofuels or other saleable chemicals.

### BRIEF SUMMARY OF THE INVENTION

Embodiments of the present invention relate generally to methods of metabolizing a biomolecule, comprising incubating the biomolecule with a recombinant microorganism, for a time sufficient to allow metabolism of at least part of the biomolecule, wherein the recombinant microorganism comprises a tether-based polynucleotide as described herein, thereby metabolizing the biomolecule.

In certain embodiments, the biomolecule comprises a polysaccharide or a lipid. In certain embodiments, the polysaccharide comprises alginate, pectin, cellulose, cellobiose, laminarin, or a mixture thereof. In certain embodiments, the lipid comprises a fatty acid, a glycolipid, a betaine lipid, a glycerolipid, a phospholipid, a glycerolphospholipid, a sphingolipid, a sterol lipid, a prenol lipid, a saccharolipid, a polyketide, or a mixture thereof.

In certain embodiments, the above methods comprise converting the polysaccharide to a monosaccharide, an oligosaccharide, or both. In certain embodiments, the methods comprise converting the lipid to a fatty acid, a monosaccharide, or both. In certain embodiments, the monosaccharide or oligosaccharide is oligoalginate, mannuronate, guluronate, mannitol, α-keto acid, 4-deoxy-L-erythro-hexoselulose uronate (DEHU), 2-keto-3-deoxy D-gluconate (KDG), glucose, glucuronate, galacturonate, galactose, xylose, arabinose, or mannose. In certain embodiments, the fatty acid is 14:0, trans-14, 16:0, 16:1n-7, trans-16, 16:2n-6, 18:0, 18:1n-9, 18:2n-6, 18:3n-6, 18:3n-3, 18:4n-3, 20:0, 20:2n-6, 20:3n-6, 20:4n-3, 20:4n-6, or 20:5n-3.

In certain embodiments, the methods comprise converting the biomolecule to a commodity chemical. In certain embodiments, the commodity chemical is ethanol, butanol, or biodiesel. In certain embodiments, the biodiesel is a fatty acid, a fatty acid ester, or a terpenoid.

In certain embodiments, the fusion polypeptide encoded by the polynucleotide is secreted by the microorganism. In certain embodiments, the secreted fusion polypeptide is attached to the cell surface of the microorganism.

Certain embodiments relate to an isolated polynucleotide, comprising a nucleotide sequence that encodes a fusion polypeptide, wherein the fusion polypeptide comprises: (a) a carrier polypeptide, or a biologically active fragment thereof; and (b) a passenger polypeptide fused thereto, comprising a lyase, a cellulase, a laminarinase, a lipase, or a biologically active fragment or variant thereof. In certain embodiments, the lyase is an alginate lyase, oligoalginate lyase, pectin lyase, pectate lyase, rhamnogalacturonan lyase, gellan lyase, xanthan lyase, polymannuronate lyase, polygluronate lyase, polygalacturonate lyase, hyaluronan lyase, or a rhamnogalacturonan hydrolyase.

In certain embodiments, the fusion polypeptide further comprises a heterologous signal peptide. In certain embodiments, the heterologous signal peptide is derived from PelB (Pectobacterium sp.), PgsA (Bacillus subtilis), OmpA, StII, EX, PhoA, OmpF, PhoE, MalE, OmpC, Lpp, LamB, OmpT, Ltb, or Ag43 (E. coli). In certain embodiments, the carrier polypeptide comprises a native signal peptide.

In certain embodiments, the carrier polypeptide directs the secretion of the passenger polypeptide, displays the passenger polypeptide on the surface of the recombinant microorganism, or both. In certain embodiments, the carrier polypeptide comprises a bacterial outer membrane porin, an ice nucleation protein, PgsA (Bacillus subtilis), an autotransporter, or biologically active fragment or variant thereof.

In certain embodiments, the bacterial outer membrane porin comprises Omp1 (Zymomonas mobilis) or OmpA (E. coli). In certain embodiments, the encoded polypeptide comprises Omp1 or OmpA fused to a signal peptide from LLP (E. coli).

In certain embodiments, the ice nucleation protein comprises InaV (Pseudomonas syringae), InaK (Pseudomonas syringae), or a biologically active fragment or variant thereof. In certain embodiments, the autotransporter comprises PhoA-EstA (Pseudomonas aeruginosa, Pseudomonas putida, or Pseudomonas fluorescence), Ag43 (a non-AIDA based autotransporter from E. coli), or a biologically active fragment or variant thereof. In certain embodiments, the passenger polypeptide comprises an alginate lyase, or a biologically active fragment or variant thereof.

In certain embodiments, the alginate lyase is from Pseudoalteromonas sp. SM0524 or Sphingomonas sp. AI. In certain embodiments, the alginate lyase from Sphingomonas sp. comprises AI-I from Sphingomonas sp. AI, ΔAI-I from Sphingomonas sp. AI, AI-II from Sphingomonas sp. AI, AI-III from Sphingomonas sp. AI, AI-II′ from Sphingomonas sp. AI, or a biologically active fragment or variant thereof.

In certain embodiments, the passenger polypeptide comprises a cellulase, or a biologically active fragment thereof. In certain embodiments, the cellulase is from Tricoderma reesei, Aspergillus aculeatus, Clostridium cellulolyticum, or Saccharophagus degradans. In certain embodiments, the cellulase comprises an endo-1,4-glucanase I from Tricoderma reesei, an endo-1,4-glucanase II from Tricoderma reesei, and endo-1,4-glucanase III from Tricoderma reesei, a cellobiohydrolase II from Tricoderma reesei, a cellulase Cel9E from Clostridium cellulolyticum, a cellulase Cel9M from Clostridium cellulolyticum, an endo-1,4-glucanase Cel9G from Clostridium cellulolyticum, an endo-1,4-glucanase Cel5A from Clostridium cellulolyticum, an endo-cellulase Cel48F from Clostridium cellulolyticum, or a glucosidase I from Aspergillus aculeatus, or a biologically active fragment or variant thereof.

In certain embodiments, the passenger polypeptide comprises a laminarinase, or a biologically active fragment thereof. In certain embodiments, the laminarinase is from Euglena gracilis or Saccharophagous degradans. In certain embodiments, the passenger polypeptide comprises a lipase, or a biologically active fragment thereof.

In certain embodiments, the polynucleotide is operably linked to a promoter. In certain embodiments, the promoter comprises P_{trc} (E. coli), P_{pdc} (Zymomonas mobilis), P_{H207} (Coliphage), P_{D/E20} (Coliphage), P_{N25} (Coliphage), P_{L} (phage lambda), P_{A1} (phage T5), P_{rrnB-2} (E. coli), or P_{LPP} (E. coli).

In certain embodiments, the carrier polypeptide is fused to the N-terminus of the passenger polypeptide. In certain embodiments, the carrier polypeptide is fused to the C-terminus of the passenger polypeptide. In certain embodiments, the heterologous signal peptide is at the N-terminus of the fusion polypeptide.

In certain embodiments, the carrier polypeptide comprises Omp1 (Zymomonas mobilis), the passenger polypeptide comprises ΔAI-I from Sphingomonas sp. AI, and wherein the isolated polynucleotide is operably linked to a P_{pdc} promoter (Zymomonas mobilis). In certain embodiments, the carrier polypeptide comprises OmpA (E. coli), the passenger polypeptide comprises alginate lyase ΔAI-I from Sphingomonas sp. AI, further comprising a signal peptide from LPP (E. coli), and wherein the isolated polynucleotide is operably linked to a P_{pdc} promoter (Zymomonas mobilis).

In certain embodiments, the carrier polypeptide comprises Ag43 (E. coli), the passenger polypeptide comprises an alginate lyase from Pseudoalteromonas sp. SM0524, and wherein the isolated polynucleotide is operably linked to a P_{pdc} promoter (Zymomonas mobilis). In certain embodiments, the carrier polypeptide comprises Ag43 (E. coli), the passenger polypeptide comprises an alginate lyase AI-I from Sphingomonas sp. AI-I, and wherein the isolated polynucleotide is operably linked to a P_{pdc} promoter (Zymomonas mobilis).

In certain embodiments, the carrier polypeptide comprises Ag43 (E. coli), the passenger polypeptide comprises an alginate lyase ΔAI-I from Sphingomonas sp. AI-L and wherein the isolated polynucleotide is operably linked to a P_{pdc} promoter (Zymomonas mobilis). In certain embodiments, the carrier polypeptide comprises Ag43 (E. coli), the passenger polypeptide comprises an alginate lyase AI-II from Sphingomonas sp. AI-I, and wherein the isolated polynucleotide is operably linked to a P_{pdc} promoter (Zymomonas mobilis).

In certain embodiments, the carrier polypeptide comprises Ag43 (E. coli), the passenger polypeptide comprises an alginate lyase AI-III from Sphingomonas sp. AI-L and wherein the isolated polynucleotide is operably linked to a P_{pdc} promoter (Zymomonas mobilis). In certain embodiments, the carrier polypeptide comprises Ag43 (E. coli), the passenger polypeptide comprises an alginate lyase from Pseudoalteromonas sp. SM0524, and wherein the isolated polynucleotide is operably linked to a P_{H207} promoter (Coliphage).

Certain embodiments include vectors, comprising a polynucleotide according as described herein. In certain embodiments, the vector comprises pTrc99a or pCCfos2 (mini-F plasmid).

Certain embodiments include recombinant microorganisms, comprising a polynucleotide or vector described herein. In certain embodiments, the recombinant microorganism is Escherichia coli, Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharophagus degradans, Sccharomyces cerevisiae, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio alginolyticus, Vibrio splendidus, Xanthomonas, yeast, Yarrowia lipolytica, Zygosaccharomyces rouxii, Zymomonas, or Zymomonus mobilis.

Certain embodiments relate generally to recombinant microorganisms that are capable of growing on a polysaccharide as a sole source of carbon, comprising one or more exogenous polynucleotides that contain a genomic region between V12B01_{—}24189 and V12B01_{—}24249 of Vibrio splendidus, and that encodes an additional outer membrane porin. In certain embodiments, the outer membrane porin is from Vibrio splendidus. In certain embodiments, the outer membrane porin comprises an amino acid sequence at least 80%, 85%, 90%, 95%, 98%, or 100% identical to the amino acid sequence set forth in SEQ ID NO:92 [see, e.g., pALG2.0].

In certain embodiments, the recombinant microorganism comprises one or more exogenous polynucleotides that encode a symporter and a porin. In certain embodiments, the symporter and porin are from Vibrio splendidus. In certain embodiments, the symporter and the porin from Vibrio splendidus, respectively, comprise an amino acid sequence at least 80%, 85%, 90%, 95%, 98%, or 100% identical to the amino acid sequence set forth in SEQ ID NO:96 (symporter), 92 (porin), or 94 (porin) [see, e.g., pALG2.5].

In certain embodiments, the recombinant microorganism comprises one or more exogenous polynucleotides that encode an ABC transporter, an oligoalginate lyase, and a DEHU hydrogenase. In certain embodiments, the ABC transporter, the oligoalginate lyase, and the DEHU hydrogenase are from Agrobacterium tumefaciens. In certain embodiments, the ABC transporter, the oligoalginate lyase, and the DEHU hydrogenase from Agrobacterium tumefaciens, respectively, comprise an amino acid sequence at least 80%, 85%, 90%, 95%, 98%, or 100% identical to the amino acid sequence set forth in SEQ ID NO:110, 112, 114, 116, 118 (ABC transporters), 120 (olioalginate lyase), or 122 (DEHU hydrogenase) [see, e.g., pALG3.0].

In certain embodiments, the recombinant microorganism comprises an exogenous polynucleotide that encodes one or more alginate lyases. In certain embodiments, the recombinant microorganism comprises one or more exogenous polynucleotides that encode two or more alginate lyases. In certain embodiments, the alginate lyases are from Vibrio splendidus. In certain embodiments, the alginate lyases Vibrio splendidus comprise an amino acid sequence at least 80%, 85%, 90%, 95%, 98%, or 100% identical to the amino acid sequence set forth in SEQ ID NO:98 or 100 [see, e.g., pALG3.5].

In certain embodiments, the recombinant microorganism comprises one or more exogenous polynucleotides that encode one or more β-glucosidases and a transporter. In certain embodiments, the recombinant microorganism comprises one or more exogenous polynucleotides that encode three or more β-glucosidases and a transporter. In certain embodiments, the β-glucosidases and the transporter are from Saccharophagous degradans. In certain embodiments, the one or more β-glucosidases from Saccharophagous degradans comprise an amino acid sequence at least 80%, 85%, 90%, 95%, 98%, or 100% identical to at least one of SEQ ID NO:124, 126, or 130, and wherein the transporter from Saccharophagous degradans comprises an amino acid sequence at least 80%, 85%, 90%, 95%, 98%, or 100% identical to SEQ ID NO:128 [see, e.g., pALG4.0].

In certain embodiments, the recombinant microorganism comprises one or more exogenous polynucleotides that encode one or more transporters. In certain embodiments, the one or more transporters are from Saccharophagous degradans. In certain embodiments, the one or more transporters from Saccharophagous degradans comprise an amino acid sequence at least 80%, 85%, 90%, 95%, 98%, or 100% identical to at least one of SEQ ID NO:108 or 128 [see, e.g., pALG4.0].

In certain embodiments, the recombinant microorganism comprises one or more exogenous polynucleotides that encode one or more cellodextrinases. In certain embodiments, the recombinant microorganism comprises one or more exogenous polynucleotides that encode two or more cellodextrinases. In certain embodiments, the cellodextrinases are from Saccharophagous degradans. In certain embodiments, the one or more cellodextrinases from Saccharophagous degradans comprise an amino acid sequence at least 80%, 85%, 90%, 95%, 98%, or 100% identical to at least one of SEQ ID NO:136 or 138 [see, e.g., pALG5.0 and pALG5.1].

In certain embodiments, the recombinant microorganism comprises one or more exogenous polynucleotides that encode one or more cellulases. In certain embodiments, the one or more cellulases are derived from Saccharophagous degradans. In certain embodiments, the one or more cellulases from Saccharophagous degradans comprise an amino acid sequence at least 80%, 85%, 90%, 95%, 98%, or 100% identical to at least one of SEQ ID NO: 134, 140, 142, 144, 146, 148, 150, 152, or 154 [see, e.g., pALG 5.0, 5.1, 5.2, and 5.3].

In certain embodiments, the recombinant microorganism comprises one or more exogenous polynucleotides that encode one or more cellobiohydrolases. In certain embodiments, the one or more cellobiohydrolases are derived from Saccharophagous degradans. In certain embodiments, the one or more cellobiohydrolases from Saccharophagous degradans comprise an amino acid sequence at least 80%, 85%, 90%, 95%, 98%, or 100% identical to at least one of SEQ ID NO:146, 148, 150, 152, or 154 [see, e.g., pALG5.2, and 5.3].

In certain embodiments, the recombinant microorganism further comprises one or more deletions in a gene that encodes for a regulator of aerobic fatty acid metabolism, wherein the microorganism has enhanced fatty acid metabolism as compared to a microorganism without said one or more deletions. In certain embodiments, the gene that encodes for a regulator of aerobic fatty acid metabolism is fadR.

In certain embodiments, the recombinant microorganism further comprises one or more deletions in a lactose dehydrogenase (ΔldhA) gene, a fumarate reductase (Δfrd) gene, a pyruvate formate lyase (ΔpflA) gene, a pyruvate formate lyase (ΔpflB) gene, a formate transporter (ΔfocA) gene, or any combination thereof. In certain embodiments, the recombinant microorganism further comprises one or more exogenous polynucleotides that encode pyruvate decarboxylase (pdc), alcohol dehydrogenase I (adhA), and alcohol dehydrogenase II (adhB). In certain embodiments, the recombinant microorganism further comprises an exogenous polynucleotide that encodes an acetaldehyde/alcohol dehydrogenase (adhE). In certain embodiments, the recombinant microorganism further comprises a polynucleotide as described herein that encodes a tether-based fusion polypeptide.

In certain embodiments, the recombinant microorganism is capable of growing on a polysaccharide as a sole source of carbon, including polysaccharides such as alginate, pectin, cellulose, cellobiose, or laminarin. In certain embodiments, the recombinant microorganism is Escherichia coli, Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharophagus degradans, Sccharomyces cerevisiae, Sclerotina libertine, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio splendidus, Vibrio alginolyticus, Yarrowia lipolytica, Xanthomonas, yeast, Zygosaccharomyces rouxii, Zymomonas, or Zymomonus mobilis.

Certain embodiments relate generally to methods of metabolizing a biomolecule, comprising contacting the biomolecule with a recombinant microorganism as described above and herein. In certain embodiments, the biomolecule comprises a polysaccharide or a lipid. In certain embodiments, the polysaccharide comprises alginate, pectin, cellulose, cellobiose, laminarin, or a mixture thereof. In certain embodiments, the lipid comprises a fatty acid, a glycolipid, a betaine lipid, a glycerolipid, a phospholipid, a glycerolphospholipid, a sphingolipid, a sterol lipid, a prenol lipid, a saccharolipid, a polyketide, or a mixture thereof.

Certain embodiments comprise converting the polysaccharide to a monosaccharide, an oligosaccharide, or both. Certain embodiments comprise converting the lipid to a fatty acid, a monosaccharide or both. In certain embodiments, the monosaccharide or oligosaccharide is oligoalginate, mannuronate, guluronate, mannitol, α-keto acid, 4-deoxy-L-erythro-hexoselulose uronate (DEHU), 2-keto-3-deoxy D-gluconate (KDG), glucose, glucuronate, galacturonate, galactose, xylose, arabinose, or mannose.

Certain embodiments comprise converting the biomolecule to a commodity chemical. In certain embodiments, the commodity chemical is ethanol, butanol, or biodiesel. In certain embodiments, the biodiesel is a fatty acid, a fatty acid ester, or a terpenoid.

Certain embodiments relate to methods of enhancing production or yield of a target molecule by a recombinant microorganism, comprising incubating the microorganism with a mixture of at least one uronic acid and at least one sugar alcohol under anaerobic fermentative conditions, for a time sufficient to allow metabolism of at least part of the mixture, wherein the at least one uronic acid and the at least one sugar alcohol have different reduction-oxidation (redox) potentials, and wherein metabolism of the mixture balances the intracellular redox potential of the microorganism, thereby enhancing production or yield of the target molecule.

In certain embodiments, the at least one uronic acid is alginate, mannuronate, guluronate, DEHU, glucuronate, galacturonate, or a mixture thereof. In certain embodiments, the at least one sugar alcohol is mannitol, glycerol, or both. In certain embodiments, the uronic acid:sugar alcohol ratio is about 5:1, 4:1, 3:1, 3:2, 2:1, 1:1, 1:2, 2:3 1:3, 1:4, 1:5.

In certain embodiments, the at least one uronic acid is alginate and the at least one sugar alcohol is mannitol. In certain embodiments, the alginate:mannitol ratio is about 5:1, 4:1, 3:1, 3:2, 2:1, 1:1, 1:2, 2:3 1:3, 1:4, or 1:5. In certain embodiments, the at least one uronic acid is galacturonate and the at least one sugar alcohol is mannitol. In certain embodiments, the galacturonate:mannitol ratio is about 5:1, 4:1, 3:1, 3:2, 2:1, 1:1, 1:2, 2:3 1:3, 1:4, or 1:5. In certain embodiments, the galacturonate:mannitol ratio is about 2:1. In certain embodiments, the at least one uronic acid is glucuronate and the at least one sugar alcohol is mannitol. In certain embodiments, the glucuronate:mannitol ratio is about 5:1, 4:1, 3:1, 3:2, 2:1, 1:1, 1:2, 2:3 1:3, 1:4, or 1:5. In certain embodiments, the glucuronate:mannitol ratio is about 1:1. In certain embodiments, the microorganism is a recombinant microorganism as described above or herein.

In certain embodiments, the recombinant microorganism comprises a tether-based fusion polypeptide encoding polynucleotide according as described herein. In certain embodiments, wherein the recombinant microorganism comprises one or more deletions in a lactose dehydrogenase (ΔldhA) gene, a fumarate reductase (Δfrd) gene, a pyruvate formate lyase (ΔpflA) gene, a pyruvate formate lyase (ΔpflB) gene, a formate transporter (ΔfocA) gene, or any combination thereof. In certain embodiments, the recombinant microorganism comprises one or more exogenous polynucleotides that encode pyruvate decarboxylase (pdc), alcohol dehydrogenase I (adhA), and alcohol dehydrogenase II (adhB). In certain embodiments, the recombinant microorganism further comprises an exogenous polynucleotide that encodes acetaldehyde/alcohol dehydrogenase (adhE).

In certain embodiments, the method enhances yield of the target molecule to at least about 30%, 35%, 40%, 45%, 50%, 55%, 60%, 65%, 70%, 75%, 80%, 85%, 88%, 90%, 91%, 92%, 93%, 94%, 95%, 96%, 97%, 98%, 99%, or 100% of a theoretical maximum yield. In certain embodiments, the method increases percentage yield of the target molecule by at least about 10%, 15%, 20%, 25%, 30%, 35%, 40%, 50%, 60%, 70%, 80%, or 90% as compared to incubating the microorganism with the uronic acid alone or the sugar alcohol alone. In certain embodiments, the method reduces intracellular NADH/NADPH accumulation as compared to incubating the microorganism with the sugar alcohol alone. In certain embodiments, the method reduces intracellular acetate accumulation as compared to incubating the microorganism with the uronic acid alone.

In certain embodiments, the target molecule is a commodity chemical. In certain embodiments, the commodity chemical is ethanol, butanol, or biodiesel. In certain embodiments, the biodiesel is a fatty acid, a fatty acid ester, or a terpenoid. In certain embodiments, the recombinant microorganism is Escherichia coli, Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharophagus degradans, Sccharomyces cerevisiae, Sclerotina libertine, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio splendidus, Vibrio alginolyticus, Yarrowia lipolytica, Xanthomonas, yeast, Zygosaccharomyces rouxii, Zymomonas, or Zymomonus mobilis.

Certain embodiments relate generally to recombinant microorganisms, comprising one or more exogenous polynucleotides that encode pyruvate decarboxylase (pdc), alcohol dehydrogenase I (adhA), and alcohol dehydrogenase II (adhB); and, at least one of the following: (a) one or more deletions in a lactose dehydrogenase (ΔldhA) gene, a fumarate reductase (Δfrd) gene, a pyruvate formate lyase (ΔpflA) gene, a pyruvate formate lyase (ΔpflB) gene, a formate transporter (ΔfocA) gene, or any combination thereof, (b) an exogenous polynucleotide that encodes an acetaldehyde dehydrogenase (adhE), or (c) both (a) and (b). Typically, these embodiments can be used to produce ethanol from biomass such as kelp. In certain embodiments, the recombinant microorganism further comprises one or more polynucleotides that contain a genomic region between V12B01_{—}24189 and V12B01_{—}24249 of Vibrio splendidus [see, e.g., pALG.1.5].

In certain embodiments, the recombinant microorganism further comprises an exogenous polynucleotide that encodes an outer membrane porin. In certain embodiments, the outer membrane porin is from Vibrio splendidus. In certain embodiments, the outer membrane porin comprises an amino acid sequence at least 80%, 85%, 90%, 95%, 98%, or 100% identical to the amino acid sequence set forth in SEQ ID NO:92 [see, e.g., pALG2.0].

In certain embodiments, the recombinant microorganism comprises one or more exogenous polynucleotides that encode a symporter and a porin. In certain embodiments, the symporter and porin are from Vibrio splendidus. In certain embodiments, the symporter and the porin from Vibrio splendidus, respectively, comprise an amino acid sequence at least 80%, 85%, 90%, 95%, 98%, or 100% identical to the amino acid sequence set forth in SEQ ID NO:96 (symporter), 92 (porin), or 94 (porin) [see, e.g., pALG2.5].

In certain embodiments, the recombinant microorganism comprises one or more exogenous polynucleotides that encode an ABC transporter, an oligoalginate lyase, and a DEHU hydrogenase. In certain embodiments, the ABC transporter, the oligoalginate lyase, and the DEHU hydrogenase are from Agrobacterium tumefaciens. In certain embodiments, the ABC transporter, the oligoalginate lyase, and the DEHU hydrogenase from Agrobacterium tumefaciens, respectively, comprise an amino acid sequence at least 80%, 85%, 90%, 95%, 98%, or 100% identical to the amino acid sequence set forth in SEQ ID NO:110, 112, 114, 116, 118 (ABC transporters), 120 (olioalginate lyase), or 122 (DEHU hydrogenase) [see, e.g., pALG3.0].

In certain embodiments, the recombinant microorganism comprises an exogenous polynucleotide that encodes one or more alginate lyases. In certain embodiments, the recombinant microorganism comprises one or more exogenous polynucleotides that encode two or more alginate lyases. In certain embodiments, the alginate lyases are from Vibrio splendidus. In certain embodiments, the alginate lyases Vibrio splendidus comprise an amino acid sequence at least 80%, 85%, 90%, 95%, 98%, or 100% identical to the amino acid sequence set forth in SEQ ID NO:98 or 100 [see, e.g., pALG3.5].

In certain embodiments, the recombinant microorganism comprises one or more exogenous polynucleotides that encode one or more β-glucosidases and a transporter. In certain embodiments, the recombinant microorganism comprises one or more exogenous polynucleotides that encode three or more β-glucosidases and a transporter. In certain embodiments, the β-glucosidases and the transporter are from Saccharophagous degradans. In certain embodiments, the one or more β-glucosidases from Saccharophagous degradans comprise an amino acid sequence at least 80%, 85%, 90%, 95%, 98%, or 100% identical to at least one of SEQ ID NO:124, 126, or 130, and wherein the transporter from Saccharophagous degradans comprises an amino acid sequence at least 80%, 85%, 90%, 95%, 98%, or 100% identical to SEQ ID NO:128 [see, e.g., pALG4.0].

In certain embodiments, the recombinant microorganism comprises one or more exogenous polynucleotides that encode one or more transporters. In certain embodiments, the one or more transporters are from Saccharophagous degradans. In certain embodiments, the one or more transporters from Saccharophagous degradans comprise an amino acid sequence at least 80%, 85%, 90%, 95%, 98%, or 100% identical to at least one of SEQ ID NO:108 or 128 [see, e.g., pALG4.0].

In certain embodiments, the recombinant microorganism comprises one or more exogenous polynucleotides that encode one or more cellodextrinases. In certain embodiments, the recombinant microorganism comprises one or more exogenous polynucleotides that encode two or more cellodextrinases. In certain embodiments, the cellodextrinases are from Saccharophagous degradans. In certain embodiments, the one or more cellodextrinases from Saccharophagous degradans comprise an amino acid sequence at least 80%, 85%, 90%, 95%, 98%, or 100% identical to at least one of SEQ ID NO:136 or 138 [see, e.g., pALG5.0 and pALG5.1].

In certain embodiments, the recombinant microorganism comprises one or more exogenous polynucleotides that encode one or more cellulases. In certain embodiments, the one or more cellulases are derived from Saccharophagous degradans. In certain embodiments, the one or more cellulases from Saccharophagous degradans comprise an amino acid sequence at least 80%, 85%, 90%, 95%, 98%, or 100% identical to at least one of SEQ ID NO:134, 140, 142, 144, 146, 148, 150, 152, or 154 [see, e.g., pALG 5.0, 5.1, 5.2, and 5.3].

In certain embodiments, the recombinant microorganism comprises one or more exogenous polynucleotides that encode one or more cellobiohydrolases. In certain embodiments, the one or more cellobiohydrolases are derived from Saccharophagous degradans. In certain embodiments, the one or more cellobiohydrolases from Saccharophagous degradans comprise an amino acid sequence at least 80%, 85%, 90%, 95%, 98%, or 100% identical to at least one of SEQ ID NO:146, 148, 150, 152, or 154 [see, e.g., pALG5.2, and 5.3].

In certain embodiments, the recombinant microorganism further comprises one or more deletions in a gene that encodes for a regulator of aerobic fatty acid metabolism, wherein the microorganism has enhanced fatty acid metabolism as compared to a microorganism without said one or more deletions. In certain embodiments, the gene that encodes for a regulator of aerobic fatty acid metabolism is fadR.

In certain embodiments, the recombinant microorganism further comprises one or more deletions in a lactose dehydrogenase (ΔldhA) gene, a fumarate reductase (Δfrd) gene, a pyruvate formate lyase (ΔpflA) gene, a pyruvate formate lyase (ΔpflB) gene, a formate transporter (ΔfocA) gene, or any combination thereof. In certain embodiments, the recombinant microorganism further comprises one or more exogenous polynucleotides that encode pyruvate decarboxylase (pdc), alcohol dehydrogenase I (adhA), and alcohol dehydrogenase II (adhB). In certain embodiments, the recombinant microorganism further comprises an exogenous polynucleotide that encodes an acetaldehyde/alcohol dehydrogenase (adhE). In certain embodiments, the recombinant microorganism further comprises a polynucleotide as described herein that encodes a tether-based fusion polypeptide.

In certain embodiments, the recombinant microorganism is capable of growing on a polysaccharide as a sole source of carbon, including polysaccharides such as alginate, pectin, cellulose, cellobiose, or laminarin. In certain embodiments, the recombinant microorganism is Escherichia coli, Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharophagus degradans, Sccharomyces cerevisiae, Sclerotina libertine, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio splendidus, Vibrio alginolyticus, Yarrowia lipolytica, Xanthomonas, yeast, Zygosaccharomyces rouxii, Zymomonas, or Zymomonus mobilis.

Certain embodiments relate generally to methods of converting a saccharide, a fatty acid, or both, to ethanol, comprising incubating the saccharide, fatty acid, or both, with a recombinant microorganism as described above and herein. In certain embodiments, the saccharide is a polysaccharide. In certain embodiments, the polysaccharide is alginate, pectin, cellulose, cellobiose, or laminarin. In certain embodiments, the saccharide is a monosaccharide or an oligosaccharide. In certain embodiments, the monosaccharide or oligosaccharide is oligoalginate, mannuronate, guluronate, mannitol, α-keto acid, 4-deoxy-L-erythro-hexoselulose uronate (DEHU), 2-keto-3-deoxy D-gluconate (KDG), glucose, glucuronate, galacturonate, galactose, xylose, arabinose, or mannose. In certain embodiments, the method comprises enhancing production or yield of ethanol by incubating the recombinant microorganism according to any of the other methods described above and herein. In certain embodiments, the polysaccharide or fatty acid is derived from biomass, such as kelp.

### BRIEF DESCRIPTION OF THE DRAWINGS

FIG. 1 shows the alginate lyase (AL) specific activity (SA) of E. coli W cells carrying the vectors described in Table 2. These experiments are described in Example 1. Each bar represents the average of 3 independent cultures, and the error bars show the standard deviation.

FIG. 2 shows the secretion of AI-IV and Atu3025 enzymes by the various secretion peptide sequences PelB, OmpA, StII, EX, PhoA, OmpF, PhoE, MalE, OmpC, LPP, LamB, OmpT, and LTB. See Example 1.

FIGS. 3A-3U diagram the components of vectors pALG1.5, pALG2.0, pALG2.5, pALG3.0, pALG3.5, pALG4.0, pALG5.0, pALG5.1, pALG5.2, and pALG5.3 vectors, among others. The construction of these vectors is described in Examples 2 and 3.

FIG. 4 shows the growth of recombinant E. coli on alginate (see Example 2). As indicated in the x-axis, E. coli was first transformed with the pALG1.5, pALG2.0, pALG2.5, or pALG3.0 vectors, and then grown on degraded alginate. The y-axis indicates the OD₆₀₀ₙₘ value. See Example 2.

FIG. 5 shows the alginate residuals after the growth of the various E. coli strains on alginate (see Example 2). FIG. 5A shows the starting media, which contains a substantial amount of oligoalginate molecules (e.g., ΔM, ΔG, ΔMM, ΔGG), represented by the four left-most peaks. FIG. 5B shows a slightly reduced concentration of oligoalginate molecules in media after incubation with the E. coli containing the pALG1.5 vector. FIGS. 5C and 5D show a significantly reduced concentration of oligoalginate molecules in media after incubation with E. coli containing the pALG2.0 and pALG2.5 vectors, respectively. See Example 2.

FIG. 6 shows the OD₆₀₀ₙₘ values for recombinant E. coli growing on cellobiose (see Example 3). pALG3.5 provides a negative control. See Example 3.

FIG. 7 shows the OD₆₀₀ₙₘ values for recombinant E. coli growing in methylcarboxycellulose (see Example 3). pALG3.5 provides a negative control. See Example 3.

FIG. 8 shows the effects of alcohol/acetaldehyde dehydrogenase (adhE) on the production of ethanol in E. coli. FIG. 8A shows the results for control (ctrl) cells having only the pdc-adhA-adhB operon, and for pTrcAdhE cells having both the pdc-adhA-adhB operon and adhE. FIG. 8B shows the effects of fadR deletions on ethanol production in the presence of adhE. Control (ctrl) cells have the pdc-adhA-adhB operon and adhE, and fadR (deletion) cells have the pdc-adhA-adhB operon and adhE, and further have a deletion in the fadR gene, a regulator of fatty acid metabolism. See Example 5.

FIGS. 9A and 9B show the effects of various deletion mutants on the production of ethanol from mixed sugar sources. See Example 6.

FIGS. 10A and 10B show that production of ethanol from mixed sugar sources can be optimized by adjusting the ratios of the sugars in the mixture. See Example 6.

FIGS. 11A-11C show the production of ethanol from recombinant E. coli growing on kelp. FIGS. 11A and 11B show the production of ethanol from Laminaria japonica, and FIG. 11C shows the production of ethanol from Macrocystis pyrifera. See Example 7.

FIG. 12 shows the production of ethanol from recombinant E. coli growing on kelp. FIG. 12A shows the effects of kelp pre-treatment on ethanol production, and FIG. 12B shows the effects of various tether-display systems on increasing the production of ethanol from Macrocystis pyrifera. See Example 7.

FIGS. 13A and 13B show the growth of various recombinant E. coli substrains on guluronate as a sole source of carbon. See Example 8.

FIGS. 14A-14C show the lyase activities of the tether/surface-display systems of (14A) ΔPaAly, (14B) ΔA1-I, and (14C) A1-II.

FIGS. 15A-15D show the lyase activities of the alginate lyase ΔPaAly tether/surface-display system having different promoters in comparison to that of BAL492 (P_{D/E20} promoter).

FIG. 16A shows the lyase activities of the secretion system of ΔpaAly, and FIG. 16B shows a comparison of lyase activities between the tether and secretion systems of ΔpaAly.

FIG. 17A shows the lyase activities of the secretion system of A1-II, and

FIG. 17B shows a comparison of lyase activities between the tether and secretion systems of A1-II.

FIGS. 18A-18B shows the lyase activity of tether (18A) and secretion (18B) systems of dual-enzyme constructs where ΔPaAly was expressed together with ΔA1-I or A1-II independently.

FIGS. 19A-19F show the growth of E. coli ATCC8739 harboring pALG1.5, pALG1.7, pALG2.1, pALG2.2, pALG2.3 pALG7.2.1, pALG7.2.2, pALG7.2.3, and pALG7.2.4 on alginate and guluronate. FIGS. 19A-19C show the results for growth on 0.2% alginate, and FIGS. 19D-19F show the corresponding results for growth on 0.2% guluronate.

FIG. 20 shows ethanol production from synthetic media (mannitol:glucuronate=2:1 ratio) using various chromosome integrated E. coli strains.

FIG. 21 shows the various cellular pathways involved in the synthesis of carbon-based molecules, and illustrates certain pathways that can be down-regulated by deletion mutation to shunt carbon resources towards the production of ethanol.

### DETAILED DESCRIPTION OF THE INVENTION

Embodiments of the present invention relate generally to improved methods for converting biomass-based biomolecules, such as polysaccharides and fatty acids, into commodity chemicals, such as biofuels and biodiesel, and to recombinant microorganisms and genetic constructs for accomplishing that end. The methods, recombinant microorganisms, and genetic constructs of the present invention can be useful in optimizing the production of biofuels or other commodity chemicals, such as ethanol, from biomass, such as kelp, and thereby provide an efficient and relatively low impact alternative for producing useful and valuable chemicals from natural and renewable resources.

In certain embodiments, the present invention relates to improved fusion polypeptide systems for directing the secretion or surface display of biomolecule-metabolizing or biomolecule-transporting enzymes in microorganisms, mainly to enhance their ability to metabolize or de-polymerize larger, often polymeric, biomolecules, and transport the smaller components of those biomolecules into the cell for use in commodity chemical-producing metabolic pathways. In certain embodiments, the present invention relates to improved vector systems, and recombinant microorganisms containing the same, which comprise a variety of newly identified lyases, hydrolyases, transporters, etc., which confer on the recombinant microorganisms the ability to grow more efficiently on biomass-based biomolecules, such as alginate, cellobiose, and methylcarboxycellulose, and fatty acids, including combinations thereof, by first converting those carbon sources to common metabolites, and then using those common metabolites to synthesize commodity chemicals.

Certain embodiments also relate to the use of deletion mutants to maximize the production of a desired carbon-based target molecule, or commodity chemical. Examples of such deletion mutants include, without limitation, deletions in the lactose dehydrogenase gene (ΔldhA), which plays a key role in the synthesis of lactate, the fumarate reductase gene (Δfrd), which converts fumarate into succinate, the pflB-focA operon (ΔpflB-focA), which encodes the central enzyme of fermentative metabolism, a pyruvate formate-lyase (PFL) gene (ΔpflA or ΔpflB), a formate/nitrite transporter (ΔFocA) gene, and fadR, a regulator of fatty acid metabolism. Without wishing to be bound by any one theory, it is believed that the production of a desired carbon-based target molecule, such as ethanol, can be enhanced by reducing the production of other carbon based molecules, such as lactate or succinate, thereby shunting the limited resources of a given bacteria cell towards the production of the desired molecule. The deletion mutants of the present invention may be used in combination with any of the other vector systems, recombinant microorganisms, or methods provided herein.

Certain embodiments relate to methods of optimizing the growth of the recombinant microorganisms described herein, mainly to enhance the yield of a desired target molecule or commodity chemical. Certain of these methods involve optimizing a growth mixture, typically comprising polysaccharides, fatty acids, or both, to achieve an optimal ratio of different polysaccharides. For instance, certain embodiments relate to the use of a growth mixture that comprises at least one uronic acid and at least one sugar alcohol, often under anaerobic fermentative conditions, wherein the at least one uronic acid and the at least one sugar alcohol have different reduction-oxidation (redox) potentials. In certain embodiments, ratio of the uronic acid to the sugar alcohol is optimized for a given microorganism or fermentation system. Without wishing to be bound by any one theory, it is believed that the use of such mixtures balances the intracellular redox potential of the microorganism, reducing the growth inhibitory effects of redox imbalance (e.g., excess NADH), and thereby enhancing production or yield of the target molecule. These methods can be used with any of the recombinant microorganisms or methods described herein.

Certain embodiments relate to improved methods and recombinant microorganisms for producing ethanol from biomolecules such as polysaccharides and lipids, including mixtures thereof, typically those derived from biomass such as kelp. These and other embodiments use sugar-dependent ethanol-synthesizing pathways, such as the pdc-adhA-adhB operon from Zymomonas mobilis (i.e., pyruvate decarboxylase (Pdc) and alcohol dehydrogenases I and II (adhA and adhB, respectively)), in combination with an acetaldehyde/alcohol dehydrogenase (adhE), to more efficiently convert polysaccharides and fatty acids into ethanol. Without wishing to be bound by any one theory, it is believed that the pdc-adhA-adhB operon does not effectively utilize the intermediates or by-products of fatty acid metabolism, such as acetyl-CoA. The addition of adhE remedies this deficiency, and improves the yield of ethanol from such recombinant microorganisms, especially when growing on mixtures of polysaccharides and fatty acids, as in biomass such as kelp.

Certain preferred embodiments relate to integrated systems to enhance or maximize the ability of recombinant microorganisms to metabolize polysaccharides and fatty acids from biomass such as kelp, and to produce ethanol therefrom. These and related embodiments typically utilize a combination of two or more of the technologies described herein, including, without limitation, improved vector systems (e.g., pALG2.0, pALG4.0) or their equivalents, improved tether-display systems to produce fusion polypeptides that comprise lyases or other enzymes, deletion mutants (e.g., ΔldhA), and/or the pdc-adhA-adhB operon in combination with adhE, including functional equivalents thereof, to maximize the production of ethanol from kelp or other biomass. Certain embodiments are capable of approaching, achieving, or even surpassing the theoretical maximum yield of ethanol production from biomass such as kelp.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein are hereby incorporated by reference in their entirety.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below. All references referred to herein are incorporated by reference in their entirety.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

The term “biologically active fragment,” as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence.

The term “reference sequence” refers generally to a nucleic acid sequence (coding or non-coding, e.g., promoter or other regulatory sequence) or amino acid sequence of any polypeptide or enzyme having a biological activity described herein (e.g., alginate lyase, cellulase, outer membrane porin, alcohol dehydrogenase, symporter, decarboxylase, secretion signal), such as a “wild-type” sequence, including those reference sequences in the Sequence Listing and in Tables C-G. A reference sequence may also include naturally-occurring, functional variants (i.e., orthologs or homologs) of the sequences described herein.

Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between, which comprise or encode a polypeptide having an enzymatic activity of a reference polynucleotide or polypeptide (see the Sequence Listing). Representative biologically active fragments generally participate in an interaction, e.g., an intra-molecular or an inter-molecular interaction. An inter-molecular interaction can be a specific binding interaction or an enzymatic interaction. Examples of enzymatic interactions or activities include alginate lyase activities, cellulase activities, alcohol dehydrogenase activities, decarboxylase activities, isomerase activities, kinase activities, among others described herein. Biologically active fragments typically comprise one or more active sites or enzymatic or binding motifs, as described herein and known in the art.

A “biomolecule” refers generally to an organic molecule that is produced by a living organism, including large polymeric molecules (biopolymers) such as proteins, polysaccharides, and nucleic acids as well, as small molecules such as primary secondary metabolites, lipids, phospholipids, glycolipids, sterols, glycerolipids, vitamins, and hormones. Organic molecules (e.g., biomolecules) consist primarily of carbon and hydrogen, nitrogen, and oxygen, and, to a smaller extent, phosphorus and sulfur, although other elements may be incorporated into a biomolecule.

A “biopolymer” refers generally to a large molecule or macromolecule composed of repeating structural units, which are typically connected by covalent chemical bonds, and which can be produced by living organisms. Examples of biopolymers include, without limitation, polysaccharides, nucleic acids, and proteins.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene. Non-coding sequences include regulatory sequences such as promoters or enhancers.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

The term “de-polymerize” relates to breaking down a polymeric macromolecule into its smaller or simpler components, such as by breaking down a polymer to an oligomer (e.g., dimer, trimer, etc.) or monomer, or breaking down an oligomer to a monomer.

By “enzyme reactive conditions” it is meant that any necessary conditions are available in an environment (e.g., temperature, pH, lack of inhibiting substances) which will permit the enzyme to function. Enzyme reactive conditions can be either in vitro, such as in a test tube, or in vivo, such as within a cell.

As used herein, the terms “function” and “functional” and the like refer to a biological or enzymatic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (see, e.g., Deveraux et al., Nucleic Acids Research 12, 387-395, 1984, herein incorporated by reference). In this way, sequences of a similar or substantially different length to those cited herein could be compared by insertion of gaps into the alignment, such gaps being determined, for example, by the comparison algorithm used by GAP.

The term “host cell” includes an individual cell or cell culture that can be or has been a recipient of any recombinant vector(s) or isolated polynucleotide(s) of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed, or infected in vivo or in vitro with a recombinant vector or a polynucleotide of the invention. A host cell which comprises a recombinant vector of the invention is a recombinant host cell, recombinant cell, or recombinant microorganism.

By “isolated” is meant material that is substantially or essentially free from components that normally accompany it in its native state. For example, an “isolated polynucleotide,” as used herein, may refer to a polynucleotide that has been isolated from the sequences that flank it in its naturally-occurring or genomic state, e.g., a DNA fragment that has been removed from the sequences that are normally adjacent to the fragment, such as by cloning into a vector. A polynucleotide is considered to be isolated if, for example, it is cloned into a vector that is not a part of the natural environment, or if it is artificially introduced in the genome of a cell in a manner that differs from its naturally-occurring state.

Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances. Preferably, such polypeptides are at least about 80% pure, 90% pure, more preferably at least about 95% pure and most preferably at least about 99% pure.

The term “exogenous” refers generally to a polynucleotide sequence or polypeptide that does not naturally occur in a wild-type cell or organism, but is typically introduced into the cell by molecular biological techniques, i.e., engineering to produce a recombinant microorganism. Examples of “exogenous” polynucleotides include vectors, plasmids, and/or man-made nucleic acid constructs encoding a desired protein or enzyme. The term “endogenous” refers to naturally-occurring polynucleotide sequences or polypeptides that may be found in a given wild-type cell or organism. For example, certain naturally-occurring bacterial or yeast species do not typically contain an alginate lyase gene, and, therefore, do not comprise an “endogenous” polynucleotide sequence that encodes an alginate lyase. In this regard, it is also noted that even though an organism may comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the pathways, genes, or enzymes described herein may utilize or rely on an “endogenous” sequence, may be provided as one or more “exogenous” polynucleotide sequences, or both.

By “enhance,” “enhancing,” “increase,” or “increasing” is meant the ability of one or more recombinant microorganisms to produce a greater amount of a given product or target molecule (e.g., monosaccharide, commodity chemical, biofuel, or intermediate product thereof) as compared to a control microorganism, such as an unmodified microorganism or a differently modified microorganism, or a microorganism grown under different conditions. An “increased” amount is typically a “statistically significant” amount, and may include an increase that is 1, 2, 3, 4, 5, 6, 7, 8, 9, 10, 15, 20, 30, 40, 50, 60, 70, 80, 90, 100, or more times (including all integers and decimal points in between, e.g., 1.5, 1.6, 1.7. 1.8, etc.) the amount produced by an unmodified microorganism or a differently modified microorganism. An increased amount may be measured according to routine techniques in the art. For instance, an “increased” amount of a commodity chemical may be measured according to a percentage of a theoretical maximum yield. For instance, in certain embodiments, the methods of the present invention may enhance the yield of a target molecule (e.g., commodity chemical) to at least about 30%, 35%, 40%, 45%, 50%, 55%, 60%, 65%, 70%, 75%, 80%, 85%, 88%, 90%, 91%, 92%, 93%, 94%, 95%, 96%, 97%, 98%, 99%, or 100% of a theoretical maximum yield. In certain embodiments, the method may be characterized by increasing the percentage of the theoretical maximum yield of the target molecule by at least about 10% (e.g., from about 30% to about 40% of the theoretical maximum yield), 15% (e.g., from about 30% to about 45%), 20%, 25%, 30%, 35%, 40%, 50%, 60%, 70%, 80%, or 90% as compared to incubating the same recombinant microorganism under control or different conditions, or as compared to incubating a control (e.g., unmodified or differently modified) microorganism under the same or similar conditions.

The term “reduce” relates generally to a “decrease” in a relevant cellular response, such as NADH or acetate production, as measured according to routine techniques in the diagnostic art. Other relevant cellular responses (in vivo or in vitro) will be apparent to persons skilled in the art. A “decrease” in a response may be “statistically significant” amount as compared to the response produced by an unmodified microorganism or a differently modified microorganism, or by a microorganism growing under different conditions, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

By “obtained from” is meant that a sample such as, for example, a polynucleotide extract or polypeptide extract is isolated from, or derived from, a particular source, such as a desired organism, typically a microorganism. “Obtained from” can also refer to the situation in which a polynucleotide or polypeptide sequence is isolated from, or “derived from”, a particular organism or microorganism. For example, a polynucleotide sequence encoding an alginate lyase enzyme may be isolated from a variety of prokaryotic or eukaryotic microorganisms, such as Sphingomonas.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., CO₂ concentration, nutrient levels, light, heat). In the absence of that condition, inducible promoters typically do not allow significant or measurable levels of transcriptional activity.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

Polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence, or may comprise a variant, or a biological functional equivalent, of such a sequence. Polynucleotide variants may contain one or more substitutions, additions, deletions and/or insertions, as further described below, preferably such that the enzymatic activity of the encoded polypeptide is not substantially diminished relative to the unmodified polypeptide, and preferably such that the enzymatic activity of the encoded polypeptide is improved (e.g., optimized) relative to the unmodified polypeptide. The effect on the enzymatic activity of the encoded polypeptide may generally be assessed as described herein.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein (see, e.g., the Sequence Listing; and Tables C-G).

The terms “polynucleotide variant” and “variant” also include naturally-occurring allelic variants that encode these enzymes. Examples of naturally-occurring variants include allelic variants (same locus), homologs (different locus), and orthologs (different organism). Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having a suitable enzymatic activity described herein (e.g., pectate lyase, alginate lyase, outer membrane porn, symporter, decarboxylase, transporter).

Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. In certain aspects, non-naturally occurring variants may have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For nucleotide sequences, conservative variants include those sequences that, because of the degeneracy of the genetic code, encode the amino acid sequence of a reference polypeptide. Variant nucleotide sequences also include synthetically derived nucleotide sequences, such as those generated, for example, by using site-directed mutagenesis but which still encode a biologically active polypeptide. Generally, variants of a particular reference nucleotide sequence will have at least about 30%, 40% 50%, 55%, 60%, 65%, 70%, generally at least about 75%, 80%, 85%, 90% to 95% or more, and even about 97% or 98% or more sequence identity to that particular nucleotide sequence as determined by sequence alignment programs described elsewhere herein using default parameters.

As used herein, the term “hybridizes under low stringency, medium stringency, high stringency, or very high stringency conditions” describes conditions for hybridization and washing. Guidance for performing hybridization reactions can be found in Ausubel et al., “Current Protocols in Molecular Biology”, John Wiley & Sons Inc, 1994-1998, Sections 6.3.1-6.3.6. Aqueous and non-aqueous methods are described in that reference and either can be used.

Reference herein to “low stringency” conditions include from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency” conditions include from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

“High stringency” conditions include from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

One embodiment of “very high stringency” conditions includes hybridizing in 0.5 M sodium phosphate, 7% SDS at 65° C., followed by one or more washes in 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al., Current Protocols in Molecular Biology (1989), at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula: Tₘ=81.5+16.6 (log₁₀ M)+0.41 (% G+C)−0.63 (% formamide)−(600/length) wherein: M is the concentration of Na⁺, preferably in the range of 0.01 molar to 0.4 molar; % G+C is the sum of guano sine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ-15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionizer formamide, 5×SSC, 5× Reinhardt's solution (0.1% fecal, 0.1% polyvinylpyrollidone and 0.1% bovine serum albumin), 0.1% SDS and 200 mg/mL denatured salmon sperm DNA) containing a labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Polynucleotides and fusions thereof may be prepared, manipulated and/or expressed using any of a variety of well established techniques known and available in the art. For example, polynucleotide sequences that encode polypeptides of the invention, or fusion proteins or functional equivalents thereof, may be used in recombinant DNA molecules to direct expression of a selected enzyme in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.” Any of the nucleotide sequences described herein may be utilized in such a “codon-optimized” form. By way of non-limiting example, the nucleotide coding sequence of the alginate lyase from Sphingomonas may be codon-optimized for expression in E. coli.

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector that contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The present invention contemplates the use in the methods described herein of variants of full-length polypeptides having any of the enzymatic activities described herein, truncated fragments of these full-length polypeptides, variants of truncated fragments, as well as their related biologically active fragments. Typically, biologically active fragments comprise a domain or motif with at least one enzymatic activity, and may include one or more (and in some cases all) of the various active domains. A biologically active fragment of a an enzyme can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence (see, e.g., Sequence Listing; and Tables C-G). In certain embodiments, a biologically active fragment comprises a conserved enzymatic sequence, domain, or motif, as described elsewhere herein and known in the art. Suitably, the biologically-active fragment has no less than about 1%, 10%, 25%, 50% of an activity of the wild-type polypeptide from which it is derived.

A polypeptide may be altered in various ways including amino acid substitutions, deletions, truncations, and insertions. Methods for such manipulations are generally known in the art. For example, amino acid sequence variants of a truncated and/or variant polypeptide can be prepared by mutations in the DNA. Methods for mutagenesis and nucleotide sequence alterations are well known in the art, and can be found, for example, in Kunkel (Proc. Natl. Acad. Sci. USA. 82: 488-492, 1985), Kunkel et al., (Methods in Enzymol, 154: 367-382, 1987), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect the selected biological activity of the protein of interest may be found in the model of Dayhoff et al. ((1978) Atlas of Protein Sequence and Structure (Natl. Biomed. Res. Found., Washington, D.C.)). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of polypeptides. Recursive ensemble mutagenesis (REM), a technique which enhances the frequency of functional mutants in the libraries, can be used in combination with the screening assays to identify polypeptide variants (Arkin and Yourvan, Proc. Natl. Acad. Sci. USA 89: 7811-7815, 1992; Delgrave et al., Protein Engineering, 6: 327-331, 1993). Conservative substitutions, such as exchanging one amino acid with another having similar properties, may be desirable as discussed in more detail below.

Variant polypeptides may contain conservative amino acid substitutions at various locations along their sequence, as compared to a reference amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., 1978, supra), a model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (Science, 256: 14430-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxylcarbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant polypeptide can readily be determined by assaying its activity, as described herein. Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
Exemplary Amino Acid Substitutions

| Original | | Preferred |
|---|---|---|
| Residue | Exemplary Substitutes | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a truncated and/or variant polypeptide is typically replaced with another amino acid residue from the same side chain family. Alternatively, mutations can be introduced randomly along all or part of a coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the wild-type sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of wild-type. An “essential” amino acid residue is a residue that, when altered from the wild-type sequence of a reference truncated polypeptide, results in abolition of an activity of the parent molecule such that less than about 20% of the wild-type activity is present.

In general, polypeptide variants will display at least about 30, 40, 50, 55, 60, 65, 70, 75, 80, 85, 90, 91, 92, 93, 94, 95, 96, 97, 98, 99% similarity or sequence identity to a reference polypeptide sequence (see, e.g., the Sequence Listing; and Tables C-G). Moreover, sequences differing from a reference or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of a parent or reference polypeptide sequence are contemplated. In certain embodiments, the C-terminal or N-terminal region of any reference sequence may be truncated by 1, 2, 3, 4, 5, 6, 7, 8, 9, 10, 15, 20, 25, 30, 35, 40, 45, 50, 60, 70, 80, 90, 100, or more amino acids, including all integers in between.

In some embodiments, variant polypeptides differ from a reference sequence by at least one but by less than 50, 40, 30, 20, 15, 10, 8, 6, 5, 4, 3 or 2 amino acid residue(s). In other embodiments, variant polypeptides differ from the corresponding reference sequences described herein by at least 1% but less than 20%, 15%, 10% or 5% of the residues. (If this comparison requires alignment, the sequences should be aligned for maximum similarity. “Looped” out sequences from deletions or insertions, or mismatches, are considered differences.) The differences are, suitably, differences or changes at a non-essential residue or a conservative substitution.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., “Current Protocols in Molecular Biology,” John Wiley & Sons Inc, 1994-1998, Chapter 15.

In certain embodiments, calculations of sequence similarity or sequence identity between sequences (the terms are used interchangeably herein) can be performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using mathematical algorithms. For instance, in one embodiment, the percent identity between two amino acid sequences can be determined using the Needleman and Wunsch, (J. Mol. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can also be determined using the algorithm of E. Meyers and W. Miller (Cabios, 4: 11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (J. Mol. Biol, 215: 403-10, 1990). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (Nucleic Acids Res, 25: 3389-3402, 1997). When utilizing BLAST and Gapped BLAST programs, the default parameters of the respective programs (e.g., XBLAST and NBLAST) can be used.

Variants of a polypeptide can be identified by screening combinatorial libraries of mutants of a reference polypeptide. Libraries or fragments, e.g., N terminal, C terminal, or internal fragments, of reference protein coding sequence can be used to generate a variegated population of fragments for screening and subsequent selection of variants of a reference polypeptide.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of reference polypeptides.

The present invention also contemplates chimeric or fusion polypeptides. As used herein, a “chimeric protein,” “fusion protein,” or “fusion polypeptide” may include, without limitation, a first polypeptide or fragment thereof linked to a second, third, or fourth (or more) polypeptide, or fragment thereof (e.g., to create multiple fragments). The second, third or fourth polypeptide may refer to the same polypeptide as the first polypeptide, such as to selectively link together certain fragments of that first polypeptide, or may refer to a “heterologous polypeptide,” which typically has an amino acid sequence corresponding to a protein that is different from the first polypeptide, and which may be derived from the same or a different organism. In certain embodiments, a fusion protein includes at least one (or two, three, four, or more) biologically active portion of a given polypeptide protein. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the activity of the polypeptide. For example, in one embodiment, a fusion partner may comprise a sequence that assists in expressing the protein (an expression enhancer) at higher yields than the native recombinant protein. Other fusion partners may be selected so as to increase the solubility of the protein, target the protein to desired intracellular compartments, secrete the protein, or tether the protein to the cell surface. As one example, the fusion protein can contain a heterologous signal peptide sequence at its N-terminus. In certain host cells, secretion or cell-surface tethering of fusion polypeptides can be increased through the use of one or more heterologous signal peptide sequences, typically fused at or near to the N-terminus of the polypeptide.

A “recombinant” microorganism typically comprises one or more exogenous nucleotide sequences, such as in a plasmid or vector. Examples of microorganisms that can be utilized as recombinant microorganisms include, without limitation, Escherichia coli, Acetobacter aceti, Achromobacter, Acidiphilium, Acinetobacter, Actinomadura, Actinoplanes, Aeropyrum pernix, Agrobacterium, Alcaligenes, Ananas comosus (M), Arthrobacter, Aspargillus niger, Aspargillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus lentus, Bacillus licheniformis, Bacillus macerans, Bacillus stearothermophilus, Bacillus subtilis, Bifidobacterium, Brevibacillus brevis, Burkholderia cepacia, Candida cylindracea, Candida rugosa, Carica papaya (L), Cellulosimicrobium, Cephalosporium, Chaetomium erraticum, Chaetomium gracile, Clostridium, Clostridium butyricum, Clostridium acetobutylicum, Clostridium thermocellum, Corynebacterium (glutamicum), Corynebacterium efficiens, Enterococcus, Erwina chrysanthemi, Gliconobacter, Gluconacetobacter, Haloarcula, Humicola insolens, Humicola nsolens, Kitasatospora setae, Klebsiella, Klebsiella oxytoca, Kluyveromyces, Kluyveromyces fragilis, Kluyveromyces lactis, Kocuria, Lactlactis, Lactobacillus, Lactobacillus fermentum, Lactobacillus sake, Lactococcus, Lactococcus lactis, Leuconostoc, Methylocystis, Methanolobus siciliae, Methanogenium organophilum, Methanobacterium bryantii, Microbacterium imperiale, Micrococcus lysodeikticus, Microlunatus, Mucor javanicus, Mycobacterium, Myrothecium, Nitrobacter, Nitrosomonas, Nocardia, Papaya carica, Pediococcus, Pediococcus halophilus, Penicillium, Penicillium camemberti, Penicillium citrinum, Penicillium emersonii, Penicillium roqueforti, Penicillum lilactinum, Penicillum multicolor, Paracoccus pantotrophus, Propionibacterium, Pseudomonas, Pseudomonas fluorescens, Pseudomonas denitrificans, Pyrococcus, Pyrococcus furiosus, Pyrococcus horikoshii, Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, Saccharophagus degradans, Sccharomyces cerevisiae, Sclerotina libertina, Sphingobacterium multivorum, Sphingobium, Sphingomonas, Streptococcus, Streptococcus thermophilus Y-1, Streptomyces, Streptomyces griseus, Streptomyces lividans, Streptomyces murinus, Streptomyces rubiginosus, Streptomyces violaceoruber, Streptoverticillium mobaraense, Tetragenococcus, Thermus, Thiosphaera pantotropha, Trametes, Trichoderma, Trichoderma longibrachiatum, Trichoderma reesei, Trichoderma viride, Trichosporon penicillatum, Vibrio alginolyticus, Vibrio splendidus, Xanthomonas, yeast, Yarrowia lipolytica, Zygosaccharomyces rouxii, Zymomonas, or Zymomonus mobilis.

“Transformation” refers generally to the permanent, heritable alteration in a cell resulting from the uptake and incorporation of foreign DNA into the host-cell genome; also, the transfer of an exogenous gene from one organism into the genome of another organism.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The terms “wild-type” and “naturally occurring” are used interchangeably to refer to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

Examples of “biomass” include without limitation aquatic or marine biomass, fruit-based biomass such as fruit waste, and vegetable-based biomass such as vegetable waste, among others, as well as combinations of biomass. Examples of aquatic or marine biomass include, but are not limited to, kelp, giant kelp, seaweed, algae, and marine microflora, microalgae, sea grass, and the like. In certain aspects, biomass does not include fossilized sources of carbon, such as hydrocarbons that are typically found within the top layer of the Earth's crust (e.g., fossil fuels, natural gas, nonvolatile materials composed of almost pure carbon, like anthracite coal, etc).

Examples of fruit and/or vegetable biomass include, but are not limited to, any source of pectin such as plant peel and pomace including citrus, orange, grapefruit, potato, tomato, grape, mango, gooseberry, carrot, sugar-beet, and apple, among others.

Examples of polysaccharides, oligosaccharides, monosaccharides or other sugar components of biomass include, but are not limited to, alginate (e.g., polyG, polyMG, polyM), oligoalginate (e.g., ΔM, ΔG, ΔMM, ΔMG, ΔGM, ΔGG, MM, MG, GM, GG, MMM, MGM, MMG, MGG, GMM, GMG, GGM, GGG), agar, carrageenan, fucoidan, pectin, gluronate, guluronate, mannuronate, mannitol, lyxose, cellulose, hemicellulose, cellobiose, glycerol, xylitol, glucose, mannose, galactose, xylose, xylan, mannan, arabinan, arabinose, glucuronate, galacturonate (including di- and tri-galacturonates), rhamnose, and the like.

Certain examples of alginate-derived polysaccharides include saturated polysaccharides, such as β-D-mannuronate, α-L-gluronate, dialginate, trialginate, pentalginate, hexylginate, heptalginate, octalginate, nonalginate, decalginate, undecalginate, dodecalginate and polyalginate, as well as unsaturated polysaccharides such as 4-deoxy-L-erythro-5-hexoseulose uronic acid, 4-(4-deoxy-beta-D-mann-4-enuronosyl)-D-mannuronate or L-guluronate, 4-(4-deoxy-beta-D-mann-4-enuronosyl)-dialginate, 4-(4-deoxy-beta-D-mann-4-enuronosyl)-trialginate, 4-(4-deoxy-beta-D-mann-4-enuronosyl)-tetralginate, 4-(4-deoxy-beta-D-mann-4-enuronosyl)-pentalginate, 4-(4-deoxy-beta-D-mann-4-enuronosyl)-hexylginate, 4-(4-deoxy-beta-D-mann-4-enuronosyl)-heptalginate, 4-(4-deoxy-beta-D-mann-4-enuronosyl)-octalginate, 4-(4-deoxy-beta-D-mann-4-enuronosyl)-nonalginate, 4-(4-deoxy-beta-D-mann-4-enuronosyl)-undecalginate, and 4-(4-deoxy-beta-D-mann-4-enuronosyl)-dodecalginate.

Certain examples of pectin-derived polysaccharides include saturated polysaccharides, such as galacturonate, digalacturonate, trigalacturonate, tetragalacturonate, pentagalacturonate, hexagalacturonate, heptagalacturonate, octagalacturonate, nonagalacturonate, decagalacturonate, dodecagalacturonate, polygalacturonate, and rhamnopolygalacturonate, as well as saturated polysaccharides such as 4-deoxy-L-threo-5-hexosulose uronate, 4-(4-Deoxy-alpha-D-gluc-4-enuronosyl)-D-galacturonate, 4-(4-Deoxy-alpha-D-gluc-4-enuronosyl)-D-digalacturonate, 4-(4-Deoxy-alpha-D-gluc-4-enuronosyl)-D-trigalacturonate, 4-(4-Deoxy-alpha-D-gluc-4-enuronosyl)-D-tetragalacturonate, 4-(4-Deoxy-alpha-D-gluc-4-enuronosyl)-D-pentagalacturonate, 4-(4-Deoxy-alpha-D-gluc-4-enuronosyl)-D-hexagalacturonate, 4-(4-Deoxy-alpha-D-gluc-4-enuronosyl)-D-heptagalacturonate, 4-(4-Deoxy-alpha-D-gluc-4-enuronosyl)-D-octagalacturonate, 4-(4-Deoxy-alpha-D-gluc-4-enuronosyl)-D-nonagalacturonate, 4-(4-Deoxy-alpha-D-gluc-4-enuronosyl)-D-decagalacturonate, and 4-(4-Deoxy-alpha-D-gluc-4-enuronosyl)-D-dodecagalacturonate.

These polysaccharide or oligosaccharide components may be converted into “monosaccharides” or other “suitable oligosaccharides” by the microorganisms described herein which are capable of growing on such polysaccharides or other sugar components as a source of carbon (e.g., a sole source of carbon).

A “monosaccharide” or “suitable oligosaccharide” refers generally to any saccharide that may be produced by a recombinant microorganism growing on pectin, alginate, or other saccharide (e.g., galacturonate, cellulose, hemi-cellulose, cellobiose) as a source or sole source of carbon, and also refers generally to any saccharide that may be utilized in a commodity chemical synthesis pathway of the present invention to produce biofuels (e.g., ethanol, biodiesel). Examples of monosaccharides or suitable oligosaccharides include, but are not limited to, 2-keto-3-deoxy D-gluconate (KDG), D-mannitol, oligoalginate, guluronate, α-keto acid, 4-deoxy-α-L-erythro-hexoselulose uronate (DEHU), gluronate, mannuronate, mannitol, lyxose, glycerol, xylitol, glucose, mannose, galactose, xylose, arabinose, glucuronate, galacturonates, xylose, arabinose, rhamnose, and the like. As noted herein, a “suitable monosaccharide” or “suitable saccharide” as used herein may be produced by an engineered or recombinant microorganism of the present invention, or may be obtained from commercially available sources.

The recitation “commodity chemical” as used herein includes any saleable or marketable chemical that can be produced either directly or as a by-product of the methods provided herein, including biofuels, such biodiesels. The term biodiesel refers generally to plant oil- or animal fat-based diesel fuel composed mainly of long-chain alkyl, methyl, propyl, or ethyl esters (i.e., fatty acid esters), though it can include other fatty acids and terpenoids. General examples of “commodity chemicals” include, but are not limited to, biofuels, minerals, polymer precursors, fatty alcohols, surfactants, plasticizers, and solvents. The recitation “biofuels” as used herein includes solid, liquid, or gas fuels derived, at least in part, from a biological source, such as a recombinant microorganism.

Examples of commodity chemicals include, but are not limited to, ethanol, biodiesel, methane, methanol, ethane, ethene, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-) ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl)pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydrxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, and the like.

The recitation “optimized” as used herein refers to a pathway, gene, polypeptide, enzyme, or other molecule having an altered biological activity, such as by the genetic alteration of a polypeptide's amino acid sequence or by the alteration/modification of the polypeptide's surrounding cellular environment, to improve its functional characteristics in relation to the original molecule or original cellular environment (e.g., a wild-type sequence of a given polypeptide or a wild-type microorganism). Any of the polypeptides or enzymes described herein may be optionally “optimized,” and any of the genes or nucleotide sequences described herein may optionally encode an optimized polypeptide or enzyme. Any of the pathways described herein may optionally contain one or more “optimized” enzymes, or one or more nucleotide sequences encoding for an optimized enzyme or polypeptide.

Typically, the improved functional characteristics of the polypeptide, enzyme, or other molecule relate to the suitability of the polypeptide or other molecule for use in a biological pathway to convert a biomolecule to a monosaccharide, oligosaccharide, or to a commodity chemical. Certain embodiments, therefore, contemplate the use of “optimized” biological pathways. An exemplary “optimized” polypeptide may contain one or more alterations or mutations in its amino acid coding sequence (e.g., point mutations, deletions, addition of heterologous sequences) that facilitate improved expression and/or stability in a given microbial system or microorganism, allow regulation of polypeptide activity in relation to a desired substrate (e.g., inducible or repressible activity), modulate the localization of the polypeptide within a cell (e.g., intracellular localization, extracellular secretion), and/or effect the polypeptide's overall level of activity in relation to a desired substrate (e.g., reduce or increase enzymatic activity). A polypeptide or other molecule may also be “optimized” for use with a given microbial system or microorganism by altering one or more pathways within that system or organism, such as by altering a pathway that regulates the expression (e.g., up-regulation), localization, and/or activity of the “optimized” polypeptide or other molecule, or by altering a pathway that minimizes the production of undesirable by-products, among other alterations. In this manner, a polypeptide or other molecule may be “optimized” with or without altering its wild-type amino acid sequence or original chemical structure. Optimized polypeptides or biological pathways may be obtained, for example, by direct mutagenesis or by natural selection for a desired phenotype, according to techniques known in the art.

In certain aspects, “optimized” genes or polypeptides may comprise a nucleotide coding sequence or amino acid sequence that is 50% to 99% identical (including all integers in between) to the nucleotide or amino acid sequence of a reference (e.g., wild-type) gene or polypeptide. In certain aspects, an “optimized” polypeptide or enzyme may have about 1, 2, 3, 4, 5, 6, 7, 8, 9, 10, 20, 30, 40, 50, 100 (including all integers and decimal points in between e.g., 1.2, 1.3, 1.4, 1.5, 5.5, 5.6, 5.7, 60, 70, etc.), or more times the biological activity of a reference polypeptide.

In certain embodiments, a recombinant microorganism is capable of growing using a polysaccharide (e.g., alginate, pectin, etc.) as a sole source of carbon and/or energy. A “sole source of carbon” refers generally to the ability to grow on a given carbon source as the only carbon source in a given growth medium.

Certain aspects of the invention also include a commodity chemical, such as a biofuel, that is produced according to the methods and recombinant microorganisms described herein. Such a biofuel (e.g., ethanol, medium to long chain alkane) may be distinguished from other fuels, such as those fuels produced by traditional refinery from crude carbon sources, by radio-carbon dating techniques. For instance, carbon has two stable, nonradioactive isotopes: carbon-12 (¹²C), and carbon-13 (¹³C). In addition, there are trace amounts of the unstable isotope carbon-14 (¹⁴C) on Earth. Carbon-14 has a half-life of 5730 years, and would have long ago vanished from Earth were it not for the unremitting impact of cosmic rays on nitrogen in the Earth's atmosphere, which create more of this isotope. The neutrons resulting from the cosmic ray interactions participate in the following nuclear reaction on the atoms of nitrogen molecules (N₂) in the atmospheric air:

n+₇¹⁴N→₆¹⁴C+p

Plants and other photosynthetic organisms take up atmospheric carbon dioxide by photosynthesis. Since many plants are ingested by animals, every living organism on Earth is constantly exchanging carbon-14 with its environment for the duration of its existence. Once an organism dies, however, this exchange stops, and the amount of carbon-14 gradually decreases over time through radioactive beta decay.

Most hydrocarbon-based fuels, such as crude oil and natural gas derived from mining operations, are the result of compression and heating of ancient organic materials (i.e., kerogen) over geological time. Formation of petroleum typically occurs from hydrocarbon pyrolysis, in a variety of mostly endothermic reactions at high temperature and/or pressure. Today's oil formed from the preserved remains of prehistoric zooplankton and algae, which had settled to a sea or lake bottom in large quantities under anoxic conditions (the remains of prehistoric terrestrial plants, on the other hand, tended to form coal). Over geological time the organic matter mixed with mud, and was buried under heavy layers of sediment resulting in high levels of heat and pressure (known as diagenesis). This process caused the organic matter to chemically change, first into a waxy material known as kerogen which is found in various oil shales around the world, and then with more heat into liquid and gaseous hydrocarbons in a process known as catagenesis. Most hydrocarbon based fuels derived from crude oil have been undergoing a process of carbon-14 decay over geological time, and, thus, will have little to no detectable carbon-14. In contrast, certain biofuels produced by the living microorganisms of the present invention will comprise carbon-14 at a level comparable to all other presently living things (i.e., an equilibrium level). In this manner, by measuring the carbon-12 to carbon-14 ratio of a hydrocarbon-based biofuel of the present invention, and comparing that ratio to a hydrocarbon based fuel derived from crude oil, the biofuels produced by the methods provided herein can be structurally distinguished from typical sources of hydrocarbon based fuels.

### Tethering System and Methods of Use

Certain embodiments relate to methods of enhancing the ability of a given microorganism to metabolize otherwise unsuitable biomolecules, such as polysaccharides or lipids, as an efficient source of carbon, energy, or both, by fusing at least one biomolecule metabolizing or transporting enzyme, such as a polysaccharide- or lipid-metabolizing enzyme, to a carrier protein, and thereby targeting the enzyme or enzymes for secretion or outer cell-surface tethering. Typically, absent the secreted or tethered enzymes, such microorganisms would be unable to metabolize the biomolecule as a source of carbon or energy, or would be relatively inefficient in metabolizing the biomolecule, mainly because of the relative inefficiencies in transporting these relatively larger, biomolecules into the cell. By directing the metabolizing or transporting enzymes to the media or outer cell surface, and by allowing these enzymes to begin the metabolic process outside of the cell, the fusion polypeptides of the present invention make it easier for microorganisms to transport the resulting metabolites into the cell, and to rely on those metabolites in their intracellular metabolic processes (e.g., glycolysis, fatty acid metabolism).

In certain embodiments, the tethering systems of the present invention include one or more isolated polynucleotides, often in the form of a vector or other genetic construct, which encode one or more fusion polypeptides, and which may be introduced into a given microorganism using standard molecular biological techniques. These fusion polypeptides comprise at least one “carrier polypeptide,” which directs the secretion of the polypeptide or its tethering to the cell surface (or both), and a “passenger polypeptide” fused thereto, the latter being capable of catalyzing the metabolism or the transport of a selected polysaccharide or lipid. As noted above, the polynucleotides may be “optimized” for use in a given microorganism, such as E. coli. Also included are variants of these polynucleotides, which are capable of hybridizing to polynucleotides that encode the various fusion polypeptides of the present invention, typically under moderate, stringent, or highly stringent conditions, as described herein.

As noted above, the secretion or tethering systems of the present invention are typically utilized in the form of a fusion polypeptide, or an isolated polynucleotide or vector that encodes a fusion polypeptide. These fusion polypeptides comprise a carrier polypeptide and a passenger polypeptide, the latter relating mainly to a polypeptide that is capable of de-polymerizing, metabolizing, or transporting a biopolymer such as a polysaccharide or its oligosaccharide components (e.g., alginate, oligoalginate, cellulose, cellobiose, laminarin, mannitol), or metabolizing another biomolecule, such as a lipid. Generally, the carrier protein directs the secretion or targeting of the passenger polypeptide to the cell surface of a microorganism, where it can catalyze the de-polymerization or metabolism of selected biomolecules, transport the biomolecule or its smaller components into the cell, and thereby contribute to the use of those biomolecules as a source of carbon or energy. In this manner, microorganisms that are either unable to use one or more selected biomolecules as a source of carbon or energy, or are inefficient in their use, can acquire the ability to efficiently grow on those biomolecules, and when combined with other commodity chemical-based pathways, can more efficiently convert those biomolecules into commodity chemicals.

“Carrier polypeptides” are typically derived from polypeptides that are naturally targeted for secretion or cell-surface attachment, or functional variants or fragments thereof. Examples of carrier polypeptides include, without limitation, autotransporter proteins (e.g., outer membrane porins), or biologically active fragments or variants thereof. Autotransporters constitute the largest family of secreted proteins in gram-negative bacteria (see, e.g., Pallen et al., Curr. Opin. Microbiol. 6:519-527, 2003). Many autotransporter proteins are very large proteins, ranging in size from 90 to 200 kDa. In certain instances, autotransporter secretion is known to involve not only the insertion into the outer membrane of a conserved carboxy-terminal beta-barrel domain, but the translocation across the outer membrane of the functional domain present at the mature amino terminus.

Autotransporter proteins have been identified in a wide range of Gram-negative bacteria, and are often associated with virulence functions such as adhesion, aggregation, invasion, biofilm formation and toxicity. The proteins secreted by autotransporter domains typically comprise an N-terminal signal peptide that plays a role in translocation to the periplasm, which may be mediated by secB or SRP pathways, passenger domain, and/or C-terminal translocation unit (UT) having a characteristic β-barrel structure. The β-barrel portion of the UT builds an aqueous pore channel across the outer membrane and helps the transportation of passenger domain to media. Autodisplayed passenger proteins are often cleaved by the autotransporter and set free to media. In certain embodiments, the autotransporter is a not an adhesin-involved-in-diffuse-interference (AIDA)-based transporter from gram negative bacteria, such as E. coli.

The type I secretion machinery may also be used for the secretion of recombinant proteins in E. coli. The type I secretion machinery consist of two inner membrane proteins (HlyB and HlyD) that are the member of the ATP binding cassette (ABC) transporter family, and an endogenous outer membrane protein (TolC). The secretion of recombinant proteins based on type I secretion machinery may utilize the C-terminal region of α-haemolysin (HlyA) as a signal sequence.

Outer membrane porins from the outer membrane of Gram-negative bacteria are generally non-selective, transmembrane channels, and may also be employed as carrier proteins. The pore structure of these proteins is formed almost entirely of a beta-barrel, and the monomeric protein is typically matured into a trimeric species that ultimately integrates into the outer membrane. Examples of outer membrane porins include, without limitation, Omp1 (Zymomonas mobilis), porin F (P. aeruginosa), OmpA (E. coli), OmpF/C (E. coli), OmpG (E. coli), and PhoE porin (E. coli), and biologically active fragments or variants thereof. Additional examples of outer membrane porins can be found, for instance, in Nguyen et al., Mol Microbiol Biotechnol. 11:291-301, 2006.

Particular examples of carrier polypeptides include, but are not limited to, PgsA from Bacillus subtilis (a poly-γ-glutamate synthetase complex that is natively displayed on the surface of Bacillus subtilis); PhoA-EstA autotransporters from P. aeruginosa, Pseudomonas putida, or Pseudomonas fluorescence; OmpA, StII, EX, PhoA, OmpF, PhoE, MalE, OmpC, Lpp, LamB, OmpT, Ltb, TolC, Ag43, and phospholipase A from E. coli; PorA, PorB, PilQ, FrpB, and OMPLA from Neisseria meningitidis; Omp1 from Zymomonas mobilis; PelB from Pectobacterium sp.; IcsA and SepA autotransporters from Shigella flexneri; pertactin from Bordetella pertussis, the adhesion; penetration protein (Hap) from Haemophilus influenzae, and biologically active fragments or variants of these polypeptides.

Examples of carrier polypeptides also include “ice nucleation proteins,” or INPs. These glycosyl phosphatidylinositol-anchored outer membrane proteins are found in certain Gram-negative bacteria, and can be useful for tethering passenger proteins to the cell surface (see, e.g., U.S. Pat. No. 6,071,725, herein incorporated by reference). Examples of INPs include, without limitation, InaV (Pseudomonas syringae INA5), InaK (Pseudomonas syringae KCTC1832), and INPs derived from Pseudomonas sp., Erwinia sp., Xanthomonas sp, including and biologically active fragments or variants thereof.

The polynucleotide and/or polynucleotide sequences of exemplary “carrier” sequences can be found in the sequence listing.

The fusion polypeptides also comprise passenger polypeptide. Examples of general classes of “passenger polypeptides” include, without limitation, lyases, cellulases, laminarinases, lipases, among other classes of enzymes that de-polymerize biopolymers or metabolize other biomolecules, including biologically active fragments and variants thereof. Also included are transporter proteins. Examples of general classes of lyases include, but are not limited to, alginate lyases, oligoalginate lyases, pectin and pectate lyases, rhamnogalacturonan lyases, gellan lyases, xanthan lyases, polymannuronate lyases, polygluronate lyases, polygalacturonate lyases, hyaluronan lyases, among others.

With regard to alginate lyases, alginate is a block co-polymer of β-D-mannuronate (M) and α-D-gluronate (G) (M and G are epimeric about the C5-carboxyl group). Each alginate polymer comprises regions of all M (polyM), all G (polyG), and/or the mixture of M and G (polyMG). ALs are mainly classified into two distinctive subfamilies depending on their acts of catalysis: endo- (EC 4.2.2.3) and exo-acting (EC 4.2.2.-) ALs. Endo-acting ALs are further classified based on their catalytic specificity; M specific and G specific ALs. The endo-acting ALs randomly cleave alginate via a β-elimination mechanism and mainly de-polymerize alginate to di-, tri- and tetrasaccharides. The uronate at the non-reducing terminus of each oligosaccharide are converted to unsaturated sugar uronate, 4-deoxy-α-L-erythro-hex-4-ene pyranosyl uronates. The exo-acting ALs catalyze further de-polymerization of these oligosaccharides and release unsaturated monosaccharides, which may be non-enzymatically converted to monosaccharides, including α-keto acid, 4-deoxy-α-L-erythro-hexoselulose uronate (DEHU). Certain embodiments of a recombinant microorganism may include a fusion polypeptide that comprises endoM-, endoG- and exo-acting ALs, mainly to degrade or depolymerize aquatic or marine-biomass polysaccharides such as alginate to a monosaccharide such as DEHU.

The fusion polypeptides of the present invention may include alginate lyases isolated from various sources, including, but not limited to, marine algae, mollusks, and wide varieties of microbes such as genus Pseudomonas, Vibrio, and Sphingomonas. Many alginate lyases are endo-acting M specific, several are G specific, and few are exo-acting. For example, ALs isolated from Sphingomonas sp. strain AI include five endo-acting ALs, AI-I, AI-II, AI-II′, AI-III, and AI-IV′ and an exo-acting AL, AI-IV.

Typically, AI-I, AI-II, and AI-III have molecular weights of 66 kDa, 25 kDa, and 40 kDa, respectively. AI-II and AI-III are self-splicing products of AI-I. AI-II may be more specific to G and AI-III may be specific to M. AI-I may have high activity for both M and G. AI-IV has molecular weight of about 85 kDa and catalyzes exo-lytic de-polymerization of oligoalginate. Although both AI-II′ and AI-IV′ are functional homologues of AI-II and AI-IV. AI-II′ has endo-lytic activity and may have no preference to M or G. AI-IV has primarily endo-lytic activity. In certain embodiments, the alginate lyase is AI-I from Sphingomonas sp. AI (SEQ ID NO:31; a fusion protein that includes AI-II and AI-III), ΔAI-I from Sphingomonas sp. AI (SEQ ID NO:29; a truncated form of AI-I), AI-II from Sphingomonas sp. AI (SEQ ID NO:33; the N-terminal half of AI-I), AI-III from Sphingomonas sp. AI (SEQ ID NO:35; the C-terminal half of AI-I), AI-II′ from Sphingomonas sp. AI (an AI-II homolog), SM0524 from Pseudoalteromonas sp. (SEQ ID NO:37 or 39, or biologically active fragments or variants thereof, including variants that share that least 60%, 70%, 80%, 90%, 95%, 98%, or more sequence identity (including all integers in between) with these sequences, as well as polynucleotides and polynucleotide variants that encode them (see also Sequence Listing).

Certain examples of alginate lyases or oligoalginate lyases that may be utilized herein include polypeptides sharing at least 60%, 70%, 80%, 90%, 95%, 98%, or more sequence identity (including all integers in between) to SEQ ID NO:120, which shows the polypeptide sequence of oligoalginate lyase Atu3025 isolated from Agrobacterium tumefaciens. Exo-lytic AL Atu3025 derived from Agrobacterium tumefaciens has high activity for de-polymerization of oligoalginate, and may be used in certain embodiments. Certain embodiments may incorporate into a recombinant microorganism an isolated polynucleotide that encodes AI-I, AI-II′, AI-IV, and/or Atu3025, and may include optimal codon usage for the suitable host organisms, such as E. coli. Other alginate lyase sequences are described in the Sequence Listing and in Table C.

Examples of alginate lyases also include lyases having specific activity toward either the mannuronic acid or the guluronic acid blocks of the alginate polymer, or both (see, e.g., Doubet et al., Appl Environ Microbiol. 44:754-756, 1982). Additional examples of alginate lyases that may be utilized herein include polypeptides sharing at least 60%, 70%, 80%, 90%, 95%, 98%, or more sequence identity (including all integers in between) to the secreted alginate lyase encoded by Vs24254 from Vibrio splendidus, as well as the alginate lyase enzymes described in Table C below, in addition to polynucleotides and polynucleotide variants that encode them.

TABLE C
Alginate Lyases

| Protein | Organism | GenBank/GenPept | |
|---|---|---|---|
| Family 5 | | | |
| alginate lyase (AlgL) | Azotobacter chroococcum | AJ223605 | CAA11481.1 |
| | ATCC 4412 | | |
| alginate lyase (AlgL) | Azotobacter vinelandii | AF027499 | AAC04567.1 |
| | | AF037600 | AAC32313.1 |
| alginate lyase (Alg) | Cobetia marina N-1 | AB018795 | BAA33966.1 |
| alginate lyase (AlgL) | Pseudomonas aeruginosa 8830 | L14597 | AAA71990.1 |
| alginate lyase (AlgL) | Pseudomonas aeruginosa FRD1 | U27829 | AAA91127.1 |
| alginate lyase (AlgL; PA3547) | Pseudomonas aeruginosa PAO1 | AE004775 | AAG06935.1 |
| | | NC_002516 | NP_252237.1 |
| alginate lyase (AlgL) | Pseudomonas sp. QD03 | AY380832 | AAR23929.1 |
| alginate lyase (AlgL) | Pseudomonas sp. QDA | AY163384 | AAN63147.1 |
| alginate lyase (AlgL) | Pseudomonas syringae pv. | AF222020 | AAF32371.1 |
| | syringae FF5 | | |
| alginate lyase (aly; | Sphingomonas sp. AI | — | 2009330A |
| AI-I/PolyG + PolyM; | | AB011415 | BAB03312.1 |
| AI-II/PolyG; AI-III/PolyM) | | | |
| Family 6 | | | |
| alginate lyase (AlyP) | Pseudomonas sp. OS-ALG-9 | D10336 | BAA01182.1 |
| Family 7 | | | |
| guluronate lyase (alyPG) | Corynebacterium sp. ALY-1 | AB030481 | BAA83339.1 |
| poly(-L-guluronate) lyase (AlyA) | Klebsiella pneumoniae subsp. | L19657 | AAA25049.1 |
| | aerogenes | | |
| alginate lyase/poly- | Photobacterium sp. ATCC | X70036 | CAA49630.1 |
| mannuronate lyase (AlxM) | 43367 | | |
| alginate lyase (PAI167) | Pseudomonas aeruginosa PAO1 | AE004547 | AAG04556.1 |
| | | NC_002516 | NP_249858.1 |
| alginate lyase (AI-II′) | Sphingomonas sp. AI | AB120939 | BAD16656.1 |
| alginate lyase (aly; | Sphingomonas sp. AI | — | 2009330A |
| AI-I/PolyG + PolyM; | | AB011415 | BAB03312.1 |
| AI-II/PolyG; AI-III/PolyM) | | | |
| poly(a-L-guluronate) lyase | Vibrio halioticoli IAM14596T | AF114039 | AAF22512.1 |
| (AlyVGI; AlyVG1) | | | |
| alginate lyase/poly- | Vibrio sp. O2 | DQ235160 | ABB36771.1 |
| mannuronate lyase (AlyVOA) | | | |
| alginate lyase/poly- | Vibrio sp. O2 | DQ235161 | ABB36772.1 |
| mannuronate lyase (AlyVOB) | | | |
| alginate lyase (AlyVI) | Vibrio sp. QY101 | AY221030 | AAP45155.1 |
| exo-oligoalginate lyase | Haliotis discus hannai | AB234872 | BAE81787.1 |
| (HdAlex; HdAlex-1) | | | |
| alginate lyase (HdAly) | Haliotis discus hannai | AB110094 | BAC87758.1 |
| polysaccharide lyase acting on | Chlorella virus CVK2 | AB044791 | BAB19127.1 |
| glucuronic acid (vAL-1) | | | |
| alginate lyase (AlyII) | Pseudomonas sp. OS-ALG-9 | AB003330 | BAA19848.1 |
| Family 18 | | | |
| alginate lyase | Pseudoalteromonas sp. 272 | | |
| alginate lyase (Aly) | Pseudoalteromonas sp. | AF082561 | AAD16034.1 |
| | IAM14594 | | |
| Family 15 | | | |
| exotype alginate lyase | Agrobacterium tumefaciens str. | AE009232 | AAL43841.1 |
| (Atu3025) | C58 | NC_003305 | NP_533525.1 |
| exotype alginate lyase | Agrobacterium tumefaciens str. | AE008381 | AAK90358.1 |
| (AGR_L_3558p) | C58 (Cereon) | NC_003063 | NP_357573.1 |
| oligo alginate lyase (AI-IV) | Sphingomonas sp. AI | AB011415 | BAB03319.1 |
| alginate lyase (AI-IV′) | Sphingomonas sp. AI | AB176667 | BAD90006.1 |

As to pectin lyases or pectate lyases (or hydrolyases), pectin is a linear chain of α-(1-4)-linked D-galacturonic acid that forms the pectin-backbone, a homogalacturonan. Into this backbone, there are regions where galacturonic acid is replaced by (1-2)-linked L-rhamnose. From rhamnose, side chains of various neutral sugars typically branch off. This type of pectin is called rhamnogalacturonan I. Over all, about up to every 25th galacturonic acid in the main chain is exchanged with rhamnose. Some stretches consisting of alternating galacturonic acid and rhamnose—“hairy regions”, others with lower density of rhamnose—“smooth regions.” The neutral sugars mainly comprise D-galactose, L-arabinose and D-xylose; the types and proportions of neutral sugars vary with the origin of pectin. In nature, around 80% of carboxyl groups of galacturonic acid are esterified with methanol. Some plants, like sugar-beet, potatoes and pears, contain pectins with acetylated galacturonic acid in addition to methyl esters. Acetylation prevents gel-formation but increases the stabilising and emulsifying effects of pectin.

Pectate lyases and hydrolases may catalyze the endolytic cleavage of pectate via β-elimination and hydrolysis, respectively, to produce oligopectates. Examples of pectate lyases that may be incorporated into the fusion polypeptides of the present invention include, but are not limited to, PelA, PelB, PelC, PelD, PelE, Pelf, PelI, PelL, and PelZ, and examples of pectate hydrolases include, but are not limited to, PehA, PehN, PehV, PehW, and PehX, including polypeptides sharing at least 60%, 70%, 80%, 90%, 95%, 98%, or more sequence identity (including all integers in between) to these sequences, as well as polynucleotides and polynucleotide variants that encode them. Further examples of pectate lyases include polypeptides sharing at least 60%, 70%, 80%, 90%, 95%, 98%, or more sequence identity (including all integers in between) to the pectate lyases described in Table D, as well as polynucleotides and polynucleotide variants that encode them.

TABLE D
Pectate lyases

| Protein | Organism | GenBank | GenPept |
|---|---|---|---|
| pectate lyase C (PelC) | Erwinia chrysanthemi strain | AJ132325 | CAA10642.1 |
| | 3937 | | |
| pectin lyase (PnlA) | Pectobacterium carotovorum | M59909 | AAA24856.1 |
| | Ecc71 | | |
| pectate lyase III (Pel3; PelC) | Pectobacterium carotovorum Er | D10064 | BAA00953.1 |
| pectate lyase B (PelB) | Pseudoalteromonas haloplanktis | AF278705 | AAF86343.1 |
| | 505 | AF278705 | AAF86343.2 |
| pectate lyase A | Pseudoalteromonas haloplanktis | AF278706 | AAF86344.2 |
| | ANT/505 | | |
| pectate lyase (Pel) | Pseudomonas fluorescens | L41673 | AAA93535.1 |
| | CY091 | L38902 | AAB46399.1 |
| pectin lyase (PnL) (fragment) | Pseudomonas marginalis N6301 | M84971 | AAA92512.1 |
| | | D32121 | BAA06847.1 |
| pectate lyase (PeL) | Pseudomonas marginalis N6301 | S65042 | AAC60448.1 |
| | | D32122 | BAA06848.1 |
| pectate lyase P (PelP) | Pseudomonas syringae pv. | U75414 | AAB17879.1 |
| | lachrymans | | |
| pectate lyase (Pel; Pstru-4) | Pseudomonas viridiflava | L38901 | AAB46398.1 |
| | | L38574 | AAC41521.1 |
| | | DQ273695 | ABB55454.1 |
| | | D44611 | BAA08077.1 |
| pectate lyase (Pel) | Pseudonocardia sp. | AF002241 | AAC38059.1 |
| pectate lyase | Streptomyces coelicolor A3 (2) | AL596030 | CAC44284.1 |
| (SCO2821; SCBAC17F8.12c) | | NC_003888 | NP_627050.1 |
| pectate lyase | Streptomyces coelicolor A3 (2) | AL591322 | CAC38815.1 |
| (SCO1880; SCI39.27c) | | NC_003888 | NP_626147.1 |
| α | Thermotoga maritima MSB8 | AE001722 | AAD35518.1 |
| pectate lyase A (PelA; TM0433) | | NC_000853 | NP_228243.1 |
| XC_1298 | Xanthomonas campestris pv. | CP000050 | AAY48367.1 |
| | campestris str. 8004 | | |
| XC_3590 | Xanthomonas campestris pv. | CP000050 | AAY50632.1 |
| | campestris str. 8004 | | |
| pectate lyase (Pel; XCC0645) | Xanthomonas campestris pv. | AE012162 | AAM39961.1 |
| | campestris str. ATCC 33913 | NC_003902 | NP_636037.1 |
| pectate lyase II | Xanthomonas campestris pv. | AE012393 | AAM42087.1 |
| (PelB; XCC2815) | campestris str. ATCC 33913 | NC_003902 | NP_638163.1 |
| pectate lyase (PelB; Pl; Pstru-3) | Xanthomonas campestris pv. | L38573 | AAC41522.1 |
| | malvacearum strain B414 | | |
| pectin lyase (AN2331.2) | Aspergillus nidulans FGSC A4 | DQ490478 | ABF50854.1 |
| | | AACD01000038 | EAA64442.1 |
| pectin lyase (AN2569.2) | Aspergillus nidulans FGSC A4 | AACD01000043 | EAA64674.1 |
| | | DQ490480 | ABF50856.1 |
| pectate lyase (PelA; AN0741.2) | Aspergillus nidulans FGSC A4 | U05592 | AAA80568.1 |
| | | DQ490468 | ABF50844.1 |
| | | EF452421 | ABO38859.1 |
| | | AACD01000012 | EAA65383.1 |
| pectate lyase (AN7646.2) | Aspergillus nidulans FGSC A4 | AACD01000130 | EAA61832.1 |
| | | DQ490513 | ABF50889.1 |
| pectin lyase A (PelA) - Pl1A | Aspergillus niger CBS 120.49/ | X55784 | CAA39305.1 |
| | N400 | X60724 | CAA43130.1 |
| pectin lyase C (PelC) | Aspergillus niger CBS 120.49/ | AY839647 | AAW03313.1 |
| | N400 | | |
| pectin lyase F (PelF) | Aspergillus niger CBS 120.49/ | AJ489943 | CAD34589.1 |
| | N400 | | |
| pectate lyase A (PlyA) | Aspergillus niger CBS 120.49/ | AJ276331 | CAC33162.1 |
| | N400 | | |
| pectin lyase B (PelB) | Aspergillus niger CBS 120.49/ | A12248 | CAA01023.1 |
| | N400 | X65552 | CAA46521.1 |
| An14g04370 (PelA) | Aspergillus niger CBS 513.88 | AM270321 | CAK48529.1 |
| An03g00190 (PelB) | Aspergillus niger CBS 513.88 | AM270043 | CAK37997.1 |
| An15g07160 (PelF) | Aspergillus niger CBS 513.88 | AM270351 | CAK48551.1 |
| An19g00270 (PelD) | Aspergillus niger CBS 513.88 | AM270415 | CAK47350.1 |
| pectate lyase I | Aspergillus niger CBS 513.88 | AM270216 | CAK40523.1 |
| (PlyA; An10g00870) | | | |
| pectin lyase D (PelD) | Aspergillus niger N756 | M55657 | AAA32701.1 |
| pectin lyase 2 (Pel2) | Aspergillus oryzae KBN616 | AB029323 | BAB82468.1 |
| pectin lyase 1 (Pel1) | Aspergillus oryzae KBN616 | AB029322 | BAB82467.1 |
| pectin lyase 1 | Aspergillus oryzae RIB 40 | EF452419 | ABO38857.1 |
| (Pel1; AO090010000504) | | AP007175 | BAE66352.1 |
| pectin lyase 2 | Aspergillus oryzae RIB 40 | AP007175 | BAE65949.1 |
| (Pel2; AO090010000030) | | | |
| pectate lyase (PelB) | Colletotrichum gloeosporioides | AF052632 | AAD09857.1 |
| pectin lyase (PnlA) | Colletotrichum aloeosporioides | L22857 | AAA21817.1 |
| pectate lyase 2 (Pel-2) | Colletotrichum aloeosporioides | AF156985 | AAD43566.1 |
| | f. sp. malvae | | |
| pectin lyase (Pnl1; Pnl-1) | Colletotrichum aloeosporioides | AF158256 | AAF22244.1 |
| | f. sp. malvae | | |
| pectin lyase 2 (Pnl2; Pnl-2) | Colletotrichum aloeosporioides | AF156984 | AAD43565.1 |
| | f. sp. malvae | | |
| pectate lyase 1 (Pel-1) | Colletotrichum aloeosporioides | AF156983 | AAD43564.1 |
| | f. sp. malvae | | |
| pectate lyase (LLP-52) | Lilium longiflorum | L18911 | AAA33398.1 |
| | | EF026017 | ABM68553.1 |
| | | Z17328 | CAA78976.1 |
| pectate lyase | Musa acuminata Williams | AF206319 | AAF19195.1 |
| (PelI; Pl1; MwPl1; Banl7) | | DQ663594 | ABG74583.1 |
| | | X92943 | CAA63496.1 |
| pectate lyase | Nicotiana tabacum | X61102 | CAA43414.1 |
| | | X67158 | CAA47630.1 |
| | | X67159 | CAA47631.1 |
| pectate lyase | Zinnia elegans | Y09541 | CAA70735.1 |
| | | AX005936 | CAC05181.1 |

Polygalacturonases, rhamnogalacturonan lyases, and rhamnogalacturonan hydrolases may also be utilized herein to degrade and metabolize pectin or other polysaccharides. Examples of rhamnogalacturonan lyases include polypeptides sharing at least 60%, 70%, 80%, 90%, 95%, 98%, or more sequence identity (including all integers in between) to the rhamnogalacturonan lyases (i.e., rhamnogalacturonases) described in Table E, as well as polynucleotides and polynucleotide variants that encode them. Examples of rhamnogalacturonate hydrolyases include polypeptides sharing at least 60%, 70%, 80%, 90%, 95%, 98%, or more sequence identity (including all integers in between) to the rhamnogalacturonate hydrolases described in Table F, as well as polynucleotides and polynucleotide variants that encode them.

TABLE E
Rhamnoglacturonan lyases

| Protein | Organism | GenBank/GenPept | |
|---|---|---|---|
| rhamnogalacturonate lyase | Erwinia chrysanthemi | AJ438339 | CAD27359.1 |
| (RhiE) | 3937 | | |
| rhamnogalacturonan lyase | Aspergillus aculeatus | L35500 | AAA64368.1 |
| (RhgB) | KSM 510 | | |
| rhamnogalacturonan lyase | Aspergillus nidulans | AACD01000108 | EAA58417.1 |
| (AN6395.2) | FGSC A4 | DQ490501 | ABF50877.1 |
| rhamnogalacturonan lyase | Aspergillus nidulans | AACD01000122 | EAA61387.1 |
| (AN7135.2) | FGSC A4 | DQ490504 | ABF50880.1 |
| rhamnogalacturonan lyase | Bacillus subtilis subsp. | Z99107 | CAB12524.1 |
| (YesW; BSU07050) | subtilis str. 168 | NC_000964 | NP_388586.1 |
| exo-unsaturated | Bacillus subtilis subsp. | Z99107 | CAB12525.1 |
| rhamnogalacturonan lyase | subtilis str. 168 | NC_000964 | NP_388587.1 |
| (YesX; BSU07060) | | | |
| rhamnogalacturonan lyase - | Cellvibrio japonicus | AY026755 | AAK20911.1 |
| Rgl11A | (formerly | | |
| | Pseudomonas cellulosa) | | |
| CJA_3559 (rhamnogalacturonan | Cellvibrio japonicus | CP000934.1 | ACE83155.1 |
| lyase) - Rgl11A | Ueda 107 | | |
| rhamnogalacturonan lyase Y - | Clostridium cellulolyticum | AF316823 | AAG45161.1 |
| Rgl11Y | ATCC 35319 | | |

TABLE F
Rhamnogalacturonate hydrolyases

| Protein | Organism | GenBank/GenPept | |
|---|---|---|---|
| GH family 105 | | | |
| unsaturated rhamnogalacturonyl | Bacillus subtilis subsp. | Z99119 | CAB14990.1 |
| hydrolase (BSU30120; YteR) | subtilis str. 168 | | |
| unsaturated rhamnogalacturonyl | Bacillus subtilis subsp. | Z99107 | CAB12519.1 |
| hydrolase (BSU07000; YesR) | subtilis str. 168 | NC_000964 | NP_388581.1 |

Cellulases refer to a class of enzymes that catalyze the cellulolysis (or hydrolysis) of cellulose. Cellulose is a biological polysaccharide composed of a linear chain of several hundred to over ten thousand β(1→4) linked D-glucose units, having the formula (C₆H₁₀O₅)ₙ. Cellulases are mainly produced by fungi, bacteria, and protozoans, but there exist cellulases that are produced by other types of organisms, such as plants and animals. These enzymes typically act through hydrolysis of 1,4-beta-D-glycosidic linkages in cellulose and other similar polysaccharides, such as lichenin and cereal β-D-glucans, which ultimately breaks down cellulose into β-glucose.

Cellulases can be characterized according to the type of reaction that they catalyze. For instance, endo-cellulases break internal bonds to disrupt the crystalline structure of cellulose and to expose individual cellulose polysaccharide chains. General examples of endocellulases include endo-1,4-β-glucanase, carboxymethyl cellulase, endo-1,4-β-D-glucanase, β-1,4-glucanase, β-1,4-endoglucan hydrolase, and celludextrinase. The fusion polypeptides of the present invention may comprise one or more of such cellulases.

Exo-cellulases typically cleave about 2-4 units from the ends of the exposed chains produced by the endocellulase, resulting in tetrasaccharides or disaccharides, such as cellobiose. There are two main types of exo-cellulases (or cellobiohydrolases), one of which works processively from the reducing end of cellulose, and one of which works working processively from the non-reducing end of cellulose. Cellobiases or beta-glucosidase hydrolyses the exo-cellulase products into individual monosaccharides. Oxidative cellulases depolymerize cellulose by radical reactions, as for instance cellobiose dehydrogenase (acceptor). Cellulose phosphorylases depolymerize cellulose using phosphates instead of water. The fusion polypeptides of the present invention may comprise one or more of such cellulases.

Generally, cellulases can also be characterized as progressive (i.e., processive) and non-progressive cellulases. Progressive cellulases establish and maintain an interaction with a given, single polysaccharide strand, whereas non-progressive cellulases interact once with a polysaccharide strand, disengage, and then engage another polysaccharide strand.

Particular examples of cellulase polypeptides that may be employed in the fusion polypeptides of the present invention include, without limitation, polypeptides sharing at least 60%, 70%, 80%, 90%, 95%, 98%, or more sequence identity (including all integers in between) to endo-cellulase I (or endo-β-1,4-glucanase I) from Tricoderma reesei (SEQ ID NO:48), endo-cellulase II (or endo-β-1,4-glucanase II) from Tricoderma reesei (SEQ ID NO:50), and an endo-cellulase III (or endo-β-1,4-glucanase III) from Tricoderma reesei (SEQ ID NO:46). Additional examples of cellulases include a cellobiohydrolase II from Tricoderma reesei (SEQ ID NO:52), a cellulase Cel9E from Clostridium cellulolyticum (SEQ ID NO:54), a cellulase Cel9M from Clostridium cellulolyticum (SEQ ID NO:56), an endo-1,4-glucanase Cel9G from Clostridium cellulolyticum (SEQ ID NO:58), an endo-1,4-glucanase Cel5A from Clostridium cellulolyticum (SEQ ID NO:60), an endo-cellulase Cel48F from Clostridium cellulolyticum (SEQ ID NO:61), and a glucosidase I from Aspergillus aculeatu (SEQ ID NO:64). These sequences are described in the Sequence Listing.

In certain embodiments, cellulases (e.g., cellulases, cellobiohydrolases, cellodextrinases, and β-glucosidases) may be derived from Saccharophagus degradans 2-40. In certain embodiments, these cellulase polypeptides may be encoded by the following Saccharophagus degradans 2-40 genes or fragments thereof (see Example 2): Bgl1A (Sde_{—}3603), Bgl1B (Sde_{—}1394), Bgl3C (Sde_{—}2674), Cel5B (Sde_{—}2490), Cel5J (Sde_{—}2494), Ced3A (Sde_{—}2497), Cel5C (Sde_{—}0325), Ced3B (Sde_{—}0245), Cel9B (Sde_{—}0649), Cel5F (Sde_{—}1572), Cel9A (Sde_{—}0636), Cel6A (Sde_{—}2272), Cel5A (Sde_{—}3003), Cel5E (Sde_{—}2929), Cel5I (Sde_{—}3420). Also included are cellulase polypeptide that share at least 60%, 70%, 80%, 90%, 95%, 98%, or more sequence identity (including all integers in between) to the cellulases described herein (see, e.g., Sequence Listing), as well as polynucleotides and polynucleotide variants that encode them.

Laminarinases refer to a class of enzymes that split the polysaccharide laminarin (β-1:3-glucosan). Laminarinases are often referred to as endo-1,3-β-glucanases, and are typically characterized as exo-β-1,3-glucanases (β-1,3-glucan glucohydrolase EC 3.2.1.58) and endo-β-1,3-glucanases (β-1,3-glucan glucanohydrolases EC 3.2.1.6 and EC 3.2.1.39). Laminarin (or laminaran) is a storage glucan (i.e., a polysaccharide of glucose) that is found mainly in brown algae. This polysaccharide is created by photosynthesis, and is composed of glucose and mannitol, or β(1→3)-glucan with β(1→6)-linkages. It is a linear polysaccharide, with a β(1→3):β(1→6) ratio of 3:1.

Genes encoding bacterial β-1,3- and β-1,3-1,4-glucanases have been cloned and sequenced from different Bacillus species, Fibrobacter succinogenes, Cellvibrio mixtus, Thermotoga neapolitana, Ruminococcus flavefaciens, Oerskovia xanthineolytica, Clostridium thermocellum, and Rhodothermus marinus, among others (see, e.g., Gueguen et al., Journal of Biological Chemistry. 272:31258-31264, 1997, herein incorporated by reference). Bacterial endo-β-1,3-glucanases (laminarinases) share sequence similarity with endo-β-1,3-1,4-glucanases (lichenases) and have been classified in the same family 16 of glycosyl hydrolases. Eukaryotic endo-β-1,3-1,4-glucanases and endo-β-1,3-glucanases have been classified in family 17 of glycosyl hydrolases. However, the first metazoan β-1,3-glucanase, obtained from a sea urchin, shares homology with both β-1,3- and β-1,3-1,4-glucanases of glycosyl hydrolase family 16. The fusion polypeptides of the present invention may comprise any of these laminarinases, including variants thereof having 60%, 70%, 80%, 90%, 95%, 98%, or more sequence identity (including all integers in between) to these sequences, as well as polynucleotides and polynucleotide variants that encode them.

Examples of particular laminarinases include, without limitation, the LamA and Lam16A polypeptides of Thermotoga neapolitana, the Lic16A endo-beta-1,3-glucanase of Clostridium thermocellum (a non-cellulosomal, highly complex endo-beta-1,3-glucanase bound to the outer cell surface), the LamA polypeptide from the hyperthermophilic archaeon Pyrococcus furiosus (see, e.g., Gueguen et al., supra), laminarinase from the protistan Euglena gracilis (see, e.g., Fellig, Science. 131:882, 1960), laminarinases from Saccharophagous degradans, and polypeptides sharing at least 60%, 70%, 80%, 90%, 95%, 98%, or more sequence identity (including all integers in between) to these laminarinases, as well as polynucleotides and polynucleotide variants that encode them.

Also included are laminarinase polypeptide that share at least 60%, 70%, 80%, 90%, 95%, 98%, or more sequence identity (including all integers in between) to the laminarinases described herein, as well as polynucleotides and polynucleotide variants that encode them.

Lipases refer to a class of enzymes that catalyze the hydrolysis of ester bonds in water-insoluble, lipid substrates, such as triglycerides, phospholipids, and sphingolipids. Lipid substrates also include fatty acids, glycolipids, glycerolipids, betaine lipids, glycerolphospholipids, sterol lipids, prenol lipids, saccharolipids, sphingolipids, polyketides, and mixtures thereof. Fatty acids are carboxylic acids composed of long unbranched aliphatic tails (chain), which are either saturated or unsaturated. Examples of particular fatty acids include, without limitation, 14:0, trans-14, 16:0, 16:1n-7, trans-16, 16:2n-6, 18:0, 18:1n-9, 18:2n-6, 18:3n-6, 18:3n-3, 18:4n-3, 20:0, 20:2n-6, 20:3n-6, 20:4n-3, 20:4n-6, and 20:5n-3.

Examples of lipases include glycerol ester hydrolases (or triacylglycerol acylhydrolases), which hydrolyze triglycerides into diglycerides, monoglycerides, fatty acids, and glycerol; phospholipases, which hydrolyzes phospholipids into fatty acids and other lipophilic substances; and sphingomyelinases (or sphingomyelin phosphodiesterases), which hydrolyse sphingomyelin into phosphocholine and ceramide. Lipases often catalyze esterification, interesterification, acidolysis, alcoholysis and aminolysis, in addition to their hydrolytic activity on lipids such as triglycerides.

Triglycerides (or triacylglycerols) refer to a class of glyceride molecules in which the glycerol is esterified with three fatty acids. Chain lengths of the fatty acids in naturally-occurring triglycerides can be of varying lengths, but 16, 18 and 20 carbons are the most common. Natural fatty acids found in plants and animals are typically composed of even numbers of carbon atoms due to the way they are bio-synthesized from acetyl-CoA. Bacteria, however, can synthesize odd- and branched-chain fatty acids. Phospholipids refer to a class of lipids that are major component of all cell membranes. Most phospholipids contain a diglyceride, a phosphate group, and a simple organic molecule such as cholin. Sphingolipids are a specific type of phospholipid, and are derived from sphingosine.

As noted above, glycerol ester hydrolases are typically characterized by their ability to hydrolyze triglycerides into diglycerides, monoglycerides, fatty acids, and glycerol. Glycerol ester hydrolases may be derived from eukaryotes or prokaryotes.

Examples of glycerol ester hydrolases include, without limitation, hydrolyases from Microbacterium thermosphactum, Propionibacterium shermanii, Myxococcus xanthus, and various lactic acid bacteria. Also included are lipase polypeptides that share at least 60%, 70%, 80%, 90%, 95%, 98%, or more sequence identity (including all integers in between) to these lipases, as well as polynucleotides and polynucleotide variants that encode them.

Generally, phospholipases are typically categorized into four major classes according to the type of reaction they catalyze. Included are phospholipase A, including phospholipase A1, which cleaves the SN-1 acyl chain, and phospholipase A2, which cleaves the SN-2 acyl chain; phospholipase B, which cleaves both SN-1 and SN-2 acyl chains, and is also known as a lysophospholipase; phospholipase C, which cleaves before the phosphate, releasing diacylglycerol and a phosphate-containing head group; phospholipase C, which cleaves phospholipids just before the phosphate group; and phospholipase D, which cleaves after the phosphate group, releasing phosphatidic acid and an alcohol. Phospholipases may be derived from eukaryotes or prokaryotes. Also included are phospholipase polypeptides that share at least 60%, 70%, 80%, 90%, 95%, 98%, or more sequence identity (including all integers in between) to these phospholipases, as well as polynucleotides and polynucleotide variants that encode them.

Generally, sphingomyelinases are categorized according to their cation dependence, their optimal pH, or both. Included are lysosomal acid sphingomyelinases, secreted zinc-dependent acid sphingomyelinases, magnesium-dependent neutral sphingomyelinases, magnesium-independent neutral sphingomyelinases, and alkaline sphingomyelinases. Sphingomyelinases may be derived from eukaryotes or prokaryotes. Also included are sphingomyelinases polypeptides that share at least 60%, 70%, 80%, 90%, 95%, 98%, or more sequence identity (including all integers in between) to these sphingomyelinases, as well as polynucleotides and polynucleotide variants that encode them.

In certain embodiments, the fusion polypeptides of the present invention also comprise at least one signal peptide. Mainly, signal peptides can be useful in directing the secretion or cell-surface targeting of the fusion polypeptides. In certain embodiments, the carrier polypeptide may comprise its own signal peptide, i.e., a “native” signal peptide. In certain embodiments, often to enhance secretion or cell-surface targeting, the fusion polypeptide may comprise one or more “heterologous” signal peptides, i.e., a signal peptide that is derived from a protein that is different from the carrier polypeptide. Merely by way of illustration, a fusion protein may comprise Omp1 as a carrier protein (which is its own signal sequence) and a signal peptide sequence from E. coli lipoprotein (LLP) fused to the N-terminus of Omp1. Hence, as illustrated herein, certain embodiments may include a fusion polypeptide that comprises both a native signal peptide and a heterologous signal peptide.

Certain embodiments may employ bacterial signal peptides. It is believed that bacterial signal peptides differ from each other only in minor aspects. For instance, signal peptides of gram-positive bacteria typically have a slightly longer and more basic N-terminus than signal peptides from other bacteria. Certain embodiments may employ eukaryotic signal peptides, in part because it is understood that signal peptides from one secretory protein are able to replace those of other proteins targeted to a comparable site. For example, it has been shown that the signal peptides of E. coli periplasmic and outer membrane proteins, of vacuolar and secreted protein of Saccharomyces cerevisiae, and of constitutively secreted and storage granule proteins of multi-cellular eukaryotes can be exchanged without affecting the targeting of the mature protein. Indeed, in certain non-naturally occurring environments (e.g., expression of eukaryotic genes in bacterial cells), signal peptides of some proteins have been shown to mediate efficient secretion. For instance, insulin and ovalbumin are secreted by E. coli upon expression of these genes, and yeast is able to efficiently secrete various secretory proteins derived from multicellular eukaryotes.

In certain embodiments, signal sequences may be derived from extracellular bacterial proteins. Examples of signal peptides or signal sequences that may be incorporated into the fusion polypeptides of the present invention include, without limitation, Bacillus subtilis PgsA signal sequence, E. coli OmpA signal sequence, E. coli Ag43 signal sequence, B. licheniformis penicillinase signal sequence, E. coli lipoprotein (lpp) signal sequence, Bacillus cereus penicillinase III signal sequence, S. aureus penicillinase signal sequence, Neisseria IgA protease signal sequence, Serratia serine proteins signal sequence, Pseudomonas endoglucanase signal sequence, E. coli ST_{A} enterotoxin signal sequence, Klebsiella or E. coli cloacin (colicin) signal sequence, Proteus or Serratia Shla/HpmA hemolysin signal sequence, Klebsiella pullalanase signal sequence, Pseudomonas, Erwinia, or Xanthomonas exoenzyme signal sequences, Aeromonas aerolysin signal sequence, Vibrio CTX toxin signal sequence, Pseudomonas or Neisseria Type IV pillin signal sequence, E. coli P-type pilin signal sequence, E. coli Type-I pilin signal sequence, Yersinia OM proteins (YOPS) signal sequences, E. coli or Caulobacter flagellin signal sequences, Shigella or Salmonella Invasion protein signal sequences, E. coli, Proteus, or Morganella HlyA hemolysin signal sequences, Bordatella pertussis CyaA cyclolysin signal sequence, Erwinia, Serratia, or Proteus protease signal sequences, Rhizobium NodO signal sequence, E. coli ColV colicin signal sequence, among others known in the art (see, e.g., Sequence Listing for the sequences of many signal peptides).

These or other signal peptide sequences can be identified according to routine techniques in the art. For instance, PrediSi (Prediction of Signal peptides) represents one exemplary tool for predicting signal peptide sequences and their cleavage positions in both bacterial and eukaryotic amino acid sequences (see, e.g., Hiller et al., Nucleic Acids Research. 32:W375-W379, 2004). This software tool is especially useful for the analysis of large datasets in real time with high accuracy, and is based on a position weight matrix approach improved by a frequency correction that takes in to consideration the amino acid bias present in proteins. Also available is a Hidden Markov Model method for the prediction of lipoprotein signal peptides of Gram-positive bacteria, trained on a set of 67 experimentally verified lipoproteins (see, e.g., Bagos et al., J. Proteome Res. 7:5082-5093, 2008).

The individual polypeptide components of the fusion polypeptides can be fused together in any order. For instance, a carrier polypeptide may by fused to the N-terminus of the passenger polypeptide (i.e., N-carrier-passenger-C), or the passenger polypeptide may be fused to the N-terminus of the carrier polypeptide (i.e., N-passenger-carrier-C). In certain embodiments, if an optional heterologous signal peptide sequence is present, then the components may be arranged as follows, from N-terminus to C-terminus: heterologous signal peptide->carrier polypeptide->passenger polypeptide; or, heterologous signal peptide->passenger polypeptide->carrier polypeptide, among other combinations apparent to persons skilled in the art.

Fusion proteins may be prepared using standard techniques. For example, DNA sequences encoding the polypeptide components of a desired fusion may be assembled separately, and ligated into an appropriate expression vector. The 3′ end of the DNA sequence encoding one polypeptide component can be ligated, with or without a peptide linker, to the 5′ end of a DNA sequence encoding the second (or third) polypeptide component so that the reading frames of the sequences are in phase. This permits translation into a single fusion protein that retains the biological activity of both component polypeptides.

If desired, one or more peptide linker sequences may be employed to separate the first and second (or third, etc.) polypeptide components by a distance sufficient to ensure that each polypeptide folds into its secondary and tertiary structures, if desired. Such peptide linker sequences may be incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180, each of which is herein incorporated by reference. The linker sequence may generally be from 1 to about 50 amino acids in length. Linker sequences are typically not required when the first and second polypeptides have non-essential N-terminal amino acid regions that can be used to separate the functional domains and prevent steric interference.

The ligated DNA sequences may be operably linked to suitable transcriptional or translational regulatory elements. The regulatory elements responsible for expression of DNA are typically located 5′ to the DNA sequence encoding the first polypeptide. Similarly, stop codons required to end translation and transcription termination signals are typically present 3′ to the DNA sequence encoding the second (or third, fourth, etc.) polypeptide.

In certain embodiments, the isolated polypeptides of the present invention, which encode a fusion polypeptide, are located 3′ to a DNA sequence that contains a promoter or enhancer region. In certain embodiments, the promoter is suitable for regulating expression in a prokaryotic cell, such as E. coli. Contemplated are constitutive and inducible promoters, as described herein and known in the art. Particular examples of promoters include, without limitation, P_{trc} (E. coli), P_{pdc} (Zymomonas mobilis), P_{H207} (Coliphage), P_{D/E20} (Coliphage), P_{N25} (Coliphage), P_{L} (phage lambda), P_{A1} (phage T5), P_{rrnB-2} (E. coli), or P_{LPP} (E. coli). The sequences of many of these promoters are described in the Sequence Listing.

In certain specific embodiments, the carrier polypeptide comprises Omp1 (Zymomonas mobilis), the passenger polypeptide comprises ΔAI-I from Sphingomonas sp. AI, and the isolated polynucleotide is operably linked to a P_{pdc} promoter (Zymomonas mobilis. In other specific embodiments, the carrier polypeptide comprises OmpA (E. coli), the passenger polypeptide comprises alginate lyase ΔAI-I from Sphingomonas sp. AI, further comprising a signal peptide from LPP (E. coli), and the isolated polynucleotide is operably linked to a P_{pdc} promoter (Zymomonas mobilis). In other specific embodiments, the carrier polypeptide comprises Ag43 (E. coli), the passenger polypeptide comprises an alginate lyase from Pseudoalteromonas sp. SM0524, and the isolated polynucleotide is operably linked to a P_{pdc} promoter (Zymomonas mobilis).

In certain specific embodiments, the carrier polypeptide comprises Ag43 (E. coli), the passenger polypeptide comprises an alginate lyase AI-I from Sphingomonas sp. AI-I, and wherein the isolated polynucleotide is operably linked to a P_{pdc} promoter (Zymomonas mobilis). In certain specific embodiments, the carrier polypeptide comprises Ag43 (E. coli), the passenger polypeptide comprises an alginate lyase ΔAI-I from Sphingomonas sp. AI-I, and wherein the isolated polynucleotide is operably linked to a P_{pdc} promoter (Zymomonas mobilis).

In certain specific embodiments, the carrier polypeptide comprises Ag43 (E. coli), the passenger polypeptide comprises an alginate lyase AI-II from Sphingomonas sp. AI-I, and wherein the isolated polynucleotide is operably linked to a P_{pdc} promoter (Zymomonas mobilis). In certain specific embodiments, the carrier polypeptide comprises Ag43 (E. coli), the passenger polypeptide comprises an alginate lyase AI-III from Sphingomonas sp. AI-I, and wherein the isolated polynucleotide is operably linked to a P_{pdc} promoter (Zymomonas mobilis). In certain specific embodiments, the carrier polypeptide comprises Ag43 (E. coli), the passenger polypeptide comprises an alginate lyase from Pseudoalteromonas sp. SM0524, and the isolated polynucleotide is operably linked to a P_{H207} promoter (Coliphage).

In certain embodiments, the isolated polynucleotides of the present invention, which encode a fusion polypeptide, are present in a vector. Examples of suitable vectors include, without limitation, pTrc99a and pCCfos2. Also included are recombinant microorganisms that comprise any one or more of the isolated polynucleotides or vectors described herein.

As noted above, embodiments of the present invention also relate to methods of metabolizing a biomolecule (e.g., de-polymerizing a biopolymer), comprising incubating the biomolecule with a recombinant microorganism, for a time sufficient to allow metabolism of at least part of the biomolecule, wherein the recombinant microorganism comprises a polynucleotide or a vector that encodes a fusion polypeptide, as described herein, thereby metabolizing a biomolecule. In certain embodiments, the biomolecule is a biopolymer, such a polysaccharide, and the method comprises converting the polysaccharide to a monosaccharide or oligosaccharide, as described herein. In certain embodiments, the biomolecule is a lipid, and the method comprises converting the lipid to a fatty acid, monosaccharide, or both. In certain embodiments, the method comprises converting the biomolecule or biopolymer to a commodity chemical, such as ethanol or biodiesel. In certain embodiments, the fusion polypeptide is fully secreted by the microorganism, and in certain embodiments the secreted fusion polypeptide is attached to (or displayed on, or tethered to) the cell surface of the microorganism.

Such a recombinant microorganism may comprise additional genetic components, such as those components (e.g., monosaccharide or oligosaccharide transporters) that facilitate its growth on a given biomolecule (e.g., a polysaccharide, such as alginate or pectin) as a sole source of carbon, energy, or both (see, e.g., U.S. Application No. 2009/0139134, herein incorporated by reference; and Examples 2-3). In certain embodiments, the recombinant microorganism may alternatively or additionally comprise one or more gene deletions, as described herein, such as one or more deletions in the lactose dehydrogenase gene (ΔldhA), which plays a key role in the synthesis of lactate, the fumarate reductase gene (Δfrd), which converts fumarate into succinate, the pflB-focA operon (ΔpflB-focA), which encodes the central enzyme of fermentative metabolism, a pyruvate formate-lyase (PFL) gene (A or B), a formate/nitrite transporter (ΔFocA) gene, or fadR, a regulator of fatty acid metabolism.

In certain embodiments, the recombinant microorganism is already capable of growing on the biomolecule as a sole source of carbon and/or energy, and the addition of a fusion polypeptide of the present invention (encoded by a polynucleotide) enhances its ability to metabolize the biomolecule, as measured, merely by way of non-limiting example, by an increase in the microorganism's total capacity (or percentage of its total theoretical yield) to produce a commodity chemical, such as ethanol. In certain embodiments, the microorganism is otherwise not capable of growing on the biomolecule as a sole source of carbon and/or energy, and the addition of the fusion polypeptide renders that microorganism capable of growing on the biomolecule as a sole source of carbon and/or energy.

Also, the fusion polypeptides of the present invention, and the polynucleotides that encode them, can be combined with any of the other methods and recombinant microorganisms provided herein, to enhance even further the total capacity of such a recombinant microorganism to grow on one or more selected biomolecules, and to produce a commodity chemical therefrom.

### Improved Metabolism of Biomolecules and Methods of Use

As noted above, embodiments of the present invention also relate to improved vector systems, and recombinant microorganisms containing the same, which confer on the recombinant microorganisms the ability to grow more efficiently on biomass-based biomolecules, such as alginate, cellobiose, methylcarboxycellulose, and fatty acids, including combinations thereof, by first converting those carbon sources to common metabolites, and then using those common metabolites to synthesize commodity chemicals. Depending on the selected biomolecule, such as alginate or cellobiose, these vector systems and recombinant microorganisms utilize the exogenous expression of one or more of a variety of newly unidentified lyases, hydrolyases, cellulases, transporters, symporters, synthases, porins, hydrogenases, glucosidases, and/or transcriptional regulators, among other components, to improve the extracellular metabolism, transport, and intracellular metabolism of biomolecules, such as biopolymers and the smaller components of biopolymers.

In certain embodiments, the improved vector systems and recombinant microorganisms comprising the same are based on a fosmid clone isolated from genomic library of V. splendidus 12B01 (e.g., pALG1.5), which provides microorganisms such as E. coli with the ability to metabolize and grown on alginate as a sole source of carbon (see, e.g., U.S. application Ser. No. 12/245,537, herein incorporated by reference). In certain embodiments, this fosmid clone has been modified to include further genetic components, to either enhance the ability of recombinant microorganism to grown on alginate as a sole source of carbon, or to provide it with the ability to grow on other carbon sources, such as cellulose, cellobiose, or hydroxymethylcellulose, etc., as a sole source of carbon, energy, or both. Exemplary vectors comprising such components are summarized in Table G below. Also included are vectors and recombinant microorganisms that comprise functional equivalents or variants of such components.

TABLE G
Exemplary vectors for improved growth on biomass.

| Vector | Modifications added to the previous version of pALG vector |
|---|---|
| pALG1.5 | Original fosmid clone isolated from genomic library of V. splendidus 12B01 |
| pALG1.6 | V12B01_24254 (alginate lyase) andV12B01_24259 (alginate lyase) are added to pALG1.5 |
| pALG1.7 | V12B01_24264 (alginate lyase), V12B01_24269 (outer membrane porin), and |
| | V12B01_24274 (alginate lyase) are added to pALG1.6 |
| pALG2.0 | V12B01_24269 (outer membrane porin) was added to pALG1.5 |
| pALG2.1 | Cm site of pALG1.7 is replaced with Km |
| pALG2.2 | V12B01_24309 (outer membrane porin) is added to pALG1.7 |
| pALG2.3 | V12B01_24309 (outer membrane porin) and V12B01_24324 (transporter) are added to pALG1.7 |
| pALG2.4 | V12B01_19706 (transporter) is added to pALG1.7 |
| pALG2.5 | V12B01_24309 (outer membrane porin), V12B01_24324 (transporter), and |
| | V12B01_24269 (outer membrane porin) were added to pALG1.5 |
| pALG3.0 | Atu_3020, Atu_3021, Atu_3022, Atu_3023, Atu_3024 (21-24: ABC transporter), Atu_3025 |
| | (oligoalginate lyase), and Atu_3026 (DEHU hydrogenase) were added to pALG2.5 |
| pALG3.5 | V12B01_24254 (alginate lyase) and V12B01_24259 (alginate lyase) were added to pALG3.5 |
| pALG4.0 | Sde_3602 (Glutathione synthetase), Sde_3603 (β-glucosidase 1A: Bgl1A), Sde_1394 (β- |
| | glucosidase 1B: Bgl1B), Sde_1395 (cellobiose transporter), Sde_2674 (β-glucosidase 3C: |
| | Bgl3C), Sde_2637 (tRNA pseudouridine synthase B), and Atu_3019 were added to the pALG3.5 |
| pALG5.0 | Sde_2491 (Transcription regulator), Sde_2490 (Cellulase 5B: Cel5B), Sde_2497 |
| | (Cellodextrinase 3A: Ced3A), Sde_2496 (Glyoxylase), Sde_2495 (Transcription |
| | regulator), and Sde_2494 (Cellulase 5J: Cel5J) were added to the pALG4.0 |
| pALG5.1 | Sde_0245 (Cellodextrinase 3B: Ced3B), Sde_0324 (Transcription regulator), Sde_0325 |
| | (Cellulase 5C: Cel5C), Sde_0649 (Cellulase 9B: Cel9B), and Sde_1572 (Cellulase 5F: |
| | Cel5F) were added to the pALG5.0 |
| pALG5.2 | Sde_0636 (Cellulase 9A: Cel9A), and Sde_2272 (Cellulase 6A: Cel6A) were added to the |
| | pALG5.1 |
| pALG5.3 | Sde_2929 (Cellulase 5E: Cel5E), Sde_3003 (Cellulase 5A: Cel5A), and Sde_3420 |
| | (Cellulase 5I: Cel5I) were added to the pALG5.2 |
| pALG7.0 | P_{D/E20}-Ag43-ΔPaAly was added to pALG4.0 |
| pALG7.1 | P_{A1}-Ag43-ΔPaAly was added to pALG4.0 |
| pALG7.2 | P_{H207}-Ag43-ΔPaAly was added to pALG4.0 |
| pALG7.2.1 | P_{H207}-Ag43-ΔPaAly is added to pALG2.1 |
| pALG7.2.2 | P_{H207}-Ag43-ΔPaAly is added to pALG2.2 |
| pALG7.2.3 | P_{H207}-Ag43-ΔPaAly is added to pALG2.3 |
| pALG7.2.4 | P_{H207}-Ag43-ΔPaAly is added to and V12B01_24324 (transporter) is excised from pALG2.3 |
| pALG7.3 | P_{LPP}-Ag43-ΔPaAly was added to pALG4.0 |
| pALG7.4 | P_{H207}-Ag43-ΔPaAly was added to pALG2.0 |
| pALG7.5 | P_{H207}-Ag43-ΔPaAly was added to pALG2.5 |
| pALG7.6 | V12B01_24249-24259 is excised from pALG7.2 |
| pALG7.8 | V12B01_24264-24274 is added to pALG7.2 |

Certain embodiments relate to recombinant microorganisms that are capable of growing on a polysaccharide such as alginate as a sole source of carbon, comprising one or more exogenous polynucleotides that contain a genomic region between V12B01_{—}24189 and V12B01_{—}24249 of Vibrio splendidus, and that encodes an additional outer membrane porin, such as an outer membrane porin from Vibrio splendidus (e.g., pALG2.0) or other organism, or a functional equivalent thereof. The one or more outer membrane porins from Vibrio splendidus (V12B01_{—}24269) may comprise an amino acid sequence at least 80%, 85%, 90%, 95%, 98%, or 100% identical to the amino acid sequence set forth in SEQ ID NO:92. As noted above, outer membrane porins are generally non-selective, transmembrane channels that are found in the outer membrane of Gram-negative bacteria. The pore structure of these proteins is formed almost entirely of a beta-barrel, and the monomeric protein is typically matured into a trimeric species that ultimately integrates into the outer membrane. Examples of outer membrane porins include, without limitation, Omp1 (Zymomonas mobilis), porin F (P. aeruginosa), OmpA (E. coli), OmpF/C (E. coli), OmpG (E. coli), and PhoE porin (E. coli), and biologically active fragments or variants thereof. Additional examples of outer membrane porins can be found, for instance, in Nguyen et al., Mol Microbiol Biotechnol. 11:291-301, 2006.

Certain vectors and recombinant microorganisms may comprise one or more exogenous polynucleotides that encode a symporter and/or a porin, such as a symporter and/or a porin from Vibrio splendidus (e.g., pALG2.5) or other organism, or a functional equivalent thereof. These and related embodiments may also comprise any one or more of the additional components described herein. The symporter (e.g., V12B01_{—}24324) and outer membrane porin (e.g., V12B01_{—}24309; V12B01_{—}24269) from Vibrio splendidus, respectively, may comprise an amino acid sequence at least 80%, 85%, 90%, 95%, 98%, or 100% identical to the amino acid sequence set forth in SEQ ID NO:96, 94, or 92. A symporter is a membrane transporter that co-transports two or more dissimilar molecules in the same direction across a membrane. Usually, the transport of one molecule (or ion) is against its electrochemical gradient, which is “powered” by the movement of the other molecule (or ion) with its electrochemical gradient. These embodiments have improved alginate metabolism.

Certain vectors and recombinant microorganisms may comprise one or more exogenous polynucleotides that encode an ABC transporter, an oligoalginate lyase, and/or a DEHU hydrogenase, or equivalents thereof, which may be derived, for example, from Agrobacterium tumefaciens (e.g., pALG3.0) or other organism. These and related embodiments may also comprise any one or more of the additional components described herein. The Agrobacterium tumefaciens-derived ABC transporter (e.g., Atu_{—}3020, Atu_{—}3021, Atu_{—}3022, Atu_{—}3023, or Atu_{—}3024), oligoalginate lyase (e.g., Atu_{—}3025), and DEHU hydrogenase (e.g., Atu_{—}3026), respectively, may comprise an amino acid sequence at least 80%, 85%, 90%, 95%, 98%, or 100% identical to the amino acid sequence set forth in SEQ ID NO:110, 112, 114, 116, 118 (ABC transporters), 120 (olioalginate lyase), or 122 (DEHU hydrogenase). Bacterial ABC transporters are transmembrane proteins that utilize adenosine triphosphate (ATP) hydrolysis to translocate a wide variety of substrates across extra- and intracellular membranes, including metabolic products, lipids, ions, amino acids, peptides, and sugars. These embodiments have improved alginate metabolism.

Certain vectors and recombinant microorganisms may comprise one or more exogenous polynucleotides that encode one or more alginate lyases, or two or more alginate lyases, such as alginate lyases from Vibrio splendidus (e.g., pALG3.5) or other organism, or functional equivalents thereof. These and related embodiments may also comprise any one or more of the additional components described herein. The one or more alginate lyases (e.g., V12B01_{—}24254; V12B01_{—}24259) from Vibrio splendidus may comprise an amino acid sequence at least 80%, 85%, 90%, 95%, 98%, or 100% identical to the amino acid sequence set forth in SEQ ID NO:98 or 100. These embodiments have improved alginate metabolism.

Certain vectors and recombinant microorganisms may comprise one or more exogenous polynucleotides that encode one or more β-glucosidases, two or more β-glucosidases, or three or more β-glucosidases, such as β-glucosidases from Saccharophagous degradans or other organism, or functional equivalents thereof. Certain of these or related embodiments may also comprise a cellobiose transporter, such as a cellobiose transporter from Saccharophagous degradans or other organism (e.g. pALG4.0), or functional equivalent thereof. Certain of these or related embodiments may also comprise a glutathione synthetase or a tRNA pseudouridine synthase B, which may be derived from Saccharophagous degradans or other organism (e.g. pALG4.0), or functional equivalent thereof. These and related embodiments may also comprise any one or more of the additional components described herein.

In certain embodiments, the Saccharophagous degradans-derived β-glucosidases may include, for example, β-glucosidase 1A (Sde_{—}3603; Bgl1A), β-glucosidase 1B (Sde_{—}1394; Bgl1B), and β-glucosidase 3C (Sde_{—}2674; Bgl3C), and may comprise an amino acid sequence at least 80%, 85%, 90%, 95%, 98%, or 100% identical to the amino acid sequence set forth in SEQ ID NO:124, 126, or 130. In certain embodiments, the Saccharophagous degradans-derived glutathione synthetase (Sde_{—}3602), cellobiose transporter (Sde_{—}1395), and tRNA pseudouridine synthase B (Sde_{—}2637) may comprise an amino acid sequence at least 80%, 85%, 90%, 95%, 98%, or 100% identical to these sequences, including the amino acid sequence set forth in SEQ ID NO:128. In certain embodiments, the vector or recombinant microorganism may comprise a polynucleotide that encodes Atu_{—}3019 (SEQ ID NO:108), or a variant thereof. These embodiments have the ability to grow on cellobiose, cellulose, or carboxymethylcellulose as a sole source of carbon.

Certain vectors and recombinant microorganisms comprise one or more exogenous polynucleotides that encode one or more cellodextrinases, or two or more cellodextrinases, including cellodextrinases from Saccharophagous degradans (e.g., pALG5.0 and pALG5.1) or other organism, or functional equivalents thereof. Certain vectors and recombinant microorganisms comprise one or more exogenous polynucleotides that encode one or more cellulases, including cellulases from Saccharophagous degradans (e.g., pALG5.0, pALG5.1, pALG5.2, and pALG5.3) or other organism, or functional equivalents thereof. Cellulases refer to a class of enzymes produced mainly by fungi, bacteria, and protozoans that catalyze the cellulolysis (or hydrolysis) of cellulose, and cellodextrinases are one class of cellulase. Certain vectors and recombinant microorganisms comprise one or more exogenous polynucleotides that encode one or more cellobiohydrolases, including cellobiohydrolases from Saccharophagous degradans (e.g., pALG5.2 and pALG 5.3) or other organism, or functional equivalents thereof. Cellobiohydrolases bind to cellulose during the initial steps of cellulose hydrolysis. These and related embodiments may also comprise any one or more of the additional components described herein. In certain embodiments, the Saccharophagous degradans-derived cellodextrinases (e.g., Sde_{—}2497; Sde_{—}0245), cellulases or cellobiohydrolases (e.g. Sde_{—}2490; Sde_{—}2494; Sde_{—}2496; Sde_{—}0325; Sde_{—}0649; Sde_{—}1572; Sde_{—}0636; Sde_{—}2272; Sde_{—}2272; Sde_{—}2929; Sde_{—}3003; Sde_{—}3420) noted above may comprise an amino acid sequence at least 80%, 85%, 90%, 95%, 98%, or 100% identical to the amino acid sequence of these polypeptides (see, e.g., the Sequence Listing). These embodiments have the ability to grow on cellobiose, cellulose, or carboxymethylcellulose as a sole source of carbon.

In certain embodiments, the recombinant microorganisms of the present invention may comprise the above-noted vector systems or components (genes), or their functional equivalents, in any combination, including, but not limited to, the combination of exemplary vector systems listed in Table G. Embodiments of the instant invention, however, are not limited to these specific combinations of components. Also, improved recombinant microorganisms comprising such vector systems or genetic components may further comprise any of the other technologies described herein, such as the tether-display vectors and the fusion polypeptides encoded by such vectors or polynucleotides. Merely by way of illustration, certain preferred embodiments may employ the above-noted pALG vectors, or their equivalents, which further contain tether systems such as Ag43-ΔPaAly under the control of different promoters (see, e.g., pALG 7.0, 7.1, 7.2, 7.3, 7.4, 7.5). These and related embodiments have enhanced ability to metabolize and grow on various biomass-derived polysaccharides, such as alginate, cellobiose, cellulose, or carboxymethylcellulose, including oligosaccharide components derived therefrom.

Certain embodiments of the present invention also relate to the use of deletion mutants to optimize the production or intracellular synthesis of certain carbon-based molecules, or commodity chemicals, such as ethanol or biodiesel. Without wishing to be bound by any one theory, it is believed the production of a desired carbon-based molecule such as ethanol can be enhanced by reducing the intracellular production of other carbon based molecules, and thereby shunting the limited resources of a given microorganism towards the production of the desired molecule. For instance, in certain exemplary embodiments, reducing the production of lactate, succinate, formate, acetate, etc., can increase the production of pyruvate, and thereby increase the production of ethanol (see, e.g., FIG. 21).

The reduced production of such undesired carbon molecules, and the increased production of desired molecules, can be accomplished by deleting one or more key genes in the synthetic pathways of the undesired molecules. One example of such a deletion mutants includes a deletion in the lactose dehydrogenase gene (ΔldhA), which plays a key role in the synthesis of lactate. Another example includes a deletion in the fumarate reductase gene (Δfrd), which converts fumarate into succinate. Other genes in the fumarate reductase complex or the succinate biosynthesis pathway may be deleted. For instance, the fumarate reductase complex includes three subunits: subunit A contains the site of fumarate reduction and a covalently bound flavin adenine dinucleotide prosthetic group, subunit B contains three iron-sulphur centres, and the menaquinol-oxidizing subunit C, which consists of five membrane-spanning, primarily helical segments and binds two haem b molecules. Any of these genes can be deleted to reduce the synthesis of succinate, and shunt the limited carbon resources towards the desired carbon molecule.

Another example includes a deletion in the pflB-focA operon (ΔpflB-focA), the pflB gene being a component of the pflABCD operon. This operon encodes the central enzymes of fermentative metabolism, pyruvate formate-lyase (pfl) gene (ΔpflA, ΔpflB, ΔpflC, or ΔpflD), and the focA gene, a formate/nitrite transporter gene. In certain embodiments, any one or more of the genes in the pflABCD operon can be deleted, either alone or in combination with the focA gene. In certain preferred embodiments, the ΔpflA or the ΔpflB gene is deleted in combination with the focA gene.

Also included are deletions in the fadR gene (ΔfadR), a regulator of fatty acid metabolism. It is believed that deleting the fadR gene enhances the metabolism of fatty acids, thereby improving the production of desired carbon based molecules or commodity chemicals such as ethanol, especially for recombinant microorganisms growing on mixtures of polysaccharides and fatty acids or other lipids. Hence, such microorganisms typically show enhanced fatty acid metabolism as compared to a microorganism without one or more deletions in fadR. Additional examples include deletion mutants of the ppc, pck, mdh, pta, sdh, and/or fumB genes.

The gene deletions described herein may be used individually or in any combination. Deletions may also be full or partial, typically as long as the reduce or eliminate expression of the corresponding protein, or express a truncated, dysfunctional variant of the protein having reduced activity. In certain embodiments, a recombinant microorganism may comprise all of the above-noted deletions (e.g., ΔldhA, Δfrd, ΔpflB-focA, ΔfadR, Δppc, Δpck, Δmdh, Δpta, Δsdh, and ΔfumB), or any combination thereof. These gene deletions can be accomplished using routine techniques in the art, as exemplified herein (see Example 4). Also, the deletion mutants of the present invention may be used in combination with any of the other vector systems, recombinant microorganisms, or methods provided herein.

Certain embodiments relate to methods of optimizing the growth of the recombinant microorganisms described herein, mainly to enhance the yield of a desired target molecule or commodity chemical. Certain of these methods involve optimizing a growth mixture, typically comprising polysaccharides, fatty acids, or both, to achieve an optimal ratio of different polysaccharides. For instance, certain embodiments relate to the use of a growth mixture that comprises at least one uronic acid and at least one sugar alcohol, often under anaerobic fermentative conditions, wherein the at least one uronic acid and the at least one sugar alcohol have different reduction-oxidation (redox) potentials.

Without wishing to be bound by any one theory, it is believed that the use of such mixtures balances the intracellular redox potential of the microorganism, reducing the growth inhibitory effects of redox imbalance (e.g., excess nicotinamide adenine dinucleotide; NADH), and thereby enhancing production or yield of the target molecule. In explanation, such as in the production of ethanol, glucose is a good sugar for ethanol fermentation, because the carbon flux is balanced, two adenosine triphosphate (ATP) molecules are produced that can be used for growth, and the redox potential is balanced. However, the production of ethanol from biomass-derived polysaccharides requires the use of more complex carbon sources, such as mannitol and alginate. When producing desired carbon molecules such as ethanol from mannitol, a sugar alcohol component of biomass such as seaweed or kelp (a typical seaweed culture is about 12.5%), the carbon flux is balanced, 2 ATP molecules are produced for growth, but the redox potential is imbalanced. This imbalance can be inhibitory to growth, and reduce the maximum yield of the desired carbon molecule. However, by growing the recombinant microorganism in the presence of both the sugar alcohol and at least one uronic acid, especially wherein the sugar alcohol and the uronic acid have different redox potentials, this imbalance can be reduced or avoided, improving both the growth characteristics of the microorganism and the overall yield of the desired carbon molecule. Hence, in certain embodiments these methods reduce intracellular NADH/NADPH accumulation as compared to incubating the microorganism with the sugar alcohol alone. Also, in certain embodiments, these methods reduce intracellular acetate accumulation as compared to incubating the microorganism with the uronic acid alone.

Uronic acids are sugar acids with both a carbonyl and a carboxylic acid group, which is typically produced by oxidation of the terminal carbon's hydroxyl group to a carboxylic acid. Examples of uronic acids include, without limitation, alginate, mannuronate, guluronate, DEHU, glucuronate, and galacturonate, including mixtures thereof. Sugar alcohols (i.e., polyol, polyhydric alcohol, or polyalcohol) are hydrogenated forms of carbohydrates, in which the carbonyl group (aldehyde or ketone) has been reduced to a primary or secondary hydroxyl group. Sugar alcohols have the general formula H(HCHO)ₙ₊₁H. Examples of sugar alcohols include, without limitation, mannitol, glycerol, glycol, erythritol, threitol, arabitol, xylitol, ribitol, sorbitol, dulcitol, iditol, isomalt, maltitol, lactitol, and polyglycitol, including mixtures thereof. Merely by way of illustration, the synthesis of ethanol from mannitol produces one molecule of NADH, which may result in the excessive production of NADH over time. Alginate and glucuronate, on the other hand, consume NADH in producing ethanol, and can be used to balance out the otherwise damaging accumulation of NADH that is produced by the metabolism of mannitol. Hence, in certain preferred embodiments the uronic acid is alginate or glucuronate or both, and the sugar alcohol is mannitol.

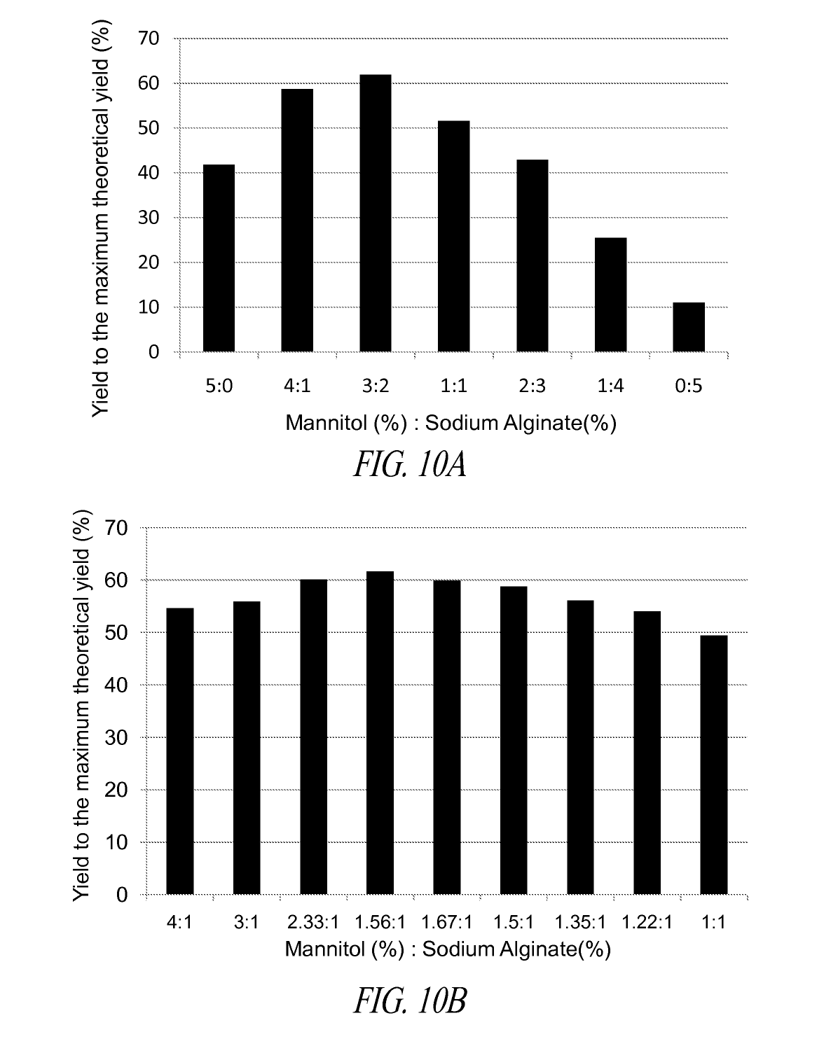

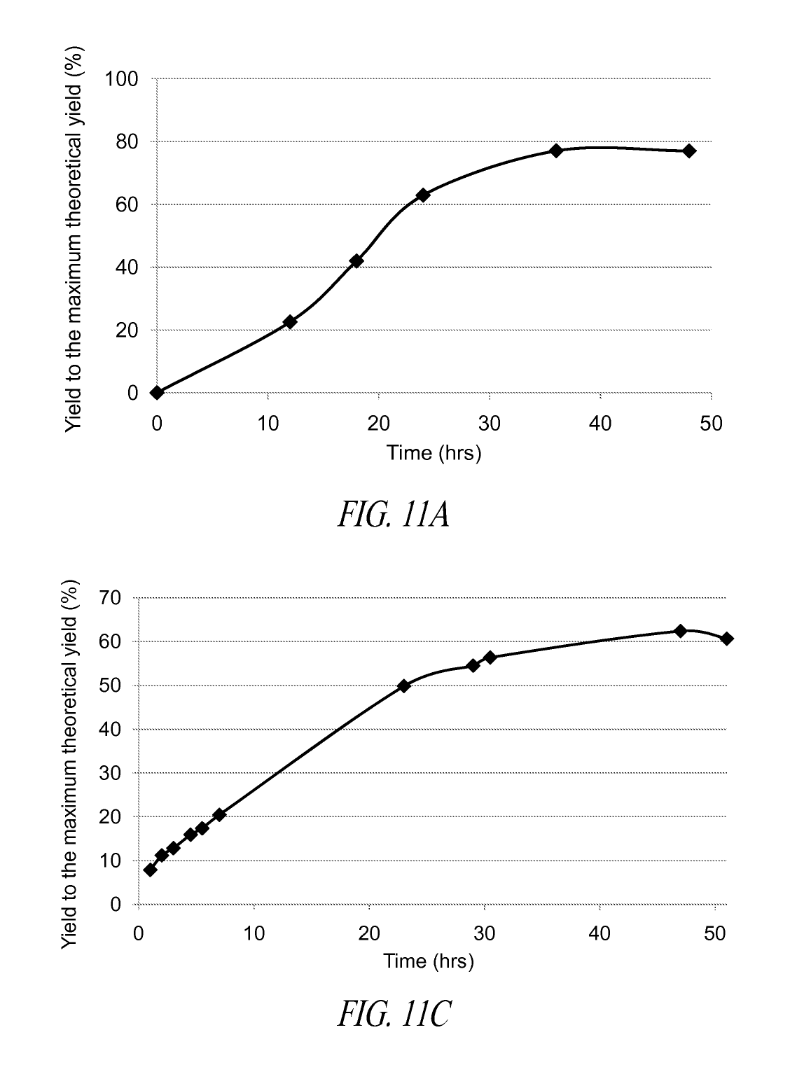

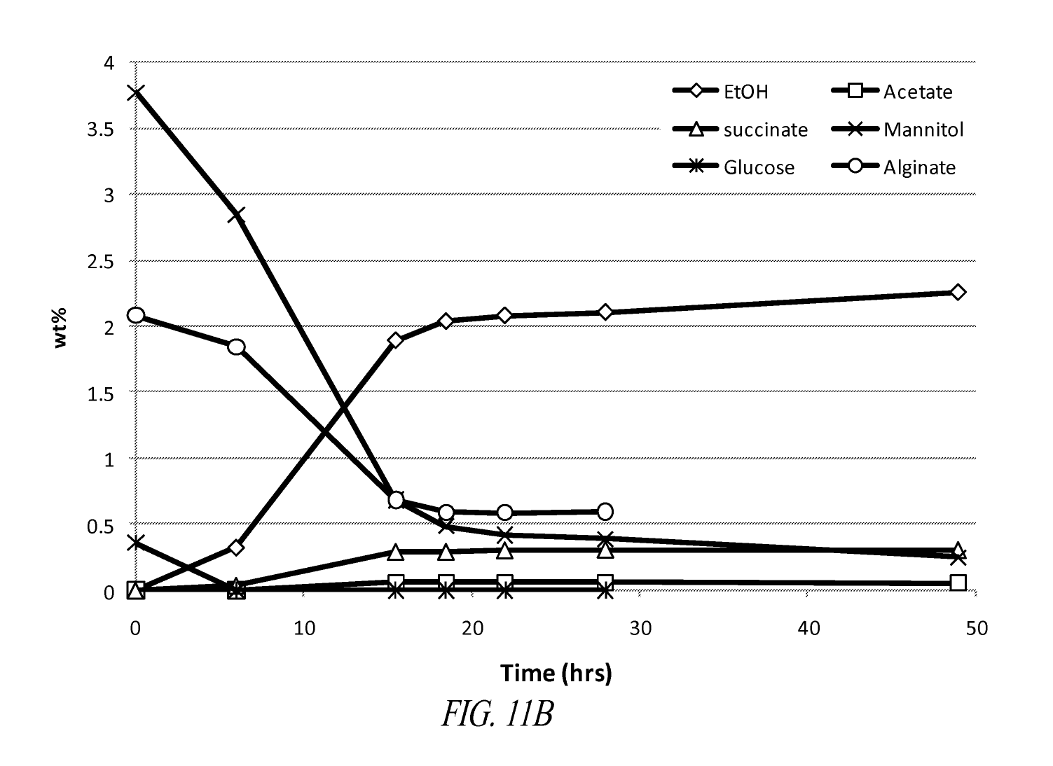

In certain embodiments, the ratio of the uronic acid to the sugar alcohol may be optimized for a given microorganism or production system. For instance, the uronic acid:sugar alcohol ratio may be about 6:1, 5:1, 4:1, 3:1, 3:2, 2:1, 1:1, 1:2, 2:3 1:3, 1:4, 1:5, or 1:6 including all decimal points in between (see, e.g., FIG. 10B). In certain embodiments, the at least one uronic acid is alginate and the at least one sugar alcohol is mannitol, and the alginate:mannitol ratio may be about 6:1, 5:1, 4:1, 3:1, 3:2, 2:1, 1:1, 1:2, 2:3 1:3, 1:4, 1:5, or 1:6, including all decimal points in between, or the alginate:mannitol ratio is about 2:3 or about 1:1.56, 1:1.67, 1:2.33, or other optimal amount (see FIG. 10B). In certain embodiments, the at least one uronic acid is galacturonate and the at least one sugar alcohol is mannitol, and the galacturonate:mannitol ratio may be about 6:1, 5:1, 4:1, 3:1, 3:2, 2:1, 1:1, 1:2, 2:3 1:3, 1:4, 1:5, or 1:6, including all decimal points in between, or the galacturonate:mannitol ratio is about 2:1. In certain embodiments, the at least one uronic acid is glucuronate and the at least one sugar alcohol is mannitol, and the glucuronate:mannitol ratio may be about 5:1, 4:1, 3:1, 3:2, 2:1, 1:1, 1:2, 2:3 1:3, 1:4, or 1:5, including all decimal points in between, or the glucuronate:mannitol ratio is about 1:1.

These methods can be used with any of the recombinant microorganisms described herein. In certain embodiments, these methods may be utilized to increase the maximum yield of a selected commodity chemical such as ethanol, often in a manner that approaches or even surpasses the theoretical maximum yield for that chemical.

### Improved Microorganisms for the Production of Ethanol, and Methods of Use

Certain embodiments of the present invention relate to the discovery that ethanol-producing recombinant microorganisms can be modified to increase their metabolism of fatty acids, mainly in a manner that shunts the products of fatty-acid metabolism towards the production of ethanol, and thereby increases their overall ethanol-producing capacity, especially when grown in a mixture that comprises polysaccharides and lipids. Briefly, the enhanced production of ethanol in this manner may be accomplished by supplementing a sugar-dependent ethanol-synthesizing pathway (pdc-adhA-adhB operon) with an acetaldehyde/alcohol dehydrogenase (adhE), to facilitate the conversion of fatty acids, as well as other sugars, into ethanol.

In this regard, the introduction of a pyruvate decarboxylase (Pdc) and alcohol dehydrogenases I and II (adhA and adhB, respectively) from Zymomonas mobilis confers on E. coli the ability to convert sugars into ethanol (see, e.g., U.S. Pat. Nos. 5,000,000; 5,028,539; 5,916,787; 5,482,846; 5,424,202; and 5,162,516, herein incorporated by reference). It is believed that this sugar-dependent ethanol-synthesizing pathway proceeds as follows: the glycolysis of sugars produces pyruvate, Pdc converts pyruvate to acetoaldehyde, and adhA and adhB convert acetoaldehyde to ethanol. However, this pathway does not effectively utilize the intermediates or by-products of fatty acid metabolism, such as acetyl-CoA. As such, even if certain recombinant microorganisms (e.g., E. coli that comprises Pdc, adhA, and adhB) are capable of metabolizing fatty acids, they are relatively limited in their ability to produce ethanol from fatty acid-containing energy sources, such as kelp or other forms of biomass.

To overcome this limitation, and to significantly increase the overall yield of ethanol, recombinant microorganisms can be generated that comprise not only Pdc, adhA, and adhB, but express high levels of acetaldehyde/alcohol dehydrogenase (adhE), or a biologically active equivalent or variant thereof. Without being bound by any one theory, the bi-functional (i.e., acetaldehyde and alcohol dehydrogenase) adhE enzyme is believed to catalyze the conversion of acetyl-CoA into acetoaldehyde, and then catalyze the conversion of acetoaldehyde into ethanol. Acetyl-CoA is a primary intermediate or by-product in the metabolism of fatty acids, but not necessarily in the metabolism of sugars, which mainly leads to pyruvate formation (which is converted to acetoaldehyde by Pdc, and then converted to ethanol by adhA and adhB). Hence, by combining these genetic features, especially when growing the recombinant microorganism on a source of energy (e.g., kelp, algae) that contains both polysaccharides and lipids/fatty acids, the efficiency of producing ethanol from that source of energy can be significantly enhanced, as can the total or theoretical maximum yield.

Included are recombinant microorganisms that comprise one or more exogenous polynucleotides that encode Pdc, adhA, adhB, and adhE, or variants thereof. Polynucleotides that encode alcohol/acetaldehyde dehydrogenases (adhE) can be obtained from a variety of organisms, such as E. coli, Thermoanaerobacter ethanolicus JW200, Clostridium acetobutylicum ATCC 824 and M5, Entamoeba histolytica, heterotrophic chlorophyte Polytomella sp., Giardia lamblia, Staphylococcus aureus, Salmonella enterica subsp., Pectobacterium atrosepticum, among others known in the art. The amino acid sequence of an exemplary adhE from E. coli strain K12 is set forth in SEQ ID NO:155.

AdhE activity can also be increased by regulating the endogenous levels of adhE. Hence, also included are recombinant microorganisms (e.g., E. coli) that comprise one or more exogenous polynucleotides that encode Pdc, adhA, adhB, and that have mutations in the adhE regulatory pathways, such as mutants that are limited in their ability to control the expression of endogenous adhE. These mutants may also comprise an exogenously expressed adhE polypeptide. Merely by way of background, expression of adhE in E. coli is about 10-fold higher in cells grown anaerobically than in cells grown aerobically, and is regulated by both transcriptional and post-transcriptional factors. Specifically, trans-regulatory elements that repress adhE expression have been characterized by genetic and biochemical approaches. For instance, mutations that down-regulate adhE expression have been chromosomally mapped, revealing a missense mutation in the cra gene, formerly known as fruR. The cra gene encodes a catabolite repressor-activator protein (Cra) involved in the modulation of carbon flow in E. coli. The mutant protein (Cra*) has an Arg148His substitution, which leads to 1.5- and 3-fold stronger repression of adhE transcription under anaerobic and aerobic conditions, respectively. By comparison, cra null mutants display 1.5- and 4-fold increased adhE transcription under those conditions. Also, disruption of the cra gene does not abolish the anaerobic activation of the adhE gene, but diminishes it about two-fold. In vitro, Cra and Cra* 6×His-fusion proteins show binding to the adhE promoter region and to the control fruB promoter region, which is a known Cra target. However, only 6×His-tagged Cra, and not 6×His-Cra*, is displaced from the DNA target by the effector, fructose-1-phosphate (F1P), suggesting that the mutant protein is locked in a promoter-binding conformation and is no longer responsive to F1P. Cra helps to tighten the control of adhE transcription under aerobic conditions by its repression. Hence, certain embodiments may employ cra deletion mutants, or other mutants in the adhE transcriptional regulatory pathway, mainly to increase adhE expression under the desired conditions (e.g., anaerobic, aerobic), either alone or in combination with exogenous polynucleotides that encode adhE.

Moreover, to further enhance ethanol production, the pdc-adhA/B-adhE operon containing recombinant microorganisms can be combined with any of the other systems or methods provided herein (e.g., tether system vectors, deletion mutants such as ΔldhA, Δfrd, ΔpflB, ΔfocA, fadR, etc.) to optimize the production of ethanol from biomass. For instance, such recombinant microorganisms may comprise one or more exogenous polynucleotides that express pdc, adhA/B, and adhE, and further comprise one or more tether systems, one or more functional deletions in the ΔldhA, Δfrd, ΔpflB, ΔfocA genes (in any combination thereof), or both. Hence, embodiments of the present invention include the use of such recombinant microorganisms to optimize the production of ethanol from biomass or other biomolecule.

In certain embodiments, the pdc-adhA/B-adhE containing recombinant microorganisms, including those already combined with the tether system vectors (e.g., secreted or surface-displayed lyases, cellulases, laminarinases, lipases) and/or deletion mutants (e.g., ΔldhA, Δfrd, ΔpflB, ΔfocA), may be combined with the various improved methods of metabolizing alginate, pectin, cellulose, cellobiose, hydroxymethylcellulose, guluronate, mannitol, lipids etc., to optimize the production of ethanol from biomass, including biomass that contains both polysaccharides and lipids. In this regard, recombinant microorganisms that comprise at least one of pALG1.0, pALG1.5 pALG2.0, pALG2,5, pALG3.0, pALG3.5, pALG4.0, pALG 5.0, pALG5.1, pALG5.2, pALG5.3, pALG 6.0, pALG7.0, pALG7.2, pALG 7.3, etc. or their functional equivalents (e.g., vectors that contain functionally related genes), may be modified to contain one or more polynucleotides that express pdc, adhA/B, and adhE. In certain embodiments, these recombinant microorganisms may be further modified to contain one or more tether systems, one or more functional deletions in the ΔldhA, Δfrd, ΔpflB, ΔfocA genes (in any combination thereof), or both. These recombinant microorganisms may also be grown in the presence of defined ratios of mixed sugars (e.g., optimized mannitol:alginate ratios; see Example 6) or fatty acids to further optimize their production capacity. Accordingly, embodiments of the present invention include the use of such recombinant microorganisms to optimize the production of ethanol from biomass or other biomolecule. In certain embodiments, the biomass is kelp. In certain preferred embodiments, the kelp comprises Laminaria japonicum or Macrocystis pyrifera. In certain preferred embodiments, the recombinant microorganism is E. coli.

In certain embodiments, the use of pdc-adhA/B-adhE containing recombinant microorganisms, alone or in combination with the other methods provided herein, may enhance the yield of ethanol (e.g., commodity chemical) to at least about 30%, 35%, 40%, 45%, 50%, 55%, 60%, 65%, 70%, 75%, 80%, 85%, 88%, 90%, 91%, 92%, 93%, 94%, 95%, 96%, 97%, 98%, 99%, or 100% of a theoretical maximum yield. In certain embodiments, the method may be characterized by increasing the percentage of the theoretical maximum yield of ethanol by at least about 10% (e.g., from about 30% to about 40% of the theoretical maximum yield), 15% (e.g., from about 30% to about 45%, from about 50% to about 65%), 20%, 25%, 30%, 35%, 40%, 50%, 60%, 70%, 80%, or 90% as compared to incubating the same recombinant microorganism under control or different conditions, or as compared to incubating a control (e.g., unmodified or differently modified) microorganism under the same or similar conditions. In certain embodiments, the recombinant microorganisms and methods of use thereof may be used to approach, achieve, or even surpass the theoretical maximum yield of ethanol production from biomass such as kelp, such as by achieving a synergistic effect between the various components or technologies described herein.

Embodiments of the present invention are illustrated by the following non-limiting examples.

### EXAMPLES

### Example 1

### Surface Display and Autotransporter Proteins for Secretion and Tethering of Polysaccharide De-Polymerizing Enzymes

To improve the secretion of polysaccharide de-polymerizing enzymes, and thereby improve the metabolism of polysaccharides, various alginate lyases (AL) were fused to carrier proteins, expressed in E. coli, and incubated with alginate. Upon expression, as summarized below, the catalytic activity of the AL fusion polypeptides was associated with the conditioned media (i.e., fully secreted), the outer membrane (i.e., secreted and tethered) of the cells, or both; and the various AL-carrier combinations showed different ratios of fully secreted AL activity vs. tethered AL activity. Nonetheless, both the fully secreted and tethered ALs effectively de-polymerized alginate without the need to break open the cells.

Bacterial cells E. coli K12 (DH5α and DH10B) and E. coli W were transformed with vector DNA carrying various combinations of the following genetic and protein-coding elements: a promoter, a heterologous or native signal peptide, a carrier polypeptide, and an alginate lyase, i.e., a “passenger” polypeptide. The order of the elements in the vector was either: a) promoter-signal peptide-carrier-passenger, or b) promoter-signal peptide-passenger-carrier. Exemplary elements are provided in Table 1 below, and certain of the specific vectors are in Table 2 below.

TABLE 1
Exemplary elements of a fusion construct

| Element | Name | Organism of origin |
|---|---|---|
| Vectors | pTrc99a | NA |
| | pCCfos2 | NA |
| Promoters | P_{trc} | E. coli (modified) |
| | P_{pdc} | Zymomonas mobilis |
| | P_{H207} | Coliphage |
| | P_{D/E20} | Coliphage |
| | P_{F30} | Coliphage |
| | P_{H22} | Coliphage |
| | P_{G25} | Coliphage |
| | P_{J5} | Coliphage |
| | P_{N25} | Coliphage |
| | P_{L} | Phage lambda |
| | P_{A1} | Phage T5 |
| | P_{rrnB-2} | E. coli |
| | P_{LPP} | E. coli |
| Signal | P_{gsA} | Bacillus subtilis |
| peptides | LPP | E. coli |
| | Ag43 | E. coli |
| Carrier | Omp1 | Zymomonas mobilis |
| proteins | OmpA | E. coli |
| | PgsA | Bacillus subtilis |
| | Ag43 | E. coli |
| Passenger | Alginate lyase | Pseudoalteromonas sp. SM0524 |
| protein | Alginate lyase AI-I | Sphingomonas sp. A1 |
| | Alginate lyase AI-II | Sphingomonas sp. A1 |
| | Alginate lyase AI-III | Sphingomonas sp. A1 |

TABLE 2
Exemplar vector constructs

| Vector | Plasmid | | Signal | | |
|---|---|---|---|---|---|
| # | skeleton | Promoter | pep tide | Display | Enzyme |
| 331 | pTrc99a | pPDC | omp1 | omp1 | AI-I |
| 443 | pTrc99a | pPDC | LPP | OmpA | AI-I |
| 445 | pTrc99a | pPDC | Ag43 | Ag43 | SM0524 |
| | | | | | (pETΔPaAly) |
| 455 | pTrc99a | pPDC/ | Ag43/ | Ag43/ | SM0524 |
| | | pPDC | PgsA | PgsA | (pETΔPaAly)/ |
| | | | | | AI-I |

Note:
Vector #455 carries two fusion proteins in tandem

Preparation of Lpp-OmpA dsDNA: First, the E. coli ompA gene was amplified by PCR using the genomic DNA of E. coli strain DH10b as a template. The reaction mixture contained 1× Phusion buffer, 2 mM dNTP, 0.2 μM forward (5′-AACCCGTATGTTGGCTTTGAAATGG-3′) (SEQ ID NO:157) and reverse (5′-GTCCGGACGAGTGCCGATGG-3′) (SEQ ID NO:158) primers, 2.5 U Phusion DNA polymerase (Finezyme), and an aliquot of E. coli DH10b genomic DNA as a template in total volume of 100 μl. The thermocycler program was 98° C. for 10 sec, 70° C. for 10 sec, and 72° C. for 15 sec, repeated for 30 times. Second, the amplified fragment was assembled with oligonucleotides encoding the Lpp signal peptides and first N′ amino acids. The reaction mixture contained 1× Phusion buffer, 2 mM dNTP, 2 μM of each the following oligonucleotides 5′-ATGAAAGCTACTAAACTGGTACTGGGCGCGGTAATCCTGGGTTCT-3′ (SEQ ID NO:159); 5′-TGCTGGAGCAACCTGCCAGCAGAGTAGAACCCAGGATTAC-3′ (SEQ ID NO:160); 5′-ACTCTGCTGGCAGGTTGCTCCAGCAACGCTAAAATCGATCAG-3′ (SEQ ID NO:161) and 5′-ACCCATTTCAAAGCCAACATACGGGTTCTGATCGATTTTAGCGT-3′ (SEQ ID NO:162), 2.5 U Phusion DNA polymerase (Finezyme) and 1 ng of purified ompA PCR product. The thermocycler program was 98° C. for 10 sec, 65° C. for 10 sec, and 72° C. for 15 sec, repeated for 30 times. Third, a nest PCR reaction was performed to amplify the full length Lpp-ompA. The reaction mixture contained 1× Phusion buffer, 2 mM dNTP, 0.2 μM forward (5′-CACACACCATGGATGAAAGCTACTAAACTGGTACTGGGC-3′) (SEQ ID NO:606) and reverse (5′-CCCTTTGGATCCGTCCGGACGAGTGCCG-3′) (SEQ ID NO:607) primers, 2.5 U Phusion DNA polymerase (Finezyme) and 0.1 ul of the assembly reaction as a template. The thermocycler program was 98° C. for 10 sec, 66° C. for 10 sec, and 72° C. for 10 sec, repeated for 30 times.

Preparation of PgsA dsDNA: The Bacillus subtilis PgsA gene was amplified by PCR using the genomic DNA of B. subtilis as a template. The reaction mixture contained 1× Phusion buffer, 2 mM dNTP, 0.2 μM forward (5′-CACACACCATGGATGAAAAAAGAACTGAGCTTTCATGAAAAGC-3′) (SEQ ID NO:163) and reverse (5′-CCCTTTGGATCCTTTAGATTTTAGTTTGTCACTATGATCAATATCAAACG-3′) (SEQ ID NO:164) primers, 2.5 U Phusion DNA polymerase (Finezyme), and an aliquot of B. subtilis genomic DNA as a template in total volume of 50 μl. The thermocycler program was 98° C. for 10 sec, 68° C. for 10 sec, and 72° C. for 40 sec, repeated for 30 times.

Preparation of InaK dsDNA: The gene of Pseudomonas syringae ice nucleation protein (inaK) was assembled from oligonucleotides 1 to 21 shown in the list below. The reaction mixture contained 1× Phusion buffer, 2 mM dNTP, 1 μl of 100 μM oligonucleotides mix solution and 2.5 U Phusion DNA polymerase (Finezyme). The thermocycler program was 40 cycles of: 98° C. for 10 sec, 45° C. with increment of 0.2° C. per cycle for 10 sec and then ramping to 55° C. in a rate of 0.2° C. per sec and finally 72° C. for 15 sec. A nest PCR reaction was performed to amplify the assembled gene. The reaction mixture contained 1× Phusion buffer, 2 mM dNTP, 0.2 μM forward (5′-GGGCCCCCATGGATGACTCTCGACAAGGCGTTGG-3′) (SEQ ID NO:165) and reverse (5′-TTTAAAGGATCCGGTCTGCAAATTCTGCGGC-3′) (SEQ ID NO:166) primers, 2.5 U Phusion DNA polymerase (Finezyme) and 1 ul of the assembly reaction as a template in total volume of 50 μl. The thermocycler program was 98° C. for 10 sec, 62° C. for 10 sec, and 72° C. for 15 sec, repeated for 30 times.

Preparation of Ag43 dsDNA: The beta domain of ag43 (including 458 bases of the alpha domain upstream to the beta domain) and the signal peptide of ag43 (ag43-sp) were amplified by PCR using the genomic DNA of E. coli strain DH10B as a template. The reaction mixture contained 1× Phusion buffer, 2 mM dNTP, 0.2 μM forward (ag43: 5′-AGAGAGTCTAGAAGCGACGGAAAGGCATTCAGTATCG-3′ (SEQ ID NO:167) ag43-sp: 5′-AAAGGGCCATGGATGAAACGACATCTGAATACCTGC-3′ (SEQ ID NO:168)) and reverse (ag43: 5′-TTTGGGAAGCTTCAGAAGGTCACATTCAGTGTGGC-3′ (SEQ ID NO:169) ag43-sp: 5′-TGTGTGGGATCCAGCCAGCACCGGGAGTG-3′(SEQ ID NO:170)) primers, 2.5 U Phusion DNA polymerase (Finezyme), and an aliquot of E. coli DH10b genomic DNA as a template in total volume of 100 μl. The thermocycler programs were 98° C. for 10 sec, 68° C. for 10 sec, and 72° C. for 60 sec, repeated for 35 times for ag43 and 98° C. for 10 sec, 65° C. for 10 sec, and 72° C. for 6 sec, repeated for 30 times for ag43-sp.

Preparation of pTrc99a_phoAestA dsDNA: The Pseudomonas aeruginosa gene estA carrying the inactivating mutation S38A was PCR amplified from the genomic DNA of P. aeruginosa and assembled with oligonucleotides encoding the leader sequence of E. coli alkaline phosphatase phoA. The estA amplification reaction contained 1× Phusion GC buffer, 20% Q solution (QIAGEN), 2 mM dNTP, 0.2 μM forward (Oligo #1 in the list below) and reverse (Oligo 2) primers, 2.5 U Phusion DNA polymerase (Finezyme), and an aliquot of P. aeruginosa genomic DNA as a template in total volume of 50 μl. The thermocycler programs were 98° C. for 10 sec, 64° C. for 10 sec, and 72° C. for 60 sec, repeated for 35 times. The purified PCR product was mixed with oligonucleotides 3-9 and assembled. The reaction mixture contained 1× Phusion buffer, 2 mM dNTP, 1 μl of 100 μM oligonucleotides and PCR product mix solution and 2.5 U Phusion DNA polymerase (Finezyme). The thermocycler program was 40 cycles of: 98° C. for 10 sec, 45° C. with increment of 0.2° C. per cycle for 10 sec and then ramping to 55° C. in a rate of 0.2° C. per sec and finally 72° C. for 15 sec. A nest PCR reaction was performed to amplify the assembled gene. The reaction mixture contained 1× Phusion buffer, 2 mM dNTP, 0.2 μM forward (5′-ATATATCCATGGATGAAACAAAGCACTATTGCACTGGC-3′) (SEQ ID NO:171) and reverse (5′-CCCTTTAAGCTTTCAGAAGTCCAGGCTCAGCG-3′) (SEQ ID NO:172) primers, 2.5 U Phusion DNA polymerase (Finezyme) and 1 ul of the assembly reaction diluted 1:1000 as a template in total volume of 50 μl. The thermocycler program was 98° C. for 10 sec, 62° C. for 10 sec, and 72° C. for 60 sec, repeated for 30 times. The PCR product was cloned into the Nco-I and HindIII of pTrc99a to form pTrc99a_phoAestA.

Preparation of Alginate Lyases AI-I, ΔAI-I, AI-II and AI-III dsDNA: The gene of alginate lyase AI-I was assembled from the oligonucleotides shown in Table 3 below. Step 1: The 5′ and the 3′ fragments of the gene were assembled separately. The 5′ fragment was assembled from oligos 1 to 22 and 56 to 76. The 3′ fragment was assembled from oligos 19 to 59. For each assembly reaction a 100 μM oligonucleotides stock solution was made by mixing equal amounts of each oligonucleotide. The reaction mixture contained 1× Phusion buffer, 2 mM dNTP, 1 μl of 100 μM oligonucleotides stock solution and 2.5 U Phusion DNA polymerase (Finezyme). The thermocycler program was 40 cycles of: 98° C. for 10 sec, 45° C. with increment of 0.2° C. per cycle for 10 sec and then ramping to 55° C. in a rate of 0.2° C. per sec and finally 72° C. for 15 sec. Step 2: A nest PCR reaction was performed to amplify the assembled 5′ and the 3′ fragments of AI-I. The reaction mixture contained 1× Phusion buffer, 2 mM dNTP, 0.2 μM forward (5′ fragment: oligo 77; 3′ fragment: oligo 80) and reverse (5′ fragment: oligo 79; 3′ fragment: oligo 78) primers, 2.5 U Phusion DNA polymerase (Finezyme) and 1 ul of the assembly reaction as a template in total volume of 50 μl. The thermocycler program was 98° C. for 10 sec, 62° C. for 10 sec, and 72° C. for 15 sec, repeated for 30 times. Step 3: The PCR products of step 2 were gel purified and assembled to create the full length AI-I gene. The reaction mixture contained 5 ul (˜100 ng) of each fragment 1× Phusion buffer, 2 mM dNTP, 2.5 U Phusion DNA polymerase (Finezyme) in total volume of 50 μl. The thermocycler program was 40 cycles of: 98° C. for 10 sec, 45° C. with increment of 0.2° C. per cycle for 10 sec and then ramping to 55° C. in a rate of 0.2° C. per sec and finally 72° C. for 30 sec. Step 4: A nest PCR reaction was performed to amplify the assembled full length AI-I gene. The reaction mixture contained 1× Phusion buffer, 2 mM dNTP, 0.2 μM forward (oligo 77) and reverse (oligo 78) primers, 2.5 U Phusion DNA polymerase (Finezyme) and 1 ul of the assembly reaction as a template in total volume of 50 μl. The thermocycler program was 98° C. for 10 sec, 63° C. for 10 sec, and 72° C. for 30 sec, repeated for 30 times. Step 5: Cloning of AI-I gene into pCR8 plasmid. The PCR product of step 4 was gel purified and cloned using pCR8/GW/TOPO TA Cloning Kit (Invitrogen) according to the manufacturer instructions. The resulting plasmid, pCR8-AI-I, was sequence verified.

TABLE 3
Oligos for assembling Alginate lyases AI-I, ΔAI-I,
AI-II and AI-III

| | | SEQ |
|---|---|---|
| | | ID |
| Oligo # | Sequence | NO: |
| 1 | ATGCCTCTGGCTTGTCTGGCTACTACTCGTGTTGGTGCTGCTCGTGAGAA | 173 |
| 2 | AAGCGGCGACTCTTCTATGTTCGACATCCCGTTTCCGGGTCACGGTCGTC | 174 |
| 3 | GTCTGGCCGTTGCGGCGCTGGCCTTCGCCGGTTGCGCGTTCGCAGGTTCT | 175 |
| 4 | CTGCAAGCTCACCCGTTCGACCAAGCAGTTGTGAAAGATCCGACTGCGTC | 176 |
| 5 | CTATGTTGACGTTAAAGCGCGTCGTACTTTCCTGCAAAGCGGTCAACTGG | 177 |
| 6 | ATGATCGCCTGAAAGCAGCGCTGCCGAAGGAATATGACTGTACCACCGAA | 178 |
| 7 | GCGACGCCGAACCCACAGCAGGGTGAAATGGTGATCCCACGCCGCTATCT | 179 |
| 8 | GTCCGGTAACCACGGCCCGGTGAATCCGGATTACGAGCCGGTTGTCACTC | 180 |
| 9 | TGTATCGCGACTTCGAAAAAATCAGCGCGACCCTGGGTAACCTGTACGTT | 181 |
| 10 | GCGACTGGTAAACCAGTGTACGCAACTTGTCTGCTGAACATGCTGGACAA | 182 |
| 11 | ATGGGCTAAAGCAGACGCGCTGCTGAACTATGACCCGAAATCTCAGAGCT | 183 |
| 12 | GGTATCAAGTAGAATGGTCCGCAGCCACGGCGGCCTTTGCCCTGAGCACT | 184 |
| 13 | ATGATGGCAGAGCCGAACGTGGACACCGCGCAGCGTGAGCGTGTTGTGAA | 185 |
| 14 | ATGGCTGAACCGTGTAGCACGTCACCAGACTTCTTTTCCGGGTGGCGACA | 186 |
| 15 | CTAGCTGCTGTAACAATCATTCTTACTGGCGTGGTCAGGAGGCTACCATC | 187 |
| 16 | ATCGGCGTTATTTCCAAGGATGATGAACTGTTCCGTTGGGGTCTGGGTCG | 188 |
| 17 | TTATGTACAGGCGATGGGTCTGATCAACGAAGATGGTTCCTTCGTTCACG | 189 |
| 18 | AAATGACTCGTCACGAACAGAGCCTGCATTATCAGAACTATGCGATGCTG | 190 |
| 19 | CCGCTGACCATGATCGCTGAGACTGCCTCTCGTCAGGGTATCGATCTGTA | 191 |
| 20 | TGCTTACAAGGAAAACGGTCGTGATATCCATTCTGCTCGTAAATTCGTAT | 192 |
| 21 | TCGCGGCCGTAAAGAATCCGGATCTGATCAAGAAATACGCGAGCGAACCG | 193 |
| 22 | CAGGACACGCGCGCTTTTAAACCGGGTCGCGGCGATCTGAACTGGATCGA | 194 |
| 23 | ATATCAGCGTGCGCGTTTCGGCTTTGCAGATGAGCTGGGCTTTATGACCG | 195 |
| 24 | TGCCAATCTTCGATCCGCGCACCGGCGGCTCTGGCACTCTGCTGGCGTAT | 196 |
| 25 | AAGCCACAGGGTGCGGCTGCTCAGGCGCCGGTTTCCGCTCCGGCGGCAGC | 197 |
| 26 | ACACTCTTCCATCGATCTGTCCAAATGGAAACTGCAGATCCCTGTTGACC | 198 |
| 27 | CGATCGATGTTGCTACCCGCGATCTGCTGAAGGGTTATCAGGACAAGTAT | 199 |
| 28 | TTCTACGTGGATAAAGATGGTTCTCTGGCCTTCTGGTGCCCAGCATCCGG | 200 |
| 29 | TTTCAAAACCACGGCGAATACTAAGTATCCGCGTAGCGAGCTGCGTGAAA | 201 |
| 30 | TGCTGGACCCGGATAATCATGCTGTTAATTGGGGCTGGCAGGGCACCCAC | 202 |
| 31 | GAAATGAACCTGCGCGGTGCAGTTATGCACGTTTCCCCGTCCGGTAAAAC | 203 |
| 32 | CATCGTCATGCAGATCCACGCAGTTATGCCGGACGGTTCCAATGCGCCAC | 204 |
| 33 | CACTGGTTAAAGGCCAGTTCTACAAAAACACGCTGGACTTCCTGGTGAAA | 205 |
| 34 | AATTCTGCGGCTGGTGGTAAAGATACTCACTACGTGTTCGAAGGCATCGA | 206 |
| 35 | ACTGGGTAAACCATACGACGCTCAGATCAAAGTTGTAGATGGTGTCCTGT | 207 |
| 36 | CTATGACCGTTAATGGTCAGACTAAAACTGTTGACTTCGTGGCTAAAGAT | 208 |
| 37 | GCGGGCTGGAAGGATCTGAAATTCTATTTCAAGGCAGGTAACTATCTGCA | 209 |
| 38 | GGACCGCCAGGCCGACGGCTCCGATACCTCTGCCCTGGTAAAGCTGTACA | 210 |
| 39 | GCTGGAATGTTTAACGTCCAGTTTGTACAGCTTTACCAGGGCAGAGGT | 211 |
| 40 | ATCGGAGCCGTCGGCCTGGCGGTCCTGCAGATAGTTACCTGCCTTGAAAT | 212 |
| 41 | AGAATTTCAGATCCTTCCAGCCCGCATCTTTAGCCACGAAGTCAACAGTT | 213 |
| 42 | TTAGTCTGACCATTAACGGTCATAGACAGGACACCATCTACAACTTTGAT | 214 |
| 43 | CTGAGCGTCGTATGGTTTACCCAGTTCGATGCCTTCGAACACGTAGTGAG | 215 |
| 44 | TATCTTTACCACCAGCCGCAGAATTTTTCACCAGGAAGTCCAGCGTGTTT | 216 |
| 45 | TTGTAGAACTGGCCTTTAACCAGTGGTGGCGCATTGGAACCGTCCGGCAT | 217 |
| 46 | AACTGCGTGGATCTGCATGACGATGGTTTTACCGGACGGGGAAACGTGCA | 218 |
| 47 | TAACTGCACCGCGCAGGTTCATTTCGTGGGTGCCCTGCCAGCCCCAATTA | 219 |
| 48 | ACAGCATGATTATCCGGGTCCAGCATTTCACGCAGCTCGCTACGCGGATA | 220 |
| 49 | CTTAGTATTCGCCGTGGTTTTGAAACCGGATGCTGGGCACCAGAAGGCCA | 221 |
| 50 | GAGAACCATCTTTATCCACGTAGAAATACTTGTCCTGATAACCCTTCAGC | 222 |
| 51 | AGATCGCGGGTAGCAACATCGATCGGGTCAACAGGGATCTGCAGTTTCCA | 223 |
| 52 | TTTGGACAGATCGATGGAAGAGTGTGCTGCCGCCGGAGCGGAAACCGGCG | 224 |
| 53 | CCTGAGCAGCCGCACCCTGTGGCTTATACGCCAGCAGAGTGCCAGAGCCG | 225 |
| 54 | CCGGTGCGCGGATCGAAGATTGGCACGGTCATAAAGCCCAGCTCATCTGC | 226 |
| 55 | AAAGCCGAAACGCGCACGCTGATATTCGATCCAGTTCAGATCGCCGCGAC | 227 |
| 56 | CCGGTTTAAAAGCGCGCGTGTCCTGCGGTTCGCTCGCGTATTTCTTGATC | 228 |
| 57 | AGATCCGGATTCTTTACGGCCGCGAATACGAATTTACGAGCAGAATGGAT | 229 |
| 58 | ATCACGACCGTTTTCCTTGTAAGCATACAGATCGATACCCTGACGAGAGG | 230 |
| 59 | CAGTCTCAGCGATCATGGTCAGCGGCAGCATCGCATAGTTCTGATAATGC | 231 |
| 60 | AGGCTCTGTTCGTGACGAGTCATTTCGTGAACGAAGGAACCATCTTCGTT | 232 |
| 61 | GATCAGACCCATCGCCTGTACATAACGACCCAGACCCCAACGGAACAGTT | 233 |
| 62 | CATCATCCTTGGAAATAACGCCGATGATGGTAGCCTCCTGACCACGCCAG | 234 |
| 63 | TAAGAATGATTGTTACAGCAGCTAGTGTCGCCACCCGGAAAAGAAGTCTG | 235 |
| 64 | GTGACGTGCTACACGGTTCAGCCATTTCACAACACGCTCACGCTGCGCGG | 236 |
| 65 | TGTCCACGTTCGGCTCTGCCATCATAGTGCTCAGGGCAAAGGCCGCCGTG | 237 |
| 66 | GCTGCGGACCATTCTACTTGATACCAGCTCTGAGATTTCGGGTCATAGTT | 238 |
| 67 | CAGCAGCGCGTCTGCTTTAGCCCATTTGTCCAGCATGTTCAGCAGACAAG | 239 |
| 68 | TTGCGTACACTGGTTTACCAGTCGCAACGTACAGGTTACCCAGGGTCGCG | 240 |
| 69 | CTGATTTTTTCGAAGTCGCGATACAGAGTGACAACCGGCTCGTAATCCGG | 241 |
| 70 | ATTCACCGGGCCGTGGTTACCGGACAGATAGCGGCGTGGGATCACCATTT | 242 |
| 71 | CACCCTGCTGTGGGTTCGGCGTCGCTTCGGTGGTACAGTCATATTCCTTC | 243 |
| 72 | GGCAGCGCTGCTTTCAGGCGATCATCCAGTTGACCGCTTTGCAGGAAAGT | 244 |
| 73 | ACGACGCGCTTTAACGTCAACATAGGACGCAGTCGGATCTTTCACAACTG | 245 |
| 74 | CTTGGTCGAACGGGTGAGCTTGCAGAGAACCTGCGAACGCGCAACCGGCG | 246 |
| 75 | AAGGCCAGCGCCGCAACGGCCAGACGACGACCGTGACCCGGAAACGGGAT | 247 |
| 76 | GTCGAACATAGAAGAGTCGCCGCTTTTCTCACGAGCAGCACCAACACGAG | 248 |
| 77 | GTGTGTGGATCCATGCCTCTGGCTTGTCTGGC | 249 |
| 78 | GGGTTTAAGCTTAGCTGGAATGTTTAACGTCCAGTTTGTAC | 250 |
| 79 | GCGTGTCCTGCGGTTCGC | 251 |
| 80 | GCTGAGACTGCCTCTCGTCAGGG | 252 |

Preparation of Alginate lyases from Pseudoalteromonas sp. SM0524: The gene of alginate lyase was assembled from the oligonucleotides shown in Table 4 below. For assembly reaction, a 100 μM oligonucleotides stock solution was made by mixing equal amounts of each oligonucleotide. The reaction mixture contained 1× Phusion buffer, 2 mM dNTP, 1 μl of 100 μM oligonucleotides stock solution and 2.5 U Phusion DNA polymerase (Finezyme).alginate lyase. The reaction mixture contained 1× Phusion buffer, 2 mM dNTP, 0.2 μM forward (oligo 1) and reverse (oligo 50) primers, 2.5 U Phusion DNA polymerase (Finezyme) and 1 ul of the assembly reaction as a template in total volume of 50 μl. This PCR product was digested with BamHI/XbaI and ligated into pET29 vector (Novagene) predigested with BamHI/XbaI with T4 DNA Ligase (NEB). The resulting plasmid, pETPaAly, was sequence verified. To clone catalytic domain of PaAly by excluding internal HindIII site (ΔPaAly), The ΔPaAly DNA fragment was amplified by overlap-PCR: 98° C. for 15 sec, 55° C. for 15 sec, and 72° C. for 30 sec repeated 30 times. The reaction mixture contained 1× Phusion buffer, 2 mM dNTP, 0.5 μM forward (5′-CGCGGATCCGATAACTCAAATGGTTCAAC-3′ (SEQ ID NO:253) for 5′ fragment and 5′-GTATAAGGTTAAAGAGAGCTTACGCGTTGCTATG-3′ (SEQ ID NO:254) for 3′-fragment) and reverse primers (5′-CATAGCAACGCGTAAGCTCTCTTTAACCTTATAC-3′ (SEQ ID NO:255) for 5′ fragment and 5′-CCCAAGCTTTTAATTAGTTTCACGCGTATAAC-3′ (SEQ ID NO:256) for 3′-fragment), and 2.5 U Phusion DNA polymerase (Finezyme). Each amplified DNA fragment was gel purified and eluted into 30 ul of Elution buffer (QIAGEN). These amplified fragments were spliced by PCR: 98° C. for 10 sec, 55° C. for 15 sec, and 72° C. for 1 min, repeated for 30 times. The reaction mixture contained 1× Phusion buffer, 2 mM dNTP, 0.5 μM forward (5′-CGCGGATCCGATAACTCAAATGGTTCAAC-3′) (SEQ ID NO:257) and reverse (5′-CCCAAGCTTTTAATTAGTTTCACGCGTATAAC-3′) (SEQ ID NO:258) primers, 5 ul of each purified DNA fragment, and 2.5 U Phusion DNA polymerase (Finezyme) in total volume of 50 μl. The amplified fragment was then digested with BamHI/HindIII (New England Biolabs) and ligated with T4 DNA ligase into pET29 predigested with the same restriction enzymes to form pETΔPaAly.

TABLE 4
Oligos for assembling Pseudoalteromonas sp. SM0524

| | | SEQ |
|---|---|---|
| | | ID |
| Oligo # | Sequence 5′ → 3′ | NO: |
| 1 | CGGGATCCATGTTCAGGTTTAAAGG | 259 |
| 2 | AATAAGGATAATGATTAACCATAAAAAACTGTTTATTTACAGCGCAATTG | 260 |
| 3 | CGACAAGTTCAGCGCTATCTCATGCTGCAACAATTAATAATGCAGGCTTT | 261 |
| 4 | GAAAGTGGCTTTAGTAACTGGAACGAAACCGACCCAGCCGCTATTTCTTC | 262 |
| 5 | AGATGCTTACAGTGGCTCAAAATCGTTAAAAATTCAGGGCAGTCCAGCAC | 263 |
| 6 | GGGTTTATCAAGTGGTAGATATACAGCCTAACACTGAATACACCCTAAGT | 264 |
| 7 | GCTTATGTGCTGGGTAAAGGGCAAATTGGTGTAAACGATTTAAATGGTTT | 265 |
| 8 | ATTTAAAAACCAAACCTTTAATGTTTCTTCGTGGACTAAAGTAACAAAAA | 266 |
| 9 | CATTTACCTCAGCAAACACCAATTCACTTCAGGTTTTTGCTAAACATTAC | 267 |
| 10 | GACAACACCAGCGATGTAAGGTTTGATAATTTTTCCTTGATTGAGGGCAG | 268 |
| 11 | CGGTAGTAATGATGGTGGCTCAGATGGCGGCAGCGATAACTCAAATGGTT | 269 |
| 12 | CAACAATTCCTAGCAGCATAACCAGTGGTAGCATTTTTGATTTAGAAGGG | 270 |
| 13 | GATAACCCAAATCCTCTCGTTGACGATAGCACCTTAGTGTTTGTGCCGTT | 271 |
| 14 | AGGGGCACAACATATTACGCCTAATGGTAATGGCTGGCGTCATGAGTATA | 272 |
| 15 | AGGTTAAAGAAAGTTTACGCGTTGCTATGACTCAAACCTATGAAGTGTTC | 273 |
| 16 | GAAGCTACGGTAAAAGTTGAGATGTCTGATGGCGGAAAAACAATTATATC | 274 |
| 17 | GCAGCACCATGCTAGTGATACCGGCACTATATCTAAAGTGTATGTGTCGG | 275 |
| 18 | ATACTGATGAATCGGGCTTTAATGATAGCGTAGCGAACAACGGGATTTTT | 276 |
| 19 | GATGTGTACGTACGTTTACGTAATACCAGCGGTAATGAAGAAAAATTTGC | 277 |
| 20 | TTTGGGTACAATGACCAGCGGTGAGACATTTAACTTGCGGGTAGTTAATA | 278 |
| 21 | ACTACGGCGATGTAGAGGTTACGGCATTCGGTAACTCGTTCGGTATACCG | 279 |
| 22 | GTAGAGGATGATTCGCAGTCATACTTTAAGTTTGGTAACTACCTGCAATC | 280 |
| 23 | GCAAGACCCGTACACATTAGATAAATGTGGTGAGGCCGGAAACTCTAACT | 281 |
| 24 | CGTTTAAAAACTGTTTTGAGGATTTAGGCATTACAGAGTCAAAAGTGACG | 282 |
| 25 | ATGACCAATGTGAGTTATACGCGTGAAACTAATTAAGCTTGGTCTAGAGC | 283 |
| 26 | TTTATGGTTAATCATTATCCTTATTCCTTTAAACCTGAACATGGATCCCG | 294 |
| 27 | GCATGAGATAGCGCTGAACTTGTCGCAATTGCGCTGTAAATAAACAGTTT | 285 |
| 28 | CGTTCCAGTTACTAAAGCCACTTTCAAAGCCTGCATTATTAATTGTTGCA | 286 |
| 29 | CGATTTTGAGCCACTGTAAGCATCTGAAGAAATAGCGGCTGGGTCGGTTT | 287 |
| 30 | TGTATATCTACCACTTGATAAACCCGTGCTGGACTGCCCTGAATTTTTAA | 288 |
| 31 | TTTGCCCTTTACCCAGCACATAAGCACTTAGGGTGTATTCAGTGTTAGGC | 289 |
| 32 | AACATTAAAGGTTTGGTTTTTAAATAAACCATTTAAATCGTTTACACCAA | 290 |
| 33 | GAATTGGTGTTTGCTGAGGTAAATGTTTTTGTTACTTTAGTCCACGAAGA | 291 |
| 34 | CAAACCTTACATCGCTGGTGTTGTCGTAATGTTTAGCAAAAACCTGAAGT | 292 |
| 35 | ATCTGAGCCACCATCATTACTACCGCTGCCCTCAATCAAGGAAAAATTAT | 293 |
| 36 | CTGGTTATGCTGCTAGGAATTGTTGAACCATTTGAGTTATCGCTGCCGCC | 294 |
| 37 | CGTCAACGAGAGGATTTGGGTTATCCCCTTCTAAATCAAAAATGCTACCA | 295 |
| 38 | ATTAGGCGTAATATGTTGTGCCCCTAACGGCACAAACACTAAGGTGCTAT | 296 |
| 39 | GCAACGCGTAAACTTTCTTTAACCTTATACTCATGACGCCAGCCATTACC | 297 |
| 40 | ACATCTCAACTTTTACCGTAGCTTCGAACACTTCATAGGTTTGAGTCATA | 298 |
| 41 | GCCGGTATCACTAGCATGGTGCTGCGATATAATTGTTTTTCCGCCATCAG | 299 |
| 42 | TCATTAAAGCCCGATTCATCAGTATCCGACACATACACTTTAGATATAGT | 300 |
| 43 | TATTACGTAAACGTACGTACACATCAAAAATCCCGTTGTTCGCTACGCTA | 301 |
| 44 | CTCACCGCTGGTCATTGTACCCAAAGCAAATTTTTCTTCATTACCGCTGG | 302 |
| 45 | GCCGTAACCTCTACATCGCCGTAGTTATTAACTACCCGCAAGTTAAATGT | 303 |
| 46 | AGTATGACTGCGAATCATCCTCTACCGGTATACCGAACGAGTTACCGAAT | 304 |
| 47 | TTTATCTAATGTGTACGGGTCTTGCGATTGCAGGTAGTTACCAAACTTAA | 305 |
| 48 | AAATCCTCAAAACAGTTTTTAAACGAGTTAGAGTTTCCGGCCTCACCACA | 306 |
| 49 | CACGCGTATAACTCACATTGGTCATCGTCACTTTTGACTCTGTAATGCCT | 307 |
| 50 | GCTCTAGACCAAGCTTAATTAGTTT | 308 |

Preparation of inaV from Pseudomonas syringae INA5: The gene of alginate lyase was assembled from the oligonucleotides shown in the list below. For assembly reaction, a 100 μM oligonucleotides stock solution was made by mixing equal amounts of each oligonucleotide. The reaction mixture contained 1× Phusion buffer, 2 mM dNTP, 1 μl of 100 μM oligonucleotides stock solution and 2.5 U Phusion DNA polymerase (Finezyme). The thermocycler program was 40 cycles of: 98° C. for 10 sec, 45° C. with increment of 0.2° C. per cycle for 10 sec and then ramping to 55° C. in a rate of 0.2° C. per sec and finally 72° C. for 15 sec. A nest PCR reaction was performed to amplify the assembled alginate lyase. The reaction mixture contained 1× Phusion buffer, 2 mM dNTP, 0.2 μM forward (oligo 1) and reverse (oligo 38) primers, 2.5 U Phusion DNA polymerase (Finezyme) and 1 ul of the assembly reaction as a template in total volume of 50 μl. The thermocycler program was 98° C. for 10 sec, 62° C. for 10 sec, and 72° C. for 15 sec, repeated for 30 times. This PCR product was digested with BamHI/XbaI and ligated into pET29 vector (Novagene) predigested with BamHI/XbaI with T4 DNA Ligase (NEB). The resulting plasmid, pTrcinaV, was sequence verified.

TABLE 5
Oligos for assembling inaV from Pseudomonas syringae INA5

| | | SEQ |
|---|---|---|
| | | ID |
| Oligo # | Sequence 5′ → 3′ | NO: |
| 1 | CGGGATCCATGAATATCGACAAAGC | 309 |
| 2 | GTTGGTACTGCGTACCTGTGCAAATAACATGGCCGATCATTGCGGCCTTA | 310 |
| 3 | TATGGCCCGCCTCCGGCACGGTGGAATCCAAATACTGGCAGTCAACCAGG | 311 |
| 4 | CGGCATGAGAATGGTCTGGTCGGTTTACTGTGGGGCGCTGGAACCAGCGC | 312 |
| 5 | TTTTCTAAGCGTGCATGCCGATGCGCGATGGAAAGTCTGTGAAGTCGCCG | 313 |
| 6 | TTGCAGACATCATCGGTCTGGAAGAGCCGGGGATGGTCAAGTTTCCGCGG | 314 |
| 7 | GCCGAGGTGGTTCATGTCGGCGACAGGATCAGCGCATCACACTTCATTTC | 315 |
| 8 | GGCACGTCAGGCCGACCCTGCATCAACGCCAACGCCAACGCCAACGCCAA | 316 |
| 9 | TGGCCACGCCCACGCCTGCGGCAGCAAATATCGCGTTACCGGTGGTAGAA | 317 |
| 10 | CAGCCCAGTCATGAAGTGTTCGATGTGGCGTTGGTCAGCGCAGCTGCCCC | 318 |
| 11 | CTCAGTAAACACCCTGCCGGTGACGACGCCGCAGAATTTGCAGACCGCTA | 319 |
| 12 | CTTACGGCAGCACGTTGAGTGGCGACAACAACAGCCGGCTCATTGCCGGT | 320 |
| 13 | TATGGCAGTAACGAGACCGCTGGCAACCACAGTGATCTGATTGCCGGTAC | 321 |
| 14 | AGGCGGGCATGACTGCACGCTGATGGCGGGAGACCAAAGCAGATTGACCG | 322 |
| 15 | CAGGAAAGAACAGTATCTTGACGGCAGGCGCGCGTAGCAAACTTATTGGC | 323 |
| 16 | AGTGAAGGCTCGACGCTCTCGGCTGGAGAAGACTCAACGCTTATTTTCAG | 324 |
| 17 | GCTCTGGGACGGGAAAAGGTACAGGCAACTGGTTGCCAGAACGGGTGAGA | 325 |
| 18 | ACGGTGTTGAAGCCGACATACCGTATTACGTGAACGAAGATGACGATATT | 326 |
| 19 | GTCGATAAACCCGACGAGGACGATGACTGGATAGAGGTCGAGTCTAGAGC | 327 |
| 20 | ATTTGCACAGGTACGCAGTACCAACGCTTTGTCGATATTCATGGATCCCG | 328 |
| 21 | TCCACCGTGCCGGAGGCGGGCCATATAAGGCCGCAATGATCGGCCATGTT | 329 |
| 22 | AACCGACCAGACCATTCTCATGCCGCCTGGTTGACTGCCAGTATTTGGAT | 330 |
| 23 | CGCATCGGCATGCACGCTTAGAAAAGCGCTGGTTCCAGCGCCCCACAGTA | 331 |
| 24 | TCTTCCAGACCGATGATGTCTGCAACGGCGACTTCACAGACTTTCCATCG | 332 |
| 25 | TGTCGCCGACATGAACCACCTCGGCCCGCGGAAACTTGACCATCCCCGGC | 333 |
| 26 | TGATGCAGGGTCGGCCTGACGTGCCGAAATGAAGTGTGATGCGCTGATCC | 334 |
| 27 | GCTGCCGCAGGCGTGGGCGTGGCCATTGGCGTTGGCGTTGGCGTTGGCGT | 335 |
| 28 | CATCGAACACTTCATGACTGGGCTGTTCTACCACCGGTAACGCGATATTT | 336 |
| 29 | CGTCACCGGCAGGGTGTTTACTGAGGGGGCAGCTGCGCTGACCAACGCCA | 337 |
| 30 | TCGCCACTCAACGTGCTGCCGTAAGTAGCGGTCTGCAAATTCTGCGGCGT | 338 |
| 31 | TGCCAGCGGTCTCGTTACTGCCATAACCGGCAATGAGCCGGCTGTTGTTG | 339 |
| 32 | CATCAGCGTGCAGTCATGCCCGCCTGTACCGGCAATCAGATCACTGTGGT | 340 |
| 33 | GCCGTCAAGATACTGTTCTTTCCTGCGGTCAATCTGCTTTGGTCTCCCGC | 341 |
| 34 | CAGCCGAGAGCGTCGAGCCTTCACTGCCAATAAGTTTGCTACGCGCGCCT | 342 |
| 35 | CCTGTACCTTTTCCCGTCCCAGAGCCTGAAAATAAGCGTTGAGTCTTCTC | 343 |
| 36 | TACGGTATGTCGGCTTCAACACCGTTCTCACCCGTTCTGGCAACCAGTTG | 344 |
| 37 | CATCGTCCTCGTCGGGTTTATCGACAATATCGTCATCTTCGTTCACGTAA | 345 |
| 38 | GCTCTAGACTCGACCTCTATCCAGT | 346 |

Construction of pTrc99a_LppOmpA-Alginate lyase plasmids and of pTrc99a_PgsA-Alginate lyase plasmids. The dsDNA of Lpp-OmpA, of PgsA and of InaK described above was cloned into the NcoI and BamHI sites of pTrc99a, using standard procedures, to form pTrc99a_LppOmpA, pTrc99a_PgsA, and pTrc99a_InaK, respectively. The ligated plasmid was used to transform E. coli DH10 cells. The constructed plasmid was sequenced (Elim Biopharmaceuticals) and the DNA sequence of the insert was confirmed. The genes encoding alginate lyase genes were cloned to said plasmids downstream to the LppOmpA, PsgA, or InaK. Alginate lyases genes AI-I, AI-I dN′, AI-II and AI-III were PCR amplified using pCR8-AI-I. The reaction mixture contained 1× Phusion buffer, 2 mM dNTP, 0.2 μM forward (AI-I: 5′-GTGTGTGGATCCCCTCTGGCTTGTCTGGC-3′ (SEQ ID NO:347); ΔAI-I: 5′-GTGTGTGGATCCCACCCGTTCGACCAAGCAG-3′ (SEQ ID NO:348); AI-II: 5′-GGGTTTGGATCCGCTCCGGCGGCAGCAC-3′ (SEQ ID NO:349); AI-III: 5′-GTGTGTGGATCCCACCCGTTCGACCAAGCAG-3′) (SEQ ID NO:350) and reverse (AI-I, ΔAI-I, and AI-II: 5′-GGGTTTAAGCTTAGCTGGAATGTTTAACGTCCAGTTTGTAC-3′ (SEQ ID NO:351); AI-III: 5′-GGGTTTAAGCTTATGGCTTATACGCCAGCAGAGTG-3′) (SEQ ID NO:352) primers, 2.5 U Phusion DNA polymerase (Finezyme), and 1 ng of pCR8-AI-I plasmid DNA as a template, in total volume of 100 μl. The thermocycler programs were 98° C. for 10 sec, 65° C. for 10 sec, and 72° C. for 30 sec (AI-I and ΔAI-I) or 15 sec (AI-II and AI-III), repeated for 30 times. Similarly, the alginate lyase from Pseudoalteromonas sp. SM0524 was PCR amplified. The reaction mixture contained 1× Phusion buffer, 2 mM dNTP, 0.2 μM forward (5′-CGCGGATCCGATAACTCAAATGGTTCAAC-3′) (SEQ ID NO:353) and reverse (5′-GGGTTTAAGCTTAATTAGTTTCACGCGTATAACTCACATTGG-3′) (SEQ ID NO:354) primers, 2.5 U Phusion DNA polymerase (Finezyme), and pETΔPaAly as a template, in total volume of 100 μl. The thermocycler programs were 98° C. for 10 sec, 65° C. for 10 sec, and 72° C. for 15 sec repeated for 30 times. The PCR products of said alginate lyases were digested with BamHI and HindIII restriction enzymes and cloned into pTrc99a_LppOmpA, pTrc99a_PgsA, or pTrc99a_inaK plasmids digested by the same enzymes to form pTrc99a_LppOmpA-AI-I, pTrc99a_LppOmpA-ΔAI-I, pTrc99a_LppOmpA-AI-II, pTrc99a_LppOmpA-AI-III, pTrc99a_LppOmpA-ΔPaAly, pTrc99a_PgsA-AI-I, pTrc99a_PgsA-ΔAI-I, pTrc99a_PgsA-AI-II, pTrc99a_PgsA-AI-III, pTrc99a_PgsA-ΔPaAly, pTrc99a_InaK-AI-I, pTrc99a_InaK-ΔAI-I, pTrc99a_InaK-AI-II, pTrc99a_InaK-AI-III and pTrc99a_InaK-ΔPaAly respectively.

Construction of pTrc99a_Ag43-Alginate lyase and pTrc99a_phoAestA-Alginate lyase plasmids. The dsDNA of Ag43-SP and of Ag43 described above were cloned into the NcoI and BamHI sites and into the XbaI and HindIII sites, respectively, of pTrc99a using standard procedures, to form pTrc99a_Ag43. The ligated plasmid was used to transform E. coli DH10 cells. The constructed plasmid was sequenced (Elim Biopharmaceuticals) and the DNA sequence of the insert was confirmed. The genes encoding alginate lyase were cloned in pTr99a_ag43 and between the phoA leader sequence and estA in pTrc99a_phoAestA. Alginate lyases genes AI-I, ΔAI-I, AI-II and AI-III were PCR amplified using pCR8-AI-I as a template. The reaction mixture contained 1× Phusion buffer, 2 mM dNTP, 0.2 μM forward (AI-I: 5′-GTGTGTGGATCCCCTCTGGCTTGTCTGGC-3′ (SEQ ID NO:347); ΔAI-I: 5′-GTGTGTGGATCCCACCCGTTCGACCAAGCAG-3′ (SEQ ID NO:348); AI-II: 5′-GGGTTTGGATCCGCTCCGGCGGCAGCAC-3′ (SEQ ID NO:349); AI-III: 5′-GTGTGTGGATCCCACCCGTTCGACCAAGCAG-3′) (SEQ ID NO:350) and reverse (AI-I, AI-I dN′ and AI-II: 5′-TTTGGGTCTAGAGCTGGAATGTTTAACGTCCAGTTTGTAC-3′ (SEQ ID NO:355); AI-III dN′: 5′-TTTGGGTCTAGATGGCTTATACGCCAGCAGAGTG-3′) (SEQ ID NO:356) primers, 2.5 U Phusion DNA polymerase (Finezyme), and 1 ng of pCR8-AI-I plasmid DNA as a template, in total volume of 100 μl. The thermocycler programs were 98° C. for 10 sec, 65° C. for 10 sec, and 72° C. for 30 sec (AI-I and ΔAI-I) or 15 sec (AI-II and AI-III), repeated for 30 times. Similarly, the alginate lyase from Pseudoalteromonas sp. SM0524 was PCR amplified using pETΔPaAly as a template. The reaction mixture contained 1× Phusion buffer, 2 mM dNTP, 0.2 μM forward (5′-CGCGGATCCGATAACTCAAATGGTTCAAC-3′) (SEQ ID NO:353) and reverse (5′-TTTGGGTCTAGAATTAGTTTCACGCGTATAACTCACATTGG-3′) (SEQ ID NO:357) primers, 2.5 U Phusion DNA polymerase (Finezyme), and pETΔPaAly as a template, in total volume of 100 μl. The thermocycler programs were 98° C. for 10 sec, 65° C. for 10 sec, and 72° C. for 15 sec repeated for 30 times. The PCR products of said alginate lyases were digested with BamHI and XbaI restriction enzymes and cloned into pTrc99a_Ag43 or pTrc99a_phoAestA plasmids digested by the same enzymes to form pTrc99a_Ag43-AI-I, pTrc99a_Ag43-ΔAI-I, pTrc99a_Ag43-AI-II, pTrc99a_Ag43-AI-III and pTrc99a_Ag43-ΔPaAly and pTrc99a_phoAestA-AI-I, pTrc99a_phoAestA-ΔAI-I, pTrc99a_phoAestA-AI-II, pTrc99a_phoAestA-AI-III, and pTrc99a_phoAestA-ΔPaAly.

Construction of various promoter regions for alginate lyases display systems. The dsDNA of promoters P_{H207}, P_{D/E20}, P_{N25}, P_{L}, P_{A1} and P_{LPP} were assembled from the oligonucleotides shown in Table 6 below. For each assembly reaction a 100 μM oligonucleotide stock solution was made by mixing equal amounts of the corresponding oligonucleotide (P_{H207}: 1-4, P_{D/E20}: 5-8, P_{N25}: 9-12, P_{L}: 13-16, P_{A1}: 17-20, P_{LPP}: 21-24). The reaction mixture contained 1× Phusion buffer, 2 mM dNTP, 1 μl of 100 μM oligonucleotides stock solution and 2.5 U Phusion DNA polymerase (Finezyme). The thermocycler program was 35 cycles of: 98° C. for 10 sec, 60° C. for 10 sec and 72° C. for 3 sec. The Z. mobilis P_{pDC} was amplified using PCR. The reaction mixture contained 1× Phusion buffer, 2 mM dNTP, 0.2 μM forward and reverse primers (oligonucleotides 25 and 26 respectively), 2.5 U Phusion DNA polymerase (Finezyme), and genome of Zymomonas mobilis as a template in total volume of 50 μl. The thermocycler program was 98° C. for 10 sec, 64° C. for 10 sec, and 72° C. for 10 sec, repeated for 25 times. The E. coli P_{rrnB-2} was amplified using PCR. The reaction mixture contained 1× Phusion buffer, 2 mM dNTP, 0.2 μM forward and reverse primers (oligonucleotides 27 and 28 respectively), 2.5 U Phusion DNA polymerase (Finezyme), and 1 ng of E. coli DH10b genomic DNA as a template in total volume of 50 μl. The thermocycler program was 98° C. for 10 sec, 69° C. for 10 sec, and 72° C. for 10 sec, repeated for 35 times.

TABLE 6
Oligos for assembling promoters.

| | | SEQ |
|---|---|---|
| | | ID |
| Oligo # | Sequence | NO: |
| 1 | GGAAATTTGCATGCGAATTCTTTTAAAAAATTCATTTGCTAAACGCTTCAAATTC | 358 |
| 2 | CAATTTATGAAGTATATTATACGAGAATTTGAAGCGTTTAGCAAATGAATT | 359 |
| 3 | TCGTATAATATACTTCATAAATTGATAAACAAAAATCACACAGATATCA | 360 |
| 4 | ATATATCCATGGTTTCTCCTCTTTAATGATATCTGTGTGATTTTTGTTTAT | 361 |
| 5 | GGAAATTTGCATGCGAATTCAACTGCAAAAATAGTTTGACACCCTAGCCGATAG | 362 |
| 6 | GAACTGGGTACATCTTAAAGCCTATCGGCTAGGGTGTCAAACTATTTTTGC | 363 |
| 7 | GCTTTAAGATGTACCCAGTTCGATGAGAGCGATAACTCACACAGATATCA | 364 |
| 8 | ATATATCCATGGTTTCTCCTCTTTAATGATATCTGTGTGAGTTATCGCTCTCATC | 365 |
| 9 | GGAAATTTGCATGCGAATTCAAGAATCATAAAAAATTTATTTGCTTTCAG | 366 |
| 10 | GAATCTATTATACAGAAAAATTTTCCTGAAAGCAAATAAATTTTTTATG | 367 |
| 11 | GAAAATTTTTCTGTATAATAGATTCATAAATTTGAGAGAGGAGTTTCACAC | 368 |
| 12 | ATATATCCATGGTTTCTCCTCTTTAATGATATCTGTGTGAAACTCCTCTCTC | 369 |
| 13 | GGAAATTTGCATGCGAATTCTTATCTCTGGCGGTGTTGACATAAATACCACTGG | 370 |
| 14 | CGTCCTGCTGATGTGCTCAGTATCACCGCCAGTGGTATTTATGTCAACACCGC | 371 |
| 15 | CGGTGATACTGAGCACATCAGCAGGACGCACTGACCTCACACAGATATC | 372 |
| 16 | ATATATCCATGGTTTCTCCTCTTTAATGATATCTGTGTGAGGTCA | 373 |
| 17 | GGAAATTTGCATGCGAATTCTTATCAAAAAGAGTATTGACTTAAAGTCTAACC | 374 |
| 18 | CGATGGCTGTAAGTATCCTATAGGTTAGACTTTAAGTCAATACTCTTTTTG | 375 |
| 19 | TATAGGATACTTACAGCCATCGAGAGGGACACGGCGATCACACAGATATC | 376 |
| 20 | ATATATCCATGGTTTCTCCTCTTTAATGATATCTGTGTGATCGCCGTGTCCCTCT | 377 |
| 21 | GGAAATTTGCATGCGAATTCATCAAAAAAATATTGACAACATAAAAAAC | 378 |
| 22 | TGTAGCGTTACAAGTATAACACAAAGTTTTTTATGTTGTCAATATTTTTT | 379 |
| 23 | TTTGTGTTATACTTGTAACGCTACATGGAGATTAACTCAATCTAG | 380 |
| 24 | ATATATCCATGGTACCCTCTAGATTGAGTTAATCTCCA | 381 |
| 25 | CCCACCTGACCCCATGCCGAACTCCATGGAATTCGAGCTCGGTACCCTTTG | 382 |
| 26 | TTTAAACCATGGTTCTCCATATATTCAAAACACTATGTCTG | 383 |
| 27 | GGAAATTTGCATGCGAATTCCACGGAACAACGGCAAACAC | 384 |
| 28 | ATATATCCATGGTTTCTCCTCTTTAATGATATCTGTGTGAGCTTTTTCTCAGCGGCGC | 385 |

Construction of single copy plasmid for the display of alginate lyase. As a plasmid backbone we used the pCC1FOS vector, which contains both the E. coli F-factor single-copy origin of replication and the inducible high-copy oriV (Epicentre Biotechnologies). pCCfos2, a modified pCC1FOS was prepared by PCR. The reaction mixture contained 1× Phusion buffer, 2 mM dNTP, 0.2 μM forward (5′-TTTAAAGGATCCCTCGAGATATGCATGCCGGAAGCATAAAGTGTAAAGCCTG G-3′) (SEQ ID NO:386) and reverse (5′ATATATGGATCCCCGGGTACCGAGCTC-3′) (SEQ ID NO:387) primers, 2.5 U Phusion DNA polymerase (Finezyme), and an aliquot of pCC1FOS DNA as a template in total volume of 100 μl. The thermocycler program was 98° C. for 10 sec, 70° C. for 10 sec, and 72° C. for 120 sec, repeated for 30 times. The PCR product was digested by the restriction enzyme BamHI, ligated and transformed to E. coli EPI300 cells (Epicentre biotechnologies). Cloning of promoter and alginate lyase display fusion was done in two steps. First, a pTrc99a based AL display vector (as described above) was linearized by Nco-I restriction enzyme, dephosphorylated and ligated to a PCR product of a promoter (above), also digested by Nco-I. Second, the ligation product was PCR amplified. The reaction mixture contained 1× Phusion buffer, 2 mM dNTP, 0.2 μM of the corresponding forward primer (from the table above—P_{H207}: 1, P_{D/E20}: 5, P_{N25}: 9, P_{L}: 13, P_{A1}:17, P_{LPP}: 21, P_{pdc}: 25 P_{rrB-2}: 27) and reverse (5′-GCGCGCCCATGCGAGAGACTCGAGGGTTATTGTCTCATGAGCGG-3′) (SEQ ID NO:388) primers, 2.5 U Phusion DNA polymerase (Finezyme), and 0.1 μl of the ligation as a template in total volume of 50 μl. The PCR products were digested with EcoR-I and Xho-I restriction enzymes and ligated to pCCfos2 digested with the same enzymes. The single-copy plasmid harboring the alginate lyase of Pseudoalteromonas sp. SM0524 or ΔPaAly surface-display system under the control of P_{D/E20} was designated as BAL492.

Construction of single-copy plasmids with different promoters for the surface display of alginate lyase: BAL492 was used as a template to construct primers harboring different promoters (PF30, PH22, PG25, and PJ5) expressing the ΔPaAly surface-display system. By using the following primer sets, linear DNA fragments of BAL492 derivatives carrying these promoters were PCR amplified in a 100 μL reaction mixture containing 1× Phusion buffer, 0.25 mM dNTPs, 0.2 μM each of the forward and reverse primers, and 2.5 U Phusion DNA polymerase (Finezyme), The thermocycler was set as 94° C. for 30 s, 55° C. for 30 s, and 72° C. for 6 min for 30 cycles. The resulting PCR products were gel purified, treated with T4 polynucleotide kinase (New England BioLabs) and ligated using T4 DNA ligase (New England BioLabs) in a total volume of 10 μL following the manufacturer's protocol. 1 μL of each ligation was transformed into EPI300 cells (Epicentre biotechnologies). Plasmids from respective positive clones were then transformed into ATCC8739 cells to characterize their lyase activities.

TABLE 7
Oligomers for assembling different promoters

| | | SEQ |
|---|---|---|
| | | ID |
| Name | Sequence | NO: |
| PF30 | AAGTTTCTGTATAATTACTTTATAAATTGATGAGAAGGAAATCACACAGAT | 389 |
| fwd | ATCATTAAAGAGGAGAAACCCATGG | |
| PF30 | AAGTTTCTGTATAATTACTTTATAAATTGATGAGAAGGAAATCACACAGAT | 390 |
| fwd | ATCATTAAAGAGGAGAAACCCATGG | |
| PH22 | GCAATCGGTAAAATATCGATTTAGGCAGTTCACACAGATATCATTAAAGA | 391 |
| fwd | GGAGAAACCCATGG | |
| PH22 | TGGGCTATTGTCAACAATTTTTTAGTAGTCTGAGTGAATTCGCCCTATAGTG | 392 |
| fwd | AGTCGTATTACAATTCAC | |
| PG25 | CAATACTATTATAATATTGTTATTAAAGAGGAGAAATTAACATGAAACGAC | 393 |
| fwd | ATCTGAATACCTGCTACAGG | |
| PG25 | CAATACTATTATAATATTGTTATTAAAGAGGAGAAATTAACATGAAACGAC | 394 |
| fwd | ATCTGAATACCTGCTACAGG | |
| PJ5 | AATTTAGAATATACTGTTAGTAAACCTAATGGATCGACCTTTCACACAGAT | 395 |
| fwd | ATCATTAAAGAGGAGAAACCCATGG | |
| PJ5 | AATTTAGAATATACTGTTAGTAAACCTAATGGATCGACCTTTCACACAGAT | 396 |
| fwd | ATCATTAAAGAGGAGAAACCCATGG | |

Construction of single copy plasmid for the secretion of alginate lyase. BAL492 was used as a base vector for the construction, expression, and screening of an alginate lyase secretion system. In the development of such a system, the passenger domain of the autotransporter protein Ag43 was randomly truncated at different amino acid sites (namely, A53, Y91, Q121, L151, T181, A211, Q241, N271, G301, F331, A376, G384, N455, S495, S543, and P552—these sites represent the leading amino acids of the respective truncated segments), and the aspartyl protease active site was rationally included in all of them. Each truncated fragment (ΔAg43#) was PCR-amplified from the genome of DH10B using the respective forward and reverse primers listed below in a 100 μL reaction mixture containing 1× Phusion buffer, 0.2 mM dNTPs, 0.2 μM each of the forward and reverse primers, and 2.5 U Phusion DNA polymerase (Finezyme), The thermocycler was set as 94° C. for 30 s, 55° C. for 30 s, and 72° C. for 1-2 min for 30 cycles.

TABLE 8
Primers for assembling truncated Ag43 fragments

| | | SEQ |
|---|---|---|
| | | ID |
| Name | Sequence | NO: |
| Ag43-A53- | AAATCTAGA GCTGACATCGTTGTGCACCCGGGAG | 397 |
| fwd(XbaI) | | |
| Ag43-Y91- | AAATCTAGA TATGGGCCGGATAACGAGGCCAATA | 398 |
| fwd(XbaI) | | |
| Ag43-Q121- | AAATCTAGA CAGAGAGTGAACCCCGGTGGAAGT | 399 |
| fwd(XbaI) | | |
| Ag43-L151- | AAATCTAGA CTGAATGGTGGCGAACAGTGGATG | 400 |
| fwd(XbaI) | | |
| Ag43-T181- | AAATCTAGA ACAGTGGCAACGGATACCGTTGTTA | 401 |
| fwd(XbaI) | | |
| Ag43-A211- | AAATCTAGA GCCGTACGCACAACCATCAATAAAAACG | 402 |
| fwd(XbaI) | | |
| Ag43-Q241- | AAATCTAGA CAGACTGTACATGGTCACGCACTGG | 403 |
| fwd(XbaI) | | |
| Ag43-N271- | AAATCTAGA AACAGTGACGGCTGGCAGATTGTCA | 404 |
| fwd(XbaI) | | |
| Ag43-G301- | AAATCTAGA GGTACAGCCACGAATGTCACCCTGA | 405 |
| fwd(XbaI) | | |
| Ag43-F331- | AAATCTAGA TTCTCTGTTGTGGAGGGTAAAGCTGATAATGTCG | 406 |
| fwd(XbaI) | | |
| Ag43-A376- | AAATCTAGA GCCACCACCGTATCCATGGGAAATG | 407 |
| fwd(XbaI) | | |
| Ag43-apas- | AAATCTAGA GGCGGTGTACTGCTGGCCGATTC | 408 |
| N455-fwd(XbaI) | | |
| Ag43-apas-S495- | AAATCTAGAGGCGGTGTACTGCTGGCCGATTCCGGTGCCGCTGTCAGTG | 409 |
| fwd(XbaI) | GTACC AATAACGGCGCCATACTTACCCTTTCC | |
| Ag43-apas-S543- | AAATCTAGAGGCGGTGTACTGCTGGCCGATTCCGGTGCCGCTGTCAGTG | 410 |
| fwd(XbaI) | GTACC TCAGGAAGTGGCACACTCACTGTCA | |
| Ag43-apas-P552- | AAATCTAGAGGCGGTGTACTGCTGGCCGATTCCGGTGCCGCTGTCAGTG | 411 |
| fwd(XbaI) | GTACC AGCACTGTGCTGAACGGTGCCATTG | |
| Ag43b-rev(Hind | AGGAAGCTTCAGAAGGTCACATTCAGTGTGGCCT | 412 |
| III) | | |

Each ΔAg43# PCR product (insert) was digested with Xba I and Hind III, ligated into pTrc99a_Ag43-ΔPaAly (vector) using these restriction sites, and transformed into DH10B. This cloning fused each ΔAg43# fragment to the C terminus of Ag43-SP+ΔPaAly, resulting in the formation of fusion proteins consisting of Ag43-SP, ΔPaAly, and ΔAg43#. Plasmids from respective positive clones were then digested with BamH I and Hind III, from which each Ag43-SP+ΔPaAly+ΔAg43# fragment was gel-purified, ligated into BAL492 using these restriction sites, and transformed into EPI300 cells (Epicentre biotechnologies). Plasmids from respective positive clones were then transformed into ATCC8739 cells to screen for secreted lyase activities. The plasmid harboring the truncated autotransporter protein fragment (ΔAg43N455) that enabled secretion of ΔPaAly was named BAL998. Following similar cloning procedures as described above, single-copy plasmids containing ΔAg43N455 were made for other lyases for the construction of their secretion systems.

Construction of dual-enzyme expression plasmids harboring ΔPaAly: BAL492 was used as a base vector for the construction of dual-enzyme expression plasmids. A dual-enzyme expression plasmid harbors two independent alginate lyase surface-display or secretion systems, i.e., each lyase has its own signal peptide and autotransporter. An alginate lyase tether or secretion system was PCR-amplified from appropriate plasmids using the following forward and reverse primers and the PCR protocol described above.

TABLE 9
Primers for constructing dual-enzyme plasmids

| | | SEQ ID |
|---|---|---|
| Oligo | Sequence | NO: |
| XhoI- | AGAA CTCGAG AACTGCAAAAATAGTTTGACACCCTAGCCGATAGG | 413 |
| PD/E20- | | |
| fwd | | |
| NsiI- | AAGG ATGCAT GGTTATTGTCTCATGAGCGGATACATATTTGAATGT | 414 |
| terminator- | | |
| rev | | |

Each tether or secretion system PCR product was digested with Xho I and Nsi I, ligated into BAL492 or BAL998, respectively using these restriction sites, and transformed into EPI300 cells (Epicentre biotechnologies). Plasmids from respective positive clones were then transformed into ATCC8739 cells to characterize their lyase activities.

Bacteria: E. coli K12 (DH5α and DH10B), E. coli W, and E. coli C2 (ATCC8739).

Alginate lyase assay: Vector-carrying bacteria as described above were grown for various time periods in media containing the appropriate antibiotics at 30° C. At each sampling time, a portion of the culture was removed and the alginate degradation kinetics were determined in various fractions. The total activity available for degradation of extracellular substrate was measured using the culture without further processing. To distinguish between tethered enzymatic activity and fully secreted enzymatic activity (i.e., activity in the conditioned media), the conditioned media was separated from the cells by centrifugation and the cell pellets were washed once and resuspended in M9 salts solution at the original volume. One part of sample was mixed with 9 parts of sodium alginate solution (0.2% w/v in M9 salts solution) and the reaction was incubated at 30° C. The β-elimination mechanism of AL activity produces a new double bond at the product, and the appearance of this bond was monitored by reading the absorbance at 232 nm. The specific activity of each sample was derived from the initial velocity of the reaction according to the following transformation: 1 Unit (U) of enzymatic activity is: dOD₂₃₂ₙₘ/dt=0.1/min.

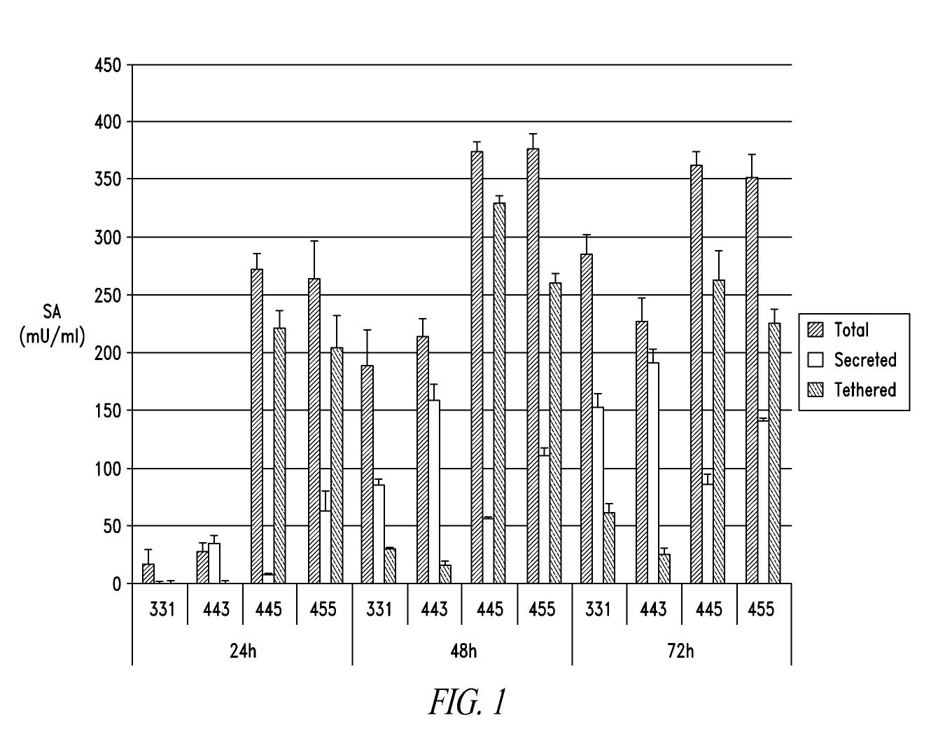

As shown in FIG. 1, each of the combinations of carrier proteins (e.g., Omp1, OmpA, PgsA and Ag43) fused to Sphingomonas sp. Al ALs (e.g., AI-I, AI-II and AI-III) were biologically active, i.e., they resulted in measurable AL activity tethered to, or in the conditioned media of, cells transformed with vectors carrying these fusion proteins. The AL of Pseudoalteromonas sp. SM0524 was most active when fused to the Ag43 carrier protein.

FIG. 1 further shows that the dynamics of AL activity over a period of 72 hours varied between the different vectors. For instance, vectors 445 and 455 reach 70% of their potential in 24 hours while vectors 331 and 443 show less activity at that time. Also, the distribution of activity between the tethered and fully secreted fractions varied between the various vectors. Mainly, most of the activity of vectors 331 and 443 was found in the media while most of the activity of vector 445 was tethered to the cells. Clone 455, which contains elements of two fusion proteins, showed a more balanced distribution of AL activity between the fully secreted fraction and the tethered fraction.

Tables 10 and 11 also illustrates the activity of certain alginate lyase-based tether display constructs.

TABLE 10

| | DISPLAY | | | | |
|---|---|---|---|---|---|
| | | | | | Ag43 |
| AL | Lpp-OmpA | PgsA | InaK | phoA-EstA | (flu) |
| AI-I | <1 | 1.32 | <1 | <1 | <1 |
| ΔAI-I | 1.68 | <1 | 0 | 0 | 3.11 |
| AI-II | 1.22 | <1 | 0 | 0 | 22.5 |
| AI-III | 1.24 | <1 | 0 | 0 | 1.7 |
| AI-II′(s) | 0 | 0 | 0 | 0 | 0 |
| Pseudoalteromonas | 0 | 0 | 0 | 0 | 436.5 |
| sp. SM0524 | | | | | |

TABLE 11

| Alginate lyase | Activity 16 h | Activity 64 h |
|---|---|---|
| construct | mU/ml | mU/ml |
| pPDC-Omp1-AI-I | <5 | 1080 |
| pPDC-LppOmpA-AI-I | <5 | 1140 |
| pPDC-Ag43-ΔPaAly | 190 | 605 |
| pPDC-Ag43-AI-II | Not tested | 1230 |

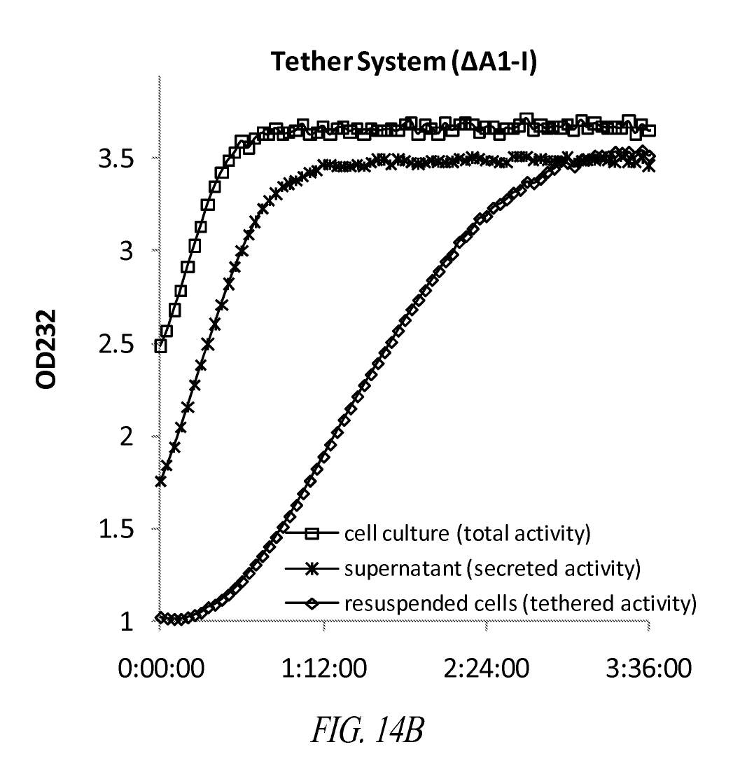

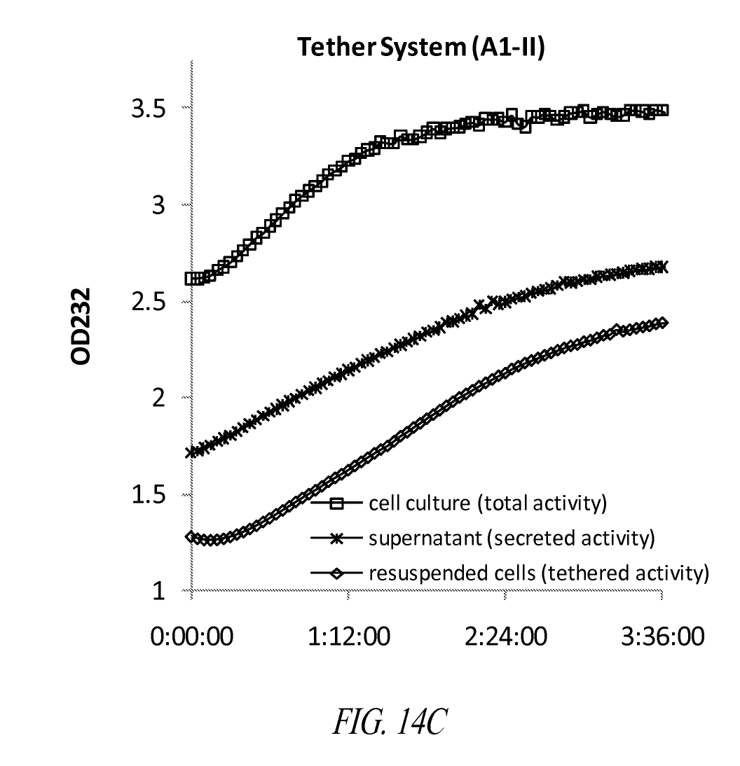

The results for pCCfos2-based alginate-lyase surface-display systems are shown in FIGS. 14A-14C. This system, consisting of the P_{D/E20} promoter, the Ag43 signal peptide, and the ΔAg43S400, was of the highest interest among all combinations investigated. This tether/surface-display system works very well for all three lyases explored to-date (ΔPaAly, ΔA1-I, and A1-II) and exhibited significant lyase activities towards the alginate polymer as their substrate. In accordance with the surface-display design, the majority of lyase activity was found as tethered activity for ΔPaAly (FIG. 14A). However, there were lyase activities distributed between the supernatant and resupsended cells for both ΔA1-I and A1-II (FIGS. 14B and 14C). This is likely due to the speculated presence of a protease processing site within ΔA1 and A1-II, which would enable detaching the lyase from the cell surface and releasing it to the growth media, hence accounting for the lyase activities observed in the supernatant.

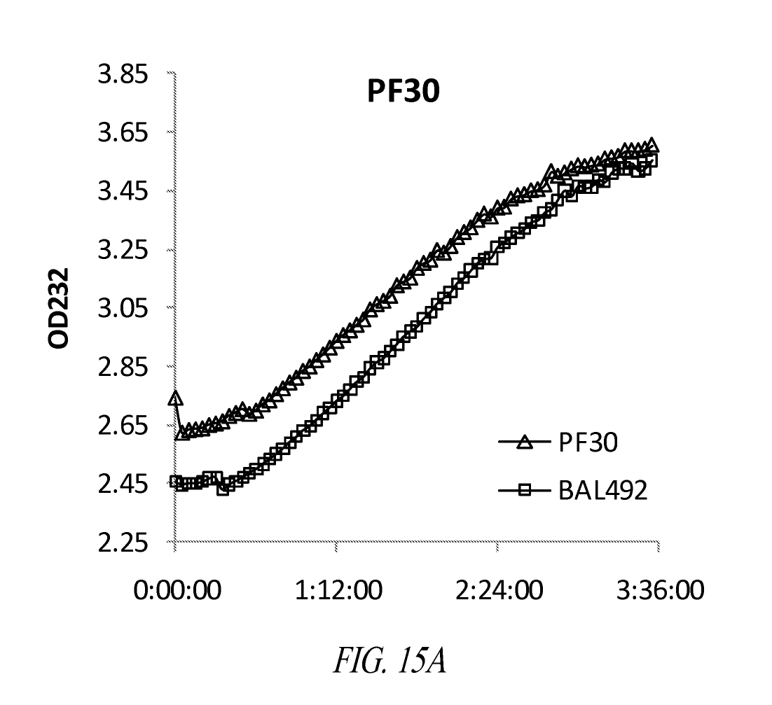

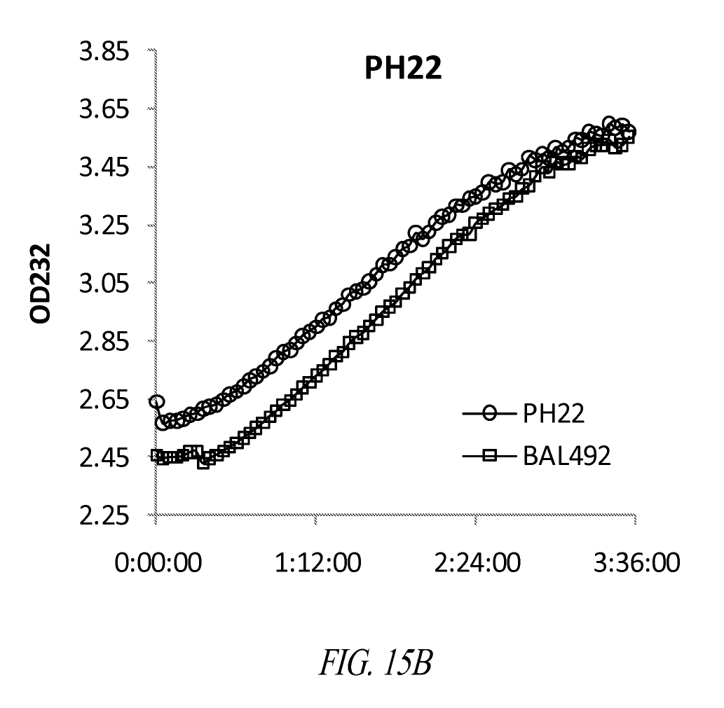

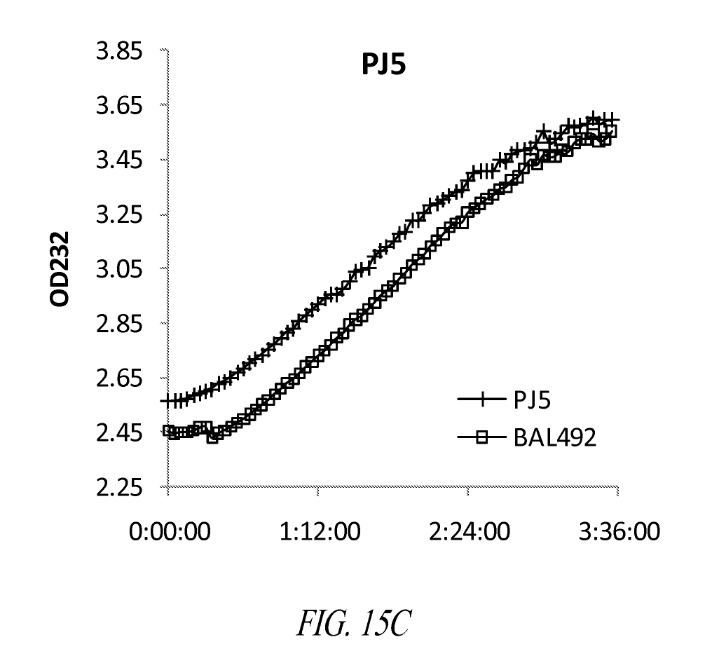

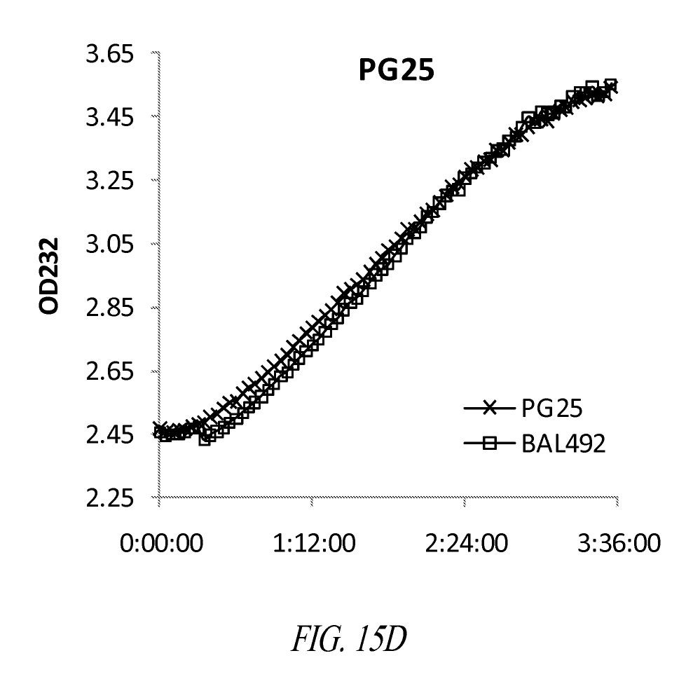

Among the tether/surface-display systems of the three different lyases characterized above, the ΔPaAly system was chosen to be further investigated for the effect of expressing under different promoters. Plasmids harboring four different promoters (P_{F30}, P_{H22}, P_{G25}, and P_{J5}), driving the expression of the tether/surface-display system of ΔPaAly, were made based on BAL492. When transformed into ATCC8739 cells, all of these constructs were active and exhibited significant lyase activities. Strains carrying P_{F30}, P_{H22}, and P_{J5} appeared to degrade alginate slightly slower than that of BAL492 (FIGS. 15A-C), whereas P_{G25}- and BAL492-harboring strains showed nearly identical activities (FIG. 15D).

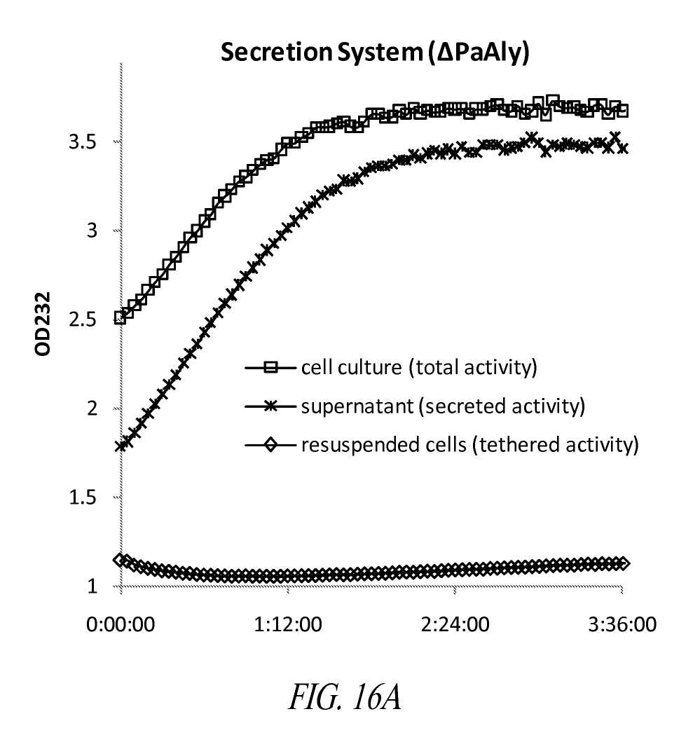

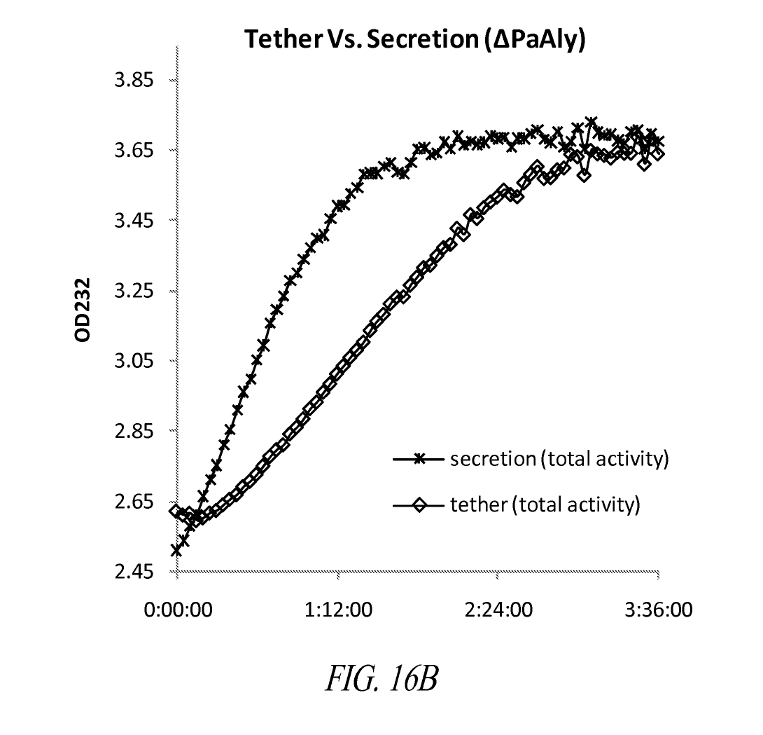

In comparison to the tether/surface-display system of alginate lyase described above, another alginate lyase system was tested that is capable of secreting the lyase outside the cell into the growth media. This system was developed based on a combination of random and rational design strategies where the passenger domain of Ag43 was randomly truncated at certain amino acid sites and the aspartyl protease active site was rationally included in all of these truncations (see Table 8 above for primers and related description). These truncated fragments were then cloned into the same expression vector as the tether/surface-display system, and thus all of these constructs contain the same promoter, signal peptide, truncated alginate lyase, autochaperone, and carrier domain of Ag43. Among the different truncation constructs screened for secretion of ΔPaAly, the construct carrying Ag43 truncated at the amino acid N455 (ΔAg43N455) showed the highest secreted lyase activity. The sequence of the secretion system construct composed of the promoter, the signal peptide, the ΔPaAly lyase, the truncated autotransporter, and the terminator is shown in SEQ ID NO:156. The majority of the lyase activity of this construct was detected in the supernatant (FIG. 16A), indicating that the enzyme was effectively secreted into the growth media. In addition, the secretion system exhibited a lyase activity that is approximately twice as fast as that of the tether system in degrading the same amount of the alginate polymer (FIG. 16B).

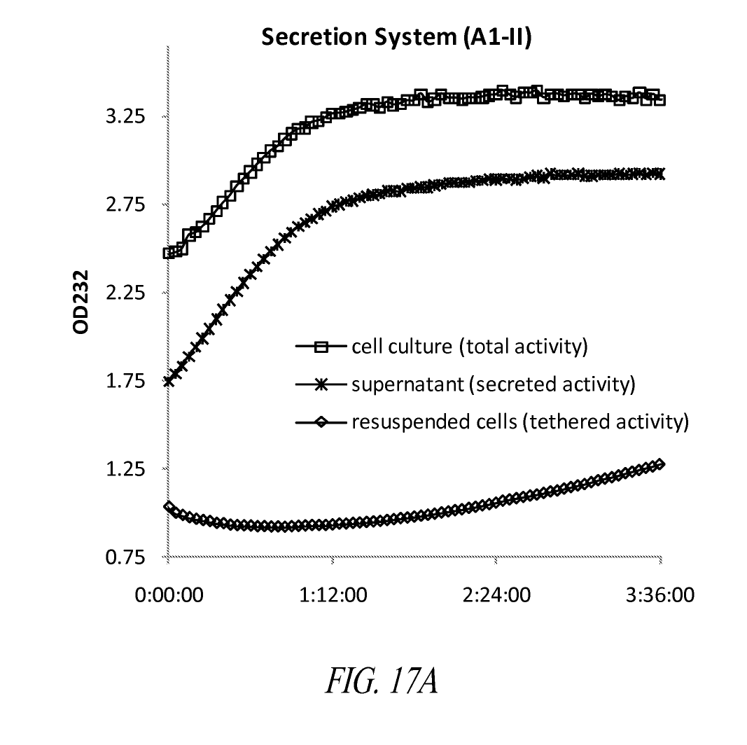

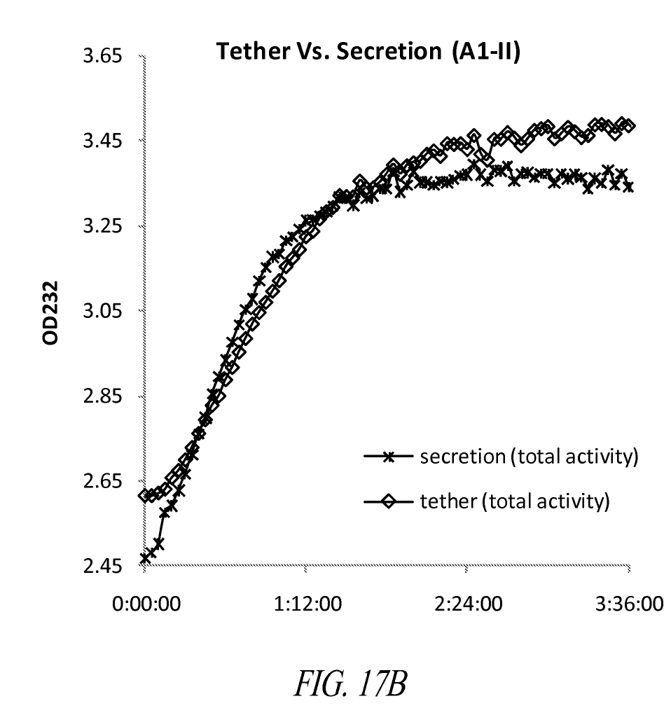

Similar to that of ΔPaAly, the secretion system construct of A1-II also showed the majority of lyase activity in the supernatant (FIG. 17A) and a faster alignate degradation rate than that of its tether counterpart in terms of total activity (FIG. 17B). These data support that the design of the secretion system is not specific to a particular lyase fused to the system and thus can be potentially implemented with various enzymes of interest to be secreted into the growth media, thereby highlighting the functional modularity of this system.

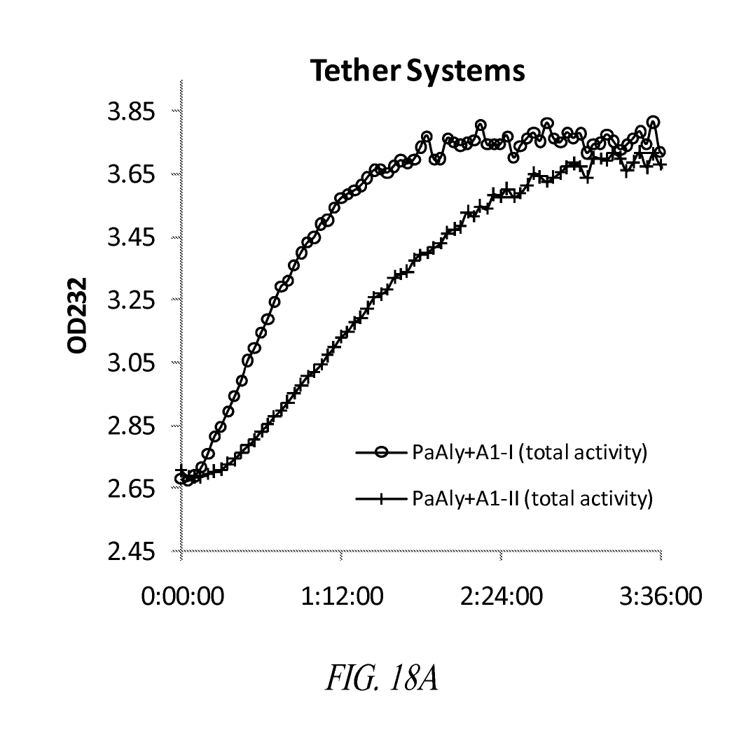

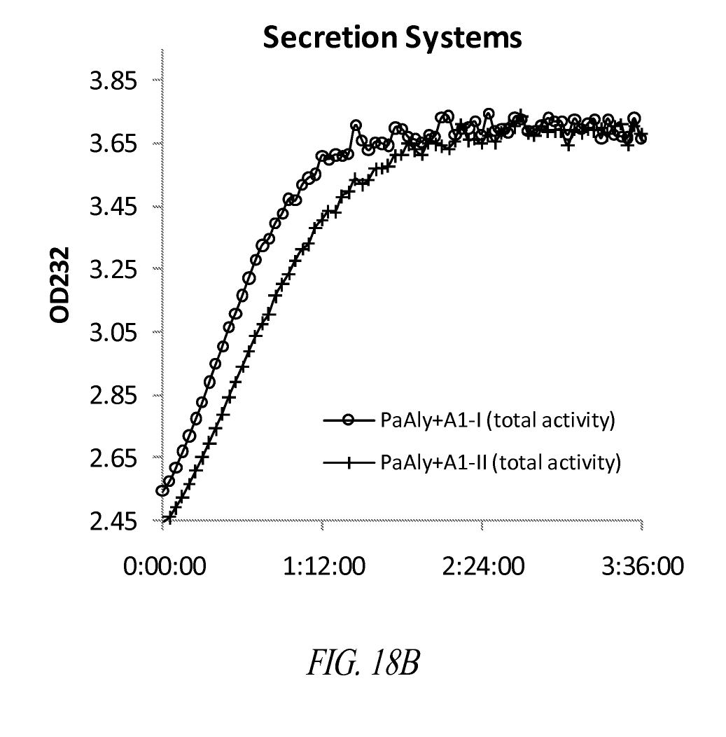

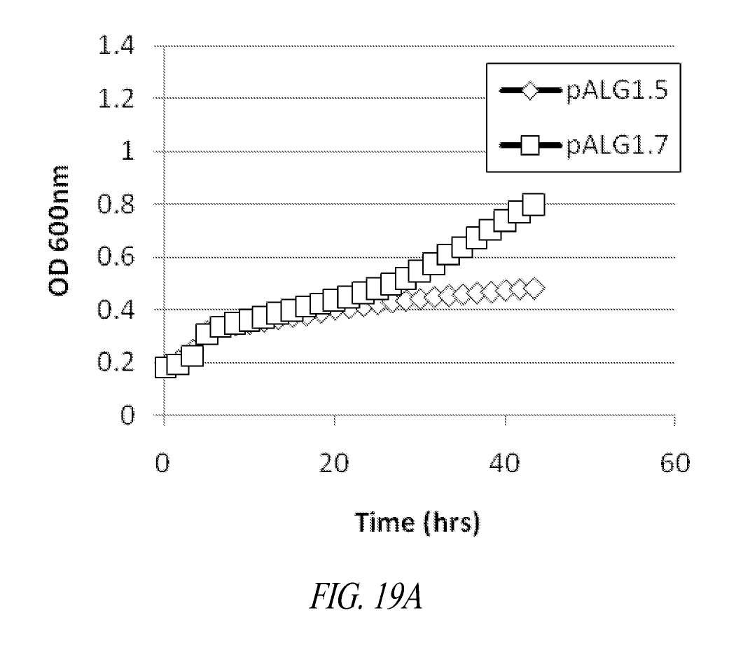

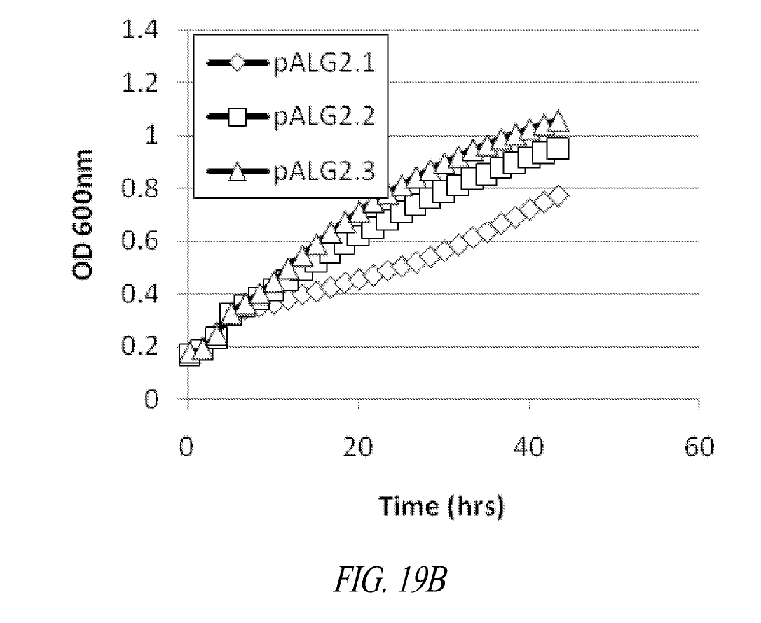

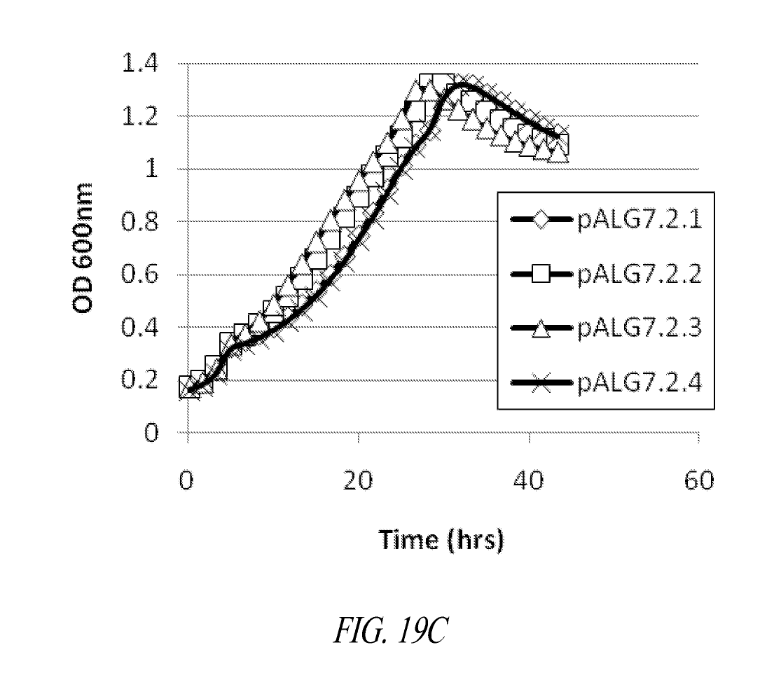

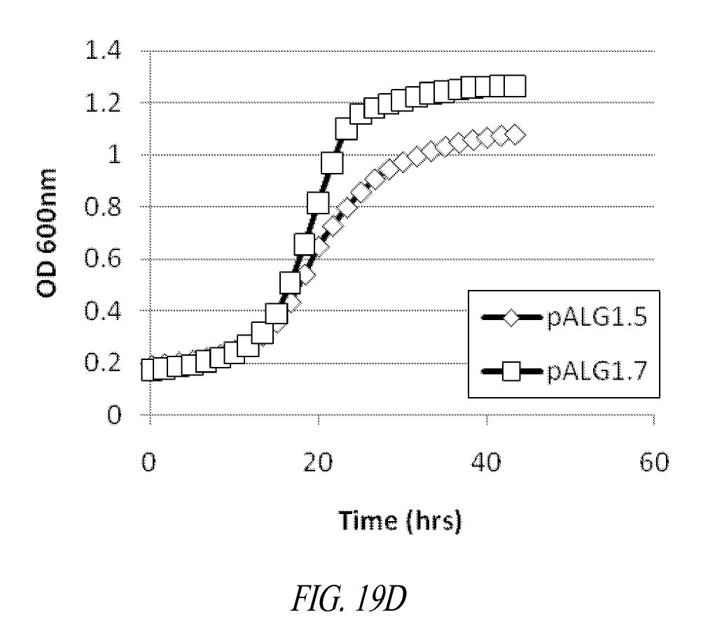

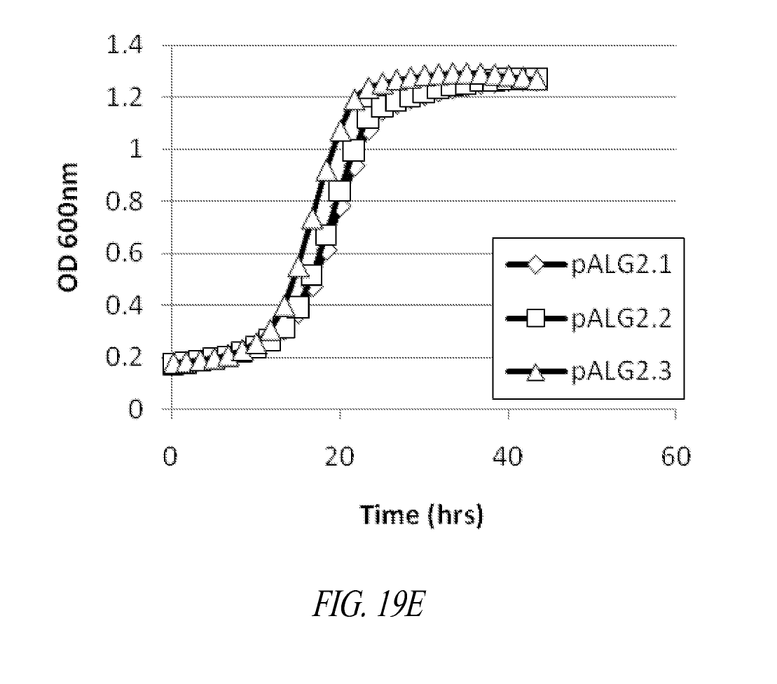

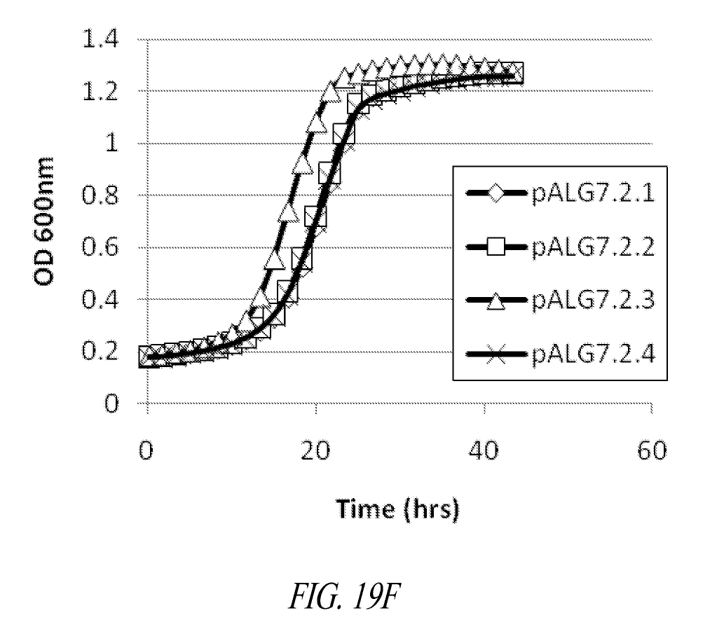

Multiple enzymes (in this case, two different lyases) were then expressed independently as a tether or secretion system within a single-copy plasmid. As shown in FIGS. 18A and 19B, cells harboring these dual-enzyme systems exhibited significant tethered and secreted lyase activities, respectively.

Preparation of Alginate Lyase Secretion Systems. to Further Test the ability of various signal sequences to direct the secretion of alginate lyases, vectors containing secretion signal sequences were constructed based on pET29 plasmid backbone (Novagen). The secretion signal sequences (secretion signal sequence from PelB, OmpA, StII, EX, PhoA, OmpF, PhoE, MalE, OmpC, LPP, LamB, OmpT, and LTB) were amplified by PCR: 98° C. for 10 sec, 55° C. for 15 sec, and 72° C. for 10 sec, repeated for 30 times. The reaction mixture contained 1× Phusion buffer, 2 mM dNTP, 0.5 μM forward and reverse primers, and 2.5 U Phusion DNA polymerase (Finezyme) in total volume of 100 μl. The amplified fragments were digested with NdeI and NcoI and ligated into pET29 pre-digested with the same enzymes using T4 DNA ligase to form pET-SP1 through pET-SP13. The constructed plasmid was sequenced (Elim Biopharmaceuticals) and the DNA sequence of the insert was confirmed.

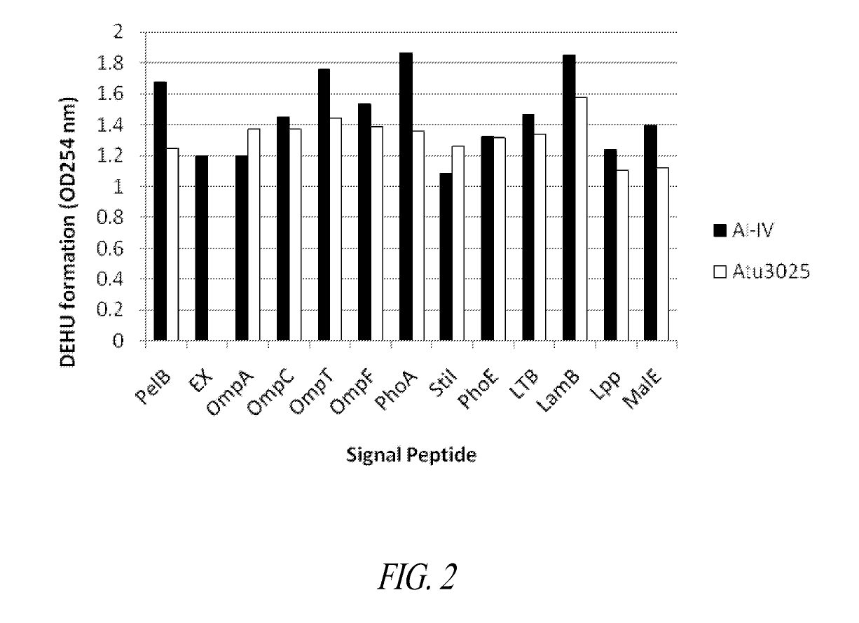

Then, the genes encoding alginate lyase AI-IV and Atu3025 were amplified by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-CATGCCATGGAGAAGCTGGAACAGCC-3′ (SEQ ID NO:415) for AI-IV and 5′-CATGCCATGGGTCCCTCTGCCCCGGC-3′ (SEQ ID NO:416) for Atu3025) and reverse primers (5′-CGGGATCCTTAGAACGGTTTGGGCAACG-3′ (SEQ ID NO:417) for AI-IV and 5′-CGGGATCCTTAGAACTGCTTGGGAAGGG-3′ (SEQ ID NO:418) for Atu3025), and 2.5 U Phusion DNA polymerase (Finezyme) in total volume of 100 μl. The amplified fragment was digested with NcoI and BamHI and ligated into pET-SP1 through pET-SP13 pre-digested with the same enzymes using T4 DNA ligase to form pET-SP1-AI-IV and pET-SP1-Atu3025 through pET-SP13-AI-IV and pET-SP13-Atu3025. The constructed plasmids were sequenced (Elim Biopharmaceuticals) and the DNA sequence of the insert was confirmed. The E. coli BL21 harboring plasmids pET-SP1-AI-IV and pET-SP1-Atu3025 through pET-SP13-AI-IV and pET-SP13-Atu3025 were grown in M9 media containing 0.2% glycerol. When OD 600 nm reached 0.6, the cultures were induced with 0.1 mM IPTG and were further grown at 15 C for overnight. The cultures were centrifuged and supernatants were taken to analyze their alginate degradation activity by measuring the increase of OD254 nm. FIG. 2 shows the secretion of AI-IV and Atu3025 by the various secretion peptide sequences PelB, OmpA, StII, EX, PhoA, OmpF, PhoE, MalE, OmpC, LPP, LamB, OmpT, and LTB.

### Example 2

### Improved Growth of E. coli on Degraded Alginate

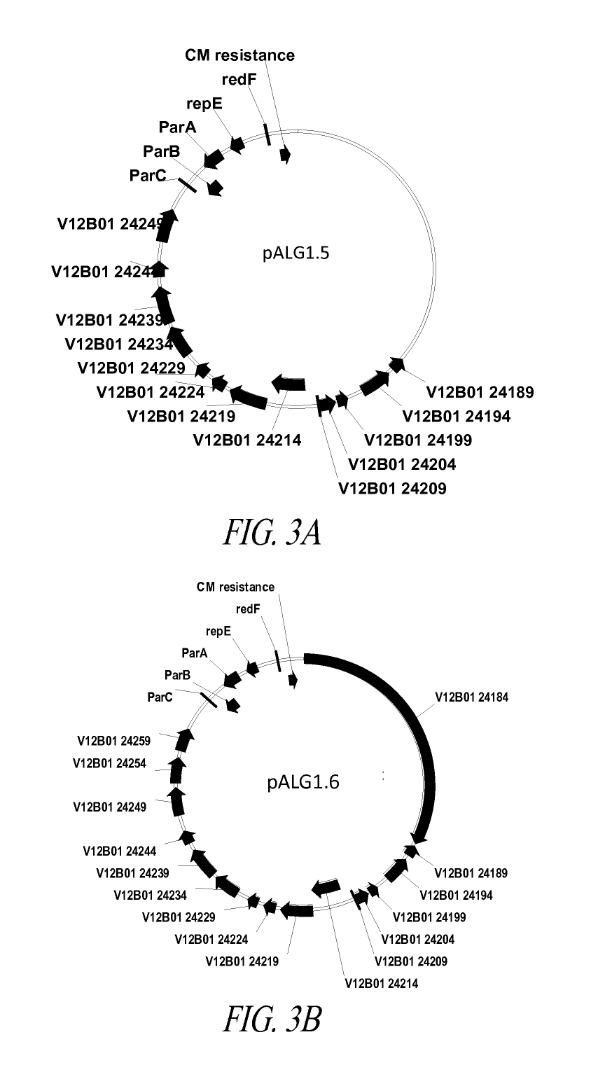

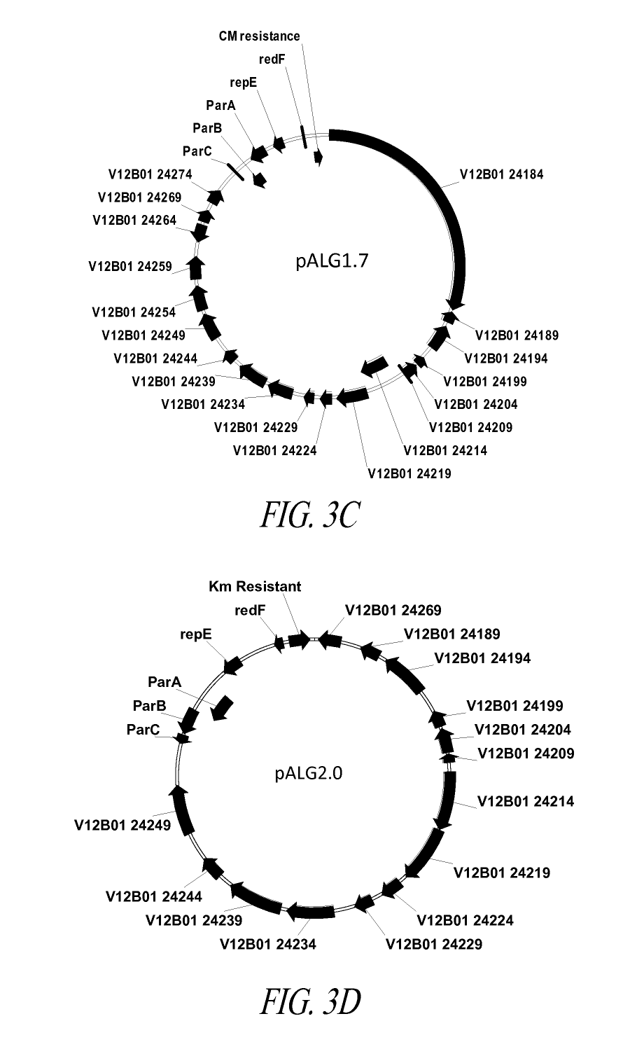

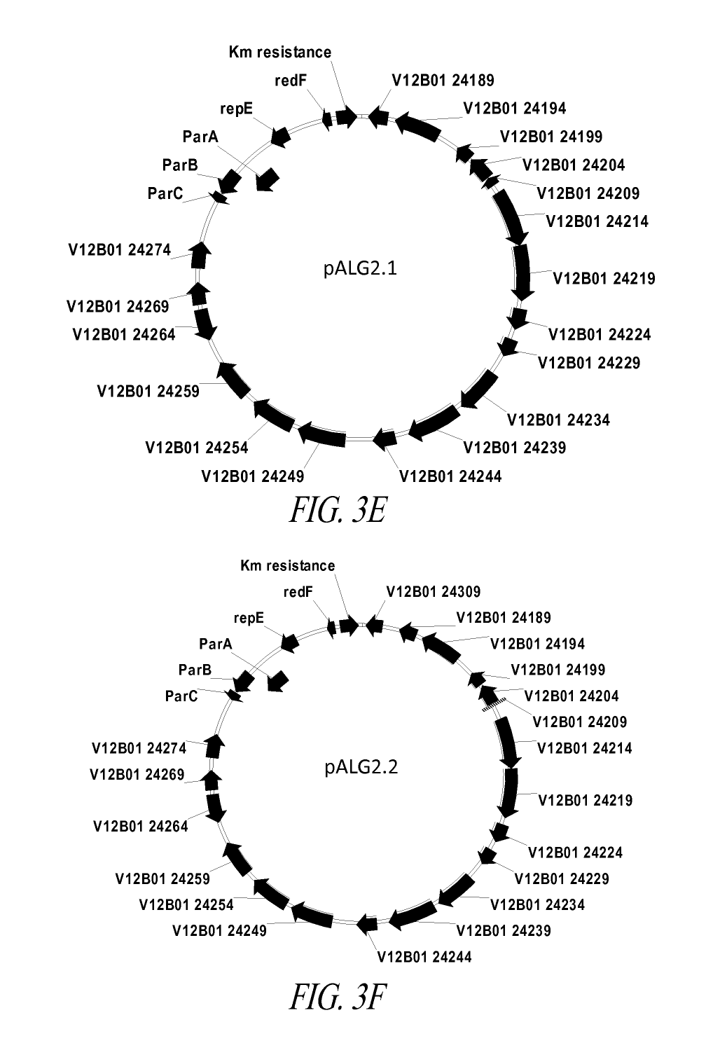

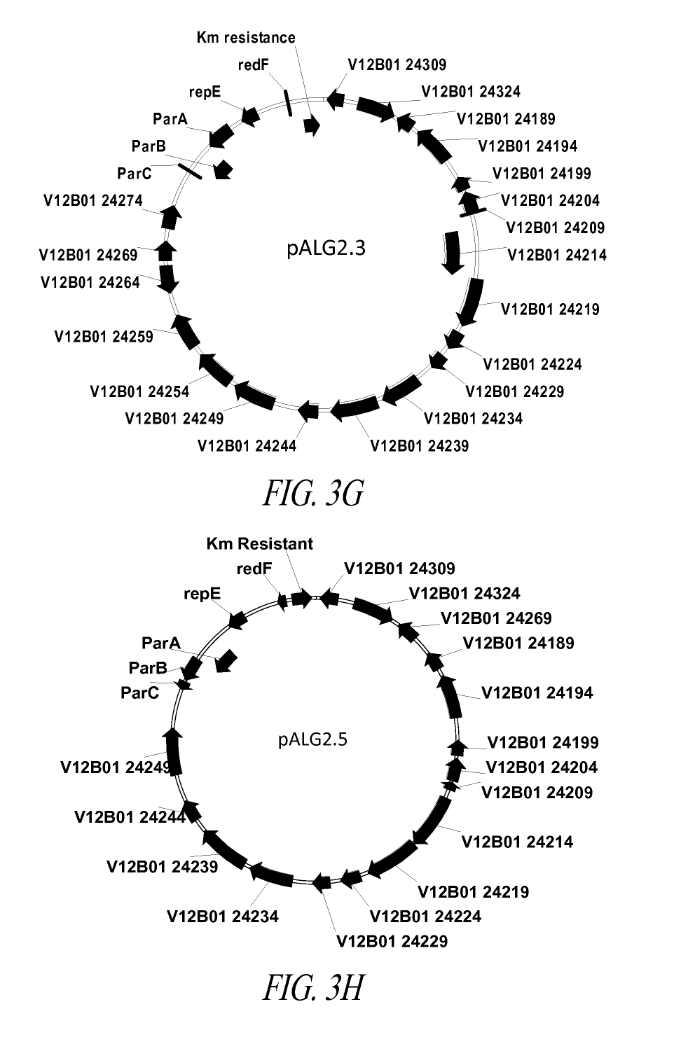

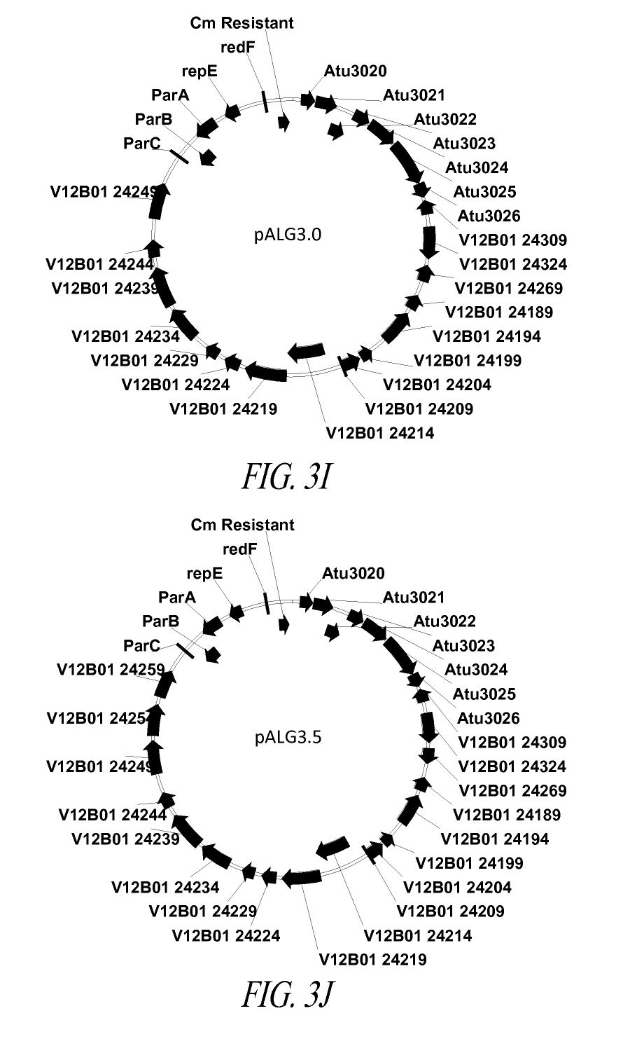

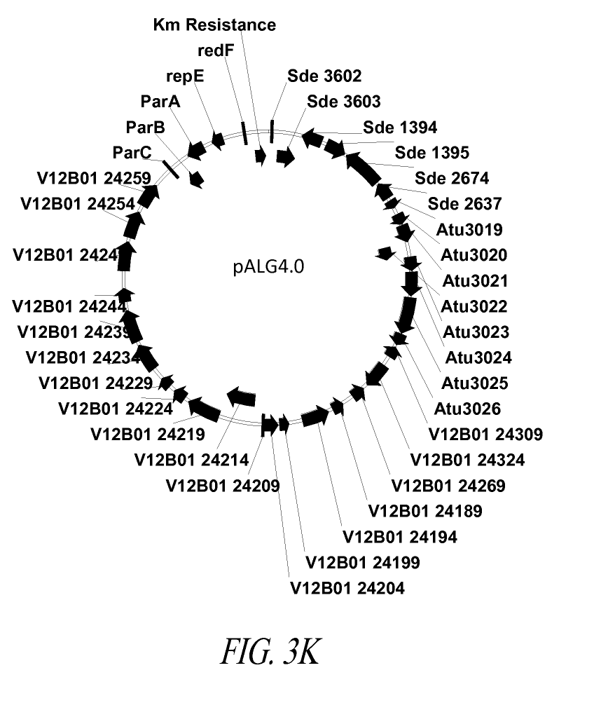

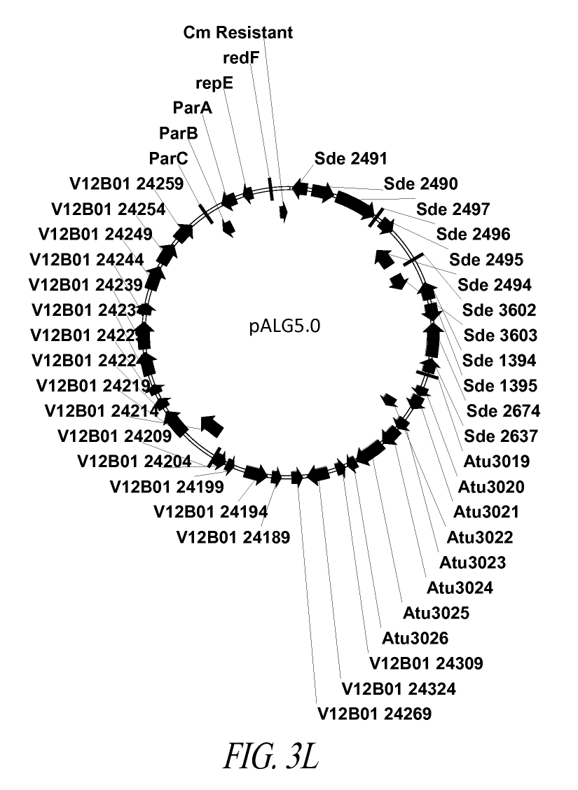

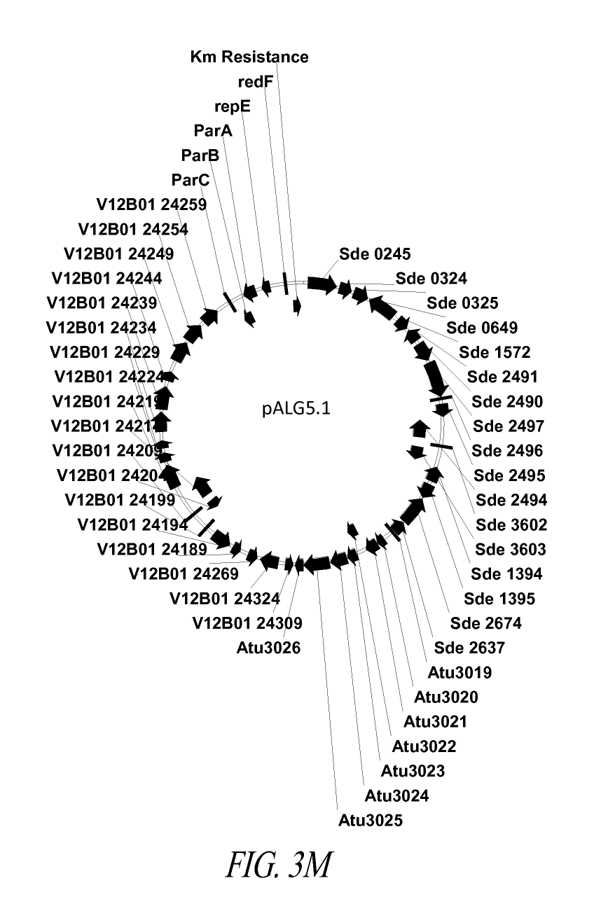

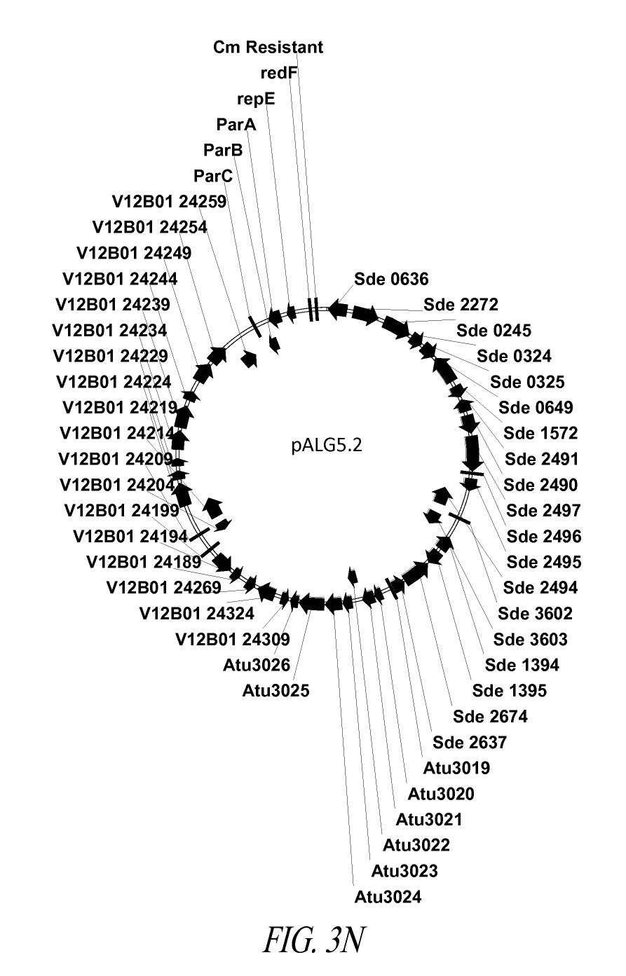

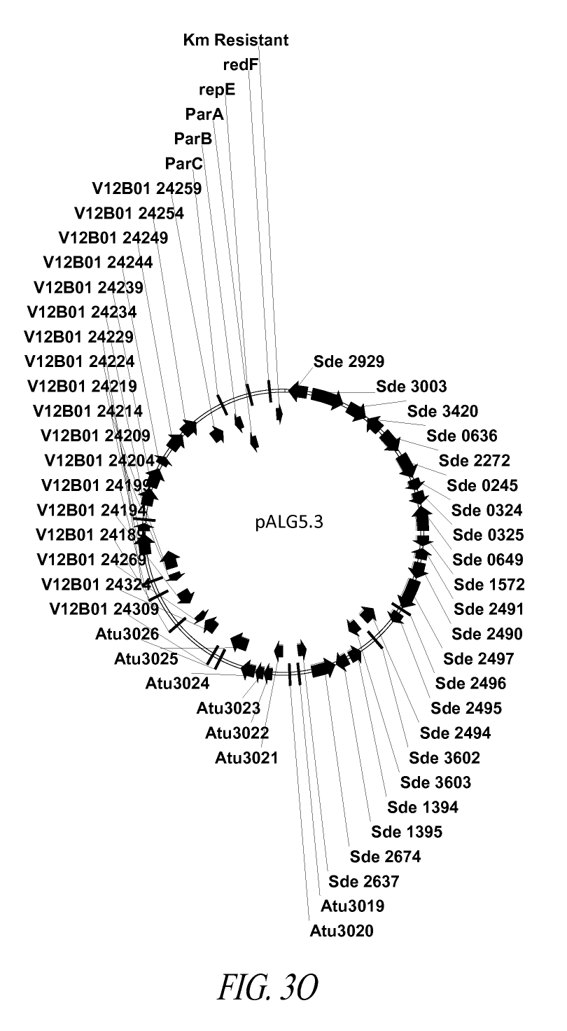

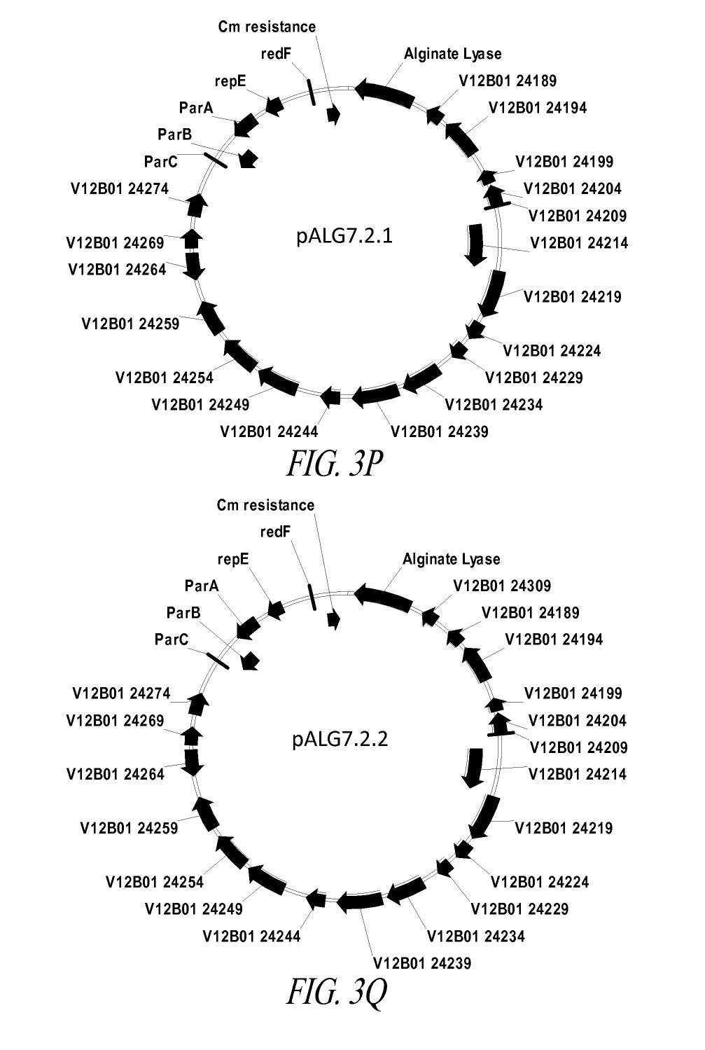

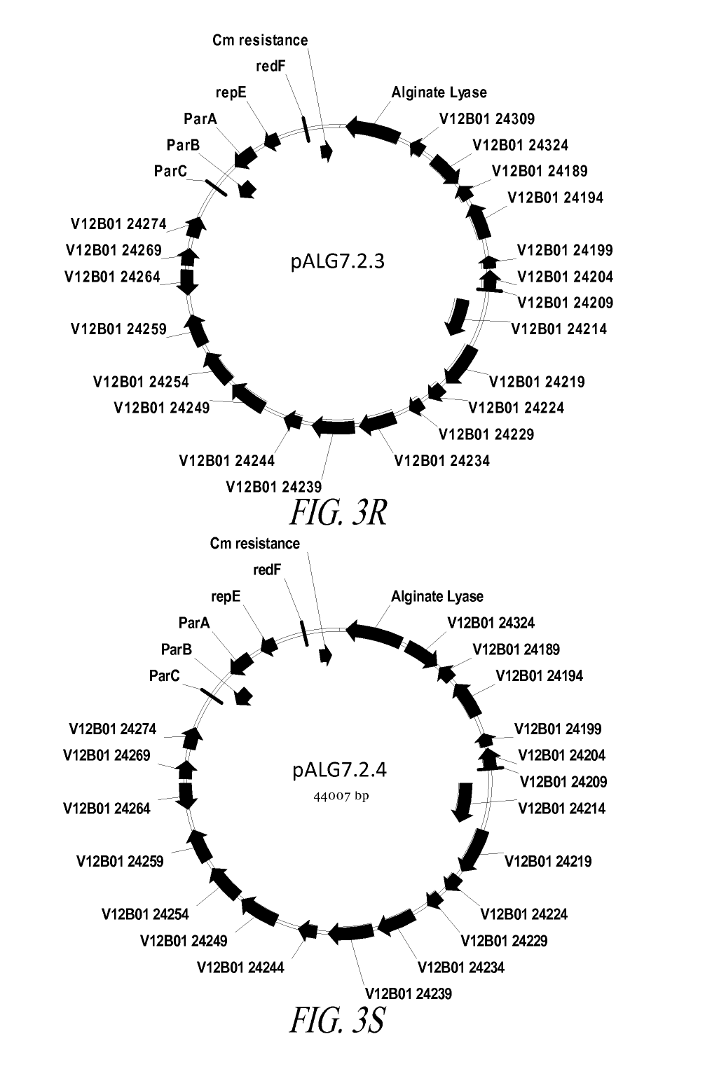

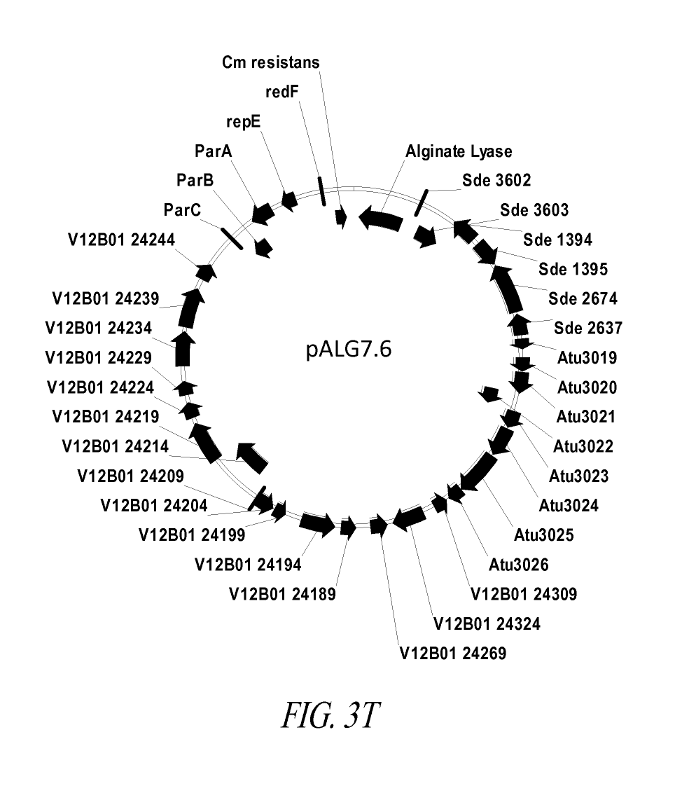

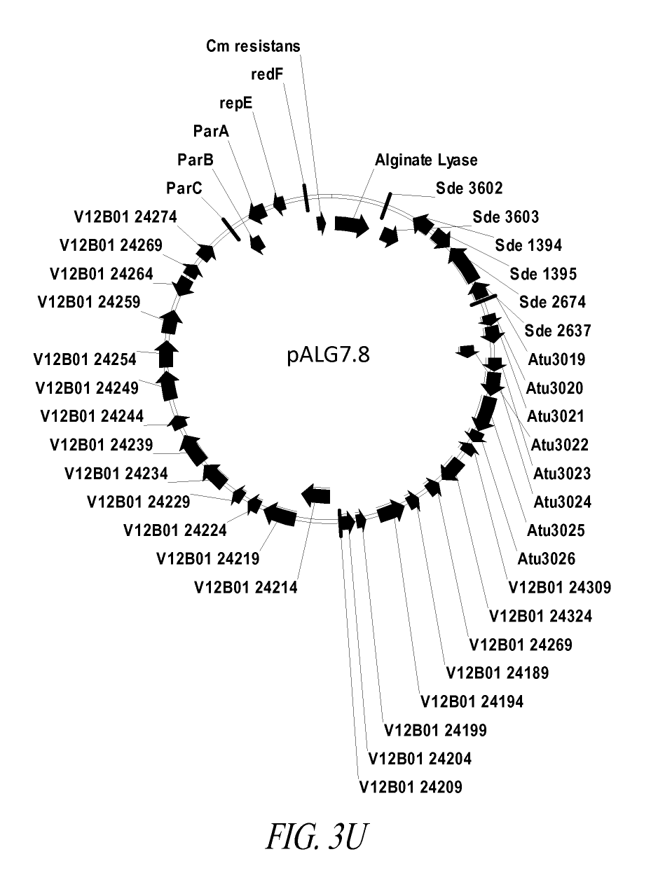

To improve the ability of recombinant E. coli to metabolize and grow on alginate as a sole source of carbon, the pALG1.5 vector was modified by incorporating additional genetic components, mainly those involved in the extracellular degradation and transport of alginate and its by-products. The pALG1.5 vector contains the genomic region between V12B01_{—}24189 and V12B01_{—}24249 of Vibrio splendidus, and confers on E. coli the ability to grow on alginate as a sole source of carbon (see, e.g., U.S. Application No. 2009/0139134, herein incorporated by reference, which describes the construction of pALG1.5). A diagram of the pALG1.5 vector is shown in FIG. 3A. A diagram of each of the following vectors is shown in FIGS. 3B-3U.

Construction of pALG 1.6. To improve alginate degradation, a vector containing V12B01_{—}24254 (alginate lyase) and V12B01_{—}24259 (alginate lyase) was constructed based on pKm2 plasmid backbone (R6Kγ-based vector containing kanamycin resistant gene (Km) flanked by FRT sites). The pKm2, and V12B01_{—}24254-24259 sequences were amplified by PCR: 98° C. for 10 sec, 55° C. for 15 sec, and 72° C. for 1 min for pKm2 and 2 min for V12B01_{—}24254-24259, repeated for 30 times. The reaction mixture contained 1× Phusion buffer, 2 mM dNTP, 0.5 μM forward (5′-CGGGATCCGTCGACCTGCAGTTCGAAG-3′ [SEQ ID NO:419] and 5′-TGTCAAACATGAGAATTAATTCCGGTTGATGAGCAGCTTTAAGGTTTAAT-3′ [SEQ ID NO:420], respectively) and reverse (5′-ATTAAACCTTAAAGCTGCTCATCAACCGGAATTAATTCTCATGTTTGACA-3′ [SEQ ID NO:421] and 5′-CGGGATCCCATACGCTTAAGCCCAACCAACAGC-3′ [SEQ ID NO:422], respectively) primers, 100 ng of purified genome of Vibrio splendidus 12B01 or 50 ng of purified pKm2 vector, and 2.5 U Phusion DNA polymerase (Finezyme) in total volume of 50 μl.

Each amplified DNA fragment was gel purified and eluted into 30 ul of Elution buffer (QIAGEN). These amplified fragments were spliced by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CGGGATCCGTCGACCTGCAGTTCGAAG-3′ [SEQ ID NO:419]) and reverse (5′-CGGGATCCCATACGCTTAAGCCCAACCAACAGC-3′ [SEQ ID NO:422]) primers, 5 ul of each purified DNA fragment, and 2.5 U Phusion DNA polymerase (Finezyme) in total volume of 50 μl. The amplified fragment was then digested with BamHI (New England Biolabs) and ligated with T4 DNA ligase to form pKm2-V12B01_{—}24254-24259. The constructed plasmid was sequenced (Elim Biopharmaceuticals) and the DNA sequence of the insert was confirmed. The Km2-V12B01_{—}24254-24259 was then amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-TTGATGAGCAGCTTTAAGGTTTAATG-3′ [SEQ ID NO:423]) and reverse (5′-CTCACTATAGGGCGAATTCGAGCTCGGTACCCGGGGATCCGTGTAGGCTGGA GCTGCTTC-3′ [SEQ ID NO:424]) primers, 50 ng of purified pKm2-V12B01_{—}24254-24259, and 2.5 U Phusion DNA polymerase (Finezyme) in total volume of 50 μl. The amplified fragment was gel purified and transformed into a strain harboring pALG1.5 to construct pALG1.6 via homologous recombination. The kanamycin selection marker was excised from the pALG1.6 through over-expression of FLP.

Construction of pALG1.7. To improve alginate degradation, a vector containing V12B01_{—}24264 (alginate lyase) V12B01_{—}24269 (outer membrane porin) and V12B01_{—}24274 (alginate lyase) was constructed based on pKm2 plasmid backbone (R6Kγ-based vector containing kanamycin resistant gene (Km) flanked by FRT sites). The pKm2, and V12B01_{—}24264-24274 sequences were amplified by PCR: 98° C. for 10 sec, 55° C. for 15 sec, and 72° C. for 1 min for pKm2 and 3 min for V12B01_{—}24264-24274, repeated for 30 times. The reaction mixture contained 1× Phusion buffer, 2 mM dNTP, 0.5 μM forward (5′-CGGGATCCGTCGACCTGCAGTTCGAAG-3′ [SEQ ID NO:419] and 5′-TGTCAAACATGAGAATTAATTCCGGTCTAATCGAATAACACTTAATATTAAA GG-3′ [SEQ ID NO:425], respectively) and reverse (5′-CCTTTAATATTAAGTGTTATTCGATTAGACCGGAATTAATTCTCATGTTTGAC A-3′ [SEQ ID NO:426] and 5′-ACTCCGTATCGAGTTGTCGTCCTAA-3′ [SEQ ID NO:427], respectively) primers, 100 ng of purified genome of Vibrio splendidus 12B01 or 50 ng of purified pKm2 vector, and 2.5 U Phusion DNA polymerase (Finezyme) in total volume of 50 μl.

Each amplified DNA fragment was gel purified and eluted into 30 ul of Elution buffer (QIAGEN). These amplified fragments were spliced by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CGGGATCCGTCGACCTGCAGTTCGAAG-3′ [SEQ ID NO:419]) and reverse (5′-ACTCCGTATCGAGTTGTCGTCCTAA-3′ [SEQ ID NO:427]) primers, 5 ul of each purified DNA fragment, and 2.5 U Phusion DNA polymerase (Finezyme) in total volume of 50 μl. The amplified fragment was then treated with T4 polynucleotide kinase (New England Biolabs) and ligated with T4 DNA ligase to form pKm2-V12B01_{—}24254-24259. The constructed plasmid was sequenced (Elim Biopharmaceuticals) and the DNA sequence of the insert was confirmed. The Km2-V12B01_{—}24254-24259 was then amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-TCTAATCGAATAACACTTAATATTAAAGG-3′ [SEQ ID NO:428]) and reverse (5′-CTCACTATAGGGCGAATTCGAGCTCGGTACCCGGGGATCCGTGTAGGCTGGA GCTGCTTC-3′[SEQ ID NO:424]) primers, 50 ng of purified pKm2-V12B01_{—}24264-24274, and 2.5 U Phusion DNA polymerase (Finezyme) in total volume of 50 μl. The amplified fragment was gel purified and transformed into a strain harboring pALG1.6 to construct pALG1.7 via homologous recombination. The kanamycin selection marker was excised from the pALG1.7 through over-expression of FLP.

Construction of pALG2.0. To enhance alginate metabolism, the pALG2.0 vector was constructed by incorporating into the pALG1.5 vector and an additional polynucleotide sequence that encodes an outer membrane porin from Vibrio splendidus, operably linked to a promoter. Specifically, a vector containing V12B01_{—}24269 (outer membrane porin) was constructed based on the pKm plasmid backbone (R6Kγ-based vector containing kanamycin resistant gene (Km)).

The V12B01_{—}24269 and pKm sequences were amplified by PCR: 98° C. for 10 sec, 55° C. for 15 sec, and 72° C. for 1 min, repeated for 30 times. The reaction mixture contained 1× Phusion buffer, 2 mM dNTP, 0.5 μM forward (5′-GAATCGTTTTCCGGGACGCCGGATGAAGCTAATTCTGATTAG-3′ (SEQ ID NO:429) and 5′-CGGGTACCGGGCCCCCCCTCGAGGTC-3′ (SEQ ID NO:430), respectively) and reverse (5′-CGGGATCCTCAGCACAGAAACTACTTTTG-3′ (SEQ ID NO:431) and 5′-CTAATCAGAATTAGCTTCATCCGGCGTCCCGGAAAACGATTC-3′ (SEQ ID NO:432), respectively) primers, 100 ng of purified genome of Vibrio splendidus 12B01 or 50 ng of purified pKm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:430) and reverse (5′-CGGGATCCTCAGCACAGAAACTACTTTTG-3′) (SEQ ID NO:431) primers, 5 ul of each purified DNA fragment, and 2.5 U Phusion DNA polymerase (Finezyme) in total volume of 50 μl. The amplified fragment was then digested with BamHI (New England Biolabs) and ligated with T4 DNA ligase to form pKm-V12B01_{—}24269. The constructed plasmid was sequenced (Elim Biopharmaceuticals) and the DNA sequence of the insert was confirmed. Km-V12B01_{—}24269 was then amplified by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:433) and reverse (5′-TCTTCAACCACAATCACCTGTTCCGTAGTGCCTAAACCATCAGCACAGAAACT ACTTTTG-3′) (SEQ ID NO:434) primers, 50 ng of purified pKm-V12B01_{—}24269, and 2.5 U Phusion DNA polymerase (Finezyme) in total volume of 50 μl. The amplified fragment was gel purified and transformed into a strain harboring pALG1.5 to construct pALG2.0 via homologous recombination.

Construction of pALG2.1. To replace Cm resistant gene on pALG1.7 with the Km registrant gene, a Km fragment was amplified by PCR: 98° C. for 10 sec, 55° C. for 15 sec, and 72° C. for 1 min, repeated for 30 times. The reaction mixture contained 1× Phusion buffer, 2 mM dNTP, 0.5 μM forward (5′-CTGACCGTTCTGTCCGTCACTTCCC-3′ [SEQ ID NO:435]) and reverse (5′-TTTAATCGTTAGATTCTAATAGCTAGCCTCCAATTAGGCGATCTAAGATAATT ACTGTCC-3′ [SEQ ID NO:436]) primers, 50 ng of purified pKm, and 2.5 U Phusion DNA polymerase (Finezyme) in total volume of 50 μl. The amplified fragment was gel purified and transformed into a strain harboring pALG1.7 to construct pALG2.1 via homologous recombination.

Construction of pALG2.2, 2.3, and 2.5. To enhance alginate metabolism, a vector containing V12B01_{—}24309 (outer membrane porin), V12B01_{—}24324 (transporter), and V12B01_{—}24269 (outer membrane porin) was constructed based on pKm plasmid backbone (R6Kγ-based vector containing kanamycin resistant gene(Km)). The V12B01_{—}24309, V12B01_{—}24324, V12B01_{—}24269, and pKm sequences were amplified by PCR: 98° C. for 10 sec, 55° C. for 15 sec, and 72° C. for 1 min, repeated for 30 times. The reaction mixture contained 1× Phusion buffer, 2 mM dNTP, 0.5 μM forward (5′-GAATCGTTTTCCGGGACGCC TTAAGTACTCGGCTCTTATTTAAATG-3′ [SEQ ID NO:437], 5′-GTTTTATTCATGGTATTAATTCCATTTTTTAATGGACGAGGGGAAAGTG-3′ [SEQ ID NO:438], 5′-GAAATAATTTTAAAAGCCCCAATAGGGGATGAAGCTAATTCTGATTAG-3′ [SEQ ID NO:439], and 5′-CGGGTACCGGGCCCCCCCTCGAGGTC-3′ [SEQ ID NO:440], respectively) and reverse (5′-CACTTTCCCCTCGTCCATTAAAAAATGGAATTAATACCATGAATAAAAC-3′ [SEQ ID NO:441], 5′-CTAATCAGAATTAGCTTCATCCCCTATTGGGGCTTTTAAAATTATTTC-3′ [SEQ ID NO:442], 5′-CGGGATCCTCAGCACAGAAACTACTTTTG-3′ [SEQ ID NO:443], and 5′-CATTTAAATAAGAGCCGAGTACTTAAGGCGTCCCGGAAAACGATTC-3′ [SEQ ID NO:444], respectively) primers, 100 ng of purified genome of Vibrio splendidus 12B01 or 50 ng of purified pKm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 4 min, repeated for 30 times. The reaction mixture contained 1× Phusion buffer, 2 mM dNTP, 0.5 μM forward (5′-CGGGTACCGGGCCCCCCCTCGAGGTC-3′ [SEQ ID NO:430]) and reverse (5′-CGGGATCCTCAGCACAGAAACTACTTTTG-3′ [SEQ ID NO:431]) primers, 5 ul of each purified DNA fragment, and 2.5 U Phusion DNA polymerase (Finezyme) in total volume of 50 μl. The amplified fragment was then digested with BamHI (New England Biolabs) and ligated with T4 DNA ligase to form pKm-V12B01_{—}24269.

pALG2.2: The constructed plasmid was sequenced (Elim Biopharmaceuticals) and the DNA sequence of the insert was confirmed. pKm-V12B01_{—}24309_{—}24324_{—}24269 was then amplified by PCR: 98° C. for 10 sec, 55° C. for 15 sec, and 72° C. for 4 min, repeated for 30 times. The reaction mixture contained 1× Phusion buffer, 2 mM dNTP, 0.5 μM forward (5′-CTGACCGTTCTGTCCGTCACTTCCC-3′ [SEQ ID NO:435]) and reverse (5′-TTTAATCGTTAGATTCTAATAGCTAGCCTCCAATTAGGCGTAATCACTCGTCG TACTTGT-3′ [SEQ ID NO:445]) primers, 50 ng of purified pKm-V12B01_{—}24309_{—}24324_{—}24269, and 2.5 U Phusion DNA polymerase (Finezyme) in total volume of 50 μl. The amplified fragment was gel purified and transformed into a strain harboring pALG1.7 to construct pALG2.2 via homologous recombination.

pALG2.3: The constructed plasmid was sequenced (Elim Biopharmaceuticals) and the DNA sequence of the insert was confirmed. pKm-V12B01_{—}24309_{—}24324_{—}24269 was then amplified by PCR: 98° C. for 10 sec, 55° C. for 15 sec, and 72° C. for 4 min, repeated for 30 times. The reaction mixture contained 1× Phusion buffer, 2 mM dNTP, 0.5 μM forward (5′-CTGACCGTTCTGTCCGTCACTTCCC-3′ [SEQ ID NO:435]) and reverse (5′-TTTAATCGTTAGATTCTAATAGCTAGCCTCCAATTAGGCGGTTGTTGATTTAG AAGGAAA-3′ [SEQ ID NO:446]) primers, 50 ng of purified pKm-V12B01_{—}24309_{—}24324_{—}24269, and 2.5 U Phusion DNA polymerase (Finezyme) in total volume of 50 μl. The amplified fragment was gel purified and transformed into a strain harboring pALG1.7 to construct pALG2.2 via homologous recombination.

pALG2.5: The constructed plasmid was sequenced (Elim Biopharmaceuticals) and the DNA sequence of the insert was confirmed. pKm-V12B01_{—}24309_{—}24324_{—}24269 was then amplified by PCR: 98° C. for 10 sec, 55° C. for 15 sec, and 72° C. for 4 min, repeated for 30 times. The reaction mixture contained 1× Phusion buffer, 2 mM dNTP, 0.5 μM forward (5′-CTGACCGTTCTGTCCGTCACTTCCC-3′ [SEQ ID NO:435]) and reverse (5′-TCTTCAACCACAATCACCTGTTCCGTAGTGCCTAAACCATCAGCACAGAAACT ACTTTTG-3′ [SEQ ID NO:447]) primers, 50 ng of purified pKm-V12B01_{—}24309_{—}24324_{—}24269, and 2.5 U Phusion DNA polymerase (Finezyme) in total volume of 50 μl. The amplified fragment was gel purified and transformed into a strain harboring pALG1.5 and pALG1.7 to construct pALG2.5 and pALG2.3 via homologous recombination.

Construction of pALG2.4. To improve alginate degradation, a vector containing PutP (transporter) was constructed based on pKm2 plasmid backbone (R6Kγ-based vector containing kanamycin resistant gene (Km) flanked by FRT sites). The pKm2, and V12B01_{—}19706 sequences were amplified by PCR: 98° C. for 10 sec, 55° C. for 15 sec, and 72° C. for 1 min for pKm2 and 3 min for V12B01_{—}19706, repeated for 30 times. The reaction mixture contained 1× Phusion buffer, 2 mM dNTP, 0.5 μM forward (5′-AATCGCTCAAGACGTGTAATGCTGC-3′ [SEQ ID NO:448] and 5′-TCAGAAAAGGTCATTTGAAGGGATATGTAGGCTGGAGCTGCTTCGAAGTT-3′ [SEQ ID NO:449], respectively) and reverse (5′-AACTTCGAAGCAGCTCCAGCCTACATATCCCTTCAAATGACCTTTTCTGA-3′ [SEQ ID NO:450] and 5′-TTCATCTCACCCTTTTAAGTTCAAT-3′ [SEQ ID NO:451], respectively) primers, 100 ng of purified genome of Vibrio splendidus 12B01 or 50 ng of purified pKm2 vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-AATCGCTCAAGACGTGTAATGCTGC-3′ [SEQ ID NO:452]) and reverse (5′-TTCATCTCACCCTTTTAAGTTCAAT-3′ [SEQ ID NO:453]) primers, 5 ul of each purified DNA fragment, and 2.5 U Phusion DNA polymerase (Finezyme) in total volume of 50 μl.

The amplified fragment was then treated with T4 polynucleotide kinase (New England Biolabs) and ligated with T4 DNA ligase to form pKm2-V12B01_{—}19706. The constructed plasmid was sequenced (Elim Biopharmaceuticals) and the DNA sequence of the insert was confirmed. The Km2-V12B01_{—}19706 was then amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CCCTGGGCCAACTTTTGGCGAAAATGAGACGTTGAATAACTTCGTATAGTAC ACATTATACGAAGTTATATCCGTCGACCTGCAGTTCGA-3′ [SEQ ID NO:454]) and reverse (5′-CTTTCAAATCAATTCATTTAAATAAGAGCCGAGTACTTAATTCATCTCACCCT TTTAAGT-3′ [SEQ ID NO:455]) primers, 50 ng of purified pKm2-V12B01_{—}19706, and 2.5 U Phusion DNA polymerase (Finezyme) in total volume of 50 μl. The amplified fragment was gel purified and transformed into a strain harboring pALG2.3 to construct pALG2.4 via homologous recombination.

Construction of pALG3.0. To further enhance alginate metabolism, the pALG3.0 vector was constructed by incorporating into the pALG2.5 vector an additional polynucleotide sequence that encodes an ATP-binding cassette (ABC) transporter from Agrobacterium tumefaciens, an oligoalginate lyase and a DEHU dehydrogenase. A vector containing Atu_{—}3020, Atu_{—}3021, Atu_{—}3022, Atu_{—}3023, Atu_{—}3024 (21-24: ABC transporter), Atu_{—}3025 (oligoalginate lyase), and Atu_{—}3026 (DEHU hydrogenase) was constructed based on pCm plasmid backbone (R6Kγ-based vector containing chloramphenicol resistant gene (Cm)).

The pCm and Atu_{—}3020-3026 sequences were amplified by PCR: 98° C. for 10 sec, 55° C. for 15 sec, and 72° C. for 1 min for pCm and 5 min for Atu_{—}3020-3026, repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:456) and 5′-GCTGTCAAACATGAGAATTGGTCGGTCCATGGAGTCAAACCGCCACGTC-3′ (SEQ ID NO:457), respectively) and reverse (5′-GACGTGGCGGTTTGACTCCATGGACCGACCAATTCTCATGTTTGACAGC-3′ (SEQ ID NO:458) and 5′-GCTCTAGAAAGAGCCGAGTACTTAAGGATCATCAGGAAAACAGGACGCCG-3′ (SEQ ID NO:459), respectively) primers, 100 ng of purified genome of Agrobacterium tumefaciens C58 or 50 ng of purified pCm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:460) and reverse (5′-GCTCTAGAAAGAGCCGAGTACTTAAGGATCATCAGGAAAACAGGACGCCG-3′) (SEQ ID NO:461) primers, 5 ul of each purified DNA fragment, and 2.5 U Phusion DNA polymerase (Finezyme) in total volume of 50 μl. The amplified fragment was then digested with XbaI (New England Biolabs) and ligated with T4 DNA ligase to form pCm-Atu3020-3026. The constructed plasmid was sequenced (Elim Biopharmaceuticals) and the DNA sequence of the insert was confirmed. The pCm-Atu3020-3026 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:435) and reverse (5′-CTGGCTTTTCTTCTTTCAAATCAATTCATTTAAATAAGAGCCGAGTACTTAAG GATCATC-3′) (SEQ ID NO:462) primers, 50 ng of purified pCm-Atu3020-3026, and 2.5 U Phusion DNA polymerase (Finezyme) in total volume of 50 μl. The amplified fragment was gel purified and transformed into a strain harboring pALG2.5 to construct pALG3.0 via homologous recombination.

Construction of pALG3.5. To improve alginate degradation, the pALG3.5 vector was constructed by incorporating into the pALG3.0 vector an additional polynucleotide sequence that encodes two alginate lyases from Vibrio splendidus 12B01. A vector containing V12B01_{—}24254 (alginate lyase) and V12B01_{—}24259 (alginate lyase) was constructed based on pKm2 plasmid backbone (R6Kγ-based vector containing kanamycin resistant gene (Km) flanked by FRT sites).

The pKm2, and V12B01_{—}24254-24259 sequences were amplified by PCR: 98° C. for 10 sec, 55° C. for 15 sec, and 72° C. for 1 min for pKm2 and 2 min for V12B01_{—}24254-24259, repeated for 30 times. The reaction mixture contained 1× Phusion buffer, 2 mM dNTP, 0.5 μM forward (5′-CGGGATCCGTCGACCTGCAGTTCGAAG-3′ (SEQ ID NO:463) and 5′-TGTCAAACATGAGAATTAATTCCGGTTGATGAGCAGCTTTAAGGTTTAAT-3′ (SEQ ID NO:464), respectively) and reverse (5′-ATTAAACCTTAAAGCTGCTCATCAACCGGAATTAATTCTCATGTTTGACA-3′ (SEQ ID NO:465) and 5′-CGGGATCCCATACGCTTAAGCCCAACCAACAGC-3′ (SEQ ID NO:466), respectively) primers, 100 ng of purified genome of Vibrio splendidus 12B01 or 50 ng of purified pKm2 vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CGGGATCCGTCGACCTGCAGTTCGAAG-3′) (SEQ ID NO:467) and reverse (5′-CGGGATCCCATACGCTTAAGCCCAACCAACAGC-3′) (SEQ ID NO:468) primers, 5 ul of each purified DNA fragment, and 2.5 U Phusion DNA polymerase (Finezyme) in total volume of 50 μl. The amplified fragment was then digested with BamHI (New England Biolabs) and ligated with T4 DNA ligase to form pKm2-V12B01_{—}24254-24259. The constructed plasmid was sequenced (Elim Biopharmaceuticals) and the DNA sequence of the insert was confirmed. The Km2-V12B01_{—}24254-24259 was then amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-TTGATGAGCAGCTTTAAGGTTTAATG-3′) (SEQ ID NO:469) and reverse (5′-CTCACTATAGGGCGAATTCGAGCTCGGTACCCGGGGATCCGTGTAGGCTGGA GCTGCTTC-3′) (SEQ ID NO:470) primers, 50 ng of purified pKm2-V12B01_{—}24254-24259, and 2.5 U Phusion DNA polymerase (Finezyme) in total volume of 50 μl. The amplified fragment was gel purified and transformed into a strain harboring pALG3.0 to construct pALG3.5 via homologous recombination. The kanamycin selection marker was excised from the pALG3.5 through over-expression of FLP.

Biological Activity. The pALG1.5, pALG2.0, pALG2.5, and pALG3.0 vectors were transformed into E. coli, and the ability of these recombinant microorganisms to grown in alginate was tested. The vector-containing E. coli were incubated for 48 hours at 30° C. in M9 media containing 1 mg/ml thiamine and 1% degraded alginate. At 48 hours, culture samples were collected and the OD₆₀₀ₙₘ values were measured.

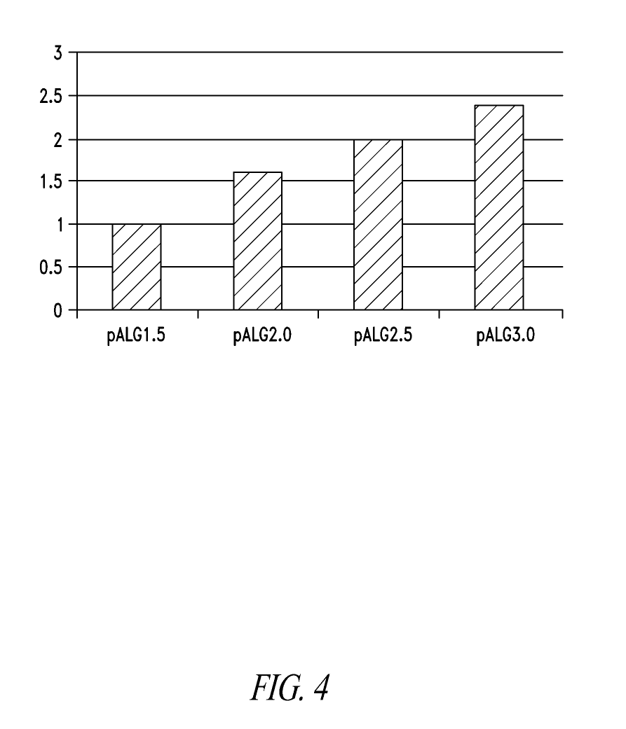

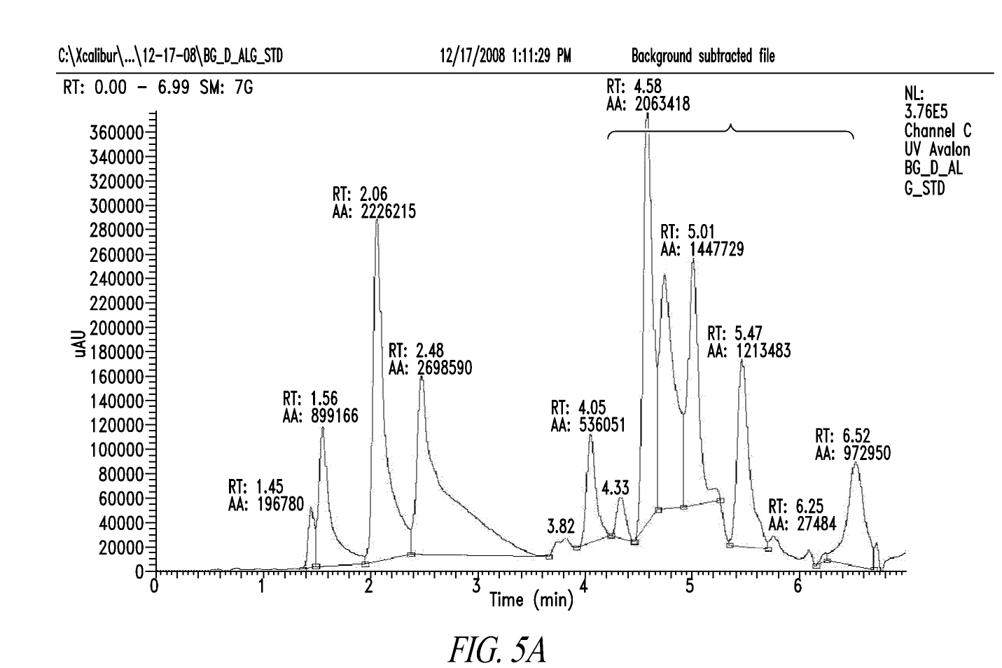

FIG. 4 shows the OD₆₀₀ₙₘ values (y-axis) for pALG vector-containing E. coli growing on alginate. The addition of each individual component (e.g., outer membrane porin, symporter, ABC transporter) incrementally enhances the ability of E. coli to grow on alginate as a sole source of carbon, with the pALG2.5 and pALG3.0 vectors unexpectedly more than doubling the growth of E. coli on alginate.

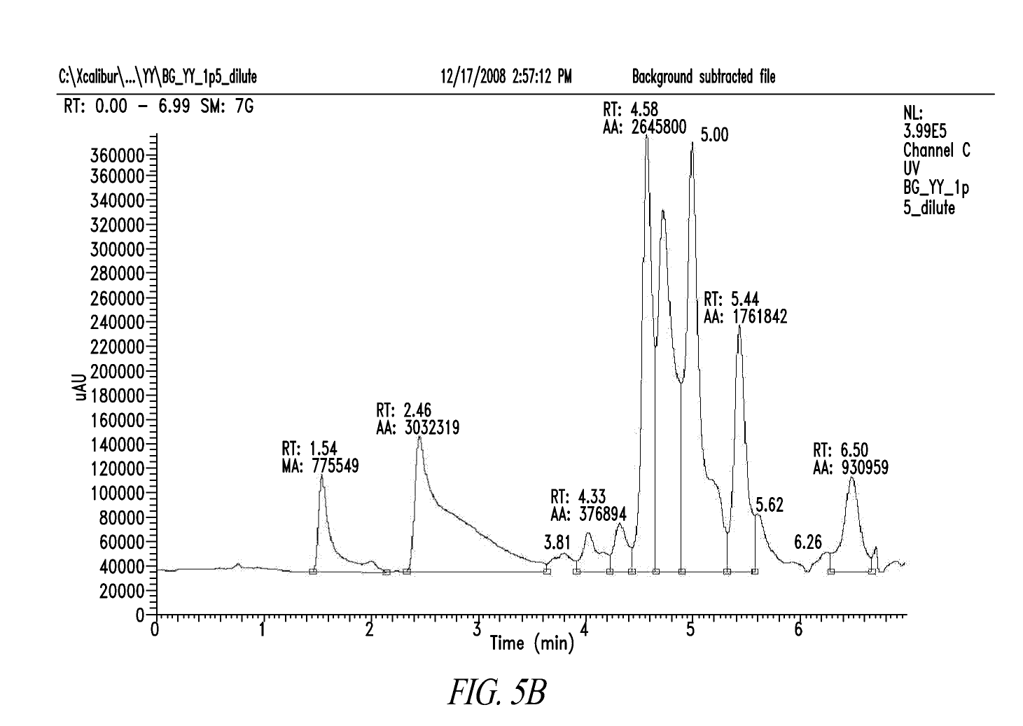

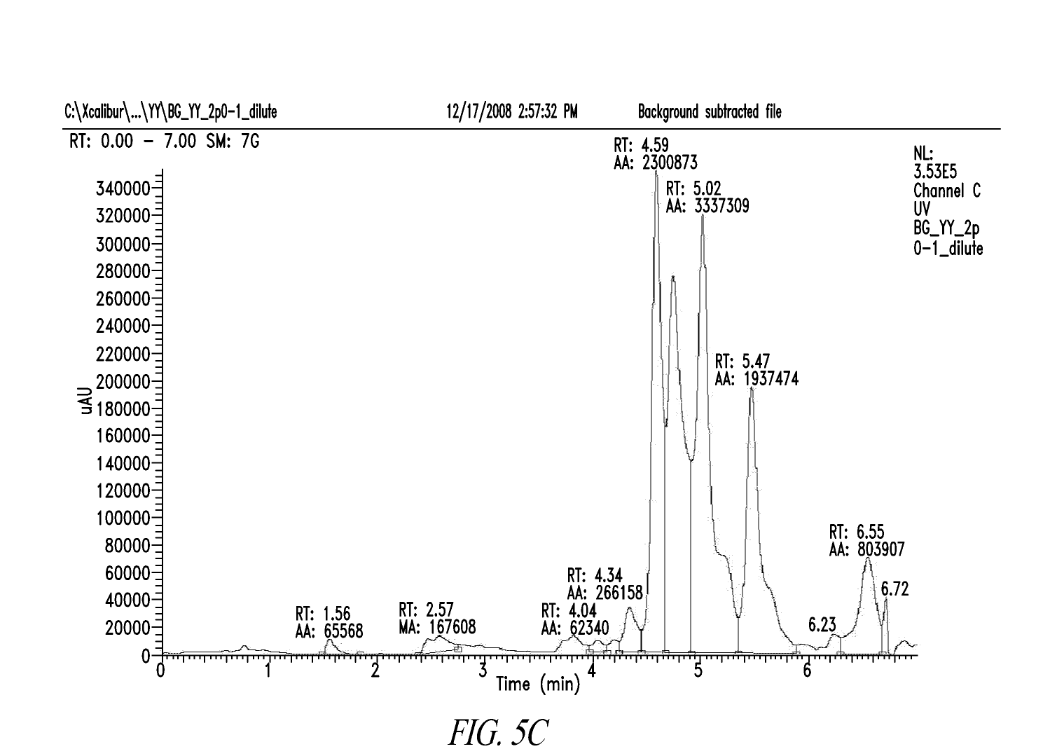

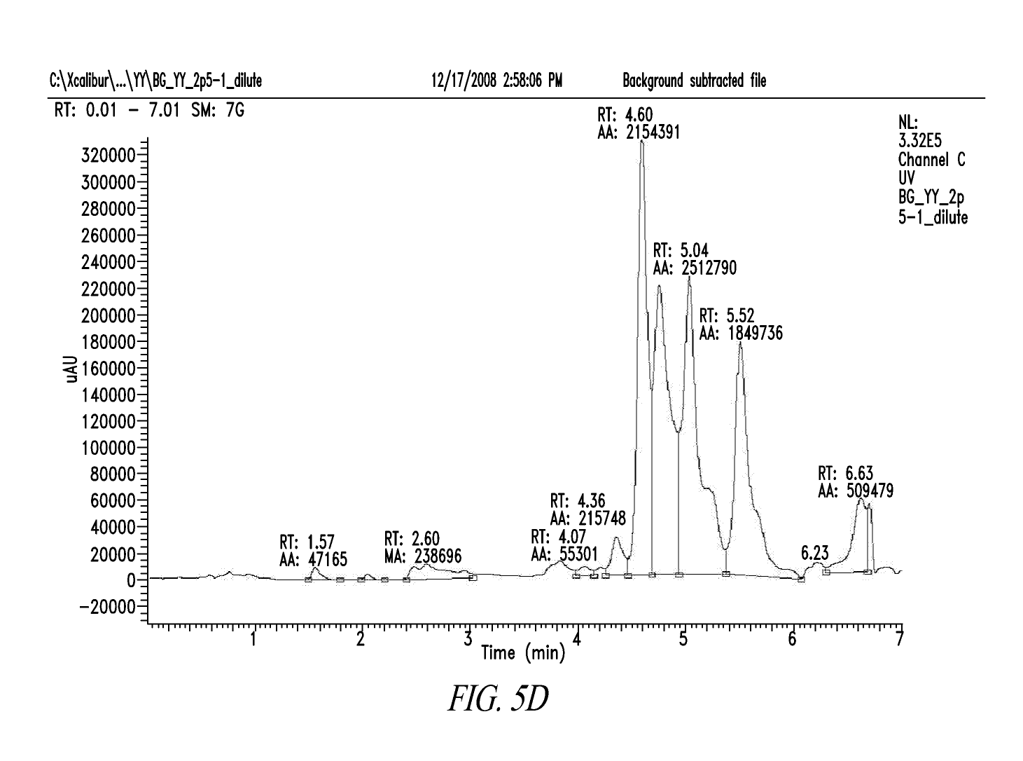

FIG. 5 illustrates the alginate residuals after the growth of the above-strains of E. coli on alginate. FIG. 5A shows the starting media, which contains a substantial amount of oligoalginate molecules (e.g., ΔM, ΔG, ΔMM, ΔGG), represented by the four left-most peaks. FIG. 5B shows a slightly reduced concentration of oligoalginate molecules in media after incubation with the E. coli containing the pALG1.5 vector. FIGS. 5C and 5D show a significantly reduced concentration of oligoalginate molecules in media after incubation with E. coli containing the pALG2.0 and pALG2.5 vectors, respectively, showing that these oligoalginate molecules are being utilized by the recombinant E. coli as a source of carbon, energy, or both.

### Example 3

### Modifying Escherichia Coli to Grow on Cellobiose and Carboxy Methyl Cellulose as a Sole Source of Carbon and Energy

To create E. coli strains that grow on cellobiose and carboxy methyl cellulose as a sole source of carbon and energy, various cellulase genes were first obtained from Saccharophagus degradans 2-40 and cloned into sub-vectors. Specifically, a variety of cellulases, cellobiohydrolases, cellodextrinases and β-glucosidases, summarized in Table 3 below, were sub-cloned into five different vector systems, pING1-Bgls, pING2-Cell, pING1-Cel2, pING2-Cel3, and pING1-Cel4. The cloning of each of these vectors is summarized below. Escherichia coli strain EC100 or DH5α was used for vector construction.

TABLE 12
Cellulases sub-cloned into vectors.

| Plasmid | |
|---|---|
| name | S. degradans genes incorporated in each plasmid |
| pING1Bgls | Bgl1A (Sde_3603), Bgl1B (Sde_1394), |
| | Bgl3C (Sde_2674) |
| pING2Cel1 | Cel5B (Sde_2490), Cel5J (Sde_2494), |
| | Ced3A (Sde_2497) |
| pING1Cel2 | Cel5C (Sde_0325), Ced3B (Sde_0245), |
| | Cel9B (Sde_0649), Cel5F (Sde_1572) |
| pING2Cel3 | Cel9A (Sde_0636), Cel6A (Sde_2272) |
| pING1Cel4 | Cel5A (Sde_3003), Cel5E (Sde_2929), |
| | Cel5I (Sde_3420) |

Construction of the pING1-Bgls vector. The pING1 vector, Bgl1A, Bgl1B, Bgl3C, and Atu2019 fragments were amplified by PCR: 98° C. for 15 sec, 55° C. for 15 sec, and 72° C. for 3 min, repeated for 30 times. The reaction mixture contained 1× Phusion buffer, 2 mM dNTP, 0.5 μM forward and reverse primers, 2.5 U Phusion DNA polymerase (Finezyme), and 100 ng of Saccharophagus degradans 2-40 genome as a template in total volume of 50 μl. These amplified fragments were spliced by over-lap PCR: 98° C. for 15 sec, 55° C. for 15 sec, and 72° C. for 6 min, repeated for 30 times. The reaction mixture contained 1× Phusion buffer, 2 mM dNTP, 0.5 μM forward (pING1-F) and reverse (Atu2019-R) primers, 2.5 U Phusion DNA polymerase (Finezyme), and the 50-100 ng of abovementioned DNA fragments as a template in a total volume of 50 μl. The spliced fragment was then digested with XbaI, ligated using T4 DNA ligase, and transformed into EC100.

Construction of the pING2Cel1 vector. The pING2 vector, Cel5B, and Ced3A/Cel5J fragments were amplified by PCR: 98° C. for 15 sec, 55° C. for 15 sec, and 72° C. for 3 min, repeated for 30 times. The reaction mixture contained 1× Phusion buffer, 2 mM dNTP, 0.5 μM forward and reverse primers, 2.5 U Phusion DNA polymerase (Finezyme), and 100 ng of Saccharophagus degradans 2-40 genome as a template in total volume of 50 μl. These amplified fragments were spliced by over-lap PCR: 98° C. for 15 sec, 55° C. for 15 sec, and 72° C. for 6 min, repeated for 30 times. The reaction mixture contained 1× Phusion buffer, 2 mM dNTP, 0.5 μM forward (pING2-F) and reverse (Ced3A/Cel5J-R) primers, 2.5 U Phusion DNA polymerase (Finezyme), and the 50-100 ng of abovementioned DNA fragments as a template in a total volume of 50 μl. The spliced fragment was then digested with XbaI, ligated using T4 DNA ligase, and transformed into EC100.

Construction of the pING1Cel2 vector. The pING1 vector, Cel5C, Ced3B, Cel9B, and Cel5F fragments were amplified by PCR: 98° C. for 15 sec, 55° C. for 15 sec, and 72° C. for 3 min, repeated for 30 times. The reaction mixture contained 1× Phusion buffer, 2 mM dNTP, 0.5 μM forward and reverse primers, 2.5 U Phusion DNA polymerase (Finezyme), and 100 ng of Saccharophagus degradans 2-40 genome as a template in total volume of 50 μl. These amplified fragments were spliced by over-lap PCR: 98° C. for 15 sec, 55° C. for 15 sec, and 72° C. for 6 min, repeated for 30 times. The reaction mixture contained 1× Phusion buffer, 2 mM dNTP, 0.5 μM forward (pING1-F) and reverse (Cel5F-R) primers, 2.5 U Phusion DNA polymerase (Finezyme), and the 50-100 ng of abovementioned DNA fragments as a template in a total volume of 50 μl. The spliced fragment was then digested with XbaI, ligated using T4 DNA ligase, and transformed into EC100.

Construction of the pING2Cel3 vector. The pING2 vector, Cel9A and Ced6A fragments were amplified by PCR: 98° C. for 15 sec, 55° C. for 15 sec, and 72° C. for 3 min, repeated for 30 times. The reaction mixture contained 1× Phusion buffer, 2 mM dNTP, 0.5 μM forward and reverse primers, 2.5 U Phusion DNA polymerase (Finezyme), and 100 ng of Saccharophagus degradans 2-40 genome as a template in total volume of 50 μl. These amplified fragments were spliced by over-lap PCR: 98° C. for 15 sec, 55° C. for 15 sec, and 72° C. for 6 min, repeated for 30 times. The reaction mixture contained 1× Phusion buffer, 2 mM dNTP, 0.5 μM forward (pING2-F) and reverse (Cel6A-R) primers, 2.5 U Phusion DNA polymerase (Finezyme), and the 50-100 ng of abovementioned DNA fragments as a template in a total volume of 50 μl. The spliced fragment was then digested with BamHI, ligated using T4 DNA ligase, and transformed into EC100.

Construction of the pING1Cel4 vector. The pING1 vector, Cel5A, Cel5E, and Cel5I fragments were amplified by PCR: 98° C. for 15 sec, 55° C. for 15 sec, and 72° C. for 3 min, repeated for 30 times. The reaction mixture contained 1× Phusion buffer, 2 mM dNTP, 0.5 μM forward and reverse primers, 2.5 U Phusion DNA polymerase (Finezyme), and 100 ng of Saccharophagus degradans 2-40 genome as a template in total volume of 50 μl. These amplified fragments were spliced by over-lap PCR: 98° C. for 15 sec, 55° C. for 15 sec, and 72° C. for 6 min, repeated for 30 times. The reaction mixture contained 1× Phusion buffer, 2 mM dNTP, 0.5 μM forward (pING1-F) and reverse (Cel5I-R) primers, 2.5 U Phusion DNA polymerase (Finezyme), and the 50-100 ng of abovementioned DNA fragments as a template in a total volume of 50 μl. The spliced fragment was then digested with BamHI, ligated using T4 DNA ligase, and transformed into EC100.

The cellulase fragments of the Bgls, Cel1, Cel2, Cel3, and Cel4 subvectors were then integrated into the pALG vectors. Cellulase fragments Bgls, Cel1, Cel2, Cel3, and Cel4, were amplified by PCR: 98° C. for 15 sec, 55° C. for 15 sec, and 72° C. for 6-7 min, repeated for 30 times. The reaction mixture contained 1× Phusion buffer, 2 mM dNTP, 0.5 μM forward and reverse primers, 2.5 U Phusion DNA polymerase (Finezyme), and 100 ng of pING1Bgls, pING2Cel1, pING1Cel2, pING2Cel3, pING1Cel4 as templates in total volume of 50 μl, respectively. A more detailed explanation of the construction of these vectors is provided below. The following vectors are diagrammed in FIGS. 3F-J.

Construction of pALG4.0. A vector containing Sde_{—}3602 (Glutathione synthetase), Sde_{—}3603 (Bgl1A), Sde_{—}1394 (Bgl1B), Sde_{—}1395 (cellobiose transporter), Sde_{—}2674 (Bgl3C), Sde_{—}2637 (tRNA pseudouridine synthase B), and Atu_{—}3019 was constructed based on pKm plasmid backbone (R6Kγ-based vector containing kanamycin resistant gene (Km)). The pKm, Sde_{—}3602, Sde_{—}3603, Sde_{—}1394, Sde_{—}1395, Sde_{—}2674, and Sde_{—}2637 sequences were amplified by PCR: 98° C. for 10 sec, 55° C. for 15 sec, and 72° C. for 1 min for pKm and 5 min for Sde_{—}3602, Sde_{—}3603, Sde_{—}1394, Sde_{—}1395, Sde_{—}2674, Sde_{—}2637, and Atu_{—}3019 repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:471), 5′-GAATCGTTTTCCGGGACGCCAATACACCTACCCAATCGCCAATTG-3′ (SEQ ID NO:472), 5′-CGGCTCGTTTCAATTTCTACACTGTTAGCTCCTACTCGAGACAAACTCAG-3′ (SEQ ID NO:473), 5′-GTATCAAAATAAAAGAGTTAATACATATGCTGCTAAGCTTAAAAAACACT-3′ (SEQ ID NO:474) and 5′-CCATATACCCCATAAGCGTTGCGGCTCACTGACTTGAACGGATATTGACG-3′ (SEQ ID NO:475), respectively) and reverse (5′-CAATTGGCGATTGGGTAGGTGTATTGGCGTCCCGGAAAACGATTC-3′ (SEQ ID NO:476), 5′-CTGAGTTTGTCTCGAGTAGGAGCTAACAGTGTAGAAATTGAAACGAGCCG-3′ (SEQ ID NO:477), 5′-AGTGTTTTTTAAGCTTAGCAGCATATGTATTAACTCTTTTATTTTGATAC-3′ (SEQ ID NO:478), 5′-CGTCAATATCCGTTCAAGTCAGTGAGCCGCAACGCTTATGGGGTATATGG-3′ (SEQ ID NO:479), and 5′-GCTCTAGAGTTGCCGCCCTCCGGCAATTCG-3′ (SEQ ID NO:480), respectively) primers, 100 ng of purified genome of Saccharophagus degradans 2-40 or Agrobacterium tumefaciens C58 or 50 ng of purified pKm vector, and 2.5 U Phusion DNA polymerase (Finezyme) in total volume of 50 μA. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:481) and reverse (5′-GCTCTAGAGTTGCCGCCCTCCGGCAATTCG-3′) (SEQ ID NO:482) primers, 5 ul of each purified DNA fragment, and 2.5 U Phusion DNA polymerase (Finezyme) in total volume of 50 μA. The amplified fragment was then digested with XbaI (New England Biolabs) and ligated with T4 DNA ligase to form pKm-Sde_{—}3602-3603-1394-1395-2674-2637-Atu_{—}3019. The constructed plasmid was sequenced (Elim Biopharmaceuticals) and the DNA sequence of the insert was confirmed. The Km-Sde_{—}3602-3603-1394-1395-2674-2637-Atu_{—}3019 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:483) and reverse (5′-GTTGCCGCCCTCCGGCAATTCG-3′) (SEQ ID NO:484) primers, 50 ng of purified pKm-Sde_{—}3602-3603-1394-1395-2674-2637-Atu_{—}3019, and 2.5 U Phusion DNA polymerase (Finezyme) in total volume of 50 μl. The amplified fragment was gel purified and transformed into a strain harboring pALG3.5 to construct pALG4.0 via homologous recombination.

Construction of pALG5.0. A vector containing Sde_{—}2491 (Transcription regulator), Sde_{—}2490 (Cel5B), Sde_{—}2497 (Ced3A), Sde_{—}2496 (Glyoxylase), Sde_{—}2495 (Transcription regulator), and Sde_{—}2494 (Cel5J) was constructed based on pCm plasmid backbone (R6Kγ-based vector containing chloramphenicol resistant gene (Cm)). The pCm, Sde_{—}2491, Sde_{—}2490, Sde_{—}2497, Sde_{—}2496, Sde_{—}2495, and Sde_{—}2494 sequences were amplified by PCR: 98° C. for 10 sec, 55° C. for 15 sec, and 72° C. for 1 min for pCm and 2 min for Sde_{—}2491, Sde_{—}2490, Sde_{—}2497, Sde_{—}2496, Sde_{—}2495, and Sde_{—}2494 repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:485), 5′-GCTGTCAAACATGAGAATTGGTCGGCCCAACTGCAGCTGCGACAAAAGC-3′ (SEQ ID NO:486), and 5′-TGTATTAGTGGCGCCAAACCCGTAGTACACTCGCCGACGGCAAATTCTAA-3′ (SEQ ID NO:487), respectively) and reverse (5′-GCTTTTGTCGCAGCTGCAGTTGGGCCGACCAATTCTCATGTTTGACAGC-3′ (SEQ ID NO:488), 5′-TTAGAATTTGCCGTCGGCGAGTGTACTACGGGTTTGGCGCCACTAATACA-3′ (SEQ ID NO:489), and 5′-GCTCTAGAAATGCCTTAAAACTTGATGCATATA-3′ (SEQ ID NO:490), respectively) primers, 100 ng of purified genome of Saccharophagus degradans 2-40 or 50 ng of purified pCm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:491) and reverse (5′-GCTCTAGAAATGCCTTAAAACTTGATGCATATA-3′) (SEQ ID NO:492) primers, 5 ul of each purified DNA fragment, and 2.5 U Phusion DNA polymerase (Finezyme) in total volume of 50 μl. The amplified fragment was then digested with XbaI (New England Biolabs) and ligated with T4 DNA ligase to form pCm-Sde_{—}2491-2490-2497-2496-2495-2494. The constructed plasmid was sequenced (Elim Biopharmaceuticals) and the DNA sequence of the insert was confirmed. The Cm-Sde_{—}2491-2490-2497-2496-2495-2494 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:493) and reverse (5′-GCCGTTAAAGATTCGCAATTGGCGATTGGGTAGGTGTATTAATGCCTTAAAA CTTGATGC-3′) (SEQ ID NO:494) primers, 50 ng of purified pCm-Sde_{—}2491-2490-2497-2496-2495-2494, and 2.5 U Phusion DNA polymerase (Finezyme) in total volume of 50 μl. The amplified fragment was gel purified and transformed into a strain harboring pALG4.0 to construct pALG5.0 via homologous recombination.

Construction of pALG5.1. A vector containing Sde_{—}0245 (Ced3B), Sde_{—}0324 (Transcription regulator), Sde_{—}0325 (Cel5C), Sde_{—}0649 (Cel9B), and Sde_{—}1572 (Cel5F) was constructed based on pKm plasmid backbone (R6Kγ-based vector containing kanamycin resistant gene (Km)). The pKm, Sde_{—}0245, Sde_{—}0324, Sde_{—}0325, Sde_{—}0649, and Sde_{—}1572 sequences were amplified by PCR: 98° C. for 10 sec, 55° C. for 15 sec, and 72° C. for 1 min for pKm and 2 min for Sde_{—}0245, Sde_{—}0324, Sde_{—}0325, Sde_{—}0649, and Sde_{—}1572 repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:495), 5′-GAATCGTTTTCCGGGACGCCACGCAACTACTGCGTAACGCTATGG-3′ (SEQ ID NO:496), 5′-TAGCGCAGCTATTAAGTGTGACTAACCCTTAAAACTGCCAGCCGCTATTA-3′ (SEQ ID NO:497), 5′-ACTGCGCCACCGTGTAATATCATTGTTACTTAACTAAACAGCTTGGCGTG-3′ (SEQ ID NO:498), and 5′-CTAGATAGAAAATAGAATTGTAAGCGAGGCGATGAGCTTCTATTAAGTAT-3′ (SEQ ID NO:499), respectively) and reverse (5′-CCATAGCGTTACGCAGTAGTTGCGTGGCGTCCCGGAAAACGATTC-3′ (SEQ ID NO:500), 5′-TAATAGCGGCTGGCAGTTTTAAGGGTTAGTCACACTTAATAGCTGCGCTA-3′ (SEQ ID NO:501), 5′-CACGCCAAGCTGTTTAGTTAAGTAACAATGATATTACACGGTGGCGCAGT-3′ (SEQ ID NO:502), 5′-ATACTTAATAGAAGCTCATCGCCTCGCTTACAATTCTATTTTCTATCTAG-3′ (SEQ ID NO:503), and 5′-GCTCTAGACGAATTGCAGACTTTTGCGTGATTG-3′ (SEQ ID NO:504), respectively) primers, 100 ng of purified genome of Saccharophagus degradans 2-40 or 50 ng of purified pKm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:505) and reverse (5′-GCTCTAGACGAATTGCAGACTTTTGCGTGATTG-3′) (SEQ ID NO:506) primers, 5 ul of each purified DNA fragment, and 2.5 U Phusion DNA polymerase (Finezyme) in total volume of 50 μl. The amplified fragment was then digested with XbaI (New England Biolabs) and ligated with T4 DNA ligase to form pKm-Sde_{—}0245-0324-0325-0649-1572. The constructed plasmid was sequenced (Elim Biopharmaceuticals) and the DNA sequence of the insert was confirmed. The Km-Sde_{—}0245-0324-0325-0649-1572 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:507) and reverse (5′-AATTAGACAAAGTATGCTTTTGTCGCAGCTGCAGTTGGGCCGAATTGCAGAC TTTTGCGT-3′) (SEQ ID NO:508) primers, 50 ng of purified pKm-Sde_{—}0245-0324-0325-0649-1572, and 2.5 U Phusion DNA polymerase (Finezyme) in total volume of 50 μl. The amplified fragment was gel purified and transformed into a strain harboring pALG5.0 to construct pALG5.1 via homologous recombination.

Construction of pALG5.2. A vector containing Sde_{—}0636 (Cel9A), and Sde_{—}2272 (Cel6A) was constructed based on pCm plasmid backbone (R6Kγ-based vector containing chloramphenicol resistant gene (Cm)). The pCm, Sde_{—}0636, and Sde_{—}2272 sequences were amplified by PCR: 98° C. for 10 sec, 55° C. for 15 sec, and 72° C. for 1 min for pCm and 2 min for Sde_{—}0636, and Sde_{—}2272 repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:509), 5′-GCTGTCAAACATGAGAATTGGTCGCCGCCGAGACGACAGCAAGCTGGAC-3′ (SEQ ID NO:510), and 5′-CGAATACATACACCACCTAAAATACAGAGGAAAAAATCATGTTGGCTTCT-3′ (SEQ ID NO:511), respectively) and reverse (5′-GTCCAGCTTGCTGTCGTCTCGGCGGCGACCAATTCTCATGTTTGACAGC-3′ (SEQ ID NO:512), 5′-AGAAGCCAACATGATTTTTTCCTCTGTATTTTAGGTGGTGTATGTATTCG-3′ (SEQ ID NO:513), and 5′-CGGGATCCATATGGAGTGTTTTTTTAATGTTGT-3′ (SEQ ID NO:514), respectively) primers, 100 ng of purified genome of Saccharophagus degradans 2-40 or 50 ng of purified pCm vector, and 2.5 U Phusion DNA polymerase (Finezyme) in total volume of 50 μA. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:515) and reverse (5′-CGGGATCCATATGGAGTGTTTTTTTAATGTTGT-3′) (SEQ ID NO:516) primers, 5 ul of each purified DNA fragment, and 2.5 U Phusion DNA polymerase (Finezyme) in total volume of 50 μl. The amplified fragment was then digested with XbaI (New England Biolabs) and ligated with T4 DNA ligase to form pCm-Sde_{—}0636-2272. The constructed plasmid was sequenced (Elim Biopharmaceuticals) and the DNA sequence of the insert was confirmed. The Cm-Sde_{—}0636-2272 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:517) and reverse (5′-ACCTTCACTTTAGTGCCATAGCGTTACGCAGTAGTTGCGTATATGGAGTGTTT TTTTAAT-3′) (SEQ ID NO:518) primers, 50 ng of purified pCm-Sde_{—}0636-2272, and 2.5 U Phusion DNA polymerase (Finezyme) in total volume of 50 μl. The amplified fragment was gel purified and transformed into a strain harboring pALG5.1 to construct pALG5.2 via homologous recombination.

Construction of pALG5.3. A vector containing Sde_{—}2929 (Cel5E) Sde_{—}3003 (Cel5A), and Sde_{—}3420 (Cel5I) was constructed based on the pKm plasmid backbone (R6Kγ-based vector containing kanamycin resistant gene (Km)). The pKm, Sde_{—}2929 Sde_{—}3003, and Sde_{—}3420 sequences were amplified by PCR: 98° C. for 10 sec, 55° C. for 15 sec, and 72° C. for 1 min for pKm and 2 min for Sde_{—}2929 Sde_{—}3003, and Sde_{—}3420 repeated for 30 times. The reaction mixture contained 1× Phusion buffer, 2 mM dNTP, 0.5 μM forward (5′-CGGGTACCGGGCCCCCCCTCGAGGTC-3′ (SEQ ID NO:519), 5′-GAATCGTTTTCCGGGACGCCGAACTAATAATGAGCGAGCAATAAC-3′ (SEQ ID NO:520), 5′-AAGCCGTAACAGTACCAATCAACATAATCGTCTCCTTGTTTGAGCGTGAT-3′ (SEQ ID NO:521), and 5′-GTGGAGGGAGGCAATCGCTAATTGAAAAATTAGAGTGTGTGGCATTTGTT-3′ (SEQ ID NO:522), respectively) and reverse (5′-GTTATTGCTCGCTCATTATTAGTTCGGCGTCCCGGAAAACGATTC-3′ (SEQ ID NO:523), 5′-ATCACGCTCAAACAAGGAGACGATTATGTTGATTGGTACTGTTACGGCTT-3′ (SEQ ID NO:524), 5′-AACAAATGCCACACACTCTAATTTTTCAATTAGCGATTGCCTCCCTCCAC-3′ (SEQ ID NO:525, and 5′-CGGGATCCCTTAGTGAACCTCTGATTGACGACC-3′ (SEQ ID NO:526), respectively) primers, 100 ng of purified genome of Saccharophagus degradans 2-40 or 50 ng of purified pKm vector, and 2.5 U Phusion DNA polymerase (Finezyme) in total volume of 50 μl. Each amplified DNA fragment was gel purified and eluted into 30 ul of Elution buffer (QIAGEN). These amplified fragments were spliced by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CGGGTACCGGGCCCCCCCTCGAGGTC-3′) (SEQ ID NO:527) and reverse (5′-CGGGATCCCTTAGTGAACCTCTGATTGACGACC-3′) (SEQ ID NO:528) primers, 5 ul of each purified DNA fragment, and 2.5 U Phusion DNA polymerase (Finezyme) in total volume of 50 μl. The amplified fragment was then digested with XbaI (New England Biolabs) and ligated with T4 DNA ligase to form pKm-Sde_{—}2929-3003-3420. The constructed plasmid was sequenced (Elim Biopharmaceuticals) and the DNA sequence of the insert was confirmed. The Km-Sde_{—}2929-3003-3420 was then amplified by PCR: 98° C. for 10 sec, 55° C. for 15 sec, and 72° C. for 6 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:529) and reverse (5′-AAAGTCGTTTATATAGTCCAGCTTGCTGTCGTCTCGGCGGCTTAGTGAACCTC TGATTGA-3′) (SEQ ID NO:530) primers, 50 ng of purified pKm-Sde_{—}2929-3003-3420, and 2.5 U Phusion DNA polymerase (Finezyme) in total volume of 50 μl. The amplified fragment was gel purified and transformed into a strain harboring pALG5.2 to construct pALG5.3 via homologous recombination.

Construction of pALG7.0, 7.1, 7.2, 7.3, 7.4, and 7.5. pALG vectors containing Ag43-ΔPaAly under the control of different promoters are constructed based on pALG2.0, 2.5 and 4.0 plasmid backbones. The fragments encoding promoter-Ag43-ΔPaAly fragments are amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CTGACCGTTCTGTCCGTCACTTCCC-3′) (SEQ ID NO:531) and reverse (5′-GCCGTTAAAGATTCGCAATTGGCGATTGGGTAGGTGTATTGAATTCAACTGC AAAAATAG-3′ (SEQ ID NO:532) for promoter P_{D/E20} into pALG4.0 to create pALG7.0, 5′-GCCGTTAAAGATTCGCAATTGGCGATTGGGTAGGTGTATTGAATTCTTATCAA AAAGAGT-3′ (SEQ ID NO:533) for promoter P_{D/E20} into pALG4.0 to create pALG7.1, 5′-GCCGTTAAAGATTCGCAATTGGCGATTGGGTAGGTGTATTGAATTCTTTTAAA AAATTCA-3′ (SEQ ID NO:534) for promoter P_{H207} into pALG4.0 to create pALG7.2, 5′-GCCGTTAAAGATTCGCAATTGGCGATTGGGTAGGTGTATTGAATTCATCAAA AAAATATT-3′ (SEQ ID NO:535) for promoter P_{LPP} into pALG4.0 to create pALG7.3, 5′-AGCCGCTGTAAAAAGTTATAGTTGTTGATTTAGAAGGAAAGAATTCTTTTAA AAAATTCA-3′ (SEQ ID NO:536) for promoter P_{H207} into pALG2.0 to create pALG7.4, and 5′-CTTTCAAATCAATTCATTTAAATAAGAGCCGAGTACTTAAGAATTCTTTTAAA AAATTCA-3′ (SEQ ID NO:537) for promoter PH207 into pALG2.5 to create pALG7.5) primers, 50 ng of purified plasmids, pCCFOS-[promoter]-Ag43-ΔPaAly. These amplified fragments were recombined with their corresponding pALG vectors to yield pALG7.0, 7.1, 7.2, 7.3, 7.4, and 7.5, respectively.

Construction of pALG7.2.1, 7.2.2, 7.2.3, and 7.2.4. Ag43-ΔPaAly was integrated into pALG2.1, 2.2, and 2.3 to make pALG7.2.1, 7.2.2, 7.2.3, and 7.2.4. The fragment encoding P_{H207}-Ag43-ΔPaAly was amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-CTGACCGTTCTGTCCGTCACTTCCC-3′ [SEQ ID NO:538]) and reverse (5′-TTTAATCGTTAGATTCTAATAGCTAGCCTCCAATTAGGCGGAATTCTTTTAAA AAATTCA-3′ [SEQ ID NO:539] for pALG2.1 to construct pALG7.2.1, 5′-CTTTCAAATCAATTCATTTAAATAAGAGCCGAGTACTTAAGAATTCTTTTAAA AAATTCA-3′ [SEQ ID NO:540] for pALG2.2 and pALG2.3 to construct pALG7.2.2 and pALG7.2.3, respectively, and 5′-TAATCACTCGTCGTACTTGTAAACGTTCGGAACATCCACCGAATTCTTTTAAA AAATTCA-3′ [SEQ ID NO:541] for pALG2.3 to construct pALG7.2.4) primers, 50 ng of purified plasmids, pCCFOS-P_{H207}-Ag43-ΔPaAly. These amplified fragments were recombined with their corresponding pALG vectors to yield pALG7.2.1, 7.2.2, 7.2.3, and 7.2.4.

Construction of pALG7.6. To investigate the effect of alginate lyases from Vibrio splendidus V12B01 over the growth of E. coli on alginate V12B01_{—}24249-24259 were excised from pALG7.2. The Km2 cassette was then amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-GGACGAGCCGTCTGGACAAACAAATGAGCAATAGTAAGTGATTCCGGGGATC CGTCGACC-3′ [SEQ ID NO:542]) and reverse (5′-CTCACTATAGGGCGAATTCGAGCTCGGTACCCGGGGATCCGTGTAGGCTGGA GCTGCT-3′ [SEQ ID NO:543]) primers, 50 ng of purified pKm2, and 2.5 U Phusion DNA polymerase (Finezyme) in total volume of 50 μl. The amplified fragment was gel purified and transformed into a strain harboring pALG7.2 to construct pALG7.6 via homologous recombination. The kanamycin selection marker was excised from the pALG7.6 through over-expression of FLP.

Construction of pALG7.8. The Km2-V12B01_{—}24254-24259 was amplified by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-TCTAATCGAATAACACTTAATATTAAAGG-3′ [SEQ ID NO:544]) and reverse (5′-CTCACTATAGGGCGAATTCGAGCTCGGTACCCGGGGATCCGTGTAGGCT-GGAGCTGCTTC-3′ [SEQ ID NO:545]) primers, 50 ng of purified pKm2-V12B01_{—}24264-24274, and 2.5 U Phusion DNA polymerase (Finezyme) in total volume of 50 μl. The amplified fragment was gel purified and transformed into a strain harboring pALG7.2 to construct pALG7.8 via homologous recombination. The kanamycin selection marker was excised from the pALG7.8 through over-expression of FLP.

Biological Activity. To test the growth of E. coli on cellobiose and carboxy methyl cellulose, E. coli cultures of DH5α harboring pALG3.5, pALG4.0, pALG5.0, pALG5.1, pALG5.2, and pALG5.3 were first grown over night in LB media at 30° C. in an orbital shaker (200 rpm). Ten percent of these cultures were then transferred to fresh LB media and grown in an incubation shaker at 30° C. to a final OD600 nm of 0.4-0.6. The cultures were centrifuged, and the pellets were resuspended in M9 media containing either 1% cellobiose or 1% carboxymethylcellulose supplemented with 1 mg/ml Thiamine to a final OD₆₀₀ₙₘ of 0.1. The cultures were then grown in an incubation shaker at 30° C. and the OD₆₀₀ₙₘ was measured at different time points.

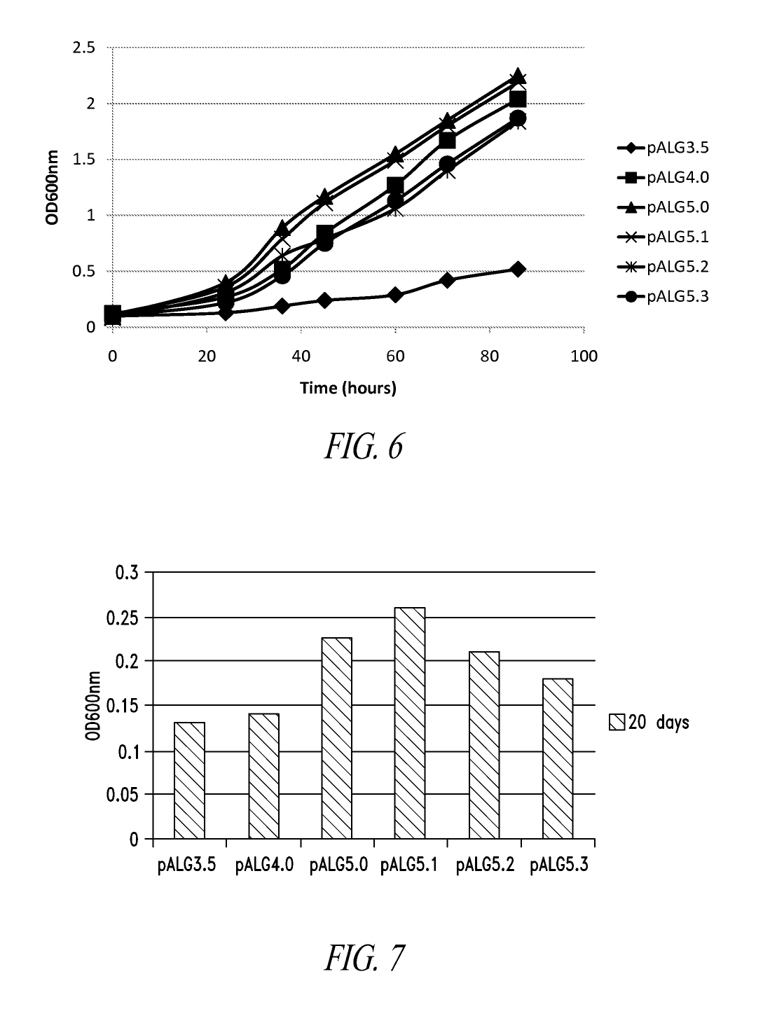

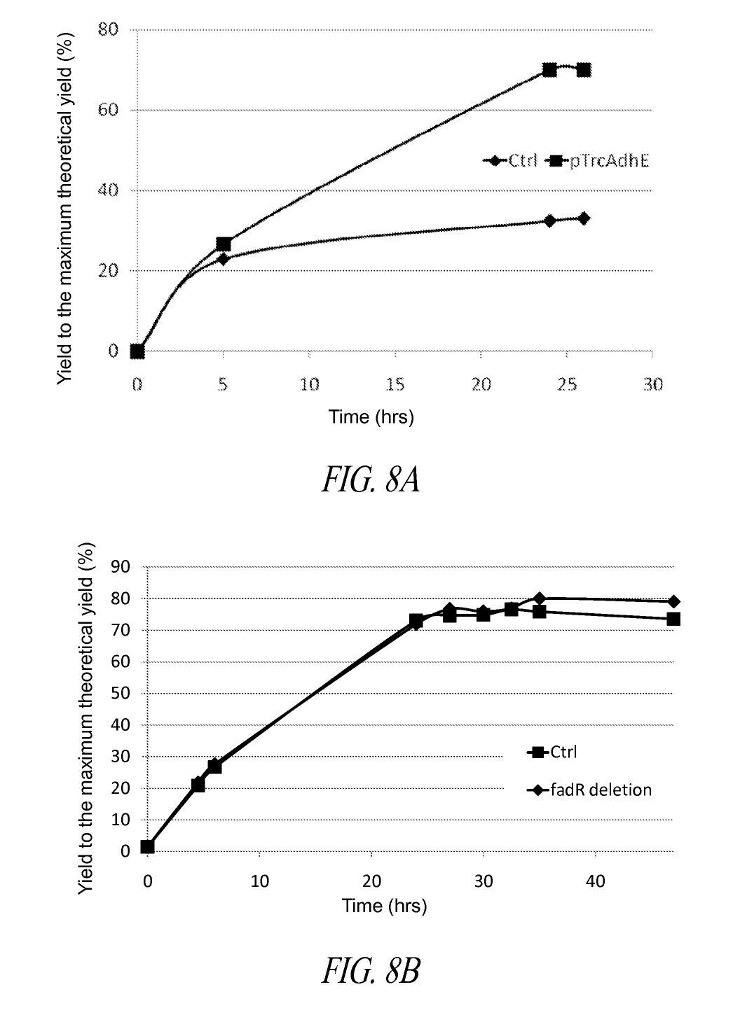

FIG. 6 shows the OD₆₀₀ₙₘ values for E. coli growing on cellobiose, and FIG. 7 shows the OD₆₀₀ₙₘ values for E. coli growing in methyl carboxy cellulose. These results show that the Bgls fragment conferred on E. coli the ability to grow on cellobiose as a sole source of carbon. The Cell-4 fragment also conferred on E. coli the ability to grow on carboxy methyl cellulose as a sole source of carbon. In this experiment, pALG3.5 and pALG4.0 do not carry endo-cellulase and cellobiohydrolases, and, thus, can be considered negative controls.

To test the growth of E. coli on alginate and guluronate, E. coli ATCC8739 harboring pALG1.5, pALG1.7, pALG2.1, pALG2.2, pALG2.3 pALG7.2.1, pALG7.2.2, pALG7.2.3, and pALG7.2.4 were first grown in LB media at 30° C. for overnight. One percent of these cultures was then inoculated into M9 media containing 0.2% alginate pre-digested with G-specific alginate lyase. Optical density 600 nm was measured 30 hours after inoculation. As shown in FIGS. 19A-19F, the genomic region encoding four alginate lyases and outer membrane porin (V12B01_{—}24254-24274) improved the growth of E. coli on alginate and guluronate. The genomic region encoding transporter and outer membrane porins (V12B01_{—}24309 and V12B01_{—}24324) also improved the growth of E. coli on alginate and guluronate.

These systems should be very useful for the production of fuels and chemicals, especially in using cellobiose, carboxy methyl cellulose, and other polysaccharides as a feed stock for microbial growth.

### Example 4

### Increased Ethanol Production from Deletion Mutants

To improve the production of ethanol, a series of deletion mutants were constructed in E. coli. As detailed below, the reduced production of other carbon based molecules was accomplished by deleting certain key genes in the biosynthesis of those molecules.

Specifically, at least three different deletion mutant versions of E. coli were created from E. coli strain W version AL1.0, which contains the pdc-adhA/B operon, and is therefore capable of producing ethanol from glucose. First, the AL2.0 version comprises a deletion in the lactose dehydrogenase gene (ΔldhA), which plays a key role in the synthesis of lactate. The AL3.0 version comprises the ΔldhA deletion, and further comprises a deletion in the fumarate reductase gene (Δfrd), which converts fumarate into succinate. The AL4.0 version comprises the ΔldhA and Δfrd deletions, and further comprises a deletion in the pflB-focA operon (ΔpflB-focA). The pflB-focA operon encodes the central enzyme of fermentative metabolism, pyruvate formate-lyase (PFL), and a membrane protein, FocA, thought to transport formate. The fadR gene encodes for a regulator of aerobic fatty acid metabolism, and it is believed that deletions in fadR enhance fatty acid metabolism. Deletion mutants of the ppc, pck, mdh, pta, sdh, and fumB genes were also constructed.

Standard procedure described by Datsenko and Wanner was used to delete the above-noted genes. The fragments to be inserted into targeted positions in genome were amplified by PCR: 98° C. for 10 sec, 55° C. for 15 sec, and 72° C. for 1 min, repeated for 30 times.

The reaction mixture contained 1× Phusion buffer, 2 mM dNTP, 0.5 μM forward (5′-ACTGGTCAGAGCTTCTGCTGTCAGGAATGCCTGGTGCCCGGTGTAGGCTGGA GCTGCTTC-3′ [SEQ ID NO:546] for ldhA deletion, 5′-CGACACCAATCAGCGTGACAACTGTCAGGATAGCAGCCAGGTGTAGGCTGGA GCTGCTTC-3′ [SEQ ID NO:547] for frd deletion, 5′-TTACATAGATTGAGTGAAGGTACGAGTAATAACGTCCTGCGTGTAGGCTGGA GCTGCTTC-3′ [SEQ ID NO:548] for pflB-focA deletion, 5′-TTAGAACATTACCTTATGACCGTACTGCTCAAGAATGCCTGTGTAGGCTGGAG CTGCTTC-3′ [SEQ ID NO:549] for pflA-focA deletion, 5′-ATGGTCATTAAGGCGCAAAGCCCGGCGGGTTTCGCGGAAGGTGTAGGCTGGA GCTGCTTC-3′ [SEQ ID NO:550] for fadR deletion, 5′-CCCCAAAAAGACTTTACTATTCAGGCAATACATATTGGCTGTGTAGGCTGGA GCTGCTTC-3′ [SEQ ID NO:551] for pck deletion, 5′-TTACTTAGTGCAGTTCGCGCACTGTTTGTTGACGATTTGCGTGTAGGCTGGAG CTGCTTC-3′ [SEQ ID NO:552] for fumB deletion, 5′-TTACTTATTAACGAACTCTTCGCCCAGGGCGATATCTTTCGTGTAGGCTGGAG CTGCTTC-3′ [SEQ ID NO:553] for mdh deletion, 5′-TCCAGGTAACAGAAAGTTAACCTCTGTGCCCGTAGTCCCCGTGTAGGCTGGA GCTGCTTC-3′ [SEQ ID NO:554] for sdh deletion, 5′-TGGCGGTGCTGTTTTGTAACCCGCCAAATCGGCGGTAACGGTGTAGGCTGGA GCTGCTTC-3′ [SEQ ID NO:555] for pta deletion, and 5′-TTAGCCGGTATTACGCATACCTGCCGCAATCCCGGCAATAGTGTAGGCTGGA GCTGCTTC-3′ [SEQ ID NO:556] for ppc deletion) and reverse (5′-TTTGGCTTTGAGCTGGAATTTTTTGACTTTCTGCTGACGGATTCCGGGGATCC GTCGACC-3′ [SEQ ID NO:557] for ldhA deletion, 5′-TCTCAAAAGTATACCCGATGCGTAGCCATACCGTTGCTGCATTCCGGGGATCC GTCGACC-3′ [SEQ ID NO:558] for frd deletion, 5′-CTGCTGCAATGGCCAAAGTGGCCGAAGAGGCGGGTGTCTAATTCCGGGGATC CGTCGACC-3′ [SEQ ID NO:559] for pflB-focA deletion, 5′-CTGCTGCAATGGCCAAAGTGGCCGAAGAGGCGGGTGTCTAATTCCGGGGATC CGTCGACC-3′ [SEQ ID NO:560] for pflA-focA deletion, 5′-TTATCGCCCCTGAATGGCTAAATCACCCGGCAGATTTTTCATTCCGGGGATCC GTCGACC-3′ [SEQ ID NO:561] for fadR deletion, 5′-TTACAGTTTCGGACCAGCCGCTACCAGCGCGGCACCCGCAATTCCGGGGATC CGTCGACC-3′ [SEQ ID NO:562] for pck deletion, 5′-ATGCACTTTGCGTGCCGCCCGTGACTACGCGGCACGCCATATTCCGGGGATC CGTCGACC-3′ [SEQ ID NO:563] for pck deletion, 5′-CGCGGCAGCGGAGCAACATATCTTAGTTTATCAATATAATATTCCGGGGATC CGTCGACC-3′ [SEQ ID NO:564] for mdh deletion, 5′-TTACGCATTACGTTGCAACAACATCGACTTGATATGGCCGATTCCGGGGATCC GTCGACC-3′ [SEQ ID NO:565] for sdh deletion, 5′-TTACTGCTGCTGTGCAGACTGAATCGCAGTCAGCGCGATGATTCCGGGGATC CGTCGACC-3′ [SEQ ID NO:566] for pta deletion, and 5′-TTGCGTAGTAATGTCAGTATGCTCGGCAAAGTGCTGGGAGATTCCGGGGATC CGTCGACC-3′ [SEQ ID NO:567] for ppc deletion) primers, 5% (v/v) DMSO, and 50 ng of purified plasmid, pKD13.

Versions AL1.0, AL2.0, and AL3.0 were tested for their capacity to produce ethanol while growing on glucose. These recombinant microorganisms were incubated at 37° C. in MP salt media containing 0.5% LB and 5% glucose. Culture samples were collected at various time-points, and the amount of ethanol in each sample was determined. The results are set forth in Table 13 below. The theoretical maximum yield was calculated by according to routine techniques in the art.

TABLE 13
Ethanol production from deletion mutants

| | | pdc-adhA/ | pdc-adhA/ |
|---|---|---|---|
| | pdc-adhA/ | B operon | B operon |
| | B operon | ΔldhA | ΔldhA/Δfrd |
| | [AL1.0] | [AL2.0] | [AL3.0] |
| % Yield of | 80.5 | 84.1 | 94.4 |
| Theoretical | | | |
| Maximum | | | |
| Yield (%) | 41.1 | 42.9 | 48.1 |
| Titer (g/L) | 20.5 | 21.5 | 24.1 |
| Productivity | 0.43 | .045 | 0.50 |
| (g/L/H) | | | |

These results show that the use of the above deletion mutants significantly increases the capacity of recombinant bacteria to produce ethanol from saccharides, polysaccharides, or other sources of carbon and energy, approaching the maximum theoretical yield for such microorganisms.

### Example 5

### The Effect of AdhE Expression on Ethanol Production

To test the effects of alcohol dehydrogenase (adhE) expression on ethanol production, E. coli was transformed with a vector that contains the pdc-adhA/B operon either alone or in combination with a vector (pTrcAdhE) that contains the adhE gene. AdhE is a CoA-linked aldehyde/alcohol dehydrogenase derived from E. coli. The pTrcAdhE vector was constructed as follows.

Construction of pTrcAdhE. The fragments encoding a plasmid, pTrc99A, and AdhE were amplified by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-CTCTAGAGTCGACCTGCAGGCATGC-3′ (SEQ ID NO:568) for pTrc99A fragment and 5′-AACAATTTCACACAGGAAACAGACCATGGCTGTTACTAATGTCGCTGAAC-3′ (SEQ ID NO:569) for AdhE fragment) and reverse (5′-GTTCAGCGACATTAGTAACAGCCATGGTCTGTTTCCTGTGTGAAATTGTT-3′ (SEQ ID NO:570) for pTrc99A fragment and 5′-TTAAGCGGATTTTTTCGCTTTTTTCTCAGC-3′ (SEQ ID NO:571) for AdhE fragment) primers, 50 ng of purified plasmids, pTrc99A and 100 ng of purified E. coli genome in 50 ul total volume. The amplified DNA fragments were gel purified and spliced by PCR: PCR: 98° C. for 10 sec, 55° C. for 15 sec, and 72° C. for 4 min, repeated for 30 times. The reaction mixture contained 1× Phusion buffer, 2 mM dNTP, 0.5 μM forward (5′-CTCTAGAGTCGACCTGCAGGCATGC-3′) (SEQ ID NO:572) and reverse (5′-TTAAGCGGATTTTTTCGCTTTTTTCTCAGC-3′) (SEQ ID NO:573) primers, 5 ul of purified fragments in 50 ul total volume. The spliced fragment was treated with polynucleotide kinase and ligated with T4 DNA ligase (NEB) to form pTrcAdhE.

The E. coli strain ATCC9637 ΔldhA/Δfrd/ΔpflB-focA harboring pBBRpdc-adhA/B with or without pTrcAdhE (see U.S. application Ser. No. 12/245,537, herein incorporated by reference) were grown overnight in LB media at 30° C. Cultures were inoculated into fresh M9 media containing 1.7% mannitol, 0.2% galacturonate, and 1% palmitate. Palmitate is the salt or ester form of palmitic acid, the latter having the chemical formula CH₃(CH₂)₁₄COOH (i.e., hexadecanoic acid in IUPAC nomenclature). Palmitic acid is one of the most common saturated fatty acids found in plants (including kelp) and animals.

Then, the ethanol production was monitored by gas chromatography (GC) over a 25 hour time period. As shown in FIG. 8A, the over expression of pTrcAdhE increased ethanol production from media containing sugars and fatty acids, increasing the percentage yield to the maximum theoretical yield from about 35% to about 70% or more. Further, these results show a synergistic effect between the use of deletion mutants (e.g., ΔldhA/Δfrd/ΔpflB-focA) and the use of the AdhE gene in increasing the overall ethanol production capacity in recombinant microorganisms, including microorganisms that are growing on mixtures of polysaccharides and fatty acids.

To further test the effects of fadR deletions on this system, E. coli strain ATCC9637 ΔldhA/Δfrd/ΔpflB-focA with or without a ΔfadR deletion and harboring both pBBRpdc-adhA/B and pTrcAdhE were grown overnight in LB media at 30° C. Cultures were inoculated into fresh M9 media containing 1.7% mannitol, 0.2% galacturonate, and 1% palmitate. Ethanol production was monitored by gas chromatography (GC) over a 40 hour time period. As shown in FIG. 8B, the over expression of pTrcAdhE in combination with ΔfadR increased ethanol production from media containing sugar and fatty acid. The cells with pTrcAdhE and ΔfadR maintained a higher theoretical maximum yield for a greater period of time (+10 hours), suggesting a cumulative effect for the combination of these two features, especially for recombinant microorganisms growing on mixtures of polysaccharides and fatty acids.

### Example 6

### Increased Ethanol Production from Mixed Polysaccharides

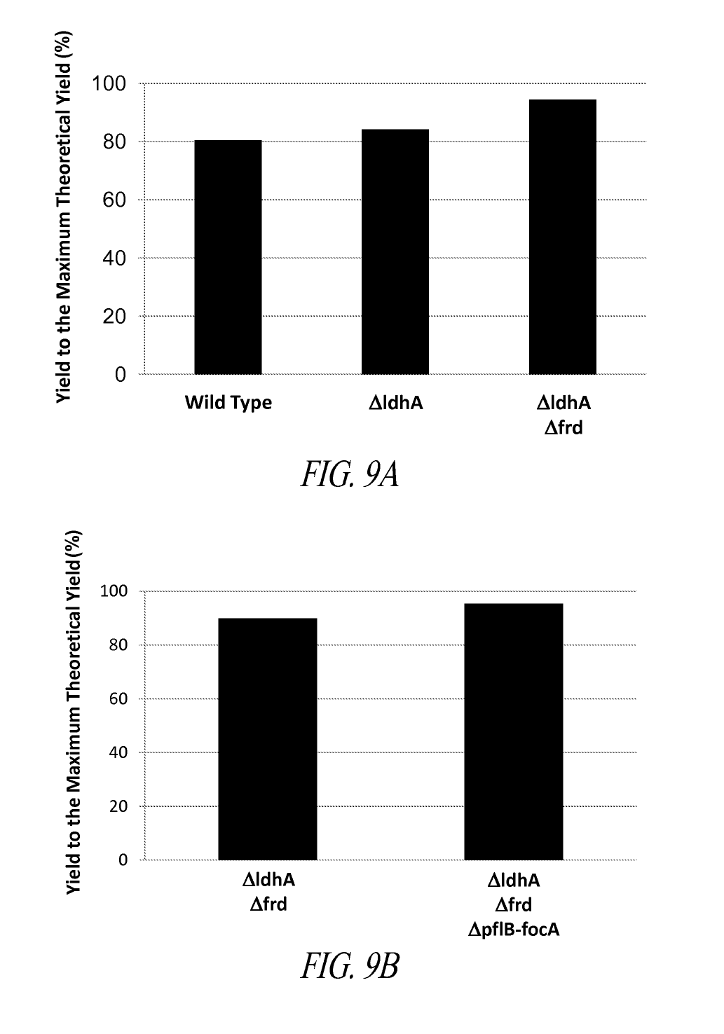

To compare the effects of various deletion mutations on ethanol production from mixed sugar sources (mannitol:uronic acid (glucuronate)), E. coli strain ATCC9637 (Wild type), ATCC9637 ΔldhA, and ATCC9637 ΔldhA/Δfrd harboring pBBRpdc-adhA/B (see U.S. application Ser. No. 12/245,537, herein incorporated by reference) were grown overnight in LB media at 30° C. Cultures were inoculated into fresh M9 media containing 5% glucose. The ethanol production at 48 hrs was monitored by gas chromatography (GC). As shown in FIG. 9A, the ΔldhA and Δfrd mutations cooperatively increased ethanol production from mixed sugars, nearly approaching the maximum theoretical yield for the production of ethanol in this system.

To compare the effects of additional deletion mutation on ethanol production from mixed sugar (mannitol:uronic acid (galacturonate)), E. coli strains ATCC9637 ΔldhA/Δfrd and ATCC9637 ΔldhA/Δfrd/ΔpflB-focA harboring pBBRpdc-adhA/B were grown overnight in LB media at 30° C. Cultures were inoculated into fresh M9 media containing 3% in total with 1:2 ratio of mannitol:galacturonate. The ethanol production at 16 hrs was monitored by gas chromatography (GC). As shown in FIG. 9B, the ΔldhA, Δfrd, and ΔpflB-focA mutations cooperatively increased ethanol production.

To test the effect of additional deletion mutations on ethanol production from various ratios of mixed sugars (mannitol:uronic acid (alginate)), E. coli strains ATCC9637 ΔldhA/Δfrd harboring pALG7.2 and pBBRpdc-adhA/B were grown overnight in LB media at 30° C. Cultures were inoculated into fresh M9 media containing 5% in total with different ratios of mannitol:alginate. These ratios are indicated in FIGS. 10A and 10B. The ethanol production at 120 hrs was monitored by gas chromatography (GC). As shown in FIGS. 10A and 10B, production of ethanol from recombinant microorganisms, especially those having deletion mutants, can be optimized by controlling the ratio of the different types of sugars, mainly sugar alcohols (e.g., mannitol) and uronic acids (e.g., alginate). Without being bound by any one theory, it is believed that the optimal combination and optimal combination of polysaccharides balances the carbon flux and the oxidation-reduction potential within the cell, thereby reducing toxicity (or reducing the cell growth inhibitory effects of an imbalanced redox potential) and increasing production.

### Example 7

### Integrated Systems to Optimize the Capacity of E. coli to Produce Ethanol from Kelp

Integrated systems were employed to optimize the capacity of E. coli to produce ethanol from kelp. For example, in one experiment, to further improve the ability of recombinant E. coli to grow on alginate as a sole source of carbon, E. coli strain W version AL3.0 was transformed with the pALG2.5 vector (see Example 2, supra) alone or in individual combination with the tether system vectors pAL1.0, pAL2.0, or pAL3.0, the components of which are described in Table 11 below. E. coli strain W version AL3.0 contains both the pdc-adhA/B operon and deletions in the lactose dehydrogenase A (ldhA) and fumarate reductase (frd) genes (pdc-adhA/B, Aldha, Δfrd)). Also, E. coli version AL3.0 was transformed with the pALG7.2 vector (see Example 3) alone or in combination with the tether system vector pAL4.0, the components of which are also described in Table 14 below.

TABLE 14
Tether System Vectors.

| Vector | Promoter/ | | Alginate |
|---|---|---|---|
| Name | Vector System | Carrier Polypeptide | Lyase |
| pAL1.0 | P_{PDC} | Omp1 from Z. mobilis | ΔAI-I |
| | pTrc99A | | |
| pAL2.0 | P_{PDC} | OmpA with signal | ΔAI-I |
| | pTrc99A | peptide from E. coli LPP | |
| pAL3.0 | P_{PDC} | Ag43 | SM0524 |
| | pTrc99A | | |
| pAL4.0 | P_{H207} | Ag43 | SM0524 |
| | pCCFos2 | | |

Generally, after transformation with the above combination of vectors, recombinant E. coli was incubated in media containing 6% (5% for pALG7.2) Macrocystis pyrifera (a species of kelp) at 30° C. and pH 7.0 for up to 50 hours. In certain experiments, E. coli strain W version AL3.0, containing the pAL1.0 and pALG2.5 vectors, was incubated in media containing Laminaria japonica (a species of kelp). Culture samples were collected at various time points, and were analyzed for the production of ethanol. Specific experiments are described below.

Ethanol production from Laminaria japonica. E. coli strain ATCC 9637 with ΔldhA/Δfrd harboring pALG2.5, pTrcOmp1-ΔAI-I (pAL1.0) and pBBRpdc-adhA/B was grown overnight. One mL of the culture was inoculated into 100 mL fresh LB media containing 0.1 mM IPTG. When the culture reached OD600 nm of 1.0, the culture was centrifuged and the cells were harvested. The pellet was then re-inoculated into fresh M9 media containing 0.1 mM IPTG, 0.5% LB, and 10% of solid brown kelp, Laminaria japonica. The ethanol production over the course of 40 hours was investigated. As shown in FIG. 11A, this strain of engineered E. coli is capable of producing ethanol from brown kelp Laminaria japonica at least with 18.3 g/L titer, 77% yield to the maximum theoretical yield, and 0.83 g/L/h productivity. In this experiment, the brown kelp Laminaria japonica was pretreated with 0.01 mg/ml alginate lyase.

As a further test, E. coli strain ATCC 8739 (ΔldhA, Δfrd, ΔpflB-focA) harboring pALG7.8, pTrcpdc-adhB was grown overnight. One mL of the culture was inoculated into 100 mL fresh LB media containing 0.1 mM IPTG. When the culture reached OD600 nm of 1.0, the culture was centrifuged and the cells were harvested. The pellet was then re-inoculated into fresh M9 media containing 0.1 mM IPTG, 0.5% LB, and 10% of solid brown seaweed, Laminaria japonica. The ethanol production over the course of 40 hours was investigated. As shown in FIG. 11B, this strain of engineered E. coli is capable of producing ethanol from brown seaweed Laminaria japonica at least with 22.5 g/L titer, 85% yield to the maximum theoretical yield (specific yield), and 1.65 g/L/h productivity. The brown seaweed Laminaria japonica was pretreated with 0.01 mg/ml alginate lyase.

Ethanol production from Macrocystis pyrifera. E. coli strain ATCC 9637 with ΔldhA/Δfrd harboring pALG7.2 and pBBRpdc-adhA/B was grown overnight. One mL of the culture was inoculated into 100 mL fresh LB media containing 0.025 mM IPTG. When the culture reached OD600 nm of 1.0, the culture was centrifuged and the cells were harvested. The pellet was then re-inoculated into fresh M9 media containing 0.025 mM IPTG, 0.5% LB, and 10% of solid kelp, Macrocystis pyrifera.

The ethanol production over the course of 50 hours were investigated. As shown in FIG. 11C, this strain of engineered E. coli is capable of producing ethanol from brown kelp Macrocystis pyrifera at least with 13 g/L titer, 62% yield to the maximum theoretical yield, 0.4 g/L/h productivity. In this experiment, the brown kelp Macrocystis pyrifera was pretreated with 0.01 mg/ml alginate lyase, 0.05 mg/ml laminarinase, 0.1 mg/ml endoglucanase, and 1 mg/ml lipase.

The effect of various pretreatment methods on the ethanol production from Macrocystis pyrifera. E. coli strain ATCC 9637 with ΔldhA/Δfrd harboring pALG7.2 and pBBRpdc-adhA/B was grown overnight. One mL of the culture was inoculated into 100 mL fresh LB media containing 0.025 mM IPTG. When the culture reached OD600 nm of 1.0, the culture was centrifuged and the cells were harvested. The pellet was then re-inoculated into fresh M9 media containing 0.025 mM IPTG, 0.5% LB, and 5% of solid brown kelp, Macrocystis pyrifera pretreated with 0.01 mg/ml alginate lyase, 0.05 mg/ml laminarinase, 0.1 mg/ml endoglucanase, or 1 mg/ml lipase. The ethanol production over the course of 10 hours was investigated.

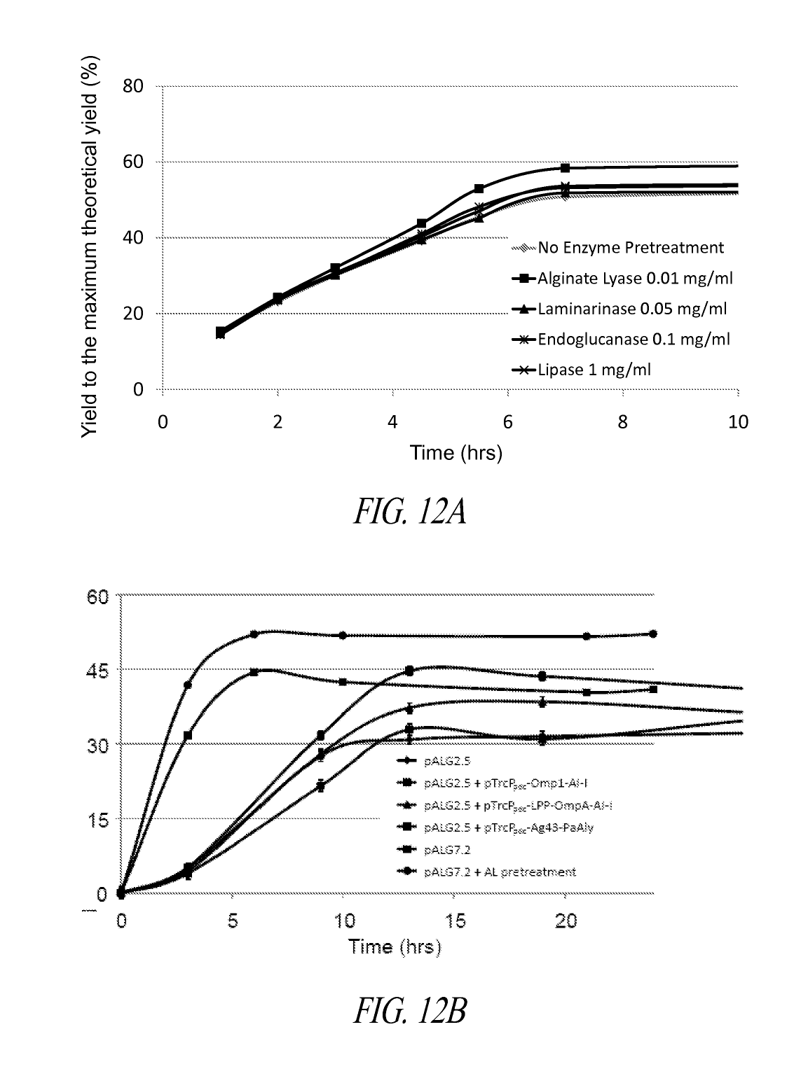

As shown in FIG. 12A, the results suggest that addition of extracellular alginate lyase, laminarinase, endoglucanase, or lipase increases production of ethanol. These results also suggest that similar or better results could be achieved by the use of these enzymes in the tether display systems described herein.

The effect of different alginate lyase surface display systems on the ethanol production from Macrocystis pyrifera. E. coli strain ATCC 9637 with ΔldhA/Δfrd harboring pALG2.5/pTrcP_{pdc}-omp1-AI-I, pTrcP_{pdc}-LPP-OmpA-AI-I, or pTrcP_{pdc}-Ag43-ΔPaAly, or pTpALG7.2 and pBBRpdc-adhA/B was grown overnight. One mL of the culture was inoculated into 100 mL fresh LB media containing 0.025 mM IPTG. When the culture reached OD600 nm of 1.0, the culture was centrifuged and the cells were harvested. The pellet was then re-inoculated into fresh M9 media containing 0.025 mM IPTG, 0.5% LB, and 5% of solid brown kelp, Macrocystis pyrifera.

The ethanol production over the course of 24 hours was investigated. As shown in FIG. 12B, the results illustrate that introducing active tethered alginate lyase into the system increases the rate of ethanol production. Also, pALG7.2 showed almost the same efficiency as it does when the kelp was pretreated with alginate lyase, illustrating that the use of an enzyme-based tether systems is equivalent to pre-treating with the isolated enzyme, as noted above.

### Example 8

### Growth of Different Subspecies of E. coli

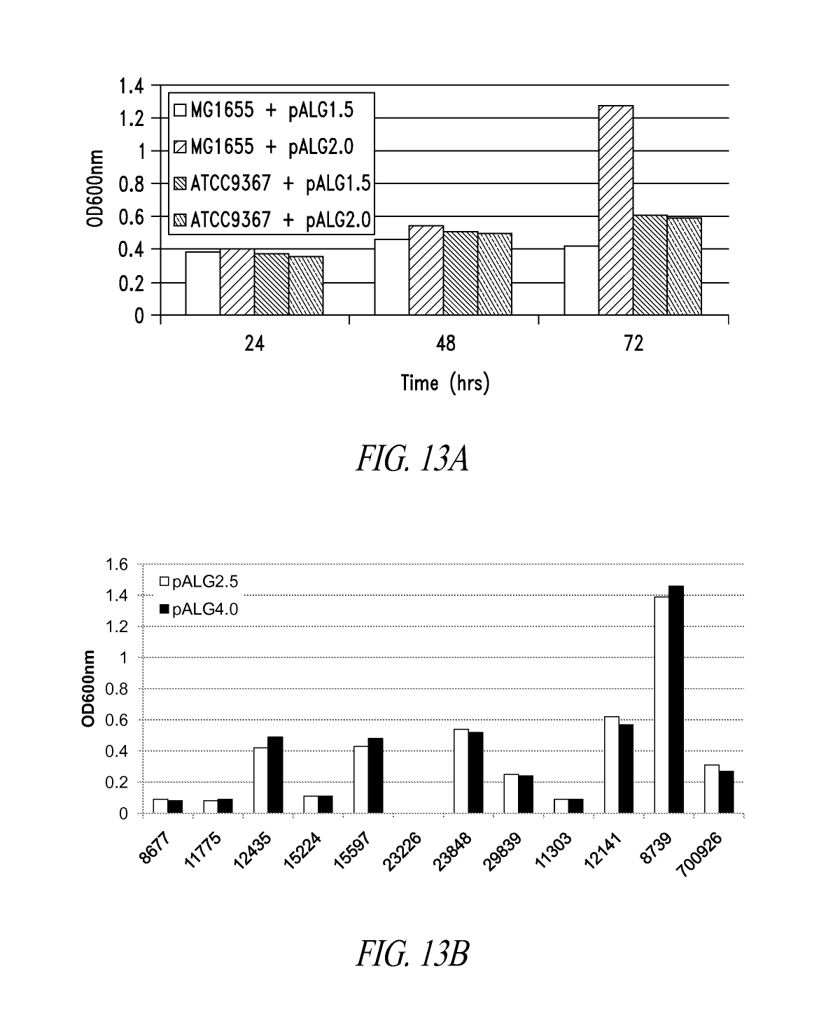

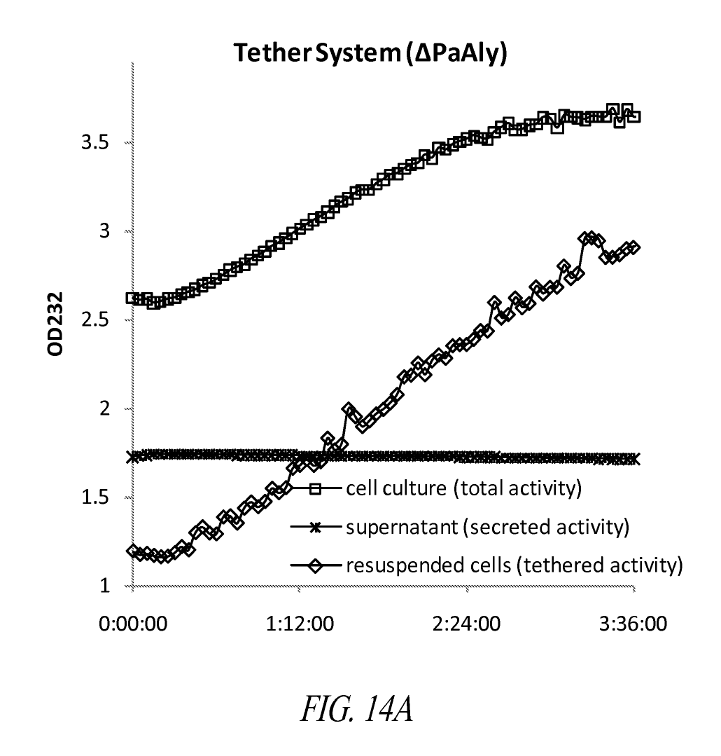

The compatibility between different E. coli strains (ATCC strains 8677, 11775, 12435, 15224, 15597, 23226, 23848, 29839, 11303, 12141, 8739, 700926, and 9637) and the pALG vectors was investigated. First, pALG1.5 or pALG2.0 were transformed into MG1655 (ATCC700926) and ATCC9637 strain, and these strains were subjected to a growth study in M9 media containing 0.5% enriched guluronate as a sole carbon source. pALG2.0 contains the guluronate outer membrane transporter on the pALG1.5 backbone (see Example 2). Growth was monitored over a 72 hour period. As shown in FIG. 13A, the pALG2.0 vector provides strain MG1655 (ATCC700926) with the ability to grown on guluronate as a sole source of carbon. However, strain ATCC9637 lacks ability to take up and efficiently utilize guluronate, even with the pALG2.0 vector.

Based on this observation, the compatibility between different E. coli strains and pALG vectors (pALG2.5 or pALG4.0) was further investigated by transforming these vectors into 12 different E. coli strains and testing their growth on M9 media containing 0.5% enriched guluronate as a sole carbon source. As shown in FIG. 13B, many of the transformed E. coli strains were able to grow on guluronate as a sole source of carbon, and strain ATCC8739 grew significantly better than other E. coli strains.

### Example 9

### ChromoSomal Integration of PDC-adhB Operon

To achieve stable ethanol production, an artificial operon comprising pdc-adhB derived from Zymomonasu mobilis was cloned into pKD13, and then integrated into the E. coli genome. First, the DNA fragment of pdc, adhB, and pKD13 were amplified by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-TTAGAAAGCGCTCAGGAAGAGTTCT-3′ [SEQ ID NO:574] for adhB, 5′-TGAAGAAGCCATTATATATACCTCCTTAGAGGAGCTTGTTAACAGGCTTA-3′ [SEQ ID NO:575] for pdc, and 5′-AGTATAACTCATTATATATACCTCCTGTAGGCTGGAGCTGCTTCGAAGTT-3′ [SEQ ID NO:576] for pKD13) and reverse (5′-TAAGCCTGTTAACAAGCTCCTCTAAGGAGGTATATATAATGGCTTCTTCA-3′ [SEQ ID NO:577] for adhB, 5′-AACTTCGAAGCAGCTCCAGCCTACAGGAGGTATATATAATGAGTTATACT-3′ (SEQ ID NO:578) for pdc, and 5′-AATCGCTCAAGACGTGTAATGCTGC-3′ (SEQ ID NO:579) for pKD13) primers, 50 ng of purified genomic DNA Zymomonas mobilis or pKDl3, and 2.5 U Phusion DNA polymerase (Finezyme) in total volume of 50 μl.

The amplified fragments were gel purified and spliced by PCR: 98° C. for 10 sec, 55° C. for 15 sec, and 72° C. for 3 min, repeated for 30 times. The reaction mixture contained 1× Phusion buffer, 2 mM dNTP, 0.5 μM forward (5′-TTAGAAAGCGCTCAGGAAGAGTTCT-3′ [SEQ ID NO:580]) and reverse (5′-AATCGCTCAAGACGTGTAATGCTGC-3′ [SEQ ID NO:581] for pKDl3) primers, 50 ng of purified genomic DNA Zymomonas mobilis of pKDl3, and 2.5 U Phusion DNA polymerase (Finezyme) in total volume of 50 μl. The amplified fragment was then treated with T4 DNA polynucleotide kinase (New England Biolabs) and ligated with T4 DNA ligase to form pKD13-pdc-adhB.

Chromosomal integration of the artificial operon comprising pcd and adhB. The artificial operon comprising pcd and adhB was amplified from pKD13-pdc-adhB by PCR: δ 98° C. for 10 sec, 55° C. for 15 sec, and 72° C. for 2 min, repeated for 30 times. The reaction mixture contained 1× Phusion buffer, 2 mM dNTP, 0.5 μM forward (5′-ACTGGTCAGAGCTTCTGCTGTCAGGAATGCCTGGTGCCCGTTAGAAAGCGCT CAGGAAGA-3′ [SEQ ID NO:582] for the integration into ldhA site, 5′-CGACACCAATCAGCGTGACAACTGTCAGGATAGCAGCCAGTTAGAAAGCGCT CAGGAAGA-3′ [SEQ ID NO:583] for the integration into frd site, and 5′-TTACATAGATTGAGTGAAGGTACGAGTAATAACGTCCTGCTTAGAAAGCGCT CAGGAAGA-3′ [SEQ ID NO:584] for the integration into pflB-focA site) and reverse (5′-TTTGGCTTTGAGCTGGAATTTTTTGACTTTCTGCTGACGGATTCCGGGGATCC GTCGACC-3′ [SEQ ID NO:585] for the integration into ldhA site, 5′-TCTCAAAAGTATACCCGATGCGTAGCCATACCGTTGCTGCATTCCGGGGATCC GTCGACC-3′ [SEQ ID NO:586] for the integration into frd site and 5′-CTGCTGCAATGGCCAAAGTGGCCGAAGAGGCGGGTGTCTAATTCCGGGGATC CGTCGACC-3′ [SEQ ID NO:587] for the integration into pflB-focA site) primers, 50 ng of pKD13-pdc-adhB, and 2.5 U Phusion DNA polymerase (Finezyme) in total volume of 50 μl. The amplified fragments were gel purified and transformed into an E. coli strain ATCC8739 via homologous recombination. The kanamycin selection marker was excised from the chromosome through over-expression of FLP. Thus, ATCC8739 (ΔldhA::pdc-adhB, Δfrd, ΔfocA-pflB), (ΔldhA, Δfrd::pdc-adhB, ΔfocA-pflB), and (ΔldhA, Δfrd, AfocA-pflB::pdc-adhB) were created.

Construction of integration cassette of strong constitutive promoters. To achieve higher-level, stable ethanol production, plasmids containing integration cassettes of five different strong constitutive promoters were cloned into pKD13. The DNA fragments of pKD13 with promoter are amplified by PCR: 98° C. for 10 sec, 55° C. for 15 sec, and 72° C. for 2 min, repeated for 30 times. The reaction mixture contained 1× Phusion buffer, 2 mM dNTP, 0.5 μM forward (5′-GCCTATCGGCTAGGGTGTCAAACTATTTTTTGCAGTTTTTTGTAGGCTGGAGC TGCTTC-3′ [SEQ ID NO:588] for P_{D/E20}, 5′-GAAACTTAAGCATTTTAGCAAATAAAACTTTTAATTAAAATGTAGGCTGGAG CTGCTTC-3′ [SEQ ID NO:589] for P_{F30}, 5′-GATTGCTGGGCTATTGTCAACAATTTTTTAGTAGTCTGAGTGTAGGCTGGAGC TGCTTC-3′ [SEQ ID NO:590] for P_{H22}, 5′-GTATTGGAAAATTTTATCAAGAAATTTTTATTTTTCCATATGTAGGCTGGAGC TGCTTC-3′ [SEQ ID NO:591] for P_{G25}, and, 5′-CTAAATTTCCACCTGTGTCAATAACGGTTTTTATATCCGCTGTAGGCTGGAGC TGCTTC-3′ [SEQ ID NO:592] for P_{J5}) and reverse (5′-TTTAAGATGTACCCAGTTCGATGAGAGCGATAACTCACACAATCGCTCAAGA CGTGTAAT-3′ [SEQ ID NO:593] for P_{D/E20}, 5′-TGTATAATTACTTTATAAATTGATGAGAAGGAAATCACACAATCGCTCAAGA CGTGTAAT-3′ [SEQ ID NO:594] for P_{F30}, 5′-GGTAAAATATCGATTTAGGCAGTTCACACAGATATCATTAAATCGCTCAAGA CGTGTAAT-3′ [SEQ ID NO:595] for P_{H22}, 5′-TATTATAATATTGTTATTAAAGAGGAGAAATTAACCACACAATCGCTCAAGA CGTGTAAT-3′ [SEQ ID NO:596] for P_{G25}, and, 5′-AATATACTGTTAGTAAACCTAATGGATCGACCTTTCACACAATCGCTCAAGAC GTGTAAT-3′ [SEQ ID NO:597] for P_{J5}) primers, 50 ng of pKD13, and 2.5 U Phusion DNA polymerase (Finezyme) in total volume of 50 μl. The amplified fragment was then treated with T4 DNA polynucleotide kinase (New England Biolabs) and ligated with T4 DNA ligase to form pKD13-P_{D/E20}, pKD13-P_{F30}, pKD13-P_{H22}, pKD13-P_{G25}, and pKD13-P_{J5}, respectively.

Integration of constitutive strong promoter into E. coli chromosome. The five promoters, P_{D/E20}, P_{F30}, P_{H22}, P_{G25}, and P_{J5}, were integrated into the E. coli chromosome the way that they control the expression of pdc-adhB. Integration cassettes for these five promoters, P_{D/E20}, P_{F30}, P_{H22}, P_{G25}, and P_{J5}, were amplified by PCR: The DNA fragments of pKD13 with promoter are amplified by PCR: 98° C. for 10 sec, 55° C. for 15 sec, and 72° C. for 2 min, repeated for 30 times. The reaction mixture contained 1×Phusion buffer, 2 mM dNTP, 0.5 μM forward (5′-AAATAGGTACCGACAGTATAACTCATTATATATACCTCCTGTGTGAGTTATCG CTCTCAT-3′ [SEQ ID NO:598] for P_{D/E20}, 5′-AAATAGGTACCGACAGTATAACTCATTATATATACCTCCTGTGTGATTTCCTT CTCATCA-3′ [SEQ ID NO:599] for P_{F30}, 5′-AAATAGGTACCGACAGTATAACTCATTATATATACCTCCTTAATGATATCTGT GTGAACT-3′ [SEQ ID NO:600] for P_{H22}, 5′-AAATAGGTACCGACAGTATAACTCATTATATATACCTCCTGTGTGGTTAATTT CTCCTCT-3′ [SEQ ID NO:601] for P_{G25}, and, 5′-AAATAGGTACCGACAGTATAACTCATTATATATACCTCCTGTGTGAAAGGTC GATCCATT-3′ [SEQ ID NO:602] for P_{J5}) and reverse (5′-TTTGGCTTTGAGCTGGAATTTTTTGACTTTCTGCTGACGGATTCCGGGGATCC GTCGACC-3′ [SEQ ID NO:603] for the integration into ldhA site, 5′-TCTCAAAAGTATACCCGATGCGTAGCCATACCGTTGCTGCATTCCGGGGATCC GTCGACC-3′ [SEQ ID NO:604] for the integration into frd site, and 5′-CTGCTGCAATGGCCAAAGTGGCCGAAGAGGCGGGTGTCTAATTCCGGGGATC CGTCGACC-3′ [SEQ ID NO:605] for the integration into focA-pflB site) primers, 50 ng of pKD13, and 2.5 U Phusion DNA polymerase (Finezyme) in total volume of 50 μl.

The amplified fragments were gel purified and transformed into an E. coli strain ATCC8739 via homologous recombination. Thus, ATCC8739 (ΔldhA::P_{D/E20}-pdc-adhB, Δfrd, ΔfocA-pflB), (ΔldhA::P_{F30}-pdc-adhB, Δfrd, ΔfocA-pflB), (ΔldhA::P_{H22}-pdc-adhB, Δfrd, ΔfocA-pflB), (ΔldhA::P_{G25}-pdc-adhB, Δfrd, ΔfocA-pflB), (ΔldhA::P_{J5}-pdc-adhB, Δfrd, ΔfocA-pflB), (ΔldhA, Δfrd::P_{D/E20}-pdc-adhB, ΔfocA-pflB), (ΔldhA, Δfrd::P_{F30}-pdc-adhB, ΔfocA-pflB), (ΔldhA, Δfrd::P_{H22}-pdc-adhB, ΔfocA-pflB), (ΔldhA, Δfrd::P_{G25}-pdc-adhB, ΔfocA-pflB), (ΔldhA, Δfrd::P_{J5}-pdc-adhB, ΔfocA-pflB), (ΔldhA, Δfrd, ΔfocA-pflB::P_{D/E20}-pdc-adhB), (ΔldhA, Δfrd, ΔfocA-pflB::P_{F30}-pdc-adhB), (ΔldhA, Δfrd, ΔfocA-pflB::P_{H22}-pdc-adhB), (ΔldhA, Δfrd, ΔfocA-pflB::P_{G25}-pdc-adhB), and (ΔldhA, Δfrd, ΔfocA-pflB::P_{J5}-pdc-adhB) were created.

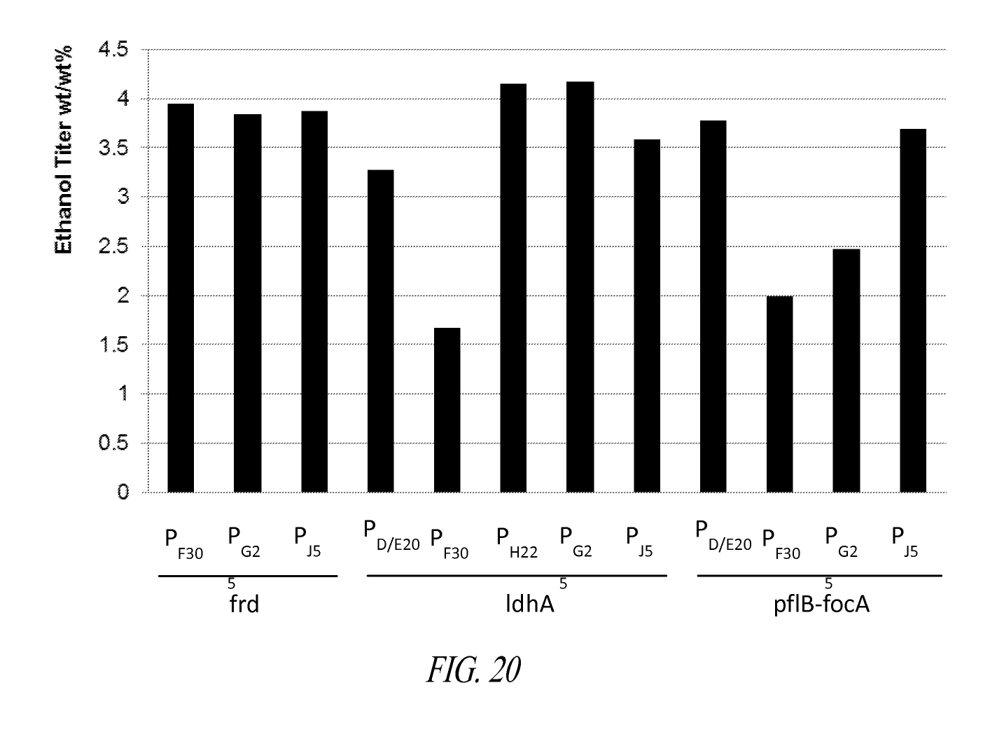

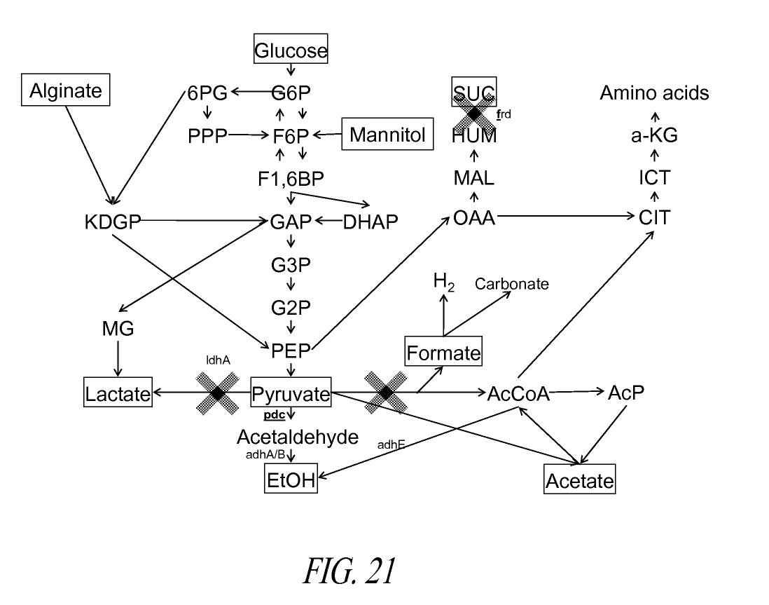

Ethanol production from synthetic media (Mannitol:Glucuronate=2:1 ratio) using chromosome integrated E. coli strain. Ethanol fermentation was carried out using strains ATCC8739 (ΔldhA::P_{D/E20}-pdc-adhB, Δfrd, ΔfocA-pflB), (ΔldhA::P_{F30}-pdc-adhB, Δfrd, ΔfocA-pflB), (ΔldhA::P_{H22}-pdc-adhB, Δfrd, ΔfocA-pflB), (ΔldhA::P_{G25}-pdc-adhB, Δfrd, ΔfocA-pflB), (ΔldhA::P_{J5}-pdc-adhB, Δfrd, ΔfocA-pflB), (ΔldhA, Δfrd::P_{D/E20}-pdc-adhB, ΔfocA-pflB), (ΔldhA, Δfrd::P_{F30}-pdc-adhB, ΔfocA-pflB), (ΔldhA, Δfrd::P_{H22}-pdc-adhB, ΔfocA-pflB), (ΔldhA, Δfrd::P_{G25}-pdc-adhB, AfocA-pflB), (ΔldhA, Δfrd::P_{J5}-pdc-adhB, ΔfocA-pflB), (ΔldhA, Δfrd, ΔfocA-pflB:: P_{D/E20}-pdc-adhB), (ΔldhA, Δfrd, ΔfocA-pflB::P_{F30}-pdc-adhB), (ΔldhA, Δfrd, ΔfocA-pflB::P_{H22}-pdc-adhB), (ΔldhA, Δfrd, ΔfocA-pflB::P_{G25}-pdc-adhB), and (ΔldhA, Δfrd, ΔfocA-pflB::P_{J5}-pdc-adhB) from M9 minimal media containing 10% sugar (mannitol: glucuronate=2:1 ratio). Ethanol concentration was measured at 60 hours after the fermentation started. As shown in FIG. 20, the chromosomally integrated strains have ability to produce ethanol as high as 90% of the theoretical maximum yield (specific productivity).

1-168. (canceled)

169. A method of increasing production of a commodity chemical in a microorganism, comprising:
a) providing a recombinant microorganism;
b) providing alginate as a first carbon source to said recombinant microorganism;
c) providing mannitol as a second carbon source to said recombinant microorganism;
d) growing said recombinant microorganism under anaerobic fermentative conditions for a time sufficient to allow metabolism of at least part of the first carbon source and metabolism of at least a part of the second carbon source to thereby increase production of the commodity chemical in the recombinant microorganism compared to the production of the commodity chemical in the recombinant microrganism when said recombinant microorganism is grown in the presence of the first carbon source but in the absence of said second carbon source.

170. The method of claim 169 wherein the recombinant microorganism is Escherichia coli.

171. The method of claim 169 wherein the recombinant microorganism is yeast.

172. The method of claim 169 wherein the commodity chemical is ethanol.

173. The method of claim 169, wherein the alginate:marmitol ratio is 5:1, 4:1, 3:1, 3:2, 2:1, 1:1, 1:2, 2:3 1:3, 1:4, or 1:5.

174. The method of claim 173, wherein the alginate:mannitol ratio is 5:1.

175. The method of claim 173, wherein the alginate:mannitol ratio is 4:1.

176. The method of claim 173, wherein the alginate:mannitol ratio is 3:1.

177. The method of claim 173, wherein the alginate:mannitol ratio is 3:2.

178. The method of claim 173, wherein the alginate:mannitol ratio is 2:1.

179. The method of claim 173, wherein the alginate:mannitol ratio is 1:1.

180. The method of claim 173, wherein the alginate:mannitol ratio is 1:2.

181. The method of claim 173, wherein the alginate:mannitol ratio is 2:3.

182. The method of claim 169, wherein the alginate:mannitol ratio is 1:3.

183. The method of claim 169, wherein the alginate:mannitol ratio is 1:4.

184. The method of claim 169, wherein the alginate:mannitol ratio is 1:5.


SEQUENCE LISTING (607 sequences)

SEQ ID NO: 1 - DNA, 74 bp, Escherichia coli
  Ptrc (trc promoter)
ttgacaattaatcatccggctcgtataatgtgtggaattgtgagcggataacaatttcacacaggaaacagacc

SEQ ID NO: 2 - DNA, 538 bp, Zymomonas mobilus
  Ppdc (pdc promoter)
ccatggaattcgagctcggtaccctttgtcagtgttgcggtataatatctgtaacagctcattgataaagccggtcgctcgcctcgggcagttttggattgatcctgccctgtcttgtttggaattgatgaggccgttcatgacaacagccggaaaaattttaaaacaggcgtcttcggctgctttaggtctcggctacgtttctacatctggttctgattcccggtttacctttttcaaggtgtcccgttcctttttcccctttttggaggttggttatgtcctataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagaaccatggg

SEQ ID NO: 3 - DNA, 70 bp, Coliphage
  PH207 (H207 promoter)
ttttaaaaaattcatttgctaaacgcttcaaattctcgtataatatacttcataaattgataaacaaaaa

SEQ ID NO: 4 - DNA, 70 bp, Coliphage
  PD/E20 (D/E20 promoter)
aactgcaaaaatagtttgacaccctagccgataggctttaagatgtacccagttcgatgagagcgataac

SEQ ID NO: 5 - DNA, 74 bp, Coliphage
  PD/E20 (D/E20 promoter) full-length
aaaaaactgcaaaaatagtttgacaccctagccgataggctttaagatgtacccagttcgatgagagcgataac

SEQ ID NO: 6 - DNA, 72 bp, Coliphage
  PG25 (G25 promoter)
tatggaaaaataaaaatttcttgataaaattcaatactattataatattgttattaaagaggagaaattaac

SEQ ID NO: 7 - DNA, 64 bp, Coliphage
  PH22 (H22 promoter)
actcagactactaaaaaattgttgacaatagcccagcaatcggtaaaatatcgatttaggcagt

SEQ ID NO: 8 - DNA, 75 bp, Coliphage
  PF30 (F30 promoter)
attttaattaaaagttttatttgctaaaatgcttaagtttctgtataattactttataaattgatgagaaggaaa

SEQ ID NO: 9 - DNA, 75 bp, Coliphage
  PJ5 (J5 promoter)
agcggatataaaaaccgttattgacacaggtggaaatttagaatatactgttagtaaacctaatggatcgacctt

SEQ ID NO: 10 - DNA, 75 bp, Coliphage
  PN25 (N25 promoter)
aagaatcataaaaaatttatttgctttcaggaaaatttttctgtataatagattcataaatttgagagaggagtt

SEQ ID NO: 11 - DNA, 70 bp, Bacteriophage lambda
  PL (L promoter)
ttatctctggcggtgttgacataaataccactggcggtgatactgagcacatcagcaggacgcactgacc

SEQ ID NO: 12 - DNA, 70 bp, Bacteriophage T5
  PA1 (A1 promoter)
ttatcaaaaagagtattgacttaaagtctaacctataggatacttacagccatcgagagggacacggcga

SEQ ID NO: 13 - DNA, 133 bp, Escherichia coli
  PrrnB-2 (rrnB-2 promoter)
cacggaacaacggcaaacacgccgccgggtcagcggggttctcctgagaactccggcagagaaagcaaaaataaatgcttgactctgtagcgggaaggcgtattatgcacaccccgcgccgctgagaaaaagc

SEQ ID NO: 14 - DNA, 49 bp, Escherichia coli
  PLPP (LPP promoter)
atcaaaaaaatattctcaacataaaaaactttgtgtaatacttgtaacg

SEQ ID NO: 15 - DNA, 1140 bp, Bacillus subtilis
  PgsA
atgaaaaaagaactgagctttcatgaaaagctgctaaagctgacaaaacagcaaaaaaagaaaaccaataagcacgtatttattgccattccgatcgtttttgtccttatgttcgctttcatgtgggcgggaaaagcggaaacgccgaaggtcaaaacgtattctgacgacgtactctcagcctcatttgtaggcgatattatgatgggacgctatgttgaaaaagtaacggagcaaaaaggggcagacagtatttttcaatatgttgaaccgatctttagagcctcggattatgtagcaggaaactttgaaaacccggtaacctatcaaaagaattataaacaagcagataaagagattcatctgcagacgaataaggaatcagtgaaagtcttgaaggatatgaatttcacggttctcaacagcgccaacaaccacgcaatggattacggcgttcagggcatgaaagatacgcttggagaatttgcgaagcaaaatcttgatatcgttggagcgggatacagcttaagtgatgcgaaaaagaaaatttcgtaccagaaagtcaacggggtaacgattgcgacgcttggctttaccgatgtgtccgggaaaggtttcgcggctaaaaagaatacgccgggcgtgctgcccgcagatcctgaaatcttcatccctatgatttcagaagcgaaaaaacatgcggacattgttgttgtgcagtcacactggggacaagagtatgacaatgatccaaatgaccgccagcgccagcttgcaagagccatgtctgatgcgggagctgacatcatcgtcggccatcacccgcacgtcttagaaccgattgaagtatataacggaaccgtcattttctacagcctcggcaactttgtctttgaccaaggctggacgagaacaagagacagtgcactggttcagtatcacctgaagaaaaatggaacaggacgctttgaagtgacaccgatcgatatccatgaagcgacacctgcgcctgtgaaaaaagacagccttaaacagaaaaccattattcgcgaactgacgaaagactctaatttcgcttggaaagtagaagacggaaaactgacgtttgatattgatcatagtgacaaactaaaatctaaa

SEQ ID NO: 16 - protein, 380 aa, Bacillus subtilis
  PgsA
MKKELSFHEKLLKLTKQQKKKTNKHVFIAIPIVFVLMFAFMWAGKAETPKVKTYSDDVLSASFVGDIMMGRYVEKVTEQKGADSIFQYVEPIFRASDYVAGNFENPVTYQKNYKQADKEIHLQTNKESVKVLKDMNFTVLNSANNHAMDYGVQGMKDTLGEFAKQNLDIVGAGYSLSDAKKKISYQKVNGVTIATLGFTDVSGKGFAAKKNTPGVLPADPEIFIPMISEAKKHADIVVVQSHWGQEYDNDPNDRQRQLARAMSDAGADIIVGHHPHVLEPIEVYNGTVIFYSLGNFVFDQGWTRTRDSALVQYHLKKNGTGRFEVTPIDIHEATPAPVKKDSLKQKTIIRELTKDSNFAWKVEDGKLTFDIDHSDKLKSK

SEQ ID NO: 17 - DNA, 87 bp, Escherichia coli
  LPP
atgaaagctactaaactggtactgggcgcggtaatcctgggttctactctgctggcaggttgctccagcaacgctaaaatcgatcag

SEQ ID NO: 18 - protein, 29 aa, Escherichia coli
  LPP
MKATKLVLGAVILGSTLLAGCSSNAKIDQ

SEQ ID NO: 19 - DNA, 157 bp, Escherichia coli
  Ag43-SP
atgaaacgacatctgaatacctgctacaggctggtatggaatcacatgacgggcgctttcgtggttgcctccgaactggcccgcgcacggggtaaacgtggcggtgtggcggttgcactgtctcttgccgcagtcacgtcactcccggtgctggctg

SEQ ID NO: 20 - protein, 52 aa, Escherichia coli
  Ag43-SP
MKRHLNTCYRLVWNHMTGAFVVASELARARGKRGGVAVALSLAAVTSLPVLA

SEQ ID NO: 21 - DNA, 1920 bp, Escherichia coli
  Ag43
agcgacggaaaggcattcagtatcggaggcggtcaggcggatgccctgatgctggaaaaaggcagttcattcacgctgaacgccggtgatacggccacggataccacggtaaatggcggactgttcaccgccaggggcggcacactggcgggcaccaccacgctgaataacggcgccatacttaccctttccgggaagacggtgaacaacgataccctgaccatccgtgaaggcgatgcactcctgcagggaggctctctcaccggtaacggcagcgtggaaaaatcaggaagtggcacactcactgtcagcaacaccacactcacccagaaagccgtcaacctgaatgaaggcacgctgacgctgaacgacagtaccgtcaccacggatgtcattgctcagcgcggtacagccctgaagctgaccggcagcactgtgctgaacggtgccattgaccccacgaatgtcactctcgcctccggtgccacctggaatatccccgataacgccacggtgcagtcggtggtggatgacctcagccatgccggacagattcatttcacctccacccgcacagggaagttcgtaccggcaaccctgaaagtgaaaaacctgaacggacagaatggcaccatcagcctgcgtgtacgcccggatatggcacagaacaatgctgacagactggtcattgacggcggcagggcaaccggaaaaaccatcctgaacctggtgaacgccggcaacagtgcgtcggggctggcgaccagcggtaagggtattcaggtggtggaagccattaacggtgccaccacggaggaaggggcctttgtccaggggaacaggctgcaggccggtgcctttaactactccctcaaccgggacagtgatgagagctggtatctgcgcagtgaaaatgcttatcgtgcagaagtccccctgtatgcctccatgctgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcgtccgtctggagggtgacctgatgagaacagaggttgccggtatgtctgtgaccgcgggggtatatggtgctgctggccattcttccgttgatgttaaggatgatgacggctcccgtgccggcacggtccgggatgatgccggcagcctgggcggatacctgaatctggtacacacgtcctccggcctgtgggctgacattgtggcacagggaacccgccacagcatgaaagcgtcatcggacaataacgacttccgcgcccggggctggggctggctgggctcactggaaaccggtctgcccttcagtatcactgacaacctgatgctggagccacaactgcagtatacctggcagggactttccctggatgacggtaaggacaacgccggttatgtgaagttcgggcatggcagtgcacaacatgtgcgtgccggtttccgtctgggcagccacaacgatatgacctttggcgaaggcacctcatcccgtgcccccctgcgtgacagtgcaaaacacagtgtgagtgaattaccggtgaactggtgggtacagccttctgttatccgcaccttcagctcccggggagatatgcgtgtggggacttccactgcaggcagcgggatgacgttctctccctcacagaatggcacatcactggacctgcaggccggactggaagcccgtgtccgggaaaatatcaccctgggcgttcaggccggttatgcccacagcgtcagcggcagcagcgctgaagggtataacggtcaggccacactgaatgtgaccttc

SEQ ID NO: 22 - protein, 640 aa, Escherichia coli
SDGKAFSIGGGQADALMLEKGSSFTLNAGDTATDTTVNGGLFTARGGTLAGTTTLNNGAILTLSGKTVNNDTLTIREGDALLQGGSLTGNGSVEKSGSGTLTVSNTTLTQKAVNLNEGTLTLNDSTVTTDVIAQRGTALKLTGSTVLNGAIDPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVTF

SEQ ID NO: 23 - DNA, 597 bp, Zymomonas mobilis
  Omp1
atgactttattttcggcttcttcggctctgattaagcggagtaaaaaaggatggcgttatcctgtaacgctattcttatcgactaatattctgttggctggatgcacgatggcaccgaaatatcatcgtccagcagcgtctgtggcaccgcaatggccgaaatcagcggcattgcctgccgctgataacacttcaatgaagccccatcctatggcagccgatttggggtggcaggattttttcaaagatgcgcgcctaaaagccctgattacaattgcgatccgcgaaaaccgcgatttgcggtcagctattcaggcaatcggtgaggcgcaagccagatatcgagtgcaacgcgcgtctttattgcccgcaatcggtggcactggcgaagtgatgtatcagcagccttcgggtaaatccggtttgagttttgccccaggtgtcggtgaagatattccgcgtttccattattattcgatgggtatcggtttttcttcttatgaaattgatatttttggccgcatccgcagtttaagcaaggaggcggctgaaagagccttgatgcaggaagagactgccagaggcaccttatcacgc

SEQ ID NO: 24 - protein, 199 aa, Zymomonas mobilis
  Omp1
MTLFSASSALIKRSKKGWRYPVTLFLSTNILLAGCTMAPKYHRPAASVAPQWPKSAALPAADNTSMKPHPMAADLGWQDFFKDARLKALITIAIRENRDLRSAIQAIGEAQARYRVQRASLLPAIGGTGEVMYQQPSGKSGLSFAPGVGEDIPRFHYYSMGIGFSSYEIDIFGRIRSLSKEAAERALMQEETARGTLSR

SEQ ID NO: 25 - DNA, 339 bp, Escherichia coli
  OmpA
aacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggac

SEQ ID NO: 26 - protein, 113 aa, Escherichia coli
  OmpA
NPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPD

SEQ ID NO: 27 - DNA, 1764 bp, Sphingomonas sp. AI
  Alginate lyase delta AI-I
caccccttcgaccaggccgtcgtgaaagaccccacggcctcgtatgtcgacgtcaaggcgcgtcgtaccttcttgcagagcgggcagctcgatgaccgcctcaaggcagcgctgcccaaggagtacgactgcacgaccgaggcaacgcctaacccgcagcaaggcgagatggtcattccgcgccgttatctttccggcaatcacggcccggtgaatccggactacgaaccggtggtgacgctttatcgtgatttcgagaaaatttccgccacgcttggaaatctctacgttgcgacgggcaagccggtgtacgccacttgtctgctgaacatgctggacaagtgggccaaggccgacgcgttgctcaactacgaccccaagtcgcagtcgtggtaccaagtcgaatggtcggcggcgaccgccgcctttgccctgtcgacgatgatggccgagccgaacgtcgacacagcccagcgcgagcgtgtggtgaagtggctcaaccgtgtggcgcgccatcagacgagctttccggggggcgacacgagttgctgcaacaatcactcgtattggcgcggtcaggaagcgaccatcatcggcgtgatcagcaaggacgatgaactcttccgttgggggctgggccgttatgtgcaggcgatggggctgatcaatgaagacggcagcttcgtgcatgaaatgacgcgtcacgaacagtccttgcactatcagaactacgccatgctgccgctgacgatgattgccgaaacggcatcacgtcagggtatcgatctgtacgcgtacaaagagaacggtcgcgatattcactcagcgcgcaagtttgtctttgctgcggtgaagaacccggacctcatcaagaagtacgcttccgagccgcaggatacgcgtgccttcaagccggggcggggcgacctgaactggatcgaataccagcgcgcgcgctttggttttgccgatgagctgggcttcatgacggtgccgatcttcgatccgcgtaccggtggttcgggcactttgctggcgtacaaaccgcaaggcgcggcagcgcaggcgccggtgtccgccccggcagcggcgcattcgtcgatcgacctgtcgaagtggaagctgcagattccggtcgacccgatcgatgtcgccacgcgtgatctgctcaagggttatcaggacaagtacttctacgtcgacaaggatggctcgcttgccttctggtgtccggccagcggtttcaagacgacggccaacacgaagtacccgcgcagcgagctgcgcgaaatgctcgatcccgacaaccacgcggtgaattggggctggcaaggcacccacgaaatgaatctgcgcggagcggtgatgcacgtgtcgcccagtggcaagaccattgtcatgcagattcatgccgtcatgcccgatggctcgaatgcgccgccgctggtcaaggggcagttctacaagaacacgctggattttcttgtgaagaactcggccgctggcggcaaggacacgcactacgtgttcgaaggcatcgaactgggcaagccgtacgatgcgcagatcaaagtggtggacggtgtgttgtcgatgacggtcaacgggcagaccaagaccgtcgatttcgtcgccaaggacgccggctggaaagacctgaagttctatttcaaggcgggcaattacttgcaggaccgccaggctgacgggtcggacaccagcgcgctggtcaagttgtacaagctcgacgtaaagcactcgagc

SEQ ID NO: 28 - DNA, 1764 bp, Artificial Sequence
  codon optimized alginate lyase delta AI-I
cacccgttcgaccaagcagttgtgaaagatccgactgcgtcctatgttgacgttaaagcgcgtcgtactttcctgcaaagcggtcaactggatgatcgcctgaaagcagcgctgccgaaggaatatgactgtaccaccgaagcgacgccgaacccacagcagggtgaaatggtgatcccacgccgctatctgtccggtaaccacggcccggtgaatccggattacgagccggttgtcactctgtatcgcgacttcgaaaaaatcagcgcgaccctgggtaacctgtacgttgcgactggtaaaccagtgtacgcaacttgtctgctgaacatgctggacaaatgggctaaagcagacgcgctgctgaactatgacccgaaatctcagagctggtatcaagtagaatggtccgcagccacggcggcctttgccctgagcactatgatggcagagccgaacgtggacaccgcgcagcgtgagcgtgttgtgaaatggctgaaccgtgtagcacgtcaccagacttcttttccgggtggcgacactagctgctgtaacaatcattcttactggcgtggtcaggaggctaccatcatcggcgttatttccaaggatgatgaactgttccgttggggtctgggtcgttatgtacaggcgatgggtctgatcaacgaagatggttccttcgttcacgaaatgactcgtcacgaacagagcctgcattatcagaactatgcgatgctgccgctgaccatgatcgctgagactgcctctcgtcagggtatcgatctgtatgcttacaaggaaaacggtcgtgatatccattctgctcgtaaattcgtattcgcggccgtaaagaatccggatctgatcaagaaatacgcgagcgaaccgcaggacacgcgcgcttttaaaccgggtcgcggcgatctgaactggatcgaatatcagcgtgcgcgtttcggctttgcagatgagctgggctttatgaccgtgccaatcttcgatccgcgcaccggcggctctggcactctgctggcgtataagccacagggtgcggctgctcaggcgccggtttccgctccggcggcagcacactcttccatcgatctgtccaaatggaaactgcagatccctgttgacccgatcgatgttgctacccgcgatctgctgaagggttatcaggacaagtatttctacgtggataaagatggttctctggccttctggtgcccagcatccggtttcaaaaccacggcgaatactaagtatccgcgtagcgagctgcgtgaaatgctggacccggataatcatgctgttaattggggctggcagggcacccacgaaatgaacctgcgcggtgcagttatgcacgtttccccgtccggtaaaaccatcgtcatgcagatccacgcagttatgccggacggttccaatgcgccaccactggttaaaggccagttctacaaaaacacgctggacttcctggtgaaaaattctgcggctggtggtaaagatactcactacgtgttcgaaggcatcgaactgggtaaaccatacgacgctcagatcaaagttgtagatggtgtcctgtctatgaccgttaatggtcagactaaaactgttgacttcgtggctaaagatgcgggctggaaggatctgaaattctatttcaaggcaggtaactatctgcaggaccgccaggccgacggctccgatacctctgccctggtaaagctgtacaaactggacgttaaacattccagc

SEQ ID NO: 29 - protein, 588 aa, Sphingomonas sp. AI
  Alginate lyase delta AI-I
HPFDQAVVKDPTASYVDVKARRTFLQSGQLDDRLKAALPKEYDCTTEATPNPQQGEMVIPRRYLSGNHGPVNPDYEPVVTLYRDFEKISATLGNLYVATGKPVYATCLLNMLDKWAKADALLNYDPKSQSWYQVEWSAATAAFALSTMMAEPNVDTAQRERVVKWLNRVARHQTSFPGGDTSCCNNHSYWRGQEATIIGVISKDDELFRWGLGRYVQAMGLINEDGSFVHEMTRHEQSLHYQNYAMLPLTMIAETASRQGIDLYAYKENGRDIHSARKFVFAAVKNPDLIKKYASEPQDTRAFKPGRGDLNWIEYQRARFGFADELGFMTVPIFDPRTGGSGTLLAYKPQGAAAQAPVSAPAAAHSSIDLSKWKLQIPVDPIDVATRDLLKGYQDKYFYVDKDGSLAFWCPASGFKTTANTKYPRSELREMLDPDNHAVNWGWQGTHEMNLRGAVMHVSPSGKTIVMQIHAVMPDGSNAPPLVKGQFYKNTLDFLVKNSAAGGKDTHYVFEGIELGKPYDAQIKVVDGVLSMTVNGQTKTVDFVAKDAGWKDLKFYFKAGNYLQDRQADGSDTSALVKLYKLDVKHSS

SEQ ID NO: 30 - DNA, 1923 bp, Artificial Sequence
  codon optimized alginate lyase AI-I
atgcctctggcttgtctggctactactcgtgttggtgctgctcgtgagaaaagcggcgactcttctatgttcgacatcccgtttccgggtcacggtcgtcgtctggccgttgcggcgctggccttcgccggttgcgcgttcgcaggttctctgcaagctcacccgttcgaccaagcagttgtgaaagatccgactgcgtcctatgttgacgttaaagcgcgtcgtactttcctgcaaagcggtcaactggatgatcgcctgaaagcagcgctgccgaaggaatatgactgtaccaccgaagcgacgccgaacccacagcagggtgaaatggtgatcccacgccgctatctgtccggtaaccacggcccggtgaatccggattacgagccggttgtcactctgtatcgcgacttcgaaaaaatcagcgcgaccctgggtaacctgtacgttgcgactggtaaaccagtgtacgcaacttgtctgctgaacatgctggacaaatgggctaaagcagacgcgctgctgaactatgacccgaaatctcagagctggtatcaagtagaatggtccgcagccacggcggcctttgccctgagcactatgatggcagagccgaacgtggacaccgcgcagcgtgagcgtgttgtgaaatggctgaaccgtgtagcacgtcaccagacttcttttccgggtggcgacactagctgctgtaacaatcattcttactggcgtggtcaggaggctaccatcatcggcgttatttccaaggatgatgaactgttccgttggggtctgggtcgttatgtacaggcgatgggtctgatcaacgaagatggttccttcgttcacgaaatgactcgtcacgaacagagcctgcattatcagaactatgcgatgctgccgctgaccatgatcgctgagactgcctctcgtcagggtatcgatctgtatgcttacaaggaaaacggtcgtgatatccattctgctcgtaaattcgtattcgcggccgtaaagaatccggatctgatcaagaaatacgcgagcgaaccgcaggacacgcgcgcttttaaaccgggtcgcggcgatctgaactggatcgaatatcagcgtgcgcgtttcggctttgcagatgagctgggctttatgaccgtgccaatcttcgatccgcgcaccggcggctctggcactctgctggcgtataagccacagggtgcggctgctcaggcgccggtttccgctccggcggcagcacactcttccatcgatctgtccaaatggaaactgcagatccctgttgacccgatcgatgttgctacccgcgatctgctgaagggttatcaggacaagtatttctacgtggataaagatggttctctggccttctggtgcccagcatccggtttcaaaaccacggcgaatactaagtatccgcgtagcgagctgcgtgaaatgctggacccggataatcatgctgttaattggggctggcagggcacccacgaaatgaacctgcgcggtgcagttatgcacgtttccccgtccggtaaaaccatcgtcatgcagatccacgcagttatgccggacggttccaatgcgccaccactggttaaaggccagttctacaaaaacacgctggacttcctggtgaaaaattctgcggctggtggtaaagatactcactacgtgttcgaaggcatcgaactgggtaaaccatacgacgctcagatcaaagttgtagatggtgtcctgtctatgaccgttaatggtcagactaaaactgttgacttcgtggctaaagatgcgggctggaaggatctgaaattctatttcaaggcaggtaactatctgcaggaccgccaggccgacggctccgatacctctgccctggtaaagctgtacaaactggacgttaaacattccagc

SEQ ID NO: 31 - protein, 641 aa, Sphingomonas sp. AI
  Alginate lyase AI-I
MPLACLATTRVGAAREKSGDSSMFDIPFPGHGRRLAVAALAFAGCAFAGSLQAHPFDQAVVKDPTASYVDVKARRTFLQSGQLDDRLKAALPKEYDCTTEATPNPQQGEMVIPRRYLSGNHGPVNPDYEPVVTLYRDFEKISATLGNLYVATGKPVYATCLLNMLDKWAKADALLNYDPKSQSWYQVEWSAATAAFALSTMMAEPNVDTAQRERVVKWLNRVARHQTSFPGGDTSCCNNHSYWRGQEATIIGVISKDDELFRWGLGRYVQAMGLINEDGSFVHEMTRHEQSLHYQNYAMLPLTMIAETASRQGIDLYAYKENGRDIHSARKFVFAAVKNPDLIKKYASEPQDTRAFKPGRGDLNWIEYQRARFGFADELGFMTVPIFDPRTGGSGTLLAYKPQGAAAQAPVSAPAAAHSSIDLSKWKLQIPVDPIDVATRDLLKGYQDKYFYVDKDGSLAFWCPASGFKTTANTKYPRSELREMLDPDNHAVNWGWQGTHEMNLRGAVMHVSPSGKTIVMQIHAVMPDGSNAPPLVKGQFYKNTLDFLVKNSAAGGKDTHYVFEGIELGKPYDAQIKVVDGVLSMTVNGQTKTVDFVAKDAGWKDLKFYFKAGNYLQDRQADGSDTSALVKLYKLDVKHSS

SEQ ID NO: 32 - DNA, 717 bp, Artificial Sequence
  codon optimized alginate lyase AI-II
cagggtgcggctgctcaggcgccggtttccgctccggcggcagcacactcttccatcgatctgtccaaatggaaactgcagatccctgttgacccgatcgatgttgctacccgcgatctgctgaagggttatcaggacaagtatttctacgtggataaagatggttctctggccttctggtgcccagcatccggtttcaaaaccacggcgaatactaagtatccgcgtagcgagctgcgtgaaatgctggacccggataatcatgctgttaattggggctggcagggcacccacgaaatgaacctgcgcggtgcagttatgcacgtttccccgtccggtaaaaccatcgtcatgcagatccacgcagttatgccggacggttccaatgcgccaccactggttaaaggccagttctacaaaaacacgctggacttcctggtgaaaaattctgcggctggtggtaaagatactcactacgtgttcgaaggcatcgaactgggtaaaccatacgacgctcagatcaaagttgtagatggtgtcctgtctatgaccgttaatggtcagactaaaactgttgacttcgtggctaaagatgcgggctggaaggatctgaaattctatttcaaggcaggtaactatctgcaggaccgccaggccgacggctccgatacctctgccctggtaaagctgtacaaactggacgttaaacattccagc

SEQ ID NO: 33 - protein, 239 aa, Sphingomonas sp. AI
  Alginate lyase AI-II
QGAAAQAPVSAPAAAHSSIDLSKWKLQIPVDPIDVATRDLLKGYQDKYFYVDKDGSLAFWCPASGFKTTANTKYPRSELREMLDPDNHAVNWGWQGTHEMNLRGAVMHVSPSGKTIVMQIHAVMPDGSNAPPLVKGQFYKNTLDFLVKNSAAGGKDTHYVFEGIELGKPYDAQIKVVDGVLSMTVNGQTKTVDFVAKDAGWKDLKFYFKAGNYLQDRQADGSDTSALVKLYKLDVKHSS

SEQ ID NO: 34 - DNA, 1047 bp, Artificial Sequence
  codon optimized alginate lyase AI-III
cacccgttcgaccaagcagttgtgaaagatccgactgcgtcctatgttgacgttaaagcgcgtcgtactttcctgcaaagcggtcaactggatgatcgcctgaaagcagcgctgccgaaggaatatgactgtaccaccgaagcgacgccgaacccacagcagggtgaaatggtgatcccacgccgctatctgtccggtaaccacggcccggtgaatccggattacgagccggttgtcactctgtatcgcgacttcgaaaaaatcagcgcgaccctgggtaacctgtacgttgcgactggtaaaccagtgtacgcaacttgtctgctgaacatgctggacaaatgggctaaagcagacgcgctgctgaactatgacccgaaatctcagagctggtatcaagtagaatggtccgcagccacggcggcctttgccctgagcactatgatggcagagccgaacgtggacaccgcgcagcgtgagcgtgttgtgaaatggctgaaccgtgtagcacgtcaccagacttcttttccgggtggcgacactagctgctgtaacaatcattcttactggcgtggtcaggaggctaccatcatcggcgttatttccaaggatgatgaactgttccgttggggtctgggtcgttatgtacaggcgatgggtctgatcaacgaagatggttccttcgttcacgaaatgactcgtcacgaacagagcctgcattatcagaactatgcgatgctgccgctgaccatgatcgctgagactgcctctcgtcagggtatcgatctgtatgcttacaaggaaaacggtcgtgatatccattctgctcgtaaattcgtattcgcggccgtaaagaatccggatctgatcaagaaatacgcgagcgaaccgcaggacacgcgcgcttttaaaccgggtcgcggcgatctgaactggatcgaatatcagcgtgcgcgtttcggctttgcagatgagctgggctttatgaccgtgccaatcttcgatccgcgcaccggcggctctggcactctgctggcgtataagcca

SEQ ID NO: 35 - protein, 349 aa, Sphingomonas sp. AI
  Alginate lyase AI-III
HPFDQAVVKDPTASYVDVKARRTFLQSGQLDDRLKAALPKEYDCTTEATPNPQQGEMVIPRRYLSGNHGPVNPDYEPVVTLYRDFEKISATLGNLYVATGKPVYATCLLNMLDKWAKADALLNYDPKSQSWYQVEWSAATAAFALSTMMAEPNVDTAQRERVVKWLNRVARHQTSFPGGDTSCCNNHSYWRGQEATIIGVISKDDELFRWGLGRYVQAMGLINEDGSFVHEMTRHEQSLHYQNYAMLPLTMIAETASRQGIDLYAYKENGRDIHSARKFVFAAVKNPDLIKKYASEPQDTRAFKPGRGDLNWIEYQRARFGFADELGFMTVPIFDPRTGGSGTLLAYKP

SEQ ID NO: 36 - DNA, 1237 bp, Pseudoaldetomonas sp. SM0524
  Alginate lyase
atgttcaggtttaaaggaataaggataatgattaaccataaaaaactgtttatttacagcgcaattgcgacaagttcagcgctatctcatgctgcaacaattaataatgcaggctttgaaagtggctttagtaactggaacgaaaccgacccagccgctatttcttcagatgcttacagtggctcaaaatcgttaaaaattcagggcagtccagcacgggtttatcaagtggtagatatacagcctaacactgaatacaccctaagtgcttatgtgctgggtaaagggcaaattggtgtaaacgatttaaatggtttatttaaaaaccaaacctttaatgtttcttcgtggactaaagtaacaaaaacatttacctcagcaaacaccaattcacttcaggtttttgctaaacattacgacaacaccagcgatgtaaggtttgataatttttccttgattgagggcagcggtagtaatgatggtggctcagatggcggcagcgataactcaaatggttcaacaattcctagcagcataaccagtggtagcatttttgatttagaaggggataacccaaatcctctcgttgacgatagcaccttagtgtttgtgccgttaggggcacaacatattacgcctaatggtaatggctggcgtcatgagtataaggttaaagaaagtttacgcgttgctatgactcaaacctatgaagtgttcgaagctacggtaaaagttgagatgtctgatggcggaaaaacaattatatcgcagcaccatgctagtgataccggcactatatctaaagtgtatgtgtcggatactgatgaatcgggctttaatgatagcgtagcgaacaacgggatttttgatgtgtacgtacgtttacgtaataccagcggtaatgaagaaaaatttgctttgggtacaatgaccagcggtgagacatttaacttgcgggtagttaataactacggcgatgtagaggttacggcattcggtaactcgttcggtataccggtagaggatgattcgcagtcatactttaagtttggtaactacctgcaatcgcaagacccgtacacattagataaatgtggtgaggccggaaactctaactcgtttaaaaactgttttgaggatttaggcattacagagtcaaaagtgacgatgaccaatgtgagttatacgcgtgaaactaattaagcttggtctagagcgctctagaccaagcttaatt

SEQ ID NO: 37 - protein, 400 aa, Pseudoaldetomonas sp. SM0524
  Alginate lyase
MFRFKGIRIMINHKKLFIYSAIATSSALSHAATINNAGFESGFSNWNETDPAAISSDAYSGSKSLKIQGSPARVYQVVDIQPNTEYTLSAYVLGKGQIGVNDLNGLFKNQTFNVSSWTKVTKTFTSANTNSLQVFAKHYDNTSDVRFDNFSLIEGSGSNDGGSDGGSDNSNGSTIPSSITSGSIFDLEGDNPNPLVDDSTLVFVPLGAQHITPNGNGWRHEYKVKESLRVAMTQTYEVFEATVKVEMSDGGKTIISQHHASDTGTISKVYVSDTDESGFNDSVANNGIFDVYVRLRNTSGNEEKFALGTMTSGETFNLRVVNNYGDVEVTAFGNSFGIPVEDDSQSYFKFGNYLQSQDPYTLDKCGEAGNSNSFKNCFEDLGITESKVTMTNVSYTRETN

SEQ ID NO: 38 - DNA, 699 bp, Pseudoaldetomonas sp. SM0524
  Truncated alignate lyase
gataactcaaatggttcaacaattcctagcagcataaccagtggtagcatttttgatttagaaggggataacccaaatcctctcgttgacgatagcaccttagtgtttgtgccgttaggggcacaacatattacgcctaatggtaatggctggcgtcatgagtataaggttaaagagagcttacgcgttgctatgactcaaacctatgaagtgttcgaagctacggtaaaagttgagatgtctgatggcggaaaaacaattatatcgcagcaccatgctagtgataccggcactatatctaaagtgtatgtgtcggatactgatgaatcgggctttaatgatagcgtagcgaacaacgggatttttgatgtgtacgtacgtttacgtaataccagcggtaatgaagaaaaatttgctttgggtacaatgaccagcggtgagacatttaacttgcgggtagttaataactacggcgatgtagaggttacggcattcggtaactcgttcggtataccggtagaggatgattcgcagtcatactttaagtttggtaactacctgcaatcgcaagacccgtacacattagataaatgtggtgaggccggaaactctaactcgtttaaaaactgttttgaggatttaggcattacagagtcaaaagtgacgatgaccaatgtgagttatacgcgtgaaactaat

SEQ ID NO: 39 - protein, 233 aa, Pseudoaldetomonas sp. SM0524
  Truncated alginate lyase
DNSNGSTIPSSITSGSIFDLEGDNPNPLVDDSTLVFVPLGAQHITPNGNGWRHEYKVKESLRVAMTQTYEVFEATVKVEMSDGGKTIISQHHASDTGTISKVYVSDTDESGFNDSVANNGIFDVYVRLRNTSGNEEKFALGTMTSGETFNLRVVNNYGDVEVTAFGNSFGIPVEDDSQSYFKFGNYLQSQDPYTLDKCGEAGNSNSFKNCFEDLGITESKVTMTNVSYTRETN

SEQ ID NO: 40 - DNA, 537 bp, Pseudomonas syringae
  inaK
atgactctcgacaaggcgttggtgctgcgtacctgtgcaaataacatggccgatcactgcggccttatatggcccgcgtccggcacggtggaatccagatactggcagtcaaccaggcggcatgagaatggtctggtcggtttactgtggggcgctggaaccagcgcttttctaagcgtgcatgccgatgctcgatggattgtctgtgaagttgccgttgcagacatcatcagtctggaagagccgggaatggtcaagtttccgcgggccgaggtggttcatgtcggcgacaggatcagcgcgtcacacttcatttcggcacgtcaggccgaccctgcgtcaacgtcaacgtcaacgtcaacgtcaacgttaacgccaatgcctacggccatacccacgcccatgcctgcggtagcaagtgtcacgttaccggtggccgaacaggcccgtcatgaagtgttcgatgtcgcgtcggtcagcgcggctgccgccccagtaaacaccctgccggtgacgacgccgcagaatttgcagacc

SEQ ID NO: 41 - protein, 179 aa, Pseudomonas syringae
  inaK
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQT

SEQ ID NO: 42 - DNA, 2007 bp, Artificial Sequence
  phoA-estA fusion construct
catggatgaaacaaagcactattgcactggcactcttaccgttactgtttacccctgtgacaaaagcccggatccagtctagctctagaggcgcacctgttccttatcctgatcctctggagccgcgtgcagcgtctgctccgtccccatactctactctggttgtttttggtgatgcgctcagcgatgccgggcagttccccgatcctgccggccccgccggaagcacctcgcgtttcaccaaccgggtcggcccgacctaccagaacggcagcggcgagatcttcggaccgaccgcgcccatgctgctcggcaatcagctcggcatcgccccgggcgacctggctgcctcgacctcgccggtcaacgcccagcagggcatcgccgacggcaacaactgggcggtgggcggctaccggaccgaccagatctacgactcgatcaccgcggccaacggctcgctgatcgagcgcgacaataccctcctgcgcagccgcgatggctacgtggtggaccgtgcccgccagggcctgggtgccgatccgaacgcgttgtactacatcaccggcggcggcaacgacttcctccaagggcgcatcctcaacgacgtccaggcacaacaggccgccggtcgcctggtggatagcgtgcaggccctgcagcaggccggcgcgcgctacatcgtggtctggctgttgcccgacctgggcctgaccccggctaccttcggtggtcccttgcagcctttcgccagccaactcagcggcacgttcaacgccgagctgaccgcccagttgagccaggccggcgccaacgtcattccgttgaacatcccgctgctgctcaaggaaggcatggccaacccggcttccttcggcctggccgccgaccagaacctgatcggcacctgtttcagcggcaacggctgtaccatgaacccgacctacgggatcaacggcagcacgcccgacccgagcaaactgctgttcaacgacagcgtgcacccgaccatcaccggccagcgcctgatcgccgactacacctattcgctgctgtcggcgccctgggagttgaccctgctgccggaaatggcccacggcaccctgcgtgcctaccaggacgaactgcgcagccagtggcaggcggactgggagaactggcagaacgtcggccagtggcgcggcttcgtcggcggcggcggccagcgcctggacttcgactcccaggacagcgccgccagcggcgacggcaatggctacaacctgacccttggcggcagctaccgcatcgacgaggcctggcgcgccggggtcgccgccggtttctaccggcagaagctggaagccggcgccaaggattccgactaccgaatgaacagctacatggccagcgccttcgtgcagtaccaggaaaaccactggtgggccgacgcggctttgaccggcggctacctcgactacgacgacctgaagcgcaagttcgccctgggcggcggcgagcgcagcgagaaaggcgacaccaacggccacctgtgggcgttcagcgcgcgcctgggctacgacatcgcccagcaggccgacagtccctggcacctgtcgccgttcgtcagcgccgactatgcacgggtcgaggtcgacggctattccgagaagggcgccagcgccaccgcgctcgactacgacgaccagaagcgcagctcgaagcgcctgggcgccggcctgcaaggcaagtacgcgttcggcagcgatactcagctgttcgccgagtacgcccacgaacgcgagtacgaggacgacacccaggacctcaccatgtccctcaacagcctgccgggcaatcgctttaccctcgaaggctacaccccgcaggaccacctcaaccgcgtctccctcggcttcagccagaagctggcaccggagctgtcgctgcgcggcggctacaactggcgcaagggcgaggacgatacccagcagagcgtcagcctggcgctgagcctggacttctgaa

SEQ ID NO: 43 - protein, 21 aa, Escherichia coli
  phoA leader peptide
MKQSTIALALLPLLFTPVTKA

SEQ ID NO: 44 - protein, 640 aa, Pseudomonas aeruginosa
  estA
SRGAPVPYPDPLEPRAASAPSPYSTLVVFGDALSDAGQFPDPAGPAGSTSRFTNRVGPTYQNGSGEIFGPTAPMLLGNQLGIAPGDLAASTSPVNAQQGIADGNNWAVGGYRTDQIYDSITAANGSLIERDNTLLRSRDGYVVDRARQGLGADPNALYYITGGGNDFLQGRILNDVQAQQAAGRLVDSVQALQQAGARYIVVWLLPDLGLTPATFGGPLQPFASQLSGTFNAELTAQLSQAGANVIPLNIPLLLKEGMANPASFGLAADQNLIGTCFSGNGCTMNPTYGINGSTPDPSKLLFNDSVHPTITGQRLIADYTYSLLSAPWELTLLPEMAHGTLRAYQDELRSQWQADWENWQNVGQWRGFVGGGGQRLDFDSQDSAASGDGNGYNLTLGGSYRIDEAWRAGVAAGFYRQKLEAGAKDSDYRMNSYMASAFVQYQENHWWADAALTGGYLDYDDLKRKFALGGGERSEKGDTNGHLWAFSARLGYDIAQQADSPWHLSPFVSADYARVEVDGYSEKGASATALDYDDQKRSSKRLGAGLQGKYAFGSDTQLFAEYAHEREYEDDTQDLTMSLNSLPGNRFTLEGYTPQDHLNRVSLGFSQKLAPELSLRGGYNWRKGEDDTQQSVSLALSLDF

SEQ ID NO: 45 - DNA, 705 bp, Trichoderma reesei
  endo-1,4-glucanase III
atgaagttccttcaagtcctccctgccctcataccggccgccctggcccaaaccagctgtgaccagtgggcaaccttcactggcaacggctacacagtcagcaacaacctttggggagcatcagccggctctggatttggctgcgtgacggcggtatcgctcagcggcggggcctcctggcacgcagactggcagtggtccggcggccagaacaacgtcaagtcgtaccagaactctcagattgccattccccagaagaggaccgtcaacagcatcagcagcatgcccaccactgccagctggagctacagcgggagcaacatccgcgctaatgttgcgtatgacttgttcaccgcagccaacccgaatcatgtcacgtactcgggagactacgaactcatgatctggcttggcaaatacggcgatattgggccgattgggtcctcacagggaacagtcaacgtcggtggccagagctggacgctctactatggctacaacggagccatgcaagtctattcctttgtggcccagaccaacactaccaactacagcggagatgtcaagaacttcttcaattatctccgagacaataaaggatacaacgctgcaggccaatatgttcttagctaccaatttggtaccgagcccttcacgggcagtggaactctgaacgtcgcatcctggaccgcatctatcaactaa

SEQ ID NO: 46 - protein, 234 aa, Trichoderma reesei
  endo-1,4-glucanase III
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 47 - DNA, 1380 bp, Trichoderma reesei
  endo-beta-1,4-glucanase I
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 48 - protein, 459 aa, Trichoderma reesei
  endo-beta-1,4-glucanase I
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 49 - DNA, 1257 bp, Trichoderma reesei
  endo-beta-1,4-glucanase II
atgaacaagtccgtggctccattgctgcttgcagcgtccatactatatggcggcgccgtcgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaagtag

SEQ ID NO: 50 - protein, 418 aa, Trichoderma reesei
  endo-beta-1,4-glucanase II
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 51 - DNA, 1416 bp, Trichoderma reesei
  Cellobiohydrolase II
atgattgtcggcattctcaccacgctggctacgctggccacactcgcagctagtgtgcctctagaggagcggcaagcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaa

SEQ ID NO: 52 - protein, 471 aa, Trichoderma reesei
  Cellobiohydrolase II
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 53 - DNA, 2658 bp, Clostridium cellulolyticum
  Cel9E
atgaaaaaaaggttagtgaagaaagttgcgatgctcatcgcaatagtgctggttctatcttcttcaataggacaagcatttgcccttgttggggcaggagatttgattcgaaaccatacctttgacaacagagtaggtcttccatggcacgtggttgaatcataccctgcaaaggcaagttttgaaattacatctgatggtaagtacaagataactgctcaaaagatcggtgaggcaggaaaaggtgaaagatgggatatacaattccgtcacagaggactcgcattgcaacaaggtcatacttatacagtaaagtttactgttactgctagcagagcttgtaaaatttatcctaaaataggtgaccagggtgatccatatgatgaatactggaatatgaatcaacaatggaatttcctggaattacaggctaatactccaaaaactgtaactcagacatttacacagactaagggagataagaagaacgttgaatttgcttttcaccttgctcccgataaaactacatctgaggcacagaatccagcaagtttccaacctataacatatacttttgatgaaatttatattcaggaccctcaatttgcaggatatactgaagatccacctgaacctactaatgttgtacgtttgaatcaggtaggtttctatcctaatgctgataagattgcaacagtagcaacaagttcaacaactccaattaactggcagttggttaatagtactggagcagctgttttaacaggtaaatcaactgttaaaggtgccgaccgtgcatcaggtgataatgtccatatcattgatttctctagttacacaacacctggtaccgactataagatagtaacagatgtatcagtaacaaaagccggagacaatgaaagtatgaagttcaatattggagatgacctttttactcaaatgaaatacgattcaatgaagtatttctatcacaacagaagtgctattccaatacaaatgccatactgtgatcaatcacaatgggcacgtcctgcaggacacacaactgatatacttgctccagatccaacaaaggattacaaggctaactacacacttgacgttacaggtggttggtatgatgccggtgaccatggtaagtatgttgttaatggtggtattgcaacctggaccgtaatgaatgcatatgagcgtgcactacacatgggtggagacacttcagttgctccatttaaagacggttcattgaacatacctgaaagcggaaatggctatcctgacatactggacgaagctcgttacaatatgaaaacattattaaatatgcaggttccagcaggaaatgaattagccggtatggctcaccacaaagctcatgacgaacgttggacagctcttgctgtacgtcccgaccaggatacaatgaaacgttggttgcagcctccaagtacagcagctacattaaatctggctgctattgctgcacaaagttcacgtctttggaaacagtttgattctgctttcgcaactaagtgtttaactgcagcagaaactgcatgggatgcagctgtagctcatccagaaatatatgcaactatggaacagggtgccggtggtggagcatacggagacaactatgttcttgatgatttctactgggcagcatgtgaattgtatgcaactacaggcagtgacaagtatttgaactacataaagagctcaaagcattatctcgaaatgcctacagaattaacaggcggtgagaatactggaattacaggggcttttgactggggttgtacagcaggtatgggaacaataacacttgcacttgtacctacaaagcttccggcagcagatgttgctacagctaaagctaatattcaagctgcagctgataagttcatatcaatttcaaaagcacaaggctatggtgtaccactagaagaaaaagtaatttcatctccttttgatgcatctgttgttaaaggtttccaatggggatcaaactcattcgttattaatgaagcaatagttatgtcatatgcttatgaattcagcgatgttaatggcacaaagaataataaatatattaatggtgctttaacagcaatggattacctcctcggacgtaacccaaatattcaaagctatataactggttatggtgacaacccacttgaaaatcctcatcaccgtttctgggcataccaggcagacaacacattcccaaaaccacctccgggatgtctgtcaggaggacctaactccggcttgcaggatccttgggttaagggttcaggctggcagccaggtgaaagacctgctgaaaaatgcttcatggacaatattgaatcttggtcaacaaacgaaataaccatcaactggaatgctcctcttgtatggatatcagcttaccttgatgaaaaggggccagagattggtgggtcagtgactcctccaactaatttaggagatgttaacggcgatggaaacaaggatgcattggacttcgctgcattgaagaaagccttgttaagccaggatacttctactataaatgttgctaatgctgatataaacaaagatggttctattgatgcagttgactttgcattactcaaatcattcttgttaggaaaaatcacactgtaa

SEQ ID NO: 54 - protein, 885 aa, Clostridium cellulolyticum
  Cel9E
MKKRLVKKVAMLIAIVLVLSSSIGQAFALVGAGDLIRNHTFDNRVGLPWHVVESYPAKASFEITSDGKYKITAQKIGEAGKGERWDIQFRHRGLALQQGHTYTVKFTVTASRACKIYPKIGDQGDPYDEYWNMNQQWNFLELQANTPKTVTQTFTQTKGDKKNVEFAFHLAPDKTTSEAQNPASFQPITYTFDEIYIQDPQFAGYTEDPPEPTNVVRLNQVGFYPNADKIATVATSSTTPINWQLVNSTGAAVLTGKSTVKGADRASGDNVHIIDFSSYTTPGTDYKIVTDVSVTKAGDNESMKFNIGDDLFTQMKYDSMKYFYHNRSAIPIQMPYCDQSQWARPAGHTTDILAPDPTKDYKANYTLDVTGGWYDAGDHGKYVVNGGIATWTVMNAYERALHMGGDTSVAPFKDGSLNIPESGNGYPDILDEARYNMKTLLNMQVPAGNELAGMAHHKAHDERWTALAVRPDQDTMKRWLQPPSTAATLNLAAIAAQSSRLWKQFDSAFATKCLTAAETAWDAAVAHPEIYATMEQGAGGGAYGDNYVLDDFYWAACELYATTGSDKYLNYIKSSKHYLEMPTELTGGENTGITGAFDWGCTAGMGTITLALVPTKLPAADVATAKANIQAAADKFISISKAQGYGVPLEEKVISSPFDASVVKGFQWGSNSFVINEAIVMSYAYEFSDVNGTKNNKYINGALTAMDYLLGRNPNIQSYITGYGDNPLENPHHRFWAYQADNTFPKPPPGCLSGGPNSGLQDPWVKGSGWQPGERPAEKCFMDNIESWSTNEITINWNAPLVWISAYLDEKGPEIGGSVTPPTNLGDVNGDGNKDALDFAALKKALLSQDTSTINVANADINKDGSIDAVDFALLKSFLLGKITL

SEQ ID NO: 55 - DNA, 1581 bp, Clostridium cellulolyticum
  Cel9M
atgaaaagtaaattgataaaattaagtgcaataataatttccggagttatgttaactacttcatttgttaacacaggtactgcatttgccgcaggaacacatgattattcaactgctttaaaggactcaataatttttttcgatgcaaacaaatgtggtcctcaagcaggagaaaataatgtatttgattggagaggtgcgtgtcatacaactgacggaagcgatgtaggtgttgatttaacaggcggctatcatgatgcgggagatcatgtgaagtttggtctgcctcaaggttattcagcagcaattttaggttggtcattgtacgaattcaaggaatcctttgatgcaaccggaaatactacaaaaatgttgcaacagctgaaatattttacagactatttccttaaatcacatcctaactcaaccacgttctactaccaggttggagagggaaatgccgatcatacttattggggtgcacccgaggagcaaacaggtcaaagaccttcattatacaaggctgacccaagctctcctgcatccgatattctgagtgaaacatctgctgctcttactctgatgtatttaaattacaaaaatattgattctgcttatgcgacaaagtgcctcaatgcagcaaaagaactgtatgcaatgggtaaggcaaatcaaggtgtaggcaacggtcagtccttttatcaggctaccagctttggcgatgacttggcttgggccgctacatggttgtacacagcaacaaatgacagtacttacataactgatgctgaacaattcataacattgggcaatacaatgaatgagaacaagatgcaggataaatggaccatgtgctgggatgacatgtatgttccggctgctttgagattggctcagattacgggaaagcagatatacaaggatgcaatagaatttaatttcaactactggaaaactcaagtaacaactactccgggaggcttaaagtggctttcgaactggggtgttctgagatatgcagcagcggaatccatggttatgctggtttactgcaagcaaaatcctgatcaatctcttttagatttagctaaaaagcaggtagactatatcctaggagataatcctgccaatatgtcctatattattggttatggaagtaattggtgcattcatcctcatcacagggcagcaaacggctacacttatgctaatggagataatgccaagccggcaaagcacttattaacaggtgcactggtggggggacctgatcagaatgacaaatttcttgacgatgcaaaccagtatcagtatacagaggtagcccttgactacaatgcgggtcttgtgggtgtacttgcaggtgccattaagttctttggaggtacgattgttaatcctcctgtaaaaaaaggtgatcttaataatgatacatttatagatgctatagaccttgcactttgcaaaaactatattcttactcaaaacggaaatattgataaaaataatgcagatatgaatggcgacggttccatagatgccatcgacttttctcttttaaagaaggctatcttaggttaa

SEQ ID NO: 56 - protein, 526 aa, Clostridium cellulolyticum
  Cel9M
MKSKLIKLSAIIISGVMLTTSFVNTGTAFAAGTHDYSTALKDSIIFFDANKCGPQAGENNVFDWRGACHTTDGSDVGVDLTGGYHDAGDHVKFGLPQGYSAAILGWSLYEFKESFDATGNTTKMLQQLKYFTDYFLKSHPNSTTFYYQVGEGNADHTYWGAPEEQTGQRPSLYKADPSSPASDILSETSAALTLMYLNYKNIDSAYATKCLNAAKELYAMGKANQGVGNGQSFYQATSFGDDLAWAATWLYTATNDSTYITDAEQFITLGNTMNENKMQDKWTMCWDDMYVPAALRLAQITGKQIYKDAIEFNFNYWKTQVTTTPGGLKWLSNWGVLRYAAAESMVMLVYCKQNPDQSLLDLAKKQVDYILGDNPANMSYIIGYGSNWCIHPHHRAANGYTYANGDNAKPAKHLLTGALVGGPDQNDKFLDDANQYQYTEVALDYNAGLVGVLAGAIKFFGGTIVNPPVKKGDLNNDTFIDAIDLALCKNYILTQNGNIDKNNADMNGDGSIDAIDFSLLKKAILG

SEQ ID NO: 57 - DNA, 2073 bp, Clostridium cellulolyticum
  Cel9G
atggcaggaacatataactatggagaagcattacagaaatcaataatgttctatgaattccagcgttcgggagatcttccggctgataaacgtgacaactggagagacgattccggtatgaaagacggttctgatgtaggagttgatcttacaggaggatggtacgatgcaggtgaccatgtgaaatttaatctacctatgtcatatacatctgcaatgcttgcatggtccttatatgaggataaggatgcttatgataagagcggtcagacaaaatatataatggacggtataaaatgggctaatgattattttattaaatgtaatccgacacccggtgtatattattaccaagtaggagacggcggaaaggaccactcttggtggggccctgcggaagtaatgcagatggaaagaccgtcttttaaggttgacgcttctaagcccggttctgcagtatgtgcttccactgcagcttctctggcatctgcagcagtagtctttaaatccagtgatcctacttatgcagaaaagtgcataagccatgcaaagaacctgtttgatatggctgacaaagcaaagagtgatgctggttatactgcggcttcaggctactacagctcaagctcattttacgatgatctctcatgggctgcagtatggttatatcttgctacaaatgacagtacatatttagacaaagcagaatcctatgtaccgaattggggtaaagaacagcagacagatattatcgcctacaagtggggacagtgctgggatgatgttcattatggtgctgagcttcttcttgcaaagcttacaaacaaacaattgtataaggatagtatagaaatgaaccttgacttctggacaactggtgttaacggaacacgtgtttcttacacgccaaagggtttggcgtggctattccaatggggttcattaagacatgctacaactcaggcttttttagccggtgtttatgcagagtgggaaggctgtacgccatccaaagtatctgtatataaggatttcctcaagagtcaaattgattatgcacttggcagtaccggaagaagttttgttgtcggatatggagtaaatcctcctcaacatcctcatcacagaactgctcacggttcatggacagatcaaatgacttcaccaacataccacaggcatactatttatggtgcgttggtaggaggaccggataatgcagatggctatactgatgaaataaacaattatgtcaataatgaaatagcctgcgattataatgccggatttacaggtgcacttgcaaaaatgtacaagcattctggcggagatccgattccaaacttcaaggctatcgaaaaaataaccaacgatgaagttattataaaggcaggtttgaattcaactggccctaactacactgaaatcaaggctgttgtttataaccagacaggatggcctgcaagagttacggacaagatatcatttaaatattttatggacttgtctgaaattgtagcagcaggaattgatcctttaagccttgtaacaagttcaaattattctgaaggtaagaatactaaggtttccggtgtgttgccatgggatgtttcaaataatgtttactatgtaaatgttgatttgacaggagaaaatatctacccaggcggtcagtctgcgtgcagacgagaagttcagttcagaattgccgcaccacagggaagaagatattggaatccgaaaaatgatttctcatatgatggattaccaaccaccagtactgtaaatacggttaccaacatacctgtttatgataacggcgtaaaagtatttggtaacgaacccgcaggtggatcggaaaaccctgatcctgaaatcttgtatggagacgtaaacagcgacaaaaatgtagatgcattggactttgctgcattgaagaaatatttacttggaggcacttccagcatagatgttaaggctgcagatacatacaaggatgggaatattgacgctatagatatggctaccttgaagaagtatttattgggaacaatcacccaattacctcaaggc

SEQ ID NO: 58 - protein, 725 aa, Clostridium cellulolyticum
  Cel9G
MLKTKRKLTKAIGVALSISILSSLVSFIPQTNTYAAGTYNYGEALQKSIMFYEFQRSGDLPADKRDNWRDDSGMKDGSDVGVDLTGGWYDAGDHVKFNLPMSYTSAMLAWSLYEDKDAYDKSGQTKYIMDGIKWANDYFIKCNPTPGVYYYQVGDGGKDHSWWGPAEVMQMERPSFKVDASKPGSAVCASTAASLASAAVVFKSSDPTYAEKCISHAKNLFDMADKAKSDAGYTAASGYYSSSSFYDDLSWAAVWLYLATNDSTYLDKAESYVPNWGKEQQTDIIAYKWGQCWDDVHYGAELLLAKLTNKQLYKDSIEMNLDFWTTGVNGTRVSYTPKGLAWLFQWGSLRHATTQAFLAGVYAEWEGCTPSKVSVYKDFLKSQIDYALGSTGRSFVVGYGVNPPQHPHHRTAHGSWTDQMTSPTYHRHTIYGALVGGPDNADGYTDEINNYVNNEIACDYNAGFTGALAKMYKHSGGDPIPNFKAIEKITNDEVIIKAGLNSTGPNYTEIKAVVYNQTGWPARVTDKISFKYFMDLSEIVAAGIDPLSLVTSSNYSEGKNTKVSGVLPWDVSNNVYYVNVDLTGENIYPGGQSACRREVQFRIAAPQGTTYWNPKNDFSYDGLPTTSTVNTVTNIPVYDNGVKVFGNEPAGGSENPDPEILYGDVNSDKNVDALDFAALKKYLLGGTSSIDVKAADTYKDGNIDAIDMATLKKYLLGTITQLPQG

SEQ ID NO: 59 - DNA, 1428 bp, Clostridium cellulolyticum
  Cel5A
atgaaaaaaacaacagcttttttattatgttttctaatgatttttacagcattattgccaatgcaaaatgctaatgcgtatgatgcttcacttattccgaatcttcagattccacaaaagaacattccgaataatgatggaatgaattttgtaaaaggtttaagactcggatggaatctgggtaatacatttgatgcttttaacggtacaaatattactaatgaattggattatgaaacatcatggagcggtatcaaaacaactaagcagatgatagatgcaataaagcaaaaaggattcaatactgttcgtattcctgtatcctggcatccacacgtaagtggttcagattacaaaatcagtgatgtatggatgaatcgtgttcaagaagtagtaaattattgtatagataataaaatgtatgtcattttaaacacacatcatgacgttgacaaagtaaaaggttatttcccaagcagtcaatatatggcaagctccaagaaatatataactagtgtctgggcacagattgctgctaggtttgcaaactatgatgagcatcttatttttgaaggaatgaacgagcctcgtcttgtaggacatgcaaatgagtggtggcctgagctgacaaattcagatgtagttgattctattaattgtattaatcaacttaatcaggattttgttaatacagtacgtgcaacaggtggaaaaaatgcaagcagatatcttatgtgtccaggatatgttgcatctcctgacggagcaacaaacgattacttcagaatgccaaatgatatttctggtaataacaacaaaataattgtatctgtacatgcatattgtccatggaattttgcagggttggcaatggctgatggaggtacaaatgcttggaatataaatgattcaaaagatcaaagtgaagttacttggtttatggataatatttataataagtatacaagcaggggtattcctgtaataatcggtgaatgtggagcagtagataagaacaatctgaagacaagagtagaatatatgtcctattatgttgcacaagctaaagcacgtggtatattatgcatattgtgggataacaataatttctcaggtactggtgaattatttggtttcttcgatagaagaagctgtcagttcaagttccctgaaattatagatggaatggtgaaatatgctttcgaagccaagacagatcctgacccagtaattgtatatggagattataacaatgatggaaatgttgatgcacttgattttgcaggcttaaagaaatatattatggctgctgaccatgcttatgtaaagaatttggatgttaatctcgacaatgaagtgaatgcatttgaccttgctattttgaaaaaatatctgcttggtatggtaagtaagcttccaagcaactaa

SEQ ID NO: 60 - protein, 475 aa, Clostridium cellulolyticum
  Cel5A
MKKTTAFLLCFLMIFTALLPMQNANAYDASLIPNLQIPQKNIPNNDGMNFVKGLRLGWNLGNTFDAFNGTNITNELDYETSWSGIKTTKQMIDAIKQKGFNTVRIPVSWHPHVSGSDYKISDVWMNRVQEVVNYCIDNKMYVILNTHHDVDKVKGYFPSSQYMASSKKYITSVWAQIAARFANYDEHLIFEGMNEPRLVGHANEWWPELTNSDVVDSINCINQLNQDFVNTVRATGGKNASRYLMCPGYVASPDGATNDYFRMPNDISGNNNKIIVSVHAYCPWNFAGLAMADGGTNAWNINDSKDQSEVTWFMDNIYNKYTSRGIPVIIGECGAVDKNNLKTRVEYMSYYVAQAKARGILCILWDNNNFSGTGELFGFFDRRSCQFKFPEIIDGMVKYAFEAKTDPDPVIVYGDYNNDGNVDALDFAGLKKYIMAADHAYVKNLDVNLDNEVNAFDLAILKKYLLGMVSKLPSN

SEQ ID NO: 61 - protein, 722 aa, Clostridium cellulolyticum
  Cel48F
MSKNFKRVGAVAVAAAMSLSIMATTSINAASSPANKVYQDRFESMYSKIKDPANGYFSEQGIPYHSIETLMVEAPDYGHVTTSEAMSYYMWLEAMHGRFSGDFTGFDKSWSVTEQYLIPTEKDQPNTSMSRYDANKPATYAPEFQDPSKYPSPLDTSQPVGRDPINSQLTSAYGTSMLYGMHWILDVDNWYGFGARADGTSKPSYINTFQRGEQESTWETIPQPCWDEHKFGGQYGFLDLFTKDTGTPAKQFKYTNAPDADARAVQATYWADQWAKEQGKSVSTSVGKATKMGDYLRYSFFDKYFRKIGQPSQAGTGYDAAHYLLSWYYAWGGGIDSTWSWIIGSSHNHFGYQNPFAAWVLSTDANFKPKSSNGASDWAKSLDRQLEFYQWLQSAEGAIAGGATNSWNGRYEAVPSGTSTFYGMGYVENPVYADPGSNTWFGMQVWSMQRVAELYYKTGDARAKKLLDKWAKWINGEIKFNADGTFQIPSTIDWEGQPDTWNPTQGYTGNANLHVKVVNYGTDLGCASSLANTLTYYAAKSGDETSRQNAQKLLDAMWNNYSDSKGISTVEQRGDYHRFLDQEVFVPAGWTGKMPNGDVIKSGVKFIDIRSKYKQDPEWQTMVAALQAGQVPTQRLHRFWAQSEFAVANGVYAILFPDQGPEKLLGDVNGDETVDAIDLAILKKYLLNSSTTINTANADMNSDNAIDAIDYALLKKALLSIQ

SEQ ID NO: 62 - DNA, 2169 bp, Clostridium cellulolyticum
  Cel48F
atgagtaagaattttaaaagagtaggagcagttgcagttgctgctgcaatgtcattatctataatggcaacaaccagcataaatgcagcttcaagtcctgcaaacaaggtgtaccaggatcgttttgaatccatgtacagcaagattaaggatcctgcaaacggatacttcagtgaacagggaattccttaccactcaattgaaacactgatggtcgaagctcctgactacggacatgttacaacaagtgaagctatgtcctattatatgtggttggaagcaatgcacggaagattttcaggagattttacaggttttgataagtcttggtctgttaccgaacagtatttgatcccaacagaaaaggatcagcccaatacaagtatgagcagatatgatgctaacaaaccggctacatacgctccggaatttcaggacccaagcaagtatccttctccgttggatactagtcaacctgttggtagagatccaatcaactcacaattgacttcagcatacggaacaagcatgctgtatggaatgcactggatacttgacgttgataactggtatggatttggagcaagagctgatggaacctccaagccatcatatatcaatactttccaaagaggtgagcaggagtcaacttgggaaaccatacctcaaccatgttgggatgaacataaatttggtggacagtacggattcctggatctctttacaaaggatacaggtactccggcaaagcaattcaaatatacaaatgctccagatgctgatgctcgtgcagttcaagcaacttactgggctgatcagtgggcaaaagaacaaggtaagagcgtaagtactagcgtaggtaaggcaacaaagatgggtgattaccttagatattcattctttgataagtatttcagaaagatcggacaaccttctcaggctggtaccggatatgatgcagcacattatctgctttcatggtactatgcatggggtggtggaattgactctacctggtcttggataatcggtagcagtcataatcatttcggttaccagaacccatttgcagcttgggtactttcaacagatgcaaacttcaagcctaagtcatcaaatggtgcatcagactgggcaaagagtttggacagacagcttgaattctatcagtggttgcagtcagcagaaggtgctattgccggtggagctacaaactcatggaacggacgttatgaagcagttccttcaggtacatcaacattctatggaatgggttatgtagaaaaccctgtatatgctgacccaggtagtaacacttggtttggtatgcaggtatggtcaatgcagcgtgtagctgaattgtactataagactggcgatgccagagctaagaaactcttagacaaatgggcaaaatggattaatggcgaaatcaagttcaatgctgacggaacattccagattcctagcacaattgattgggaaggacagccggatacttggaatccaacacagggatacaccggaaatgcaaacttgcatgttaaagttgttaactatggtactgacctaggttgtgcttcttcacttgcaaacacattgacttactatgctgctaaatcaggagatgaaacttcaaggcagaatgcacagaaattacttgacgctatgtggaataactatagcgattcaaagggtatatcaactgttgaacagcgtggtgattaccatagattccttgatcaggaagtttttgtaccagctggttggactggaaaaatgcctaacggcgacgtaatcaaatctggtgtcaagttcatagacattcgttccaagtacaagcaggatcctgaatggcagacaatggttgctgcattacaggcaggacaggttccaactcagagattacaccgtttctgggctcagagtgaatttgcagttgcaaatggagtttatgcaatactcttcccagatcaaggtccagaaaaattattgggtgatgtaaacggtgacgaaactgtagacgctattgaccttgctatacttaaaaaatatcttttaaacagcagtactacaataaatactgcaaatgcagatatgaatagtgataatgctattgacgctattgactatgctcttttaaagaaagcacttctttctatccaatag

SEQ ID NO: 63 - DNA, 2522 bp, Aspergillus aculeatus
  Beta-glucosidase 1
atgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggaattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaagcttctgaaggccgagctcggcttccagggctttgtgatgtctgactggggtgctcaccacagtggtgttggctctgctttggccggcttggatatgtcaatgcctggcgatatcaccttcgattctgccactagtttctggggtaccaacctgaccattgctgtgctcaacggtaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggatccaactcgtacggtgccaatggctgctctgaccgtggctgtgacaacggtactcttgctatggcttggggtagcggcactgccgaattcccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccgggcgccaaatctccattcacctggggcaagacgagggaggcgtacggggattaccttgtccgtgagctcaacaacggcaacggagctccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaa

SEQ ID NO: 64 - protein, 860 aa, Aspergillus aculeatus
  Beta-glucosidase 1
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 65 - DNA, 66 bp, Pectobacterium sp.
  PelB signal peptide
atgaaatatctgctgccaactgcggctgcgggtctgctgctgctggccgcacaaccagcgatggca

SEQ ID NO: 66 - protein, 22 aa, Pectobacterium sp.
  PelB signal peptide
MKYLLPTAAAGLLLLAAQPAMA

SEQ ID NO: 67 - DNA, 63 bp, Escherichia coli
  OmpA signal peptide
atgaaaaagaccgctatcgcaatcgctgttgctctggctggtttcgcgactgtggcccaagcc

SEQ ID NO: 68 - protein, 21 aa, Escherichia coli
  OmpA signal peptide
MKKTAIAIAVALAGFATVAQA

SEQ ID NO: 69 - DNA, 69 bp, Escherichia coli
  stll signal peptide
atgaagaaaaatatcgcgttcctgctggcatctatgtttgtattctccatcgccactaatgcgtatgct

SEQ ID NO: 70 - protein, 23 aa, Escherichia coli
  Stll signal peptide
MKKNIAFLLASMFVFSIATNAYA

SEQ ID NO: 71 - DNA, 84 bp, Escherichia coli
  EX signal peptide
atgttcaagttcaaaaagaaattcctggtaggtctgaccgccgctttcatgtccatctccatgttttccgcgacggcctccgcg

SEQ ID NO: 72 - protein, 28 aa, Escherichia coli
  EX signal peptide
MFKFKKKFLVGLTAAFMSISMFSATASA

SEQ ID NO: 73 - DNA, 63 bp, Escherichia coli
  PhoA signal peptide
atgaaacaatctactattgcgctggccctgctgccactgctgttcaccccggtgactaaagct

SEQ ID NO: 74 - protein, 21 aa, Escherichia coli
  PhoA signal peptide
MKQSTIALALLPLLFTPVTKA

SEQ ID NO: 75 - DNA, 66 bp, Escherichia coli
  OmpF signal peptide
atgatgaaacgtaacattctggccgtaattgtacctgctctgctggttgcgggcactgctaacgct

SEQ ID NO: 76 - protein, 22 aa, Escherichia coli
  OmpF
MMKRNILAVIVPALLVAGTANA

SEQ ID NO: 77 - DNA, 63 bp, Escherichia coli
  PhoE signal peptide
atgaaaaagtccactctggctctggttgttatgggtatcgtagcgagcgcgtctgtacaagcg

SEQ ID NO: 78 - protein, 21 aa, Escherichia coli
  PhoE signal peptide
MKKSTLALVVMGIVASASVQA

SEQ ID NO: 79 - DNA, 78 bp, Escherichia coli
  MalE signal peptide
atgaaaatcaaaactggcgcacgtatcctggctctgtctgctctgaccaccatgatgttctctgcatctgcgctggct

SEQ ID NO: 80 - protein, 26 aa, Escherichia coli
  MalE signal peptide
MKIKTGARILALSALTTMMFSASALA

SEQ ID NO: 81 - DNA, 63 bp, Escherichia coli
  OmpC signal peptide
atgaaagttaaagttctgagcctgctggttcctgcactgctggttgctggtgcggccaacgct

SEQ ID NO: 82 - protein, 21 aa, Escherichia coli
  OmpC signal peptide
MKVKVLSLLVPALLVAGAANA

SEQ ID NO: 83 - DNA, 60 bp, Escherichia coli
  Lpp signal peptide
atgaaagctactaaactggttctgggtgcggttattctgggttctaccctgctggctggt

SEQ ID NO: 84 - protein, 20 aa, Escherichia coli
  Lpp signal peptide
MKATKLVLGAVILGSTLLAG

SEQ ID NO: 85 - DNA, 75 bp, Escherichia coli
  LamB signal peptide
atgatgattactctgcgtaaactgcctctggctgttgcggtagcggcaggtgttatgtccgcgcaagcgatggct

SEQ ID NO: 86 - protein, 25 aa, Escherichia coli
  LamB signal peptide
MMITLRKLPLAVAVAAGVMSAQAMA

SEQ ID NO: 87 - DNA, 60 bp, Escherichia coli
  OmpT signal peptide
atgcgcgcaaaactgctgggtatcgtgctgaccaccccaatcgcgatttcctccttcgca

SEQ ID NO: 88 - protein, 20 aa, Escherichia coli
  OmpT signal peptide
MRAKLLGIVLTTPIAISSFA

SEQ ID NO: 89 - DNA, 63 bp, Escherichia coli
  Ltb signal peptide
atgaataaagttaaatgttacgttctgttcaccgcactgctgtctagcctgtatgcgcacggt

SEQ ID NO: 90 - protein, 21 aa, Escherichia coli
  Ltb signal peptide
MNKVKCYVLFTALLSSLYAHG

SEQ ID NO: 91 - DNA, 861 bp, Vibri splendidus
  V12B01_24269
atgaattctgttacaaaaattgctgcagctgttgcatgtactcttttagcgggcacagctgctggtgcatctcttgattatcgttacgagtatcgtgctgcgacggattatacaaagactaatggtgatacggctcacgtagacgctcgccatcaacaccgagttaagctaggtgaaagctttaagctgtcagacaagtggaagcactctactggtctagaacttaagttccacggtgatgactcttactatgatgaagattcaggttctgttaaatcagcaaacagccagagtttttacgatggcaattggtacatctatggtatggagatcgataacactgcgacatacaaaatagacaataattggtatctacaaatgggtatgcctattgcttgggattgggatgagcctaatgctaacgatggcgactggaagatgaaaaaggttacgtttaaacctcagttccgcgttggctataaagcagatatgggtttaacaactgctattcgttaccgtcatgaatatgctgacttccgtaaccacacacaatttggcgacaaagattctgaaactggcgagcgtttagaatcagctcaaaagtctaaagttacactgacgggctcttacaaaattgaatctctacctaagcttggcctttcttacgaagcaaactatgtaaaatctttggataacgtacttctttataatagtgatgactgggaatgggatgctggcttaaaggtaaactacaagttcggttcttggaaaccttttgctgaaatctggtcttctgatatcagttcatcttcaaaagatcgtgaagctaaataccgtgttggtattgcttactcattctaa

SEQ ID NO: 92 - protein, 286 aa, Vibrio splendidus
  V12B01_24269
MNSVTKIAAAVACTLLAGTAAGASLDYRYEYRAATDYTKTNGDTAHVDARHQHRVKLGESFKLSDKWKHSTGLELKFHGDDSYYDEDSGSVKSANSQSFYDGNWYIYGMEIDNTATYKIDNNWYLQMGMPIAWDWDEPNANDGDWKMKKVTFKPQFRVGYKADMGLTTAIRYRHEYADFRNHTQFGDKDSETGERLESAQKSKVTLTGSYKIESLPKLGLSYEANYVKSLDNVLLYNSDDWEWDAGLKVNYKFGSWKPFAEIWSSDISSSSKDREAKYRVGIAYSF

SEQ ID NO: 93 - DNA, 726 bp, Vibrio splendidus
  V12B01_24309
atgaataaaactaaaatcgtgattgctgtggcatctgtactcgctgctggctctgtatctgctgcgtctttcgatatgagacacgaatacaagagtcatacagatcaacatgctactcgtgtaaaactgggcgatagcattgacaacttccttgtagatatcgaagcgaaattcaagggagaagacggtaaattcatggaagatttgaagaataacggttgggagcttggtctaaattaccgccatgtactaaacgataactggacaatgacttacggtatgccaattgaaggtcgcgaatctggtgttacgtacaaacctcaagttcgtgcaacatacaaagtggatagcgttgatggcctaagcctaagtgctcgttaccgttacgatatgagacaaaatactagcacgactgagtacgagctcgacggtaatggtgacattgtcggtgttgagaaaaacattgaaaaccaacgccgccaccgtctaacggcaaacgttaactactcaatggaaaactggcgctttggtttagagggtaactactacaaagcggatggatatgacatctacgacaacgacgataccaactacgagttgaacgcaagtattcgccacatgatgggtcaatgggcaccatatgtagagtttggtgatgtgagtacttcatcgactaaagcgactcgtgaactacgtagccgtgttggtctaacttacagcttctaa

SEQ ID NO: 94 - protein, 241 aa, Vibrio splendidus
  V12B01_24309
MNKTKIVIAVASVLAAGSVSAASFDMRHEYKSHTDQHATRVKLGDSIDNFLVDIEAKFKGEDGKFMEDLKNNGWELGLNYRHVLNDNWTMTYGMPIEGRESGVTYKPQVRATYKVDSVDGLSLSARYRYDMRQNTSTTEYELDGNGDIVGVEKNIENQRRHRLTANVNYSMENWRFGLEGNYYKADGYDIYDNDDTNYELNASIRHMMGQWAPYVEFGDVSTSSTKATRELRSRVGLTYSF

SEQ ID NO: 95 - DNA, 1659 bp, Vibrio splendidus
  V12B01_24324
atgctgtggtggatggtaggtgcaaccgcatttatgacccagttcagtgcgtggacatttactggtgcagcaggtaaagcctttaccgatggttttgccgtagcgattatctttatcgcgaacgcattcggttacctaatgaactacctttacttcgctccgaagttccgccaactgcgcgttgtgacggttattgaagcgattcgtatgcgttttggtaaggtgaatgagcaagtgtttacttggtctggcatgccaaacagcgttatctccgcaggtatctggttgaatggccttgcgatcatcgcatcgggtatcttcggctttgatatgacaacgacgattgtcctaacgggtctggttgtactggtgatgtcagtaacgggcggctcttgggcggtaatcgcatctgactttatgcagatggttatcataatggcagtgaccgtaacgtgtgctgtagtagcggtctaccacggtggcggtgttacacaaatcatcaatgacttcccaaccgattcatttatcacgggtgataaccttaactacttaagcatcttcagcatctgggcggtgttcatcttcttgaagcagttcagtatcaccaataacatgctgaactcttaccgttaccttgcagcgaaagactcaaacaacgcacgtaaagcagcactgcttgcttgtgtactaatgacactgggtccaatcatttggttcatgccttcatggttcatggcaggtcaaggtgttgatttagcagcagcttacccagaagctggcagtaaagccgctgacttcgcttacctatatttcgttcaagagtacatgccggttggtatggttggccttctgattgccgcgatgttcgcagcaacaatgtcttcaatggactctggtctaaaccgtaactcaggtatcttcgttaagaacttctacgagccaattcttcgccctaaagcgacagagaaagagctaatggttgtttctaaactaacgtctactttcttcggcatcgcgatcatcttggttgctctgttcattaactctcttaaaggtctgagccttttcgacaccatgatgtacgtgggcgcattgatcggcttcccaatgacgattccagcattctgtggcttctttatccgtaaaactccggattgggctggctggggcacgctagtcgttggtggtgtggtttcttacttcgttggtttcgttatcacagcggacatgattcaaaactggttcggtctaaacgaactaactggccgtgagtgggctgacctgaaagtggctatcggccttatcggtcacatcgtatttactgctggcttcttcgttctttcaacattgttctacaagccattaccggaacaccgtgagaaagatgtcgacaagttcttcaacaacctagcgacaccgttagttgctgaaagtaatgaacagaagaaactggataacaagcaacgtcgtatgttgggttcacttatcgcggtagcgggtgtgggcgtaatgactatgttcgtactgccaaaccctatgtggggacgcatggtattcgttctgtgtggtttgattgtgttctctgttggtctgttacttgttaaagcggtcgatgacaaagtcgagcaacaagacgaagtgactccagccgaaggctaa

SEQ ID NO: 96 - protein, 552 aa, Vibrio splendidus
  V12B01_24324
MLWWMVGATAFMTQFSAWTFTGAAGKAFTDGFAVAIIFIANAFGYLMNYLYFAPKFRQLRVVTVIEAIRMRFGKVNEQVFTWSGMPNSVISAGIWLNGLAIIASGIFGFDMTTTIVLTGLVVLVMSVTGGSWAVIASDFMQMVIIMAVTVTCAVVAVYHGGGVTQIINDFPTDSFITGDNLNYLSIFSIWAVFIFLKQFSITNNMLNSYRYLAAKDSNNARKAALLACVLMTLGPIIWFMPSWFMAGQGVDLAAAYPEAGSKAADFAYLYFVQEYMPVGMVGLLIAAMFAATMSSMDSGLNRNSGIFVKNFYEPILRPKATEKELMVVSKLTSTFFGIAIILVALFINSLKGLSLFDTMMYVGALIGFPMTIPAFCGFFIRKTPDWAGWGTLVVGGVVSYFVGFVITADMIQNWFGLNELTGREWADLKVAIGLIGHIVFTAGFFVLSTLFYKPLPEHREKDVDKFFNNLATPLVAESNEQKKLDNKQRRMLGSLIAVAGVGVMTMFVLPNPMWGRMVFVLCGLIVFSVGLLLVKAVDDKVEQQDEVTPAEG

SEQ ID NO: 97 - DNA, 1743 bp, Vibrio splendidus
  V12B01_24254
gtgaataagccaatctttgtcgtcgtactcgcttcgcttacgtatggctgcggtggaagcagctccagtgactctagtgacccttctgataccaataactcaggagcatcttatggtgttgttgctccctatgatattgccaagtatcaaaacatcctttccagctcagatcttcaggtgtctgatcctaatggagaggagggcaataaaacctctgaagtcaaagatggtaacttcgatggttatgtcagtgattatttttatgctgacgaagagacggaaaatctgatcttcaaaatggcgaactacaagatgcgctctgaagttcgtgaaggagaaaacttcgatatcaatgaagcaggcgtaagacgcagtctacatgcggaaataagcctacctgatattgagcatgtaatggcgagttctcccgcagatcacgatgaagtgaccgtgctacagatccacaataaaggtacagacgagagtggcacgggttatatccctcatccgctattgcgtgtggtttgggagcaagaacgagatggcctcacaggtcactactgggcagtcatgaaaaataatgccattgactgtagcagtgccgctgactcttcggattgttatgccacttcatataatcgctacgatttgggagaggcggatctcgataacttcaccaagtttgatctttctgtttatgaaaataccctttcgatcaaagtgaacgatgaagttaaagtcgacgaagacatcacctactggcagcatctactgagttactttaaagcgggtatctacaatcaatttgaaaatggtgaagccacggctcactttcaggcactgcgatacaccaccacacaggtcaacggctcaaacgattgggatattaatgattggaagttgacgattcctgcgagtaaagacacttggtatggaagtgggggtgacagtgcggctgaactagaacctgagcgctgcgaatcgagcaaagaccttctcgccaacgacagtgatgtctacgacagcgatattggtctttcttatttcaataccgatgaagggagagtgcactttagagcggatatgggatatggcacctctaccgaaaattctagctatattcgctctgagctcagggagttgtatcaaagcagtgttcaaccggattgtagcaccagcgatgaagatacaagttggtatttggacgacactagaacgaacgctaccagtcacgagttaaccgcaagcttacgaattgaagactacccgaacattaataaccaagacccgaaagtggtgcttgggcaaatacacggttggaagatcaatcaagcattggtgaagttgttatgggaaggcgagagtaagccagtaagagtgatactgaactctgattttgagcgcaacaaccaagactgtaaccattgtgacccgttcagtgtcgagttaggtacttattcggcaagtgaagagtggcgatatacgattcgagccaatcaagacggtatctacttagcgactcatgatttagatggaactaatacggtttctcatttaatcccttggggacaagattacacagataaagatggggacacggtctcgttgacgtcagattggacatcgacagacatcgctttctatttcaaagcgggcatctacccacaatttaagcctgatagcgactatgcgggtgaagtgtttgatgtgagctttagttctctaagagcagagcataactga

SEQ ID NO: 98 - protein, 580 aa, Vibrio splendidus
  V12B01_24254
VNKPIFVVVLASLTYGCGGSSSSDSSDPSDTNNSGASYGVVAPYDIAKYQNILSSSDLQVSDPNGEEGNKTSEVKDGNFDGYVSDYFYADEETENLIFKMANYKMRSEVREGENFDINEAGVRRSLHAEISLPDIEHVMASSPADHDEVTVLQIHNKGTDESGTGYIPHPLLRVVWEQERDGLTGHYWAVMKNNAIDCSSAADSSDCYATSYNRYDLGEADLDNFTKFDLSVYENTLSIKVNDEVKVDEDITYWQHLLSYFKAGIYNQFENGEATAHFQALRYTTTQVNGSNDWDINDWKLTIPASKDTWYGSGGDSAAELEPERCESSKDLLANDSDVYDSDIGLSYFNTDEGRVHFRADMGYGTSTENSSYIRSELRELYQSSVQPDCSTSDEDTSWYLDDTRTNATSHELTASLRIEDYPNINNQDPKVVLGQIHGWKINQALVKLLWEGESKPVRVILNSDFERNNQDCNHCDPFSVELGTYSASEEWRYTIRANQDGIYLATHDLDGTNTVSHLIPWGQDYTDKDGDTVSLTSDWTSTDIAFYFKAGIYPQFKPDSDYAGEVFDVSFSSLRAEHN

SEQ ID NO: 99 - DNA, 1569 bp, Vibrio spendidus
  V12B01_24259
atgaaacaaattactctaaaaactttactcgcttcttctattctacttgcggttggttgtgcgagcacgagcacgcctactgctgattttccaaataacaaagaaactggtgaagcgcttctgacgccagttgctgtttccgctagtagccatgatggtaacggacctgatcgtctcgttgaccaagacctaactacacgttggtcatctgcgggtgacggcgagtgggcaacgctagactatggttcagtacaggagtttgacgcggttcaggcatctttcagtaaaggtaatcagcgccaatctaaatttgatatccaagtgagtgttgatggcgaaagctggacaacggtactagaaaaccaactaagctcaggtaaagcgatcggcctagagcgtttccaatttgagccagtagtgcaagcacgctacgtaagatacgttggtcacggtaacaccaaaaacggttggaacagtgtgactggattagcggcggttaactgtagcattaacgcatgtcctgctagccatatcatcacttcagacgtggttgcagcagaagccgtgattattgctgaaatgaaagcggcagaaaaagcacgtaaagatgcgcgcaaagatctacgctctggtaacttcggtgtagcagcggtttacccttgtgagacgaccgttgaatgtgacactcgcagtgcacttccagttccgacaggcctgccagcgacaccagttgcaggtaactcgccaagcgaaaactttgacatgacgcattggtacctatctcaaccatttgaccatgacaaaaatggcaaacctgatgatgtgtctgagtggaaccttgcaaacggttaccaacaccctgaaatcttctacacagctgatgacggcggcctagtattcaaagcttacgtgaaaggtgtacgtacctctaaaaacactaagtacgcgcgtacagagcttcgtgaaatgatgcgtcgtggtgatcagtctattagcactaaaggtgttaataagaataactgggtattctcaagcgctcctgaatctgacttagagtcggcagcgggtattgacggcgttctagaagcgacgttgaaaatcgaccatgcaacaacgacgggtaatgcgaatgaagtaggtcgctttatcattggtcagattcacgatcaaaacgatgaaccaattcgtttgtactaccgtaaactgccaaaccaagaaacgggtgcggtttacttcgcacatgaaagccaagacgcaactaaagaggacttctaccctctagtgggcgacatgacggctgaagtgggtgacgatggtatcgcgcttggcgaagtgttcagctaccgtattgacgttaaaggcaacacgatgactgtaacgctaatacgtgaaggcaaagacgatgttgtacaagtggttgatatgagcaacagcggctacgacgcaggcggcaagtacatgtacttcaaagccggtgtttacaaccaaaacatcagcggcgacctagacgattactcacaagcgactttctatcagctagatgtatcgcacgatcaatacaaaaagtaa

SEQ ID NO: 100 - protein, 522 aa, Vibrio splendidus
  V12B01_24259
MKQITLKTLLASSILLAVGCASTSTPTADFPNNKETGEALLTPVAVSASSHDGNGPDRLVDQDLTTRWSSAGDGEWATLDYGSVQEFDAVQASFSKGNQRQSKFDIQVSVDGESWTTVLENQLSSGKAIGLERFQFEPVVQARYVRYVGHGNTKNGWNSVTGLAAVNCSINACPASHIITSDVVAAEAVIIAEMKAAEKARKDARKDLRSGNFGVAAVYPCETTVECDTRSALPVPTGLPATPVAGNSPSENFDMTHWYLSQPFDHDKNGKPDDVSEWNLANGYQHPEIFYTADDGGLVFKAYVKGVRTSKNTKYARTELREMMRRGDQSISTKGVNKNNWVFSSAPESDLESAAGIDGVLEATLKIDHATTTGNANEVGRFIIGQIHDQNDEPIRLYYRKLPNQETGAVYFAHESQDATKEDFYPLVGDMTAEVGDDGIALGEVFSYRIDVKGNTMTVTLIREGKDDVVQVVDMSNSGYDAGGKYMYFKAGVYNQNISGDLDDYSQATFYQLDVSHDQYKK

SEQ ID NO: 101 - DNA, 1230 bp, Vibrio splendidus
  V12B01_24264
atgcaaatttctaaagtcgctacagctgtcgctctttcgacaggtttattatttggttgtaacagtgatggtttacctattccaacagatccaggcggaacagaccctgttgaacctgttgaagtttactctatagaaaacgtctattgggatctgacaggtggtgctgttgctgcacagtcactcagcggaacttcaccatatcgctttgataataatgaggaaggtactcgtgctctaagcatttacagtggagacgtagctaatggcttcacttttgagagttcaatatatactgctgaagaagaaggtgttgtttcctttgaaggtaaggactgtacttacacagtgactgagcaacagctagatatgacctgtgaaaaagatgacgtagaaacagcttactcagcaacagagattacagatgaatctgttataactgcattagaaaatgccgatgatggaaaacctaaatcagtcgatgatgtgaacgctgcgattgcatcagcagaagatggcgcgattattgatttatcatctgaaggtacgtttgataccggtgttattgagctaaataaagctgtcacaattgatggtgctggtttagcaaccattaccggagatgcttgtattgatgtcactgcacccggtgcaggtatcaaaaacatgacttttgctaacgacaatttggccgggtgttttggtagggagtcagctggtacttcagataatgaaactggtgcgatcgttattggtaaaattggtaaagattcagatcctgtagcacttgaaaacctaaagttcgatgcaaacggcattaccgaagatgatctaggtactaaaaaagcaagttggttattctctcgaggttactttacattagacaatagcgaatttgtcggtttaagtggcagtttccaaaataatgcaattcgtattaactgtagtagtgacaacgggcgatttggttcacaaatcacaaataatacattcactattaactctggtggtagtgatgtgggcggaattaaagttggtgattctagcagtgccgtcataaagaatagtgatgataaccttggctgtaatgtcactattgaaagcaatacgttcaatggttacaaaaccctactttcagctgacaacggtaaagatataagaaatacagccatctacgcacaaccatctgcagtgaacactgcggcaggtaaagaaaatatcttgaactaa

SEQ ID NO: 102 - protein, 409 aa, Vibrio splendidus
  V12B01_24264
MQISKVATAVALSTGLLFGCNSDGLPIPTDPGGTDPVEPVEVYSIENVYWDLTGGAVAAQSLSGTSPYRFDNNEEGTRALSIYSGDVANGFTFESSIYTAEEEGVVSFEGKDCTYTVTEQQLDMTCEKDDVETAYSATEITDESVITALENADDGKPKSVDDVNAAIASAEDGAIIDLSSEGTFDTGVIELNKAVTIDGAGLATITGDACIDVTAPGAGIKNMTFANDNLAGCFGRESAGTSDNETGAIVIGKIGKDSDPVALENLKFDANGITEDDLGTKKASWLFSRGYFTLDNSEFVGLSGSFQNNAIRINCSSDNGRFGSQITNNTFTINSGGSDVGGIKVGDSSSAVIKNSDDNLGCNVTIESNTFNGYKTLLSADNGKDIRNTAIYAQPSAVNTAAGKENILN

SEQ ID NO: 103 - DNA, 1038 bp, Vibrio splendidus
  V12B01_24274
atgtttaagaaaaacatattagcagtggcgttattagcgactgtgccaatggttactttcgcaaataacggtgtttcttaccccgtacctgccgataaattcgatatgcataattggaaaataaccataccttcagatattaatgaagatggtcgcgttgatgaaatagaaggggtcgctatgatgagctactcacatagtgatttcttccatcttgataaagacggcaaccttgtatttgaagtgcagaaccaagcgattacgacgaaaaactcgaagaatgcgcgttctgagttacgccagatgccaagaggcgcagatttctctatcgatacggctgataaaggaaaccagtgggcactgtcgagtcacccagcggctagtgaatacagtgctgtgggcggaacattagaagcgacattaaaagtgaatcacgtctcagttaacgctaagttcccagaaaaatacccagctcattctgttgtggttggtcagattcatgctaaaaaacacaacgagctaatcaaagctggaaccggttatgggcatggtaatgaaccactaaagatcttctataagaagtttcctgaccaagaaatgggttcagtattctggaactatgaacgtaacctagagaaaaaagatcctaaccgtgccgatatcgcttatccagtgtggggtaacacgtgggaaaaccctgcagagccgggtgaagccggtattgctcttggtgaagagtttagctacaaagtggaagtgaaaggcaccatgatgtacctaacgtttgaaaccgagcgtcacgataccgttaagtatgaaatcgacctgagtaagggcatcgatgaacttgactcaccaacgggctatgctgaagatgatttttactacaaagcgggcgcatacggccaatgtagcgtgagcgattctcaccctgtatgggggcctggttgtggcggtactggcgatttcgctgtcgataaaaagaatggcgattacaacagtgtgactttctctgcgcttaagttaaacggtaaatag

SEQ ID NO: 104 - protein, 345 aa, Vibrio splendidus
  V12B01_24274
MFKKNILAVALLATVPMVTFANNGVSYPVPADKFDMHNWKITIPSDINEDGRVDEIEGVAMMSYSHSDFFHLDKDGNLVFEVQNQAITTKNSKNARSELRQMPRGADFSIDTADKGNQWALSSHPAASEYSAVGGTLEATLKVNHVSVNAKFPEKYPAHSVVVGQIHAKKHNELIKAGTGYGHGNEPLKIFYKKFPDQEMGSVFWNYERNLEKKDPNRADIAYPVWGNTWENPAEPGEAGIALGEEFSYKVEVKGTMMYLTFETERHDTVKYEIDLSKGIDELDSPTGYAEDDFYYKAGAYGQCSVSDSHPVWGPGCGGTGDFAVDKKNGDYNSVTFSALKLNGK

SEQ ID NO: 105 - DNA, 1779 bp, Vibrio splendidus
  V12B01_19706
atggaactcaatactctgatcgtaggaatttatttccttttccttatcgcaataggttggatgtttagaacgttcaccagtacaaccagtgactatttccgcgggggcggcaacatgctttggtggatggttggcgcaactgcgtttatgactcagttcagtgcttggaccttcactggcgcagcgggtaaagcgtatgccgatggtatggctgtcgcggttatcttcttagcgaacgcatttggttacttgatgaactacctgtacttcgctccgaaattccgtcaactacgcgtggtaacgccaattgacgcgattcgtatgcgttttggtagcttcaacgagcaagtgtttacttggtctggcatgccgaacagtattgtttctgcaggtatctggctgaacggtttggcgattatcgcatcgggcatatttggcttcgatatgacaactaccatcgttctaaccggtcttgttgtactagtgatgtcggtaacgggcggttcatgggcgtttatcgcgtctgacttcatgcagatggttatcatcatggcagtaaccgtaacgtgtgcagctgtggctatcgtgcaaggtggcggcgttactgaaatcatcaacgatttccctgtagcagaaggcgcatcattcgtttctggtaacaacctgaactacgtaagtatcttcggaatctgggcattattcatcttcttcaaacagttcagtatcaccaacaacatgttgaactcttaccgttacctagcggcaaaagactccaagaacgcgaagaaagcagcgctgttggcgtgtattctgatgacaattggtccaatcatttggttcatgccaccttggtttgtagcaggacaaggcgttgaccttgcagcacattacccagatgcaggttctaaagcaggcgacttcgcttacctttacttcgtagagcaatacatgccagcgggtatggtggggcttctaatcgcagcaatgttcgcagcaaccatgtcttctatggactcaggcttaaaccgtaactcaggtatctttgtaatcaacttctaccaaccgattcttcgtccaaacgcgacagagaaagagttgatggttgtgtctaagttgatgtcttcggttttcggtgtcgctatcatccttatcgcactgtttattaactcactgaaaggcttgagcttgttcgacaccatgatgtacgtaggtgcattaatcggtttcccaatgacgattccagcattctgtggcttcttcattaagaagactccggactgggcaggttggggcacgttagtagttggcgcgattgtgtcttacatcgtaggcttcgtgattatcgctgaaatgctacaaaattggttcaacctagagccgctaacaggtcgtgaatggtctgatttgaaagtagcggtaggtctgattgctcacttaacgtttacaggcggcttctttattctttcgactttgttctataaaccactggaagcgtctcgtcagaaagatgttgataccttcttcactaacctatcgacgcctttggtttctgaatcaaccgcgcagaagaaactggataacaaacaacgtcacatgctgggttcattgatcgcagtgtctggtgtagctgtgatggcgatgtttgctctaccaaacccattctggggaagaatgatgttcgtactgtgtggcggtatcgtgtttatcgttggtctactgttagtgaaagctgtagacgactcggttgaggatgcgaaacaagcgaaaaaaacagcttaa

SEQ ID NO: 106 - protein, 592 aa, Vibrio splendidus
  V12B01_19706
MELNTLIVGIYFLFLIAIGWMFRTFTSTTSDYFRGGGNMLWWMVGATAFMTQFSAWTFTGAAGKAYADGMAVAVIFLANAFGYLMNYLYFAPKFRQLRVVTPIDAIRMRFGSFNEQVFTWSGMPNSIVSAGIWLNGLAIIASGIFGFDMTTTIVLTGLVVLVMSVTGGSWAFIASDFMQMVIIMAVTVTCAAVAIVQGGGVTEIINDFPVAEGASFVSGNNLNYVSIFGIWALFIFFKQFSITNNMLNSYRYLAAKDSKNAKKAALLACILMTIGPIIWFMPPWFVAGQGVDLAAHYPDAGSKAGDFAYLYFVEQYMPAGMVGLLIAAMFAATMSSMDSGLNRNSGIFVINFYQPILRPNATEKELMVVSKLMSSVFGVAIILIALFINSLKGLSLFDTMMYVGALIGFPMTIPAFCGFFIKKTPDWAGWGTLVVGAIVSYIVGFVIIAEMLQNWFNLEPLTGREWSDLKVAVGLIAHLTFTGGFFILSTLFYKPLEASRQKDVDTFFTNLSTPLVSESTAQKKLDNKQRHMLGSLIAVSGVAVMAMFALPNPFWGRMMFVLCGGIVFIVGLLLVKAVDDSVEDAKQAKKTA

SEQ ID NO: 107 - DNA, 537 bp, Agrobacterium tumetaciens
  Atu_3019
atgagaaacgcagagcgagcaggtccgcttcggtatcttgaggtatcggccatcggcagtttttgctctgcatttttttatttcggggtggccatggcaatgccggcggacatgacggcggagcgccttctcaatatctgtgaagcgcccaccatgcaggccgcgatgatcaagggtgacgaacttggctggccgcggctgaccgccgcggaaacggaggaatggcgtcgtagtttcgtcgcatataatgagggttcggtggtggtcgtgggctggcggggcgagaacgccggcagagccgagtcattgtctttttgggttgcgactggtccaaacggacacaaggcatgcgcctattccacggcaaggcctgccggttttctggatgccttgtcggagcggcttggtgcaccggataatctcgacaaaaatgacgcgatagaaagtacgacagcctggtggaaacgaggtgcggtcgagtattcttttgtccagatcggctcatccgctgtcgtcaatatccgttcaagtcagtga

SEQ ID NO: 108 - protein, 178 aa, Agrobacterium tumetaciens
  Atu_3019
MRNAERAGPLRYLEVSAIGSFCSAFFYFGVAMAMPADMTAERLLNICEAPTMQAAMIKGDELGWPRLTAAETEEWRRSFVAYNEGSVVVVGWRGENAGRAESLSFWVATGPNGHKACAYSTARPAGFLDALSERLGAPDNLDKNDAIESTTAWWKRGAVEYSFVQIGSSAVVNIRSSQ

SEQ ID NO: 109 - DNA, 711 bp, Agrobacterium tumetaciens
  Atu_3020
atgaccaatcggatccatgcgctgctccgcaagctcattgtggaagtcaaactacttcctgggcgcgcgctgtcggaaaaagagatcgccgcacttttgaatgtcagcaaaacgcctgtacgcgaagcgatcatccgcctgtcggaagagggcttcgtcaccgttgttccacagggcggtacatacatctcaccgatcgatgtgcagcggtacatggaagcctgcttcatacggttcaaactggaagagggcgcggtcatagaggctacgaagcgccattcgcttgaagacatcgcgcgcctcaagacctgcctctcaaatcagcgcattgctgcaaaagacgaagaattcacgaatttcttcctgcttgacgaggaattccacaagaccatctttgcagccgcgcgccttccgggcgcctggagcgtggtcaaccaggccaagggcgaaatggaccgtatgcggcatctgaaacgcgtatttgcggtcagacgcacagaaaaggttatcgaggagcacgaggcaatcgtggatggaatcgagtcaggcgacgtcgaagcggcacgcgaagccgtccagcgccatctcgggtcactcgaaaccaaaatcgcagaactctccaagaacccgaagatctggaccttcatcgagcaggtcaacacgcgcgtcacgaggaagcgcgcatcgcgggggtcgaagacacctgactga

SEQ ID NO: 110 - protein, 236 aa, Agrobacterium tumetaciens
  Atu_3020
MTNRIHALLRKLIVEVKLLPGRALSEKEIAALLNVSKTPVREAIIRLSEEGFVTVVPQGGTYISPIDVQRYMEACFIRFKLEEGAVIEATKRHSLEDIARLKTCLSNQRIAAKDEEFTNFFLLDEEFHKTIFAAARLPGAWSVVNQAKGEMDRMRHLKRVFAVRRTEKVIEEHEAIVDGIESGDVEAAREAVQRHLGSLETKIAELSKNPKIWTFIEQVNTRVTRKRASRGSKTPD

SEQ ID NO: 111 - DNA, 1116 bp, Agrobacterium tumetaciens
  Atu_3021
atgtccgcagttcaaatcaacgacgtcgtcaagcgctatggcgccctagaagtcgtccacggtatcaatctttccatacagccgcaggaattcgttgtcctcgttggtccttcgggatgcggaaaatccaccacgctgcgtatgctcgccggactggaggacatctcggacggcaccgtttcgatggacggccgggtggtcaacaaggtcgcgccgaaagaccgcgatgtcgccatggtattccagaactatgcgctctacccccatctgagcgtggccgagaacatcgcgttcggcctgcgcgtcaggggcgagaaacgggccgtcgtcgacaaggccgttgcggaagccgcacagatattgggacttaccgaatatctggcccgcaagccggccgatctttccggtggccagcgccagcgcgtcgccatgggccgcgccatcgtgcgccaaccgaaaatcttcctgttcgacgaacccctctccaatcttgacgccaagctgcgcacccatatgcgcgccgagatcaagctgctgcacaaacggatgcaggccaccagcatctacgtgacccacgaccaggtggaggcgatgacgcttgccgaccgcatcgtcatcatgaatggcggccatatcgaacaggtcggctcgccaatggaggttttcctcgaaccggcgaacacatttgtcgcaagcttcatcggctcgccaccaatgaacctactcgacggaaccatcgaaaagcaggatggcaccgtgaaggtcaggctcacgggcggctccgggcagcgcttcagcatccccgatgccttcgcgaagaacgcaatcgaaggacagaccgtcaagctcggcctgcggccggaaatcatgtcggtggaagatagcgatggccgcgaggttctccattgcagcattgaccttgtggagccgctgggcgctgaagcactgctgcatggaaaggcggatggccagccatttatcgccaaagccgaaacgctttatggcgatcatgcccttaacggcgtcaaccggctttccatcgataccgcccgcgtccacgtctttgacgcggcgaccggaagatcgctcaaaatccgggatggagcgcagccatga

SEQ ID NO: 112 - protein, 371 aa, Agrobacterium tumetaciens
  Atu_3021
MSAVQINDVVKRYGALEVVHGINLSIQPQEFVVLVGPSGCGKSTTLRMLAGLEDISDGTVSMDGRVVNKVAPKDRDVAMVFQNYALYPHLSVAENIAFGLRVRGEKRAVVDKAVAEAAQILGLTEYLARKPADLSGGQRQRVAMGRAIVRQPKIFLFDEPLSNLDAKLRTHMRAEIKLLHKRMQATSIYVTHDQVEAMTLADRIVIMNGGHIEQVGSPMEVFLEPANTFVASFIGSPPMNLLDGTIEKQDGTVKVRLTGGSGQRFSIPDAFAKNAIEGQTVKLGLRPEIMSVEDSDGREVLHCSIDLVEPLGAEALLHGKADGQPFIAKAETLYGDHALNGVNRLSIDTARVHVFDAATGRSLKIRDGAQP

SEQ ID NO: 113 - DNA, 930 bp, Agrobacterium tumetaciens
  Atu_3022
atgaaacaggattttcaagggcgccttcacgaccttcaggaagacgtgcgccgtgactggcaactctacctgctgctggcgccaatgctcatctggtttgcggttttcctctacaaacccatgtacggattggtcatcgcattccaggatttctcgatcttccgcggcatcgaaaagagcccctgggtcggttttgcgaatttcgtggagctcttccgcaacgacatgttcgtgcgttccttctggaacaccatcaccatcagcggcctcggcctgatcttcgcctttccggtgccaatcattctggccctgatgtttaatgaggttcagaacggcactgcccggagctgggcgcagaccgtcgtttacctgccgcatttcatctctgtggtgatcgtcgccggcattgtcatcaatttcctctcgccctcgataggcatcgtcaatctcatgctgaaaggccttggatttgagccgatctatttcctgacccagccggaatggttccgtcccgtctatatcggctcatcggtctggaaggaggcaggcttcgaatccatcgtctatctcgccgccatcgccggcgtgagcccgacgctttacgaatccgcccgggtggatggcgcatcgcgctggcagatgatgtggcgtatcaccctgccctgcatcctgccgacgatcgtgatcatgctgatcatccgcatcggcaatctggtcgaagtcgggttcgaatatatcatcctgctctatcgcccatccacctacgagacggcggacgtcgtttccaccttcatttaccgcaccggcctgcagggcacccaatacgatcttgcgactgccgccggcctgttcaacgcggtcatcgccttcgtcctcgtctattcggcaaatcgcatcagccgaaaagtctcgtcgacatcgctgtggtga

SEQ ID NO: 114 - protein, 309 aa, Agrobacterium tumetaciens
  Atu_3022
MKQDFQGRLHDLQEDVRRDWQLYLLLAPMLIWFAVFLYKPMYGLVIAFQDFSIFRGIEKSPWVGFANFVELFRNDMFVRSFWNTITISGLGLIFAFPVPIILALMFNEVQNGTARSWAQTVVYLPHFISVVIVAGIVINFLSPSIGIVNLMLKGLGFEPIYFLTQPEWFRPVYIGSSVWKEAGFESIVYLAAIAGVSPTLYESARVDGASRWQMMWRITLPCILPTIVIMLIIRIGNLVEVGFEYIILLYRPSTYETADVVSTFIYRTGLQGTQYDLATAAGLFNAVIAFVLVYSANRISRKVSSTSLW

SEQ ID NO: 115 - DNA, 882 bp, Agrobacterium tumetaciens
  Atu_3023
atggcaaacttcaatctctattcccgtggcgacaggattttcggctgggcaaacgccattcttctcggcctgttcgtcctgtcgacactctatcctttcatctatgtgctgtcggtctcgctctcctcgggcgctgccgtcaccgccggccgcgtcaccctctggcccgtcgatgtcacgcttgccgcctacgatcgggtcttgtccgacaggatgttctgggtcgcctatggcaacaccttcatctataccttcggcggcacgctcatgagcctgctgatcatgatacccggcgcctatgccctgtcgcgcaaacgcctgcgtggccgcacgttcttcaacatcgccatcgcgttcacgctctggttcaacgccggcatgatcccgttcttcctgaatatgcgcgatctcgggcttctcgacagccgtttcggcatcatcctcggtttcgcggccaatgccttcaacgtcatcctgctgcggaattttttcgaagcggtgccgaaatccttcgaagaggccgcgaagatggatggcgccagcgaattgcagctgctttggaaggtgttcattccgctgtcgaagcccgcaatcgtcacggtcgcgctgttctgcatcgtatcgcgctggaacggttatttctgggccatggtgctgctgcgcggcgaggagaaaattccgcttcaggtctatctgaagcgcgtcatcgtcgatctcaattccaatgacgaattcgcctcgagcctgctgaccgcccactattcgttcgagacggtgacatcggcgatcatgatcgcttccattattcccgtcctcatcatttacccctacgcgcagaagtatttcaccaaaggaattctgctgggcggagtcaaagagtag

SEQ ID NO: 116 - protein, 293 aa, Agrobacterium tumetaciens
  Atu_3023
MANFNLYSRGDRIFGWANAILLGLFVLSTLYPFIYVLSVSLSSGAAVTAGRVTLWPVDVTLAAYDRVLSDRMFWVAYGNTFIYTFGGTLMSLLIMIPGAYALSRKRLRGRTFFNIAIAFTLWFNAGMIPFFLNMRDLGLLDSRFGIILGFAANAFNVILLRNFFEAVPKSFEEAAKMDGASELQLLWKVFIPLSKPAIVTVALFCIVSRWNGYFWAMVLLRGEEKIPLQVYLKRVIVDLNSNDEFASSLLTAHYSFETVTSAIMIASIIPVLIIYPYAQKYFTKGILLGGVKE

SEQ ID NO: 117 - DNA, 1530 bp, Agobacterium tumefaciens
  Atu_3024
atgacagccgcaaccctggctttgccggtatcggcgcaggattcaggcaagatcaccgaagacccgctggaactgactatccattttcatttccgcgacaaatacgtctattcggaaaaatggccggtggagcagaaggcagcggaaatcaccaatatccatgtccgcaatgttgcatccctggcaaccaccagcagccgtgatgccttcaatcttctgatcgcctcaggcaaccttcccgacatcgttggcggtgacgccaacggtggtttgaaggaggatttcatccgctacgggctggagggtgcgttcattccgctcgatgagctgatcgaggaaaacgcccccaaccttaaggccttcttcgacagccacaaggaaatccgcaatgcgatttccggaccggacggcaagctttatttcatcccctatgtgcctgacggcaagttctcgcgcggctggttcatccgccaggactggctcgacaagctcggcctgaagcagccggagacaccggaagaggtctacgaagttctgaaagccttccgcgacaaggacccgaacggcaatggtctcaaggatgaagtgccttattttgcccgtgaggaaatcgatgccgtccgcctcatcaacctgtgggacgctcgtgtgtcgggatccgaacgttatggcgactttgccgtttacgacgggcagctgcgtcacccgtgggcagaggaaaactacaagacgggcatttccaatgtcgccaagtggtacaaggaaggcctgatcgacaaggagatattcacccgcggcgcccgttcgcgcgaatatctcctgggcaacaatctcggcgggatgacgcatgactggttcgccagcacgtcgacctacaatgacgccctgtcgcccaagattgaaggctttgcgctgaagccgatgcttccgcccaagaccgtctcgggccaacgcttcgaggattcgcggcgcctgcgtgtccagcccgtcggctgggcaatttcggcgaccaatgaacacccggtcgaaaccatcaaatatttcgacttctggttctcacagcagggccgcatcctgtcgaatttcggcgttgagggtctgacctacgaaatggtcgatggcaaaccgcagttcaagaaggaaatcctcgacaacaaggatccggtcaatgcccagctctgggcggtcggtgcggcgatcccgcgcggatactggatggattacgaatatgagcgccagtggacgaacaagatcgcgcttgagggcatcgacctctacgagaagtgcaactgcctcaaggacgagttcatgggcgtttccctgaacgccgaggaaaaagcgaccttcgaccgctactggcctagcatcctcacctacatgaccgagatgcagcagacctggattctcggcgcgcaggacgtcgataccggctgggacgcctacaaaaagcgcctcactgctcttggttacgacgaggtcatcaagatgatgcagtcggcctatgaccgccagtacaaggcggcgcaataa

SEQ ID NO: 118 - protein, 509 aa, Agobacterium tumefaciens
  Atu_3024
MTAATLALPVSAQDSGKITEDPLELTIHFHFRDKYVYSEKWPVEQKAAEITNIHVRNVASLATTSSRDAFNLLIASGNLPDIVGGDANGGLKEDFIRYGLEGAFIPLDELIEENAPNLKAFFDSHKEIRNAISGPDGKLYFIPYVPDGKFSRGWFIRQDWLDKLGLKQPETPEEVYEVLKAFRDKDPNGNGLKDEVPYFAREEIDAVRLINLWDARVSGSERYGDFAVYDGQLRHPWAEENYKTGISNVAKWYKEGLIDKEIFTRGARSREYLLGNNLGGMTHDWFASTSTYNDALSPKIEGFALKPMLPPKTVSGQRFEDSRRLRVQPVGWAISATNEHPVETIKYFDFWFSQQGRILSNFGVEGLTYEMVDGKPQFKKEILDNKDPVNAQLWAVGAAIPRGYWMDYEYERQWTNKIALEGIDLYEKCNCLKDEFMGVSLNAEEKATFDRYWPSILTYMTEMQQTWILGAQDVDTGWDAYKKRLTALGYDEVIKMMQSAYDRQYKAAQ

SEQ ID NO: 119 - DNA, 2331 bp, Agobacterium tumefaciens
  Atu_3025
atgcgtccctctgccccggccatctccagacagacacttctcgatgaaccccgcccgggctcattgaccattggctacgagccgagcgaagaagcacaaccgacggagaaccctccgcgcttttcatggctacccgatattgacgacggcgcgcgttacgtgctgcgcatttcgaccgatcccggttttacagacaaaaaaacgctcgtcttcgaggatctcgcctggaatttcttcaccccggatgaagcactgccggacggccattatcactggtgttatgcgctatgggatcagaaatccgcaacagcgcattccaactggagcaccgtacgcagtttcgagatcagtgaagcactgccgaaaacgccgctgcccggcaggtctgcccgccatgctgccgcgcaaaccagccaccctcggctgtggctcaactccgagcaattgagtgccttcgccgatgccgttgcgaaggaccccaaccattgtggctgggccgagttttacgaaaaatcggtcgagccgtggctcgagcggccggtcatgccggaaccgcagccctatcccaacaacacgcgtgtcgccacgctctggcggcagatgtatatagactgccaggaagtgatctatgcgatccggcacctggccattgccggccgcgtgctcggacgcgacgaccttctcgatgcatcccgcaaatggctgctggccgtcgccgcctgggacacgaaaggtgcgacctcacgcgcctataatgacgaggcggggttccgcgtcgtcgtcgcactcgcctggggttatgactggctgtacgaccatctgagcgaagacgaacgcaggaccgtgcgatccgttcttctcgaacggacgcgggaagttgccgatcatgtcatcgcacacgcccgcattcacgtctttccctatgacagccatgcggtgcgctcgctttcggctgtattgacgccggcctgcatcgcacttcagggagaaagcgacgaggctggcgaatggctcgactataccgtcgaattccttgccacgctctattctccctgggcgggaaccgatggtggttgggcggaaggtccgcattactggatgaccggcatggcctatctcatcgaggccgccaatctgatccgctcctatattggttatgacctctatcaacggccgtttttccagaataccggtcgcttcccgctttacaccaaggcgccgggaacccgccgcgccaacttcggcgacgactccacccttggcgaccttcccggcctgaagctgggatacaacgtccggcaattcgccggcgtcaccggcaatggccattaccagtggtatttcgatcacatcaaggccgatgcgacaggcacggaaatggccttttacaattacggctggtgggacctcaacttcgacgatctcgtctatcgccacgattacccgcaggtggaagccgtgtctcccgccgacctgccggcactcgccgttttcgatgatattggttgggcgaccatccaaaaagacatggaagacccggaccggcacctgcagttcgtcttcaaatccagcccttacggttcgctcagccacagtcacggcgaccagaatgcctttgtgctttatgcccatggcgaggatctggcgatccagtccggttattacgtggcgttcaattcgcagatgcatctgaattggcggcgtcagacacggtcgaaaaatgccgtgctgatcggcggcaaaggccaatatgcggaaaaggacaaggcgcttgcacgccgcgccgccggccgcatcgtctcggtggaggaacagcccggccatgttcgtatcgtcggcgatgcaaccgccgcctaccaggttgcgaacccgctggttcaaaaggtgctgcgcgaaacccacttcgttaatgacagctatttcgtgattgtcgacgaagtcgaatgttcggaaccccaggaactgcaatggctttgccatacactcggagcgccgcagaccggcaggtcaagcttccgctacaatggccggaaagccggtttctacggacagttcgtttactcttcgggcggcacgccgcaaatcagcgccgtggagggttttcccgatatcgacccgaaagaattcgaagggctcgacatacaccaccatgtctgcgccacggttccggccgccacccggcatcgccttgtcacccttctggtgccttacagcctgaaggagccgaagcgcattttcagcttcatcgatgatcagggtttttccaccgacatctacttcagtgatgtcgatgacgagcgtttcaagctctcccttcccaagcagttctaa

SEQ ID NO: 120 - protein, 776 aa, Agobacterium tumefaciens
  Atu_3025
MRPSAPAISRQTLLDEPRPGSLTIGYEPSEEAQPTENPPRFSWLPDIDDGARYVLRISTDPGFTDKKTLVFEDLAWNFFTPDEALPDGHYHWCYALWDQKSATAHSNWSTVRSFEISEALPKTPLPGRSARHAAAQTSHPRLWLNSEQLSAFADAVAKDPNHCGWAEFYEKSVEPWLERPVMPEPQPYPNNTRVATLWRQMYIDCQEVIYAIRHLAIAGRVLGRDDLLDASRKWLLAVAAWDTKGATSRAYNDEAGFRVVVALAWGYDWLYDHLSEDERRTVRSVLLERTREVADHVIAHARIHVFPYDSHAVRSLSAVLTPACIALQGESDEAGEWLDYTVEFLATLYSPWAGTDGGWAEGPHYWMTGMAYLIEAANLIRSYIGYDLYQRPFFQNTGRFPLYTKAPGTRRANFGDDSTLGDLPGLKLGYNVRQFAGVTGNGHYQWYFDHIKADATGTEMAFYNYGWWDLNFDDLVYRHDYPQVEAVSPADLPALAVFDDIGWATIQKDMEDPDRHLQFVFKSSPYGSLSHSHGDQNAFVLYAHGEDLAIQSGYYVAFNSQMHLNWRRQTRSKNAVLIGGKGQYAEKDKALARRAAGRIVSVEEQPGHVRIVGDATAAYQVANPLVQKVLRETHFVNDSYFVIVDEVECSEPQELQWLCHTLGAPQTGRSSFRYNGRKAGFYGQFVYSSGGTPQISAVEGFPDIDPKEFEGLDIHHHVCATVPAATRHRLVTLLVPYSLKEPKRIFSFIDDQGFSTDIYFSDVDDERFKLSLPKQF

SEQ ID NO: 121 - DNA, 750 bp, Agobacterium tumefaciens
  Atu_3026
atgcagcgttttaccaacagaaccatcgttgtcgccggggccggccgggatatcggccgggcatgcgccatccgtttcgcacaggaaggcgccaatgtcgttcttacctataatggcgcggcagagggcgcggccacagccgttgccgaaatcgaaaagcttggtcgttcggctctggcgatcaaggcggatctcacaaacgccgccgaagtcgaggctgccatatctgcggctgcggacaagtttggggagatccacggcctcgtccatgttgccggcggcctgatcgcccgcaagacaatcgcagaaatggatgaagccttctggcatcaggtcctcgacgtcaatctgacatcgctgttcctgacggccaagaccgcattgccgaagatggccaagggcggcgcgatcgtcactttctcgtcgcaggccggccgtgatggcggcggcccgggcgctcttgcctatgccacttccaagggtgccgtgatgaccttcacccgcggacttgccaaagaagtcggccccaaaatccgcgtcaacgccgtttgccccggtatgatctccaccaccttccacgataccttcaccaagccggaggtgcgcgaacgggtggccggcgcgacgtcgctcaagcgcgaagggtcgagcgaagacgtcgccggtctggtggccttcctcgcgtctgacgatgccgcttatgtcaccggcgcctgctacgacatcaatggcggcgtcctgttttcctga

SEQ ID NO: 122 - protein, 249 aa, Agobacterium tumefaciens
  Atu_3026
MQRFTNRTIVVAGAGRDIGRACAIRFAQEGANVVLTYNGAAEGAATAVAEIEKLGRSALAIKADLTNAAEVEAAISAAADKFGEIHGLVHVAGGLIARKTIAEMDEAFWHQVLDVNLTSLFLTAKTALPKMAKGGAIVTFSSQAGRDGGGPGALAYATSKGAVMTFTRGLAKEVGPKIRVNAVCPGMISTTFHDTFTKPEVRERVAGATSLKREGSSEDVAGLVAFLASDDAAYVTGACYDINGGVLFS

SEQ ID NO: 123 - DNA, 1386 bp, Saccharophagus degradans
  Sde_3603
atgaaaacctttaacccagatttcgtatggggagcagccagttccgcctatcaggtagaaggcgccaccaccaccgatggcagaggccccagtatttgggatgcgttcagttccattcccggtaaaacctaccacaaccaaaacgccgacatagcctgcgaccactacaaccgctggcaagaagacgtggccataatgaaagagatggggctaaaggcttaccgcttttctatttcttggtcgcgcatattccctactgggcgcggcgaagttaacgaaaaaggcgtagccttttacaacaaccttatcgacgaattaataaaaaacgacattaccccttgggtaaccctatttcactgggactttcctctggcactgcaaatggaaatggacggcctacttaaccccgccatcgccgacgaattcgccaactacgccaagctgtgtttcgcgcgctttggcgaccgcgttacccactggattaccctaaacgaaccttggtgcagtgccatgcttggccacggcatgggcagcaaagcccctggccgcgtatctaaggatgaaccctatatagccgcccacaacttgctgcgtgcacacggcaaaatggtagatatttaccggcgcgaatttcagcccacacaaaaaggcatgataggcatagccaacaattgcgactggcgcgaacccaaaaccgattctgaattagataaaaaagcagccgagcgcgccctagaattttttgtaagctggtttgccgaccccatttatttgggcgactacccagccagcatgcgcgagcgcttgggtgagcgtttacccacctttagcgacgaagacattgcgctaataaaaaactctagcgacttttttggtttgaatcactacaccaccatgcttgccgaacaaacccacgaaggtgacgttgttgaagatactattcgcggcaacggcggcatatcggaagaccaaatggtcaccctctccaaagacccaagctgggaacaaaccgacatggagtggagcattgtgccctggggctgtaaaaaattattaatctggttaagcgagcgctacaactaccccgacatttacattaccgaaaacggctgcgccctacccgacgaagacgacgtaaacatagccattaacgatacacgccgcgtagatttttaccgcggttatatcgatgcgtgtcaccaagcaatagaggccggcgtaaaactaaaaggctattttgcatggacacttatggataactacgaatgggaagaaggctacaccaaacgctttggcttaaaccatgtagatttcaccacaggcaaacgcacacctaaacagtctgcaatttggtatagcacgttaattaaagatggtgggttctag

SEQ ID NO: 124 - protein, 461 aa, Saccharophagus degradans
  Sde_3603
MKTFNPDFVWGAASSAYQVEGATTTDGRGPSIWDAFSSIPGKTYHNQNADIACDHYNRWQEDVAIMKEMGLKAYRFSISWSRIFPTGRGEVNEKGVAFYNNLIDELIKNDITPWVTLFHWDFPLALQMEMDGLLNPAIADEFANYAKLCFARFGDRVTHWITLNEPWCSAMLGHGMGSKAPGRVSKDEPYIAAHNLLRAHGKMVDIYRREFQPTQKGMIGIANNCDWREPKTDSELDKKAAERALEFFVSWFADPIYLGDYPASMRERLGERLPTFSDEDIALIKNSSDFFGLNHYTTMLAEQTHEGDVVEDTIRGNGGISEDQMVTLSKDPSWEQTDMEWSIVPWGCKKLLIWLSERYNYPDIYITENGCALPDEDDVNIAINDTRRVDFYRGYIDACHQAIEAGVKLKGYFAWTLMDNYEWEEGYTKRFGLNHVDFTTGKRTPKQSAIWYSTLIKDGGF

SEQ ID NO: 125 - DNA, 1335 bp, Saccharophagus degradans
  Sde_1394
atgaatagacttacactaccgccttcttctcgtttgcgcagcaaagagtttacctttggtgttgcaacgtcgtcttaccaaattgaaggcggcatagattctcgcctgccctgtaattgggatacgttctgtgagcagcccaataccattattgataacaccaacggcgccattgcttgcgaccacataaatagatggcaagacgatatagaacttattgccaacctaggggtagatgcctaccgcttttctattgcgtggggccgtgttattaatttagacggcagcctcaataatgaaggcgttacattttacaaaaatattttaactaagcttcgcgaaaagaatttaaaagcttatataacgctataccactgggacttgccacaacatttagaagatgctggcggctggcttaaccgcgataccgcctacaagtttcgcgactatgtaaaccttataacccaagcgcttgatgacgatgtattttgctacacaacgttaaacgagcccttttgcagtgcctaccttggctatgaaattggtgtacacgcaccgggtataaaagacttagccagtgggcgcaaagccgcacaccatttattacttgcccatggcttagctatgcaagtgctgcgaaaaaactgccccaatagtttaagcggcatagtgttaaacatgagcccttgttacgccggcagcaacgcacaagcagatatagatgcagcaaaacgcgcggacgatttattatttcagtggtatgcacaaccgctacttactggctgctaccctgatgcaataaacagcctgccagacaatgccaaaccacctatttgtgaaggcgacatggcgttaataagccaacctttagattatttaggccttaactactatacccgcgcagtattttttgccgacggtaatggcggttttaccgaacaagtacctgagggtgtagagctaaccgatatgggctgggaagtttacccgcaaggcttaaccgatttactaatagacctaaaccaacgctataccctacccccgttacttattaccgaaaacggcgcagcaatggtggacgaacttgttaacggcgaagttaacgatattgcccgaataaattattttcaaacccatttacaagcggtacacaacgccattgaacaaggtgttgatgtacgcggttattttgcttggagcctaatggataattttgagtgggcactgggttacagcaaacgattcggtattacctatgtagattaccaaacacaaaagcgaacgctaaaagccagcggccacgcatttgctgagtttgtctcgagtaggagctaa

SEQ ID NO: 126 - protein, 444 aa, Saccharophagus degradans
  Sde_1394
MNRLTLPPSSRLRSKEFTFGVATSSYQIEGGIDSRLPCNWDTFCEQPNTIIDNTNGAIACDHINRWQDDIELIANLGVDAYRFSIAWGRVINLDGSLNNEGVTFYKNILTKLREKNLKAYITLYHWDLPQHLEDAGGWLNRDTAYKFRDYVNLITQALDDDVFCYTTLNEPFCSAYLGYEIGVHAPGIKDLASGRKAAHHLLLAHGLAMQVLRKNCPNSLSGIVLNMSPCYAGSNAQADIDAAKRADDLLFQWYAQPLLTGCYPDAINSLPDNAKPPICEGDMALISQPLDYLGLNYYTRAVFFADGNGGFTEQVPEGVELTDMGWEVYPQGLTDLLIDLNQRYTLPPLLITENGAAMVDELVNGEVNDIARINYFQTHLQAVHNAIEQGVDVRGYFAWSLMDNFEWALGYSKRFGITYVDYQTQKRTLKASGHAFAEFVSSRS

SEQ ID NO: 127 - DNA, 1332 bp, Saccharophagus degradans
  Sde_1395
atgttgtcagtaaaagaaaaagtagcatacggcttgggcgatacagccagtaacatagtgtttcaaaccgttatgctgtttcttgccttcttctataccgatatttttggcatttcgccggcggtggtaggcaccatgtttatagtggtgcgggtattagatgcaataaccgacccgttaatggggggcttggcagacagaacaaatactaaatggggtaagtttaggccctatttactttggttggccgtaccgttcggtttgataagtgttttggcatttactactcccgaattaggtgaagatggcaaggtgtattatgcctatgctacctacgcactattaatgatggcgtatacggctataaacattccttattccgcattgggtggggtgctaactgcagacccaaaacaacgtgtttcggttcaatcttaccgctttgtatttggcatgctgggcggcctaattgttactgcagcaacgctgcctcttgtgcagttttttggtaagggcgataaggcgcttggctatcagctcaccattgcggctatgagtgcactgggggtaatactatttttattgtgttttgctggtaccaaagagcgtattgcgccgccgccccagcagaaaacgagcttgtttaaagacttatccctaatgtgggtaaatgaccaatggcgagtgctatgcgtagcagcgttctttttgcttattggtatggtaatgcgttccactttggcgatttactacgttaagtactatttattacgcgaagacctggtaacagcgtttgttacattgggcatgataggcaacatagtgggttgcgcgttagcccagccgctaagtaagcgtgtatgcaaggttaaggcttatatagcgctgcaaattatcgcggcagttttgtgcgctgcggcatatttcgtggggcaagcgcaggtagttgctgcttttgtgctgtatgttttatggtgcttttttctacagatggccacccctttactgtgggcaaaaatggccgataccgtcgattatggtcactggaagaccggtattcgcataacgggtatggtatattcctccgtcgtttttttcatcaagcttggcctagcgttaggcggggcagtagctagctggctattggcctattacggctaccaagcagataccgcccaaacgccagatacattgcacggtattttgctgtcatttaccgtattcccagcggtgttttctctgcttgtggcttgggctatgcgttggtatatcctaaacaacgatgaggtggcgcgtattcagcaagcgctaaatttaaaaactgtaaactaa

SEQ ID NO: 128 - protein, 443 aa, Saccharophagus degradans
  Sde_1395
MLSVKEKVAYGLGDTASNIVFQTVMLFLAFFYTDIFGISPAVVGTMFIVVRVLDAITDPLMGGLADRTNTKWGKFRPYLLWLAVPFGLISVLAFTTPELGEDGKVYYAYATYALLMMAYTAINIPYSALGGVLTADPKQRVSVQSYRFVFGMLGGLIVTAATLPLVQFFGKGDKALGYQLTIAAMSALGVILFLLCFAGTKERIAPPPQQKTSLFKDLSLMWVNDQWRVLCVAAFFLLIGMVMRSTLAIYYVKYYLLREDLVTAFVTLGMIGNIVGCALAQPLSKRVCKVKAYIALQIIAAVLCAAAYFVGQAQVVAAFVLYVLWCFFLQMATPLLWAKMADTVDYGHWKTGIRITGMVYSSVVFFIKLGLALGGAVASWLLAYYGYQADTAQTPDTLHGILLSFTVFPAVFSLLVAWAMRWYILNNDEVARIQQALNLKTVN

SEQ ID NO: 129 - DNA, 2601 bp, Saccharophagus degradans
  Sde_2674
atgctgctaagcttaaaaaacactcaactcaaaagaagtatgaacatgaaccttaaacacctctttctggttgctttggcgctaaatattgctgcgtgcaatgtaaaagagcccgcggcgacaaatgataaccacattagctaccaagccgctcgcgaagcgcgcttggcaaaagttgaagccgaagttgaacgcctgctgccactattaacactagaagaaaaagcctctttggttcatgcgaacagcaaattctctatcgcctctatcgagcggctaggcattcacgaaatgtggatgtctgatggcccccacggcgtgcgctatcaaatcgaacgccacggctgggcaccagcaggctggacagatgacaactccacttacttaccaccgcttactaccgtagccgccagctggaaccccgaaatagctgcccttcacggcgatgtactcggcgcagaagctcgccaccgccgtaaagatgtaatattaggcccaggcgtaaacttagctcgcctgccactttatggtcgtaactttgaatatatgggtgaagaccccttcttggcatcacgtcttgctgtggcagaaattaaagccattcaagaaaatgacgtggccgcctgtatcaaacatttcgcgcttaacaatcaagagctgaatcgcaccggcgtaaacgccaaacccgatgaacgcacattacgcgaagtgtatttacccgccttcgaagccgccgttaaagaagcgggcgtgcacaccataatgggggcctacaatgaatttcgcggtaccaacgccaaccaaagcaaacatttagtaatggatattctaaaaggcgaatggggctacaaaggcgtgttactcacagactggaacgtagatatcaacacttacgatgccgctgttaacggcctcgatatcgaaatgggtacaaatgtagatagctacgacgactacatgcttgcccaaccaatgatcgacatgattaaagcgggcagcattccagagtcagtacttgatgataaagttcgtcgcatactgcgcgtgcaactcagcataggcatgatggacaaataccgcttatctggtgagcgcaatactgccaagcatcacgaagctgcacgcaaaattgcatctgaaggtattgtgctactaaaaaatgaaaacattctgccgctaaataaaaacaaaattaaaaacgtattggtgcttggccccaacgcagacaaagtgcacggtttaggcggtggctcgtcagaagtgccagcactttatgaaataaccccgttacaagggttaaaacagaagctgggagataatgtaaacattaccgttatgcgcgcacgctatgacggtgtgttaatgcctatcgccagtgattatgttacttctcgtcactggaccggcacacctgcatggaacatggtgcgttactcggatgctgcgcgcacccaagctattggcgactccgccattgttgattcggcttattcttcgcctgcaggcacgactaaagaatacgtcaccatgaccgccacaattaaaccgttaaaatcgggcgagcacacactcaaaacatcggtgatgggcgatttcgaattaaaaattaacggtaaaaccacagtaaaacatagcagcactagcggcgatgtagtaacccaaaaaatcgccctcaacggcggtgaaacatacagcttcgaaattttatacagcggcaataaaaactttaccttgggctgggatgcaccgggagatttatttaccgcagaaaaagaatacatagccgccgcgaaaaaagcggatgtagtgttttactttggcggcctaacccacggcgacgaccgcgaagcaattgaccgccctcacatgaagctgcctaaccatcaagacccagttattagcaaagtattagctgcaaacccgaacacggttgtatttttaattgcaggctctgctgtagaaatgccgtgggccgataaagctaaagctattgtgtggggctggtatggcggtatggaggccggtaacgcctacgccgatatgctatttggcgataccaaccccagcggcaaaatgccaataactttaccaaaggcactggaagatactgctccaatcgcactgaatgattacaaccctgttgaatcactctacaccgagggcgtgtttattggttaccgctggttcgaaaaacaaaacatcgagccgctattcccgttcggtcatggtttgtcttatacccagtttaagtacaacaatataaagctctctagcgcgaacattaaaggcgaccaaaccgtcaccgtaagcgcaaccattaccaatactggcaaagtggccggcgctgaagttgtacaactgtatttgcatgacgagcaagcaagcgtagaacgcccagcaaaagaacttaaaggtttccaaaaagtgtttttaaagccgggtgaaagcaaagcggtaaatattacgcttaataaacgcgccctttcattttgggatgaaaacagcaacgactggcttgcagaaacaggtaaatttaatgtgctattgggcgcatcagtaagcgatatacgcttacaaactagcttccaataccagcagtaa

SEQ ID NO: 130 - protein, 866 aa, Saccharophagus degradans
  Sde_2674
MLLSLKNTQLKRSMNMNLKHLFLVALALNIAACNVKEPAATNDNHISYQAAREARLAKVEAEVERLLPLLTLEEKASLVHANSKFSIASIERLGIHEMWMSDGPHGVRYQIERHGWAPAGWTDDNSTYLPPLTTVAASWNPEIAALHGDVLGAEARHRRKDVILGPGVNLARLPLYGRNFEYMGEDPFLASRLAVAEIKAIQENDVAACIKHFALNNQELNRTGVNAKPDERTLREVYLPAFEAAVKEAGVHTIMGAYNEFRGTNANQSKHLVMDILKGEWGYKGVLLTDWNVDINTYDAAVNGLDIEMGTNVDSYDDYMLAQPMIDMIKAGSIPESVLDDKVRRILRVQLSIGMMDKYRLSGERNTAKHHEAARKIASEGIVLLKNENILPLNKNKIKNVLVLGPNADKVHGLGGGSSEVPALYEITPLQGLKQKLGDNVNITVMRARYDGVLMPIASDYVTSRHWTGTPAWNMVRYSDAARTQAIGDSAIVDSAYSSPAGTTKEYVTMTATIKPLKSGEHTLKTSVMGDFELKINGKTTVKHSSTSGDVVTQKIALNGGETYSFEILYSGNKNFTLGWDAPGDLFTAEKEYIAAAKKADVVFYFGGLTHGDDREAIDRPHMKLPNHQDPVISKVLAANPNTVVFLIAGSAVEMPWADKAKAIVWGWYGGMEAGNAYADMLFGDTNPSGKMPITLPKALEDTAPIALNDYNPVESLYTEGVFIGYRWFEKQNIEPLFPFGHGLSYTQFKYNNIKLSSANIKGDQTVTVSATITNTGKVAGAEVVQLYLHDEQASVERPAKELKGFQKVFLKPGESKAVNITLNKRALSFWDENSNDWLAETGKFNVLLGASVSDIRLQTSFQYQQ

SEQ ID NO: 131 - DNA, 1701 bp, Saccharophagus degradans
  Sde_2490
atgtttcttttagactttacccgcactgcgtttagctgtacacgaaagcttacctttttgaaatccgcgctacttataagctgctttgccgggcttactgcctgtggtggcgggagtgatggcggtgctgcaagtggctcatcctctagctcgtctagcagcagttcgtctagtagctcttcgagcagttcttcaactagttcctcaagctcctcttcaagctctagttcgtccagttccagctcttcgtctaatagttcctctagctcctctggtggcgatgctttagcgtgccagcatgaaatggcaccagcgctattatctgcaagtgatactaccatggtgcaagcggagtattacgatacctgtgcttcttcggcattagataacaccactggtaacagtggcggtgagttgcgaactgacgatgtagatatagtggccattgcggacggctatgctattacggatatgcagtcaggcgagtacgtagaatattcactaacagtgcaaacttccggtttgtttgacattagttttgcggtacagccgcacgcagctaatactgccggtttggcgctgagtgtagatggcgcagtgttaggcacagttgatattgccgctaatgacagcaccgcatttggcgaatatacgcttaacggcgtgtacataagcgatggcgcgcaagtaataagggtaaccatggccggcgaaggcgctgctattgggttagattccattgcctttaattacaccgataataccgtttacaccccagaaaacgccgtgttgggtatgggaataggtattaacctaggcaataccttagatgccttccccaacgaaggtgactgggcaccggctgcgcaggaatactattttaaagcctacaaggatgcaggtttccgccatgtacgcatcccagcaacttgggatgatcacacggctgatacagccccctacgctgtaaatgcagcacgtatggatcgcactgagcagattgtagattgggccttggcgcagggctatttcgtaattcttaatgcccaccacgaacactggctaaaagaaaactacggcaatcaaacataccgcgatcgctttgatgcaatttggcagcaaattgccgaacgctttaagaataagtcggctcgcttaatgtttgagatactcaatgagccaaacggcatgacagtggccgatgtggatgacctcaacccacgtattctcgatattattcgcgaaaccaatcccacgcgattggtagtgttctctggtaatgggtatacccctgtggatgccttacttgcggctgcaatccctaatgatgattaccttattggtaactttcactcctacgacccttggcagtttggcggtcagtgcgtacgatcgtggggtacagagcaagattacaccgacctagagaacatatataagcgcgcaaatacttggtctgagcagcacgacatacccgttatggtgaacgaatttggcgctgcccattacgattttactgcaccgcagaatgtatgtaaccagcaggctcgtttggcttatttaggtgcccatgccacatttgctattcagtacggctttggcgcaagtgtatgggacgacggtggatcatttgaggtgtacaagcgcggtgaaaatagctggcgcgaagctaaagatgtattagtggcgccaaacccgtag

SEQ ID NO: 132 - protein, 566 aa, Saccharophagus degradans
  Sde_2490
MFLLDFTRTAFSCTRKLTFLKSALLISCFAGLTACGGGSDGGAASGSSSSSSSSSSSSSSSSSSSTSSSSSSSSSSSSSSSSSSNSSSSSSGGDALACQHEMAPALLSASDTTMVQAEYYDTCASSALDNTTGNSGGELRTDDVDIVAIADGYAITDMQSGEYVEYSLTVQTSGLFDISFAVQPHAANTAGLALSVDGAVLGTVDIAANDSTAFGEYTLNGVYISDGAQVIRVTMAGEGAAIGLDSIAFNYTDNTVYTPENAVLGMGIGINLGNTLDAFPNEGDWAPAAQEYYFKAYKDAGFRHVRIPATWDDHTADTAPYAVNAARMDRTEQIVDWALAQGYFVILNAHHEHWLKENYGNQTYRDRFDAIWQQIAERFKNKSARLMFEILNEPNGMTVADVDDLNPRILDIIRETNPTRLVVFSGNGYTPVDALLAAAIPNDDYLIGNFHSYDPWQFGGQCVRSWGTEQDYTDLENIYKRANTWSEQHDIPVMVNEFGAAHYDFTAPQNVCNQQARLAYLGAHATFAIQYGFGASVWDDGGSFEVYKRGENSWREAKDVLVAPNP

SEQ ID NO: 133 - DNA, 1833 bp, Saccharophagus degradans
  Sde_2494
atgatgtacacaaacctctttaatttaaaaaagcacctctttcaaacctcacttaaactactggcctgcgccacattaattggcggcaccctaaacgcagccgctgacgtgccagcaatgtccgtacaaggcaataaagtactggtgggcggtgaagttaaaagccttggaggtatgagctatttttggtctaacaacggctggggcggcgagaaatactacaacgcttctaccgttagttacttcaagcaagactggaaggcatccattgttcgagctgcaatgggggtagaagatgccggcggctacttcgatgacccgcagggctctaagcaaaaagttcgtacaatagtagatgccgccattgcgaatgatatgtacgtcattatcgattggcactcacattacgccaacacccacgactgggcagccgctgtgcaatttttccaagaaatggcacgtgactatggccaatacaataatgtgatttatgaggtatacaacgaaccactggatatcccttggggccacataaaaagctacgccgaaacggtaattgatgccattcgcgcaattgacccagataacgtgatcgtagtaggcactcctcgctggtcgcagggggtaaaagaagcgtcatgggacccaatcaaccgcaataatattgcctacacgctgcacttctattcaggtagtcatggccaatggctgcgcaacgacgcagcagaagctatgagtaatggtattgccttgtttgttactgaatggggcagcgtaaatgccaatggcgatggcgcagtcaacgaaggcgaaaccgcagcgtggatgaacttcatgcgcgataacggtatccatcacgcaaactggtctgtaaacgacaaagcagagggtgcatctgcacttaaccctggcgccagtgccacaggtggttggggcgacggcgatttgacttggtctggccatgttgtgcgcggctacctgcgcgactggaaccaaattggttctggcaatggtaacggcaacggcacaggctgcaccgaggttagcctaccaggcacgatagaagcggaagcctactgcgcaatggatggtatccaaaccgaaaacaccaacgacaccaacggcggcagtaacgtgggctacatagatgctggcgactggatgagctacagcgtaaacgttgctaacgcaggcacttataccgtgtcttaccgcgtggctagccttggcggcggcggtgttctaagcattgaaaatgccggcggctcgcccgtttatggcacgctgaatgtaccgcaaactggcggctggcaagaatggaccactgtatctcacgatattagcttgcaagccggccaacaaaacattggcatagcggcaatagaaggtggttttaacatcaactggatagccctaacccctgctggcaccaaccccaacccagtgcaaagtattaccttacaagcagaagactactcctttatgagtggcgtgcaggtagaaaatactagcgacaatggcggcggtatgaacgtaggctggttagatgctggcgactggcttgcctaccacggcgtaaacattccaacctctggccaatacaccataacttaccgagtagccagccaaagcggtggtggaagcctgcagctagaacaagcaggtggcggcgttgtttacggtaacctgaacgtaccaagcactggcggctggcagaactgggtagacgtaagccataccgttacccttaacgctggtgtacaagattttgggttaggtattactagtggtggcttcaatattaactggataaaagtcgaggcaattcactaa

SEQ ID NO: 134 - protein, 610 aa, Saccharophagus degradans
  Sde_2494
MMYTNLFNLKKHLFQTSLKLLACATLIGGTLNAAADVPAMSVQGNKVLVGGEVKSLGGMSYFWSNNGWGGEKYYNASTVSYFKQDWKASIVRAAMGVEDAGGYFDDPQGSKQKVRTIVDAAIANDMYVIIDWHSHYANTHDWAAAVQFFQEMARDYGQYNNVIYEVYNEPLDIPWGHIKSYAETVIDAIRAIDPDNVIVVGTPRWSQGVKEASWDPINRNNIAYTLHFYSGSHGQWLRNDAAEAMSNGIALFVTEWGSVNANGDGAVNEGETAAWMNFMRDNGIHHANWSVNDKAEGASALNPGASATGGWGDGDLTWSGHVVRGYLRDWNQIGSGNGNGNGTGCTEVSLPGTIEAEAYCAMDGIQTENTNDTNGGSNVGYIDAGDWMSYSVNVANAGTYTVSYRVASLGGGGVLSIENAGGSPVYGTLNVPQTGGWQEWTTVSHDISLQAGQQNIGIAAIEGGFNINWIALTPAGTNPNPVQSITLQAEDYSFMSGVQVENTSDNGGGMNVGWLDAGDWLAYHGVNIPTSGQYTITYRVASQSGGGSLQLEQAGGGVVYGNLNVPSTGGWQNWVDVSHTVTLNAGVQDFGLGITSGGFNINWIKVEAIH

SEQ ID NO: 135 - DNA, 3219 bp, Saccharophagus degradans
  Sde_2497
atgaaaaatactttatcctttaaaacatccttgcttgcgggcttggtggcatccagtttactggttgcggcctgtcagggtgttaaacagcaaacggaagctactcagacaaagcacaatattaccttatggccgcaggcgtctagccctgtaataaagtcgccagattacgaagcggaagtggaagccaaggtagaagcgttgttaggacaaatgacgctagagcaaaaagtagggcaaatcctacagccagaaattcaatctattaagccgcatgaagtaaaagaataccacattggctctgtactaaatggtggtggctctatgcctaaccgcatagaaaatgcgccgcccattgaatgggtaaaattggccgatgccttttacgatgcctctatggacgattctgacggtggaatcgcaattcccattatttggggtaccgatgccgtacacggtcacggcaatgtaactggcgcaaccatattcccgcataacataggccttggtgctgcacgcaacccagcgcttatcgaaaaaattggcgaaataacggcaaaagaagtacgcgcaaccggcattgaatggatatttggcccaactttggccgtagcgcaaaacgatttatggggccgcacttacgaaagctactcggaagacccagccatagtggccgactacgccagtgccatggtggtaggtatgcagggcaaagtggacgacagcgattttctgtccactaatcgcgtagttgccacagcaaagcactttttagctgacggcggtaccttaggaggcaacgatcaaggtgatgcgcgcataagcgaagaagagttggtgcaaattcataatgcgggctatgtgcctgccattgaatcgggcgtgcaaacggttatggccagtttctctttgtggaatggcgtaaaaatgcatggtaacaactacctacttacccaagcacttaaagagcgtatggggtttgatggttttatagtaggggattggaatggccacgggcaggtacctgggtgcaccaacgaatcttgccctcaatcgctaaacgccggtttagatatgtacatggtgccttacgattggaaaaaactgtacagaaacttaattagccaagtgcaatcgggtgaaattgccccaagccgtttagatgacgctgtacgccgtattcttcgggtaaaaattcgcgctaatttgtgggctgcgaaaccttcagagcgaattaatctagccactattgacgaggtggttggccacgcaaaccaccgtgaggtagcgcggcaggcggtgcgagaaagtttagtattgttaaaaaataaaaatagcgtactgcctattgctgccaataaaaccgtgctggttgcaggtgacggcgccgataatattggcaaacaatctggcggttggagtgtaagctggcagggcactggtaacaccaatgcatccttccccggtggtacatctatttataaaggtattgccgatgcagtcactcagggcggcggtaaagctacgctttctgtggatggcagctacaaaactaaacccgatgttgccattgtggtaataggcgaagacccttacgccgaaggccaaggcgaccgcaatagtttagagttcgagccggtgaataaaaaatcgcttgagctattaaaaaaattaaaagcagatggcatacccgttgtaacagtatttatttctggccgacctatgtgggctaacccagaaattaacgcgtctgatgcatttgttgccgcgtggttacctggctctgaagggcagggcgtagcagatgtacttataggcaacgccaacggcaagcctcgttttgatttcaagggcaccttgtcgttctcttggcctaagctgccgacccaaggcttgctcaacccaacgcaccccaactacgacccgttatttaaattgggatacgggctaacttatgcctcgagtgaaactggcccagagcaattggcggaagatgttgaaggtgtagataaaggctcaaccggcgacattaatttttatgttggccgcacattagagccgtgggaagtgtttgttcgaactcctgaaagttcgcagcgtttaagtggcccatttgcagacttaggcaatgccagtgtgcgtaccagtgatatgcaggtacaagaagatgcccttacttttacttggggcggtagctggatgtctattctgggaatagaaggagggcgcggttacgacctttcttcgcaatataaagaaggcggagtaataagctttaacttcaattcaatagatatggctaaaggcgatttaaaagtacaaatggcctgtggtgaaggttgcacgcgtgaagtagatatcacaactatcgcacgcgacttggaaggcaaaggctggcagtcgttaacagtgcccttagcgtgctttgcacacgaaggcgacgatttcacccatattactgcgccgtttaacttatttgccggtggaaaaggtcaagttgctgtagccaacattcgcatactgcgcgccggtacacaaaccgtgccgtgtgtattgcctaaagatgtttccgtaacgccagagccgctgaatgctagctgggcgatagattggtggatgccgcgccacaaagaaaaactggcgcgtatccagcaaggtaatgtggatttactaatgattggcgattccattacccacggctgggaagatgcaggtaaagacgtgtgggcgcaatattacgcgcaccgcaatgcagtggacttaggctttagtggcgaccgaaccgaaaacgtattgtggcgcttacagcacggcgaagcagacggtattaagcctaaagtggcagtggttatgattggtaccaacaatgccggccatcgtcacgagccttcgcactacacagccaagggtgttgcggctgtcgttgctgaattgcaaaaacgattgcctgaaacaaagatattattactgggtatattccctcgcggcgaaaccagtgaagaccctttgcgggtattaaatgccaaaaccaatactcttttggcgaaaatggccgacggagagaaggtggtgtatttgaatatcaataaaacgtttttagatgaaaacggcgtattgcctaaagatataatgcccgacctattgcaccccaatgaaaaggggtacgcattgtgggcgaaagcgatggaacccacccttaaaaaaatgctgggcgaatag

SEQ ID NO: 136 - protein, 1072 aa, Saccharophagus degradans
MKNTLSFKTSLLAGLVASSLLVAACQGVKQQTEATQTKHNITLWPQASSPVIKSPDYEAEVEAKVEALLGQMTLEQKVGQILQPEIQSIKPHEVKEYHIGSVLNGGGSMPNRIENAPPIEWVKLADAFYDASMDDSDGGIAIPIIWGTDAVHGHGNVTGATIFPHNIGLGAARNPALIEKIGEITAKEVRATGIEWIFGPTLAVAQNDLWGRTYESYSEDPAIVADYASAMVVGMQGKVDDSDFLSTNRVVATAKHFLADGGTLGGNDQGDARISEEELVQIHNAGYVPAIESGVQTVMASFSLWNGVKMHGNNYLLTQALKERMGFDGFIVGDWNGHGQVPGCTNESCPQSLNAGLDMYMVPYDWKKLYRNLISQVQSGEIAPSRLDDAVRRILRVKIRANLWAAKPSERINLATIDEVVGHANHREVARQAVRESLVLLKNKNSVLPIAANKTVLVAGDGADNIGKQSGGWSVSWQGTGNTNASFPGGTSIYKGIADAVTQGGGKATLSVDGSYKTKPDVAIVVIGEDPYAEGQGDRNSLEFEPVNKKSLELLKKLKADGIPVVTVFISGRPMWANPEINASDAFVAAWLPGSEGQGVADVLIGNANGKPRFDFKGTLSFSWPKLPTQGLLNPTHPNYDPLFKLGYGLTYASSETGPEQLAEDVEGVDKGSTGDINFYVGRTLEPWEVFVRTPESSQRLSGPFADLGNASVRTSDMQVQEDALTFTWGGSWMSILGIEGGRGYDLSSQYKEGGVISFNFNSIDMAKGDLKVQMACGEGCTREVDITTIARDLEGKGWQSLTVPLACFAHEGDDFTHITAPFNLFAGGKGQVAVANIRILRAGTQTVPCVLPKDVSVTPEPLNASWAIDWWMPRHKEKLARIQQGNVDLLMIGDSITHGWEDAGKDVWAQYYAHRNAVDLGFSGDRTENVLWRLQHGEADGIKPKVAVVMIGTNNAGHRHEPSHYTAKGVAAVVAELQKRLPETKILLLGIFPRGETSEDPLRVLNAKTNTLLAKMADGEKVVYLNINKTFLDENGVLPKDIMPDLLHPNEKGYALWAKAMEPTLKKMLGE

SEQ ID NO: 137 - DNA, 2589 bp, Saccharophagus degradans
  Sde_0245
atgctcaaaaagataaacaagaaaggtcttgctttaagcttagcaattgcagcaatgctaagcggctgcaacgaaggcgacagcaacaaaaccaaaccaagtgcggaaaccctctccgctactcaagccagtaacactgtagccaaccccagcatttggcccaaggtaactagcaaggttgccaaagacgccaaaatggaagcagatataagcgcaatactcagcggtatgacccttgagcaaaaagtagcccaaatgatccaacccgaaattcgtgccttcagcaaagaagacatgaaaaagtatggttttggctcctaccttaacggtggcggcgcattccctaacgacaacaaacattccaccatggccgactgggttgccctagccgacgacatgtatgaagcctctatagacgacagcatagacggcagcactattccaaccatgtggggtaccgatgcagtacacggccacaacaacgtggttaaagcgactattttcccacacaacattggccttggcgccatgcataaccccaagctcatgcagcaaataggcgctgccacggctaaagtggtacaagttactggtatcgactgggtatttgcgcccactgttgcggtagtgcgcgacgaccgctggggccgtacttacgagggctactctgaagaccccgccatagtaaaagaatacgctcgcgccatggttattggcatgcagggcgaagccaatagcgaagcgtttatgggtgacggcactgttatagccaccgccaaacactttttgggcgatggcggcaccgacaaaggcgacgaccaaggcaacaacttatccaccgaacaagaattaattgatattcacgcccaaggctatataagcgccattgaagaaggtgtgcaaactatcatggcatctttcaatagctggaatggcgaaaagatgcacggcaataaatctctgcttaccgatgtccttaaaaagcaaatgggctttgacggtttggtggttggcgattgggatggccacggccaagtaaaaggttgctctaatgcaagctgtgcccaagccatcaacgccggtgtcgatatcatcatggtacccaatgagtggaaacccatgttcgaaaacaccgttgcacaagttaaaagcggcgaaatctctgaagcgcgaattaacgatgcagttacccgtattttacgtgtaaaaatgcgcgctggtattttcgacggtgttaaaccatcggatcgcgccttcgcagcagaagaaaaatacctaggctctgccgaaaaccgcgctatcgctcgtcaagctgtacgcgaatcgttagtgttgcttaaaaaccaaaacaaactgctgccattagaccgcaaaatgaacgttttaatggcgggttctggcgcagacaacatcggcaagcaaagtggtggttggacattaagctggcagggtactggcaacgtgaacagcgacttccctggcgcaacatctatttacgacggcgttaaccaagtagtgagcagcgctggcggtaaagtagagctaagcgaaaacggcaactaccaagccaaaccagatgtagcgattgtagtatttggtgaaaacccttacgcagaaggcgtaggcgatattgaaggtattgaataccaactaaacaataagcgcgatatcaatttgttacaaaaactcaaagccgatggcattcctgttgtatcggtattcttaaccggtcgtccactttgggtaaacaaagagcttaatgcctccgatgcttttgttgcagcttggctgccaggctctgaaggtgtaggcgtttctgatgtgctattcaaaaaagccgacggtagtattaactacgactttaaaggcaagctaacttactcttggccaaagtatgatgaccaagtagtaataaacaaaggcgacaaagattacgccccgctttacccttatggttacggcttaacctacagcgatgttgacacccaaggtgacgacttacctgaagaaaccaaagttaaaattggccgcgctgacgacgagccaatggccatcttcgacagcctaccccaaagcgacctcggcttcttccttggcgacaaagccaactgggtagtacctattgcaacaagtgtagttacaacgcacaacagcgataacctaaccatgcgcacctacaactggaaagtacaagaagatgctcgccagttaatttggaaaggcgacagcaaagccaatgccttctttgcatggccagacccacacaatatgcaaggcatgttagaacacaaagcggcttacagctttagcattaaagtagataaagcacccgctggcgacctaacactaggcatacactgcatggaagaatgcggtaaaaaacttgtgcttaacgaagcgcttagcaaaattcctgctggtgagtggggagagctaacaatagatctagcttgcatagcagatgccgaagccttggccgaagttcgctcacccttcatgctaagcaccgatgcacccgcatctatcgtgtttggcgatgtgaagttagtacctggcggtgcagatagcgcagctattaagtgtgactaa

SEQ ID NO: 138 - protein, 862 aa, Saccharophagus degradans
  Sde_0245
MLKKINKKGLALSLAIAAMLSGCNEGDSNKTKPSAETLSATQASNTVANPSIWPKVTSKVAKDAKMEADISAILSGMTLEQKVAQMIQPEIRAFSKEDMKKYGFGSYLNGGGAFPNDNKHSTMADWVALADDMYEASIDDSIDGSTIPTMWGTDAVHGHNNVVKATIFPHNIGLGAMHNPKLMQQIGAATAKVVQVTGIDWVFAPTVAVVRDDRWGRTYEGYSEDPAIVKEYARAMVIGMQGEANSEAFMGDGTVIATAKHFLGDGGTDKGDDQGNNLSTEQELIDIHAQGYISAIEEGVQTIMASFNSWNGEKMHGNKSLLTDVLKKQMGFDGLVVGDWDGHGQVKGCSNASCAQAINAGVDIIMVPNEWKPMFENTVAQVKSGEISEARINDAVTRILRVKMRAGIFDGVKPSDRAFAAEEKYLGSAENRAIARQAVRESLVLLKNQNKLLPLDRKMNVLMAGSGADNIGKQSGGWTLSWQGTGNVNSDFPGATSIYDGVNQVVSSAGGKVELSENGNYQAKPDVAIVVFGENPYAEGVGDIEGIEYQLNNKRDINLLQKLKADGIPVVSVFLTGRPLWVNKELNASDAFVAAWLPGSEGVGVSDVLFKKADGSINYDFKGKLTYSWPKYDDQVVINKGDKDYAPLYPYGYGLTYSDVDTQGDDLPEETKVKIGRADDEPMAIFDSLPQSDLGFFLGDKANWVVPIATSVVTTHNSDNLTMRTYNWKVQEDARQLIWKGDSKANAFFAWPDPHNMQGMLEHKAAYSFSIKVDKAPAGDLTLGIHCMEECGKKLVLNEALSKIPAGEWGELTIDLACIADAEALAEVRSPFMLSTDAPASIVFGDVKLVPGGADSAAIKCD

SEQ ID NO: 139 - DNA, 1356 bp, Saccharophagus degradans
  Sde_0325
atgagaataataacggcgtttgcagttatgctgctatgcataacaggctgtagcggatcgggcgcgagtgatagcccgcaagcatccaattcgtcttcgggcagttcttctagctctagcagttcgtcaagttcgagcagttcctctagttcgtcgtctagctcttcaacaagctctagcagctcatctagctccagctcatcatcaagctctagcagttcttcgggcggcgaagcgctttacccaagctacaatacaaacccgccagcgccagatatgaccggcatgacaagtactgccacacaactagcagatcgtataaccgtgggctggaatattggtaacacgctagaggcaataggcggcgaaaccaactggggtaacccgctggttactaacgaattaattcaagcggtaaaagccagtggctttgattccattcgtatacccgccgcgtgggatcaatacgccaaccaagaaacggccgcaatagatataaactggctaaaccgcgttaaacaagttgtgcaatacagcatagataacgacatggtggtagtgctaaacatccactgggatggcggttggctagagcgcaatgtagagcccagcgagcaagtagcagtaaatgcaaaacaaaaagcctattgggaacaaattgccactcacctgcgcgactttgacgagcgcctaatatttgccagcgccaacgaaccccatgtagaaaccgaagcacaaatggccgtactaaacgtataccatcaaacgtttgtagatacagtgcgtgcaactggcggtaaaaatgcttaccgcgtactggtattgcaggggccaaaaacagatatagaaaccacctcgctattgtggacccaaatgccgcaagatagcgccgtaaataaacttatggcagagctacacttctataccccgtacaactttacgttaatgaatgtagatgaaagctggggcaaccagttctactactggggcgaaggtaatcattccactaccgacacaggccgcaacccaacctggggcgaagaagcaacagtagattcactgctggcaattaccaaacaacagtttgtggaccaaggtatacccgtaattattggcgaatacggtgcacaacgccgcgataaccttaccggcgatgaattggccctgcacttacaatcgcgcaactactacttaaaatacgttactcaaaaatgtgtagagctaggcttaaaacctttttattgggataccggcggcttagacaacaatcaatctggcctgtttaatcgcagtacctaccaagtatttgatcaaaatgccctagatgccattatggaaggggccagaggggaataa

SEQ ID NO: 140 - protein, 451 aa, Saccharophagus degradans
  Sde_0325
MRIITAFAVMLLCITGCSGSGASDSPQASNSSSGSSSSSSSSSSSSSSSSSSSSSSTSSSSSSSSSSSSSSSSSSGGEALYPSYNTNPPAPDMTGMTSTATQLADRITVGWNIGNTLEAIGGETNWGNPLVTNELIQAVKASGFDSIRIPAAWDQYANQETAAIDINWLNRVKQVVQYSIDNDMVVVLNIHWDGGWLERNVEPSEQVAVNAKQKAYWEQIATHLRDFDERLIFASANEPHVETEAQMAVLNVYHQTFVDTVRATGGKNAYRVLVLQGPKTDIETTSLLWTQMPQDSAVNKLMAELHFYTPYNFTLMNVDESWGNQFYYWGEGNHSTTDTGRNPTWGEEATVDSLLAITKQQFVDQGIPVIIGEYGAQRRDNLTGDELALHLQSRNYYLKYVTQKCVELGLKPFYWDTGGLDNNQSGLFNRSTYQVFDQNALDAIMEGARGE

SEQ ID NO: 141 - DNA, 2604 bp, Saccharophagus degradans
  Sde_0649
atgaatcttacttcaatcatgtttgaacaatcagtaaaaaaagtcgctaagtcagccattgccgtggcagttgcttcggcggttaccttaagtgcggcgcaggccgaggtgggtaacccacgtgttaaccaagtaggctatatacccaatggtgccaaagttgccagttatgttgcgccatcaaatacggcacaaacgtggcagttactgcgtaatggcagtgtggttgcaagtggcactacaaccccaaagggtacagatgcagcctcgggtgacaatattcaccatatcgatttttctgcggtgagtgcaaccggcgaaggttttagtttgcttgtgggcggcgatgaaagttacccctttgaaatttctgccgacgcatttacaccggttttatacgattccatccgttacttttatcacaaccgttcgggtatcgcgattgaaacgcagtacaccggtggcggtaacggtagctacgcggcgaatgctcagtgggctaggcccgcaggtcacattaatcaaaatgctaaccaaggcgataatgcggtgccgtgttggtcgggcagtggttgcaactacgccttagacgtaactaaaggttggtacgatgccggtgaccacggtaaatatgttgtaaacggtggcatttccgtatggaagctattaaacatgtacgagcgtgccttgcacattagtggcagccaaaataaatacgccgacggtacattaaatattcctgaaagcggcaatggcgtggcggatattttggatgaagctcgctggcaaatggagtttttattagccatgcaagtgccagagggcgaagcgaaagctggcatggtgcaccacaaaatgcacgatgtgggttggacaggcttgccactagcaccccatgaagataatcgcgagcgcgcgcttgtgccgccttcggttactgcaacccttaacgttgcggccacaggcgcgcagtgtgcgcgtttatttgacgaaatagatgcgagttttgcagcaagttgtttaactgccgcagagcgcgcatgggatgcagccctgcaaaaccctaacgatgtttacactggcggctacgataatggcggcggtggttacggcgatgaagtggcggacgacgagtttttctgggctgctgctgagttatacattaccactggcgatagcaaatatctttcaaccattaacaactacaatgtaacgcgcattgattggggctggccagataccgagttgcctgcgttgatgtcgttagcggttgtgcctgctaatcacaccgcaaatttgcgtgcgactgctcgtgcaaaaattgtagaaattgcagatacccatgtcgctaccagtaatgctgccggctatttaacaccatcgtccgcgctggattactactggggttctaacaatggcgtagccaataaaattgcgttacttggtttggcatacgattttactggcgatgacgtttacgcgaaaacggtgtcgaaagcagttaactatttatttggtaataataccttatcgttttcttatatttctgggcatggcgaaaatgctttgcaacagccgcatcaccgcttttgggctggggcattaaatggaagttacccatggttgccgcctggtgcgctttctggtggccctaacgcagggttagaagatggcgttgccgccgccgcgctaagtgcttgtgtttcaacgcctgccaaatgctatatggatgatattgaatcttggtcgaccaacgaaattactattaactggaatggtgcattggtttgggcaatggcgttttatgatgactacgccgattcgggtagcggttctagctcgtcaagttcttctagctcatctagctcttcgtcaagttcttccagttcgacttctagctcgtcgtcttctagtagtagctcttcgtcgagcggctcgagttcttctagcagctcttccacatccagttccagctcttcgagttcatcgggtggggagtgtgtagaaatgtgtaagtggtatcaagatgcaccgcgccctctatgcaataaccaaaacagcggttggggatgggagaaccagcagagttgtattggtagaacaacttgcgaaagtcaaagtggcaatggtggagtgattaattcgtgcggcacgtctagctcgagctcttcatctagctctagcagtagctcttcgagttcatccagctcttctagcagttcttccacatcaagctcgtcgagtagttcgtcttctagctcttctagttcgacttcaagttcttcgtcgagcagttcagggggcgttgcaggtgtggcttgtgcggtaaccaaaatgaaccattggggcagcggatatcaattagatgtaacagtttctaataatggtgctgcagcggtaagtggttggagtattgaactcgattttggtgaatcgccacagcttactggtagttggaatgctgctgtatcggcatctggtaatactgtatcggctactaacattagttggaacggtaatttaagcgctgggcaatctacctcttttggtatgcagggtaattcagatggttcgctgagcacgccaagctgtttagttaagtaa

SEQ ID NO: 142 - protein, 867 aa, Saccharophagus degradans
  Sde_0649
MNLTSIMFEQSVKKVAKSAIAVAVASAVTLSAAQAEVGNPRVNQVGYIPNGAKVASYVAPSNTAQTWQLLRNGSVVASGTTTPKGTDAASGDNIHHIDFSAVSATGEGFSLLVGGDESYPFEISADAFTPVLYDSIRYFYHNRSGIAIETQYTGGGNGSYAANAQWARPAGHINQNANQGDNAVPCWSGSGCNYALDVTKGWYDAGDHGKYVVNGGISVWKLLNMYERALHISGSQNKYADGTLNIPESGNGVADILDEARWQMEFLLAMQVPEGEAKAGMVHHKMHDVGWTGLPLAPHEDNRERALVPPSVTATLNVAATGAQCARLFDEIDASFAASCLTAAERAWDAALQNPNDVYTGGYDNGGGGYGDEVADDEFFWAAAELYITTGDSKYLSTINNYNVTRIDWGWPDTELPALMSLAVVPANHTANLRATARAKIVEIADTHVATSNAAGYLTPSSALDYYWGSNNGVANKIALLGLAYDFTGDDVYAKTVSKAVNYLFGNNTLSFSYISGHGENALQQPHHRFWAGALNGSYPWLPPGALSGGPNAGLEDGVAAAALSACVSTPAKCYMDDIESWSTNEITINWNGALVWAMAFYDDYADSGSGSSSSSSSSSSSSSSSSSSSTSSSSSSSSSSSSSGSSSSSSSSTSSSSSSSSSGGECVEMCKWYQDAPRPLCNNQNSGWGWENQQSCIGRTTCESQSGNGGVINSCGTSSSSSSSSSSSSSSSSSSSSSSSSTSSSSSSSSSSSSSSTSSSSSSSSGGVAGVACAVTKMNHWGSGYQLDVTVSNNGAAAVSGWSIELDFGESPQLTGSWNAAVSASGNTVSATNISWNGNLSAGQSTSFGMQGNSDGSLSTPSCLVK

SEQ ID NO: 143 - DNA, 1098 bp, Sacchrophagus degradans
  Sde_1572
atgcgcacaaccaaatttcttgcgcttgcactctgcttgctggcctcagccagtgcactgagtgcaaataacagcgccccatcaaacgactggtgggatataccctacccgagccaattcgatgtaaaaagccttaaaacgcaaagttttatatcggtaaaaggtaacaagttcattgatgataagggcaaaaccttcacttttagaggggtaaacattgccgatacaggtaagctacttagccaaaatcaatggcaaaaatcgctgtttgaagagctggctaataactggggggtaaatactattcgcctgcctattcaccctgtaagttggcgtaaacttgggccagacgtttatttaggccacatcgatgaggcggtacgctgggcgaatgatttaggtatttaccttattcttgattggcactccattggctatttgcccaccgagcaataccaacaccccatgtacgacaccaccattaaagaaacccgcgacttttggcgcagaattacgttccgctacaaaaacgtgcccaccgtagcggtatacgaattatttaatgagccaaccaccatgggtaacaccctaggcgaacgcaactgggccgagtggaaaaccttaaatgaaagcctaattgatatgatatatgccagtgacaaaaccgtcattccgctggttgcaggcttcaactgggcctatgatttatcgccaatcaaaaaggcacctatcgagcgtgaaggcattgcttacgccgcacacccctacccgcaaaaggcgaaaccagaggttaagaacgataaaaacttcttcaaactgtgggacgaaaagtggggctttgctgcagacacctaccctgtaatagcaacagagctaggctgggtacaacccgatggttatggtgcccacatacccgttaaagacgacggcagttacggcccccgcatagtgaagtatatgcagaaaaaaggcgtttcttacacggtatgggtattcgaccccgactggagcccaacaatgattaacgactgggattttacccccagcgagcaaggcgcgttttttaaacaggttatgctagaagctaaaaaacgctaa

SEQ ID NO: 144 - protein, 365 aa, Sacchrophagus degradans
  Sde_1572
MRTTKFLALALCLLASASALSANNSAPSNDWWDIPYPSQFDVKSLKTQSFISVKGNKFIDDKGKTFTFRGVNIADTGKLLSQNQWQKSLFEELANNWGVNTIRLPIHPVSWRKLGPDVYLGHIDEAVRWANDLGIYLILDWHSIGYLPTEQYQHPMYDTTIKETRDFWRRITFRYKNVPTVAVYELFNEPTTMGNTLGERNWAEWKTLNESLIDMIYASDKTVIPLVAGFNWAYDLSPIKKAPIEREGIAYAAHPYPQKAKPEVKNDKNFFKLWDEKWGFAADTYPVIATELGWVQPDGYGAHIPVKDDGSYGPRIVKYMQKKGVSYTVWVFDPDWSPTMINDWDFTPSEQGAFFKQVMLEAKKR

SEQ ID NO: 145 - DNA, 1737 bp, Sacchrophagus degradans
  Sde_0636
atgaacaaagttaaagttttagcgctgtgtgccagtgtggctgtaatgataggttgcagtgatgccgacactaaattagctaactcggccaaggccgaggtgggctttaccaaagtgaatcagctgggttatttgcccgcggccaaaaagctggcggtggtacccgccgttgcagctgcaaaattcgacataatcgatgtaactagcggtaaagtagcgtttacggggagtttaagcgacgtaaaaagctggagcgcgatgggggacgaatctttcaagttggcagactttagcgccctgcaagccgaagggagttaccgcttagttgttcagggtgtgagtgattcttacaccttcgatattagcccaagtgtatatagccaagcgcacgatggagcccttaaagcctattactataatcgagcgagcacagagttaacagaacagtacgccggggtgtatgcgcgacctgcggggcacccagataccgacgtacgcatattcgataacgccgcctcagccgcgcgcccagcagatacaagctttgctgcaccaaagggttggtacgatgctggcgattacggcaagtacattgttaacagtggtatttccacttacaccctaatggctgcgtacgagcatttcccgtcgttttacaagcaacgcgatatagatattcccgaatctggcgatgccgtaccggatattctcgacgaggtaatgtggaaccttgaatggatgcaggtcatgcaagacccgaacgacggcggtgtgtaccacaagcttaccaccctgaatttttctggcgcagtcatgccgcacgaagcgactgcgcagcgctattttattaaaaaatctaccgctgcaacgctagattttgccgcggttatggccactgcaagccgagtatacgcaccgttcgaaggtgcttttcctggtaaatcagctgcttatcgacaggcggccattgctgcgtgggagtgggcacaagcaaaccctagtgagacatattcgcagacaccgctgagcaaagttcaaaccggcgcctatggtgataaaaagttaaacgatgaatttgcgtgggcggccgcagagttgtttatattgaccggcgagcaaaaatactggcaggcgtttaacaagcaaaaagtgcaggcgggtgagtctagctgggcgaatgttgcggggttggggtttatttccttggccaataatgcgcgcagcctgttaaacgaagctcaatacaaaaccgttaccgattcaattgttcgcgctgcagatagcttgcttgttacttacaaagagaatgcctaccaagtacccattggcaacaaagattttttctggggtggcaattccggcacgttaaatcgcgcttgggttttgcttgaggccaataaaattaaaccgcagcaagaatacatcgatgctgcacttgccgcggtggattatatttatggtcgcaaccctaccaactactcttttgtcactgggtttggcgataaccctgcggtgggtatccatcatcgtccatcctatgccgatggcattaaagcccctgtgcctggttggcttgcgggcggtgcgcacaatggcaagcaagatggttgtgagtacccttccgatgcaccggcaaaatcctatctagacgactggtgcagttactccaccaacgaaattgctattaattggaatgcgccgttagtttacatactggctgcggtaaataatttgtag

SEQ ID NO: 146 - protein, 578 aa, Sacchrophagus degradans
  Sde_0636
MNKVKVLALCASVAVMIGCSDADTKLANSAKAEVGFTKVNQLGYLPAAKKLAVVPAVAAAKFDIIDVTSGKVAFTGSLSDVKSWSAMGDESFKLADFSALQAEGSYRLVVQGVSDSYTFDISPSVYSQAHDGALKAYYYNRASTELTEQYAGVYARPAGHPDTDVRIFDNAASAARPADTSFAAPKGWYDAGDYGKYIVNSGISTYTLMAAYEHFPSFYKQRDIDIPESGDAVPDILDEVMWNLEWMQVMQDPNDGGVYHKLTTLNFSGAVMPHEATAQRYFIKKSTAATLDFAAVMATASRVYAPFEGAFPGKSAAYRQAAIAAWEWAQANPSETYSQTPLSKVQTGAYGDKKLNDEFAWAAAELFILTGEQKYWQAFNKQKVQAGESSWANVAGLGFISLANNARSLLNEAQYKTVTDSIVRAADSLLVTYKENAYQVPIGNKDFFWGGNSGTLNRAWVLLEANKIKPQQEYIDAALAAVDYIYGRNPTNYSFVTGFGDNPAVGIHHRPSYADGIKAPVPGWLAGGAHNGKQDGCEYPSDAPAKSYLDDWCSYSTNEIAINWNAPLVYILAAVNNL

SEQ ID NO: 147 - DNA, 2376 bp, Sacchrophagus degradans
  Sde_2272
atgttggcttctaataaaaatagtaagctggcaaactctgagcaacaccgcccttataaaacccgcacagcgcgctggttaaccgggtctggggttattgcttcaagtttgcttttttctgcgcagagttttgcggcgcaatgtgaatacatcattagcaatgaatggaacagcggctttactggcgcagttcgcattactaataatggcactactcccatcaatggctgggatgttagctggcagtatgccggcgatgcagtcaccagcagctggaacgcgaatgtttctggctcgaaccccgtttctgctacaccattaagctggaatgccaacattcaacccggtcaaagcgttgagtttggttttcagggcagcaaagccggctccaatgcagaaattccaaccgttaccggcgcggtatgtgatagcggctctagctcttccagctccagctcatcatctagttcatcaagctcttctagtagctcaagcagcactagcagctcctcgtccagctcttcaagcacctcttcgtctagctcatcatctggctccagtggcacaggtggtattgcgtgtactgtaggcaatgcgaatatttggggctcgggctaccagctggacatgcaagttgttaacaacggcaccgctgcagtaagcagttgggacgtaaccatggcattcggcgaggcaccacagcgcaccggtggctggaacgcaaactttgtagagtcaggcaataccattgttgcgagcaacattagctggaacggcaacctcgcaccggggcaatcagcttcgtttggtattcaagggaaccacgacggctcttttggcggcgtaacctgtaacggcgcttcaagctctggctcgtctagttctggctctagcaccagctcatcaagtagctcatccagttcgtctggctctagcacttctagctctagctcaagctcctctactggttctacctctagctctagtagctcttcaacttctagcacttcttcaagttctagctctagcaccagctccacgagttctagctccagttctagctcgagtacatcgtctagttccagctcatcttcctcaagctctagctcttctacttcaggcagtggcgcaggttttgacaacccgttcattggcggcaagtggtatgtagacccagtatggtcagcaaaagctgcagcagagccaaacggttcacttattgccaactacaacacggcagtttggatggatcgcattggtgcgattgaaggcccagaagatggcgatggtatgggcttagaagaacacttagatgaagctttagcacaaggtgcagacatctttatgttcgtggtatacgacctaccaaaccgcgactgtgcagctttggcctcaagtggcgaactactcattgccgagaacggttttgagcgctatcaaaatgagtacattggcccaatcgtagatatactcagcaagcccgcgtattctagcttgcgtattatcgcgattattgaagtggattctctacccaacctcgttaccaacctcaacattcaaaaatgtgttgaagcgaatggcccgggtgggtacgtagacggtatccaacatgcacttaacgagctaaacacgcttgataatgtgtacccatacgtcgatattgctcactcaggctggctaggctggagcgacaacttcgccggcgccaccaagcttattggtgatgcaattaaaggcacaaacaaaggtgtaaacagtattgcaggctttgtaagtaactcttctaactacacacctgtgactgaaccatacctacctaaccctaccttgcaaattggtagcaaccaagttcgatctgcggatttctacgagtggaccatgtacttcgaagaacttagctttgtacaagattggcgccaagccatgattcagcaaggcttcccagaatcaattggtatgcttattgataccgcacgtaatggctggggtggacctgaccgtccaactggtgagtctacatctaccgacctcaacacctatgtgaatgaatcgcgtatagaccgccgtcagcatcgcggaaactggtgtaaccagcccggtggtgttggcttccgtccgcaagcggcaccagaaccaggtgtagacgcttacgtttgggttaagccacaaggtgagtcggatggtattagtgatcctaacttccctatcgaccctaacgacccagctaaacagcacgacccaatgtgtgatccaaacgcacctaaccgcgataacaatgcggttggcacaggcgcgctagataacgctccacatgctggtcgctggttcccagaagcattccaaatacttatagaaaacgcctacccaccgctatag

SEQ ID NO: 148 - protein, 791 aa, Sacchrophagus degradans
  Sde_2272
MLASNKNSKLANSEQHRPYKTRTARWLTGSGVIASSLLFSAQSFAAQCEYIISNEWNSGFTGAVRITNNGTTPINGWDVSWQYAGDAVTSSWNANVSGSNPVSATPLSWNANIQPGQSVEFGFQGSKAGSNAEIPTVTGAVCDSGSSSSSSSSSSSSSSSSSSSSSTSSSSSSSSSTSSSSSSSGSSGTGGIACTVGNANIWGSGYQLDMQVVNNGTAAVSSWDVTMAFGEAPQRTGGWNANFVESGNTIVASNISWNGNLAPGQSASFGIQGNHDGSFGGVTCNGASSSGSSSSGSSTSSSSSSSSSSGSSTSSSSSSSSTGSTSSSSSSSTSSTSSSSSSSTSSTSSSSSSSSSTSSSSSSSSSSSSSSTSGSGAGFDNPFIGGKWYVDPVWSAKAAAEPNGSLIANYNTAVWMDRIGAIEGPEDGDGMGLEEHLDEALAQGADIFMFVVYDLPNRDCAALASSGELLIAENGFERYQNEYIGPIVDILSKPAYSSLRIIAIIEVDSLPNLVTNLNIQKCVEANGPGGYVDGIQHALNELNTLDNVYPYVDIAHSGWLGWSDNFAGATKLIGDAIKGTNKGVNSIAGFVSNSSNYTPVTEPYLPNPTLQIGSNQVRSADFYEWTMYFEELSFVQDWRQAMIQQGFPESIGMLIDTARNGWGGPDRPTGESTSTDLNTYVNESRIDRRQHRGNWCNQPGGVGFRPQAAPEPGVDAYVWVKPQGESDGISDPNFPIDPNDPAKQHDPMCDPNAPNRDNNAVGTGALDNAPHAGRWFPEAFQILIENAYPPL

SEQ ID NO: 149 - DNA, 2022 bp, Sacchrophagus degradans
  sDE_2929
atgttgattggtactgttacggcttcagcactggttggtcgaggccgtggcacccctaaaaaaataatcaacaagggttctattatgtggcaaatcaacaaatcggctttagcggccgtggtattagtgtgttcctcatctagctttgcgcaatctgcatgtgacactcaacgcattgaagccgaaaattacgtggcaatgagtggtattcaaaccgaaagcacggcagacactggtggcggtttaaatgtgggctggatagacgccggcgactggcttagttaccaagttaacctacctgctgcagggcagtacgaggtgcgctatcgcgttgccagtagaaatggcggcggtgtacttcggttagagggcaatgccggtcaaaccttgtatggaactatgaatgtacccaacacgggtggctggcaaaattggcaaaccctttctcattcagtgacattagcggcaggagagcagtctattggtattggtgtgccaagcggcgggtttaatattaattggctggagttcgtacctttagattgcagtgggccaatcgacccgcccattaacccaccttcgaactgcgcgagcattgtattcgaggccgaaaattacgatcaaatgagcggcattagaacgcaaaccacaagtgataccggaggcggcttaaatgtggggtggatagatgctggcgactggcttagctatgccactgtgaatatccccagcacgcaggtgtacaattttgaataccgtgtggctagccctaatggcggcagttttaatttgcagggttcggctggcgcagagaattttgataccgctactttgcccaatacgggtggttggcaaaattggacaacggtaacaggctcggcgcttttacctgctggcaatgtgaatttcggtattagtgcgattactggtggctggaatataaactggtttaaagctacaccagagagctgtgatgatataaaccctccaagtaccggtattactgctaagcaagcagcggcagccatgggcaaggggtttaatttggggcaaatgttcgaaagtacgcaacacccaagaacatttaatgctgcaaaaagtaaaatagatgcttactacaatatgggctacagaaatgtgcgcatccctattacttggactgaagccgtaggcggaaacaggcttgttgcagatgcaaatgtaggcgcagtcaatcgcaaccactctcgcttagctgtaattactcaagtagtagattacgcgctttcgctacccggcatgtacgtggttattaatgcgcatcacgaaggtggattaaaaaccaataatcgctggtgggtgttagaaactctgtgggcagatattgccgatatatttaaagacagagatcaccgtttgctatttgaaatattaaacgagccacacctaagcgatgccaataagtcgcctatgccccccgccaatttgcgttttatgacgggcaaagcctataacaaaattcgcgcgatagatgcgcagcgaatcgttattattggtggcaaccagtggtttggtgcaggtgaaatggcaaacgtatggccaaaccttaatgatgttggcggcggttccgatgcatatgtaatggctacttttcaccattacgacccgtggtcgtttagtggcgataaccaaggcgattacgccgatgcttggacgctatctaacgtgggtaacccaatggatataatgcaaagctgggcaaacggcgtaggccaaggtatgcctgtgtatattggcgagtggggcgtaggttggggcagccgctacagcgccatgcagtgcaataatattcgctattggtaccagctgttcgacgcgagctatgcctcggcaaaaggccagcctacggcagtgtgggatgacggcggttggtttaaaatattcgaccacggtaccaacagcttcaataataatttagcccaatgtattggtggaaactgcgcttgggatggcgccgatagatttaattctggctgtaattaa

SEQ ID NO: 150 - protein, 673 aa, Sacchrophagus degradans
  Sde_2929
MLIGTVTASALVGRGRGTPKKIINKGSIMWQINKSALAAVVLVCSSSSFAQSACDTQRIEAENYVAMSGIQTESTADTGGGLNVGWIDAGDWLSYQVNLPAAGQYEVRYRVASRNGGGVLRLEGNAGQTLYGTMNVPNTGGWQNWQTLSHSVTLAAGEQSIGIGVPSGGFNINWLEFVPLDCSGPIDPPINPPSNCASIVFEAENYDQMSGIRTQTTSDTGGGLNVGWIDAGDWLSYATVNIPSTQVYNFEYRVASPNGGSFNLQGSAGAENFDTATLPNTGGWQNWTTVTGSALLPAGNVNFGISAITGGWNINWFKATPESCDDINPPSTGITAKQAAAAMGKGFNLGQMFESTQHPRTFNAAKSKIDAYYNMGYRNVRIPITWTEAVGGNRLVADANVGAVNRNHSRLAVITQVVDYALSLPGMYVVINAHHEGGLKTNNRWWVLETLWADIADIFKDRDHRLLFEILNEPHLSDANKSPMPPANLRFMTGKAYNKIRAIDAQRIVIIGGNQWFGAGEMANVWPNLNDVGGGSDAYVMATFHHYDPWSFSGDNQGDYADAWTLSNVGNPMDIMQSWANGVGQGMPVYIGEWGVGWGSRYSAMQCNNIRYWYQLFDASYASAKGQPTAVWDDGGWFKIFDHGTNSFNNNLAQCIGGNCAWDGADRFNSGCN

SEQ ID NO: 151 - DNA, 3504 bp, Sacchrophagus degradans
  Sde_3003
atgacaattaaacgttggccgttcgaccgaaaaggcccacctaaaaaacctaacgctaaaaaattactcgcaagcttagcggctgcactaagcttaaccgccatgcaaagcactgcagcggtagagccattacaaaccagcggcaatcaaattcttgttggcaaccaagccaaagcccttggcggccacagcttgttttggcataacgtgccggcagcaggcagcttatacaatgcagatacagtaagcaggcttaagaatgattggaactccaaggttattcgggccgcaattggggttgaagtacctttcaattcagaaaacacctacataggcaataagggcagctcgctggccgcaatagaccgcgtagttaatgccgctgttgccaacgatatgtatgtgattatcgattttcatactcaccatgcagatcaagtagaaaacgttgcccacgactttttcaacgaagtttctagccgttacggtcatttaaacaatgttatttatgaagtatttaacgagccagaatggtgtggcgagcacggtcggtgggcatctaccattaagccctacgccgagcgcgttatccaaaccattcgcaacaatgacccagacaacctagtaatagtaggcactacctgtttctcgcaagatgtagatgtagccgcagccgaccccattaacgatgtaaacgtggcctatacgctacacttttacgcagccacccctgcccaccagcaacccttgcgcgacaaggcccaaaccgcgctcgaccgcggcgcgccactatttgtaaccgaatggggtacaaccacatttacaggtgatggttttgtagatgaggcgcaaacgcgcacatggattaactggttaaacgaacgcggtattagccacgttaactggtcggcgtctacccagccagaaagctcagctatatggaatggcgacatgacctacaagcattcgggcttattggttggcgaactggtgcaacaaacaaatggcacaaccacgccaccaaccggtgaaataagtggcccgtgcgatttacattttgtacctgccaaagccgaggctgaaagcttctgtaccgccaaaggcattcaatttgaaaccaccaccgacacgggcggcggccaaaacatgggctggctagatgccggcgactgggtaacttttgatgtagatgtacctgctagcggccaatatttaatagattaccgcgtagcatcagagctaggtgatggtcggttccgcaccgaagccgccaacggcactgcccttggcacaatatctgtacccaataccggcggctggcagaattggcaaacgcacacacacacagtgcaactctcgcaaggcacacaaaccgttaaactagttgccgaaactggtggctggaacttaaattggtttgaagtgcgcgcaggtgaggtgtgcgaaggcgctgactgcccatgtgaaggagccgaatgcccttgcccagattgcaacggcacaccggttaagtttgaggcagaaacgtttgtggctatgcaaggcgtgcagctagaaaacacatccgatgtgggcggcggccaaaacgttggctacattgatagcggcgactggataacttacaacggggccttgcccgcaagtgcagacaaccgctatgtagtgtcttatagagtagcgcgtcaacctagcggcaatgccaaatttaaaatagaacagccaggtggagcagcggtatatggcgaaatttcggtgcccagcaccggcggctggcaaacatggacaaccattagccacaccataacaattcccgctaacgcaaacggctttgcactagcagcaatagatggcggttggaatataaactggatagaaataaaaccggcgaccactcaaccacccgagccaatcaacccgttaaaacttcaagctgaagattacatcaactttaacgacaccacccccggtaacgaaggcggtgcacacagaagcgatgatgtagatattcaagcaactaccgataccggtggcggttttaatgttggctgggtagacgctggcgaatggctagagtatgagttctttttagagtctcctgatttttatgcagctgatgtacgggttgcttcagaccaaactggcggcgcactgcaactacaaatagatggccaaaacgttggccaagccattaccgttggcaacaccggtggctggcaagcgtggacaaccaaaaacacactcattggcgacctaagtgcaggcacccacacgttgcgtgtatacgcgcaaagcggcccattaaatttaaactgggtagagctaaagcgtacaacgcccgcaccagccacttcgtgttttaatattgccgaagaccgcttaaacgttcacctagatgcgcactgtactgcaggcagcaacctgcaatacaattgggattttggtgacggcaacagcgcaaccggcgtagccactagccacagctactacactagcggcacttacaccattaccttaaccgttagtgatacccgcaccacagacacctctagccaacaggtaacggtagatttttctgcccctgcaggccctgtggatttttacggcgaactaatggtgaatggcaaccgcattcacggcgaaaaaaccggcgaacccgcacaagtacgcggcatgagctttttttggagcaacaccggttggggccaagaaaaatggtggaacgccagcaccgtggaccgcatggttgatgagttcaaagtagaacttgtgcgcggcgcaatgggcactgatgaaggcggcggttatttacacgacgcgtctaataaggctcgcttacaagcagttgttgaacaagccattgcacgcaatgtgtatgtaattatcgactggcacacccaccatgccgaagataacattgccgaagccattacattctttagcgaaatggcgcagctttatggccaccacgacaacgtgattttcgagatttacaacgagccattaaacaccacaagctggggcactattaagcactacgctgaacaagttattcctgctattcgcgctcattccgataatttaattgttgtgggcacgcgcacctggtcgcaaaacgtagacgaagccgcgttcgataaaattaacgacagcaacaccgcctacgccctgcacttttatgttggctcgcacggcaaccacgttcgcaacctagcacaaaccgcactaaacaacggcgcggctatttttgctagcgaatggggaatttggccaaacaacaactacgatggcatgaacgccgacgattggatgaactttttagaccaaaacaaaatatcttgggctaactgggccatatccgacaaagtagaccccaacacaggccaactagaaccacccagcatgttcaacccagacggcagcctaagcagtaatggtcaatatgtagtgaacaaactaaatgaatacgcagcacaagcaccgtggagggaggcaatcgctaattga

SEQ ID NO: 152 - protein, 1167 aa, Sacchrophagus degradans
  Sde_3003
MTIKRWPFDRKGPPKKPNAKKLLASLAAALSLTAMQSTAAVEPLQTSGNQILVGNQAKALGGHSLFWHNVPAAGSLYNADTVSRLKNDWNSKVIRAAIGVEVPFNSENTYIGNKGSSLAAIDRVVNAAVANDMYVIIDFHTHHADQVENVAHDFFNEVSSRYGHLNNVIYEVFNEPEWCGEHGRWASTIKPYAERVIQTIRNNDPDNLVIVGTTCFSQDVDVAAADPINDVNVAYTLHFYAATPAHQQPLRDKAQTALDRGAPLFVTEWGTTTFTGDGFVDEAQTRTWINWLNERGISHVNWSASTQPESSAIWNGDMTYKHSGLLVGELVQQTNGTTTPPTGEISGPCDLHFVPAKAEAESFCTAKGIQFETTTDTGGGQNMGWLDAGDWVTFDVDVPASGQYLIDYRVASELGDGRFRTEAANGTALGTISVPNTGGWQNWQTHTHTVQLSQGTQTVKLVAETGGWNLNWFEVRAGEVCEGADCPCEGAECPCPDCNGTPVKFEAETFVAMQGVQLENTSDVGGGQNVGYIDSGDWITYNGALPASADNRYVVSYRVARQPSGNAKFKIEQPGGAAVYGEISVPSTGGWQTWTTISHTITIPANANGFALAAIDGGWNINWIEIKPATTQPPEPINPLKLQAEDYINFNDTTPGNEGGAHRSDDVDIQATTDTGGGFNVGWVDAGEWLEYEFFLESPDFYAADVRVASDQTGGALQLQIDGQNVGQAITVGNTGGWQAWTTKNTLIGDLSAGTHTLRVYAQSGPLNLNWVELKRTTPAPATSCFNIAEDRLNVHLDAHCTAGSNLQYNWDFGDGNSATGVATSHSYYTSGTYTITLTVSDTRTTDTSSQQVTVDFSAPAGPVDFYGELMVNGNRIHGEKTGEPAQVRGMSFFWSNTGWGQEKWWNASTVDRMVDEFKVELVRGAMGTDEGGGYLHDASNKARLQAVVEQAIARNVYVIIDWHTHHAEDNIAEAITFFSEMAQLYGHHDNVIFEIYNEPLNTTSWGTIKHYAEQVIPAIRAHSDNLIVVGTRTWSQNVDEAAFDKINDSNTAYALHFYVGSHGNHVRNLAQTALNNGAAIFASEWGIWPNNNYDGMNADDWMNFLDQNKISWANWAISDKVDPNTGQLEPPSMFNPDGSLSSNGQYVVNKLNEYAAQAPWREAIAN

SEQ ID NO: 153 - DNA, 2178 bp, Sacchrophagus degradans
  Sde_3420
atgaaaatcaacactctctttacgcctttgcgtactgtgggtgctgcagttgcgatagctttatcgcctgtagcctttgcagacgtaacgtgcgaagtaacgaactttaaccagtggaatagtggctaccaagccgatgttcgtgttacaaacagcggtagcgctgttagtggctggaccgtaaatttaaattttgcctcagccccgcaaatgacaaatggctggaacgcagctttgagtactagcggcaatacaattagtgcatctaatattagttggaatggcaatttgggtaatggtcagtccaccagctttggttttcagggcaattcaaatggtaacttggcaacgccaacgtgtgtaggtagcggtacggggtcttctagcagctcttcatccagctctacttctagcacaagctcatcatctacaagttcttctagcacgtcttctactagctctagcagttcatcctctggtggtgaatgtgtagaaatgtgtaagtggtatcaagatgcaccgcgcccattatgtaataatcaagacagtggttggggttgggaaaacaatcaaagctgtattggtcgcactacttgtaacagccaatctggcaatggtggtgtaattaatagttgcccaagttcttcaagttcttcaagttcttctagcacttcgtctaccagctcatctagtacttcaagtacttcatcgagctcaacaagtagtacttcaagcacttcatcaagttccacaagctctactagcagcagctcaacctctagcactagctcgtcgtcttcaagtggtggtggagtattccgcgtagatgctaccggtaatattactaaaaatggtgaagtactgcctgttcgttgtggtaactggtttggtctagagggccagcacgagccttcagatgcgcaaaataacccaggcggtgcgccgcttgaattatatgttggcaacatgtggtgggtagatagtggccgcactattcagcaaaccatgagcgaaattaccgcccaaggtatcaacatggttcgcttgcctattgcaccgcaaacattaaaccctaacgaccctcaaggtgtgggtgatgtgcgcaacggcggcgtgcttaaaaatcacgaatctgtgcagcaaaccaatgcacgtcaagcgttagaagacttcattgttcaagctaacgaaaatgacattcaagtgctaattgatattcactcttgtagtaactacgtgggttggcgtgcaggccgtttagatgcagagcctccttatgtggatgcaacgcgagtgggttatgactttacccgtgaagattattcttgtggcaccaatgtgggcccaggtgtaactgtgcacgagtacaacgaggaaatttggttaaacaacttgcgtgagattgctggtttatctgaatccttgggcgttgataatattatcggtatcgatatttttaacgaaccatgggattacacttgggaagagtggaaagcactttctgaaagcgcttatcaagccattagcgaagttaacccagatattctaatctttgttgagggtgttgcaggcggcacgggtgctggtgttgatgtgccacatggagacgagtcttctaaccctaactggggcgaaaacttttatcctgcgcaaactgctccgcttaatattccaaaagatcgtctagttatttcaccgcatacctatggcccatctgtatttgttcagcgtcaatttatggacccgaatgatccagagtgtgttggtttagaaggtgatgaggcggctgaagctggctgtcaaattgttatcgattatgcaaccttagcagctggttgggatgagcatttcggcttcttacgtgagcaaggctttgccatggtagtgggtgagtttggtggcaacatggattggccaaatggcacgcgccaagcagaaaaagatatgtggagccacatcacccctggaatcgacagacagtggcaagaagcgtttgttgactacatggttgagaaaaacatccaagcttgttactggtcaattaacccagagtctggcgacactggcggttggtatggtcacgagtacgaccctgtttctaacgatgcaggttgggggcgttggttagacttcgattctcgcaaaactaacttacttaaagagctttggggtatttaa

SEQ ID NO: 154 - protein, 725 aa, Sacchrophagus degradans
  Sde_3420
MKINTLFTPLRTVGAAVAIALSPVAFADVTCEVTNFNQWNSGYQADVRVTNSGSAVSGWTVNLNFASAPQMTNGWNAALSTSGNTISASNISWNGNLGNGQSTSFGFQGNSNGNLATPTCVGSGTGSSSSSSSSSTSSTSSSSTSSSSTSSTSSSSSSSGGECVEMCKWYQDAPRPLCNNQDSGWGWENNQSCIGRTTCNSQSGNGGVINSCPSSSSSSSSSSTSSTSSSSTSSTSSSSTSSTSSTSSSSTSSTSSSSTSSTSSSSSSGGGVFRVDATGNITKNGEVLPVRCGNWFGLEGQHEPSDAQNNPGGAPLELYVGNMWWVDSGRTIQQTMSEITAQGINMVRLPIAPQTLNPNDPQGVGDVRNGGVLKNHESVQQTNARQALEDFIVQANENDIQVLIDIHSCSNYVGWRAGRLDAEPPYVDATRVGYDFTREDYSCGTNVGPGVTVHEYNEEIWLNNLREIAGLSESLGVDNIIGIDIFNEPWDYTWEEWKALSESAYQAISEVNPDILIFVEGVAGGTGAGVDVPHGDESSNPNWGENFYPAQTAPLNIPKDRLVISPHTYGPSVFVQRQFMDPNDPECVGLEGDEAAEAGCQIVIDYATLAAGWDEHFGFLREQGFAMVVGEFGGNMDWPNGTRQAEKDMWSHITPGIDRQWQEAFVDYMVEKNIQACYWSINPESGDTGGWYGHEYDPVSNDAGWGRWLDFDSRKTNLLKELWGI

SEQ ID NO: 155 - protein, 891 aa, Escherichia coli strain K12
  AdhE
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 156 - DNA, 3268 bp, Artificial Sequence
  deltaPaAly lyase and truncated autotransporter construct
aactgcaaaaatagtttgacaccctagccgataggctttaagatgtacccagttcgatgagagcgataactcacacagatatcattaaagaggagaaacccatggatgaaacgacatctgaatacctgctacaggctggtatggaatcacatgacgggcgctttcgtggttgcctccgaactggcccgcgcacggggtaaacgtggcggtgtggcggttgcactgtctcttgccgcagtcacgtcactcccggtgctggctggatccgataactcaaatggttcaacaattcctagcagcataaccagtggtagcatttttgatttagaaggggataacccaaatcctctcgttgacgatagcaccttagtgtttgtgccgttaggggcacaacatattacgcctaatggtaatggctggcgtcatgagtataaggttaaagagagcttacgcgttgctatgactcaaacctatgaagtgttcgaagctacggtaaaagttgagatgtctgatggcggaaaaacaattatatcgcagcaccatgctagtgataccggcactatatctaaagtgtatgtgtcggatactgatgaatcgggctttaatgatagcgtagcgaacaacgggatttttgatgtgtacgtacgtttacgtaataccagcggtaatgaagaaaaatttgctttgggtacaatgaccagcggtgagacatttaacttgcgggtagttaataactacggcgatgtagaggttacggcattcggtaactcgttcggtataccggtagaggatgattcgcagtcatactttaagtttggtaactacctgcaatcgcaagacccgtacacattagataaatgtggtgaggccggaaactctaactcgtttaaaaactgttttgaggatttaggcattacagagtcaaaagtgacgatgaccaatgtgagttatacgcgtgaaactaattctagaggcggtgtactgctggccgattccggtgccgctgtcagtggtaccaataacggcgccatacttaccctttccgggaagacggtgaacaacgataccctgaccatccgtgaaggcgatgcactcctgcagggaggctctctcaccggtaacggcagcgtggaaaaatcaggaagtggcacactcactgtcagcaacaccacactcacccagaaagccgtcaacctgaatgaaggcacgctgacgctgaacgacagtaccgtcaccacggatgtcattgctcagcgcggtacagccctgaagctgaccggcagcactgtgctgaacggtgccattgaccccacgaatgtcactctcgcctccggtgccacctggaatatccccgataacgccacggtgcagtcggtggtggatgacctcagccatgccggacagattcatttcacctccacccgcacagggaagttcgtaccggcaaccctgaaagtgaaaaacctgaacggacagaatggcaccatcagcctgcgtgtacgcccggatatggcacagaacaatgctgacagactggtcattgacggcggcagggcaaccggaaaaaccatcctgaacctggtgaacgccggcaacagtgcgtcggggctggcgaccagcggtaagggtattcaggtggtggaagccattaacggtgccaccacggaggaaggggcctttgtccaggggaacaggctgcaggccggtgcctttaactactccctcaaccgggacagtgatgagagctggtatctgcgcagtgaaaatgcttatcgtgcagaagtccccctgtatgcctccatgctgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcgtccgtctggagggtgacctgatgagaacagaggttgccggtatgtctgtgaccgcgggggtatatggtgctgctggccattcttccgttgatgttaaggatgatgacggctcccgtgccggcacggtccgggatgatgccggcagcctgggcggatacctgaatctggtacacacgtcctccggcctgtgggctgacattgtggcacagggaacccgccacagcatgaaagcgtcatcggacaataacgacttccgcgcccggggctggggctggctgggctcactggaaaccggtctgcccttcagtatcactgacaacctgatgctggagccacaactgcagtatacctggcagggactttccctggatgacggtaaggacaacgccggttatgtgaagttcgggcatggcagtgcacaacatgtgcgtgccggtttccgtctgggcagccacaacgatatgacctttggcgaaggcacctcatcccgtgcccccctgcgtgacagtgcaaaacacagtgtgagtgaattaccggtgaactggtgggtacagccttctgttatccgcaccttcagctcccggggagatatgcgtgtggggacttccactgcaggcagcgggatgacgttctctccctcacagaatggcacatcactggacctgcaggccggactggaagcccgtgtccgggaaaatatcaccctgggcgttcaggccggttatgcccacagcgtcagcggcagcagcgctgaagggtataacggtcaggccacactgaatgtgaccttctgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctcgag

SEQ ID NO: 157 - DNA, 25 bp, Artificial Sequence
  Primer
aacccgtatgttggctttgaaatgg

SEQ ID NO: 158 - DNA, 20 bp, Artificial Sequence
  Primer
gtccggacgagtgccgatgg

SEQ ID NO: 159 - DNA, 45 bp, Artificial Sequence
  Primer
atgaaagctactaaactggtactgggcgcggtaatcctgggttct

SEQ ID NO: 160 - DNA, 40 bp, Artificial Sequence
  Primer
tgctggagcaacctgccagcagagtagaacccaggattac

SEQ ID NO: 161 - DNA, 42 bp, Artificial Sequence
  Primer
actctgctggcaggttgctccagcaacgctaaaatcgatcag

SEQ ID NO: 162 - DNA, 44 bp, Artificial Sequence
  Primer
acccatttcaaagccaacatacgggttctgatcgattttagcgt

SEQ ID NO: 163 - DNA, 43 bp, Artificial Sequence
  Primer
cacacaccatggatgaaaaaagaactgagctttcatgaaaagc

SEQ ID NO: 164 - DNA, 50 bp, Artificial Sequence
  Primer
ccctttggatcctttagattttagtttgtcactatgatcaatatcaaacg

SEQ ID NO: 165 - DNA, 34 bp, Artificial Sequence
  Primer
gggcccccatggatgactctcgacaaggcgttgg

SEQ ID NO: 166 - DNA, 31 bp, Artificial Sequence
  Primer
tttaaaggatccggtctgcaaattctgcggc

SEQ ID NO: 167 - DNA, 37 bp, Artificial Sequence
  Primer
agagagtctagaagcgacggaaaggcattcagtatcg

SEQ ID NO: 168 - DNA, 36 bp, Artificial Sequence
  Primer
aaagggccatggatgaaacgacatctgaatacctgc

SEQ ID NO: 169 - DNA, 35 bp, Artificial Sequence
  Primer
tttgggaagcttcagaaggtcacattcagtgtggc

SEQ ID NO: 170 - DNA, 29 bp, Artificial Sequence
  Primer
tgtgtgggatccagccagcaccgggagtg

SEQ ID NO: 171 - DNA, 38 bp, Artificial Sequence
  Primer
atatatccatggatgaaacaaagcactattgcactggc

SEQ ID NO: 172 - DNA, 32 bp, Artificial Sequence
  Primer
ccctttaagctttcagaagtccaggctcagcg

SEQ ID NO: 173 - DNA, 50 bp, Artificial Sequence
  Primer
atgcctctggcttgtctggctactactcgtgttggtgctgctcgtgagaa

SEQ ID NO: 174 - DNA, 50 bp, Artificial Sequence
  Primer
aagcggcgactcttctatgttcgacatcccgtttccgggtcacggtcgtc

SEQ ID NO: 175 - DNA, 50 bp, Artificial Sequence
  Primer
gtctggccgttgcggcgctggccttcgccggttgcgcgttcgcaggttct

SEQ ID NO: 176 - DNA, 50 bp, Artificial Sequence
  Primer
ctgcaagctcacccgttcgaccaagcagttgtgaaagatccgactgcgtc

SEQ ID NO: 177 - DNA, 50 bp, Artificial Sequence
  Primer
ctatgttgacgttaaagcgcgtcgtactttcctgcaaagcggtcaactgg

SEQ ID NO: 178 - DNA, 50 bp, Artificial Sequence
  Primer
atgatcgcctgaaagcagcgctgccgaaggaatatgactgtaccaccgaa

SEQ ID NO: 179 - DNA, 50 bp, Artificial Sequence
  Primer
gcgacgccgaacccacagcagggtgaaatggtgatcccacgccgctatct

SEQ ID NO: 180 - DNA, 50 bp, Artificial Sequence
  Primer
gtccggtaaccacggcccggtgaatccggattacgagccggttgtcactc

SEQ ID NO: 181 - DNA, 50 bp, Artificial Sequence
  Primer
tgtatcgcgacttcgaaaaaatcagcgcgaccctgggtaacctgtacgtt

SEQ ID NO: 182 - DNA, 50 bp, Artificial Sequence
  Primer
gcgactggtaaaccagtgtacgcaacttgtctgctgaacatgctggacaa

SEQ ID NO: 183 - DNA, 50 bp, Artificial Sequence
  Primer
atgggctaaagcagacgcgctgctgaactatgacccgaaatctcagagct

SEQ ID NO: 184 - DNA, 50 bp, Artificial Sequence
  Primer
ggtatcaagtagaatggtccgcagccacggcggcctttgccctgagcact

SEQ ID NO: 185 - DNA, 50 bp, Artificial Sequence
  Primer
atgatggcagagccgaacgtggacaccgcgcagcgtgagcgtgttgtgaa

SEQ ID NO: 186 - DNA, 50 bp, Artificial Sequence
  Primer
atggctgaaccgtgtagcacgtcaccagacttcttttccgggtggcgaca

SEQ ID NO: 187 - DNA, 50 bp, Artificial Sequence
  Primer
ctagctgctgtaacaatcattcttactggcgtggtcaggaggctaccatc

SEQ ID NO: 188 - DNA, 50 bp, Artificial Sequence
  Primer
atcggcgttatttccaaggatgatgaactgttccgttggggtctgggtcg

SEQ ID NO: 189 - DNA, 50 bp, Artificial Sequence
  Primer
ttatgtacaggcgatgggtctgatcaacgaagatggttccttcgttcacg

SEQ ID NO: 190 - DNA, 50 bp, Artificial Sequence
  Primer
aaatgactcgtcacgaacagagcctgcattatcagaactatgcgatgctg

SEQ ID NO: 191 - DNA, 50 bp, Artificial Sequence
  Primer
ccgctgaccatgatcgctgagactgcctctcgtcagggtatcgatctgta

SEQ ID NO: 192 - DNA, 50 bp, Artificial Sequence
  Primer
tgcttacaaggaaaacggtcgtgatatccattctgctcgtaaattcgtat

SEQ ID NO: 193 - DNA, 50 bp, Artificial Sequence
  Primer
tcgcggccgtaaagaatccggatctgatcaagaaatacgcgagcgaaccg

SEQ ID NO: 194 - DNA, 50 bp, Artificial Sequence
  Primer
caggacacgcgcgcttttaaaccgggtcgcggcgatctgaactggatcga

SEQ ID NO: 195 - DNA, 50 bp, Artificial Sequence
  Primer
atatcagcgtgcgcgtttcggctttgcagatgagctgggctttatgaccg

SEQ ID NO: 196 - DNA, 50 bp, Artificial Sequence
  Primer
tgccaatcttcgatccgcgcaccggcggctctggcactctgctggcgtat

SEQ ID NO: 197 - DNA, 50 bp, Artificial Sequence
  Primer
aagccacagggtgcggctgctcaggcgccggtttccgctccggcggcagc

SEQ ID NO: 198 - DNA, 50 bp, Artificial Sequence
  Primer
acactcttccatcgatctgtccaaatggaaactgcagatccctgttgacc

SEQ ID NO: 199 - DNA, 50 bp, Artificial Sequence
  Primer
cgatcgatgttgctacccgcgatctgctgaagggttatcaggacaagtat

SEQ ID NO: 200 - DNA, 50 bp, Artificial Sequence
  Primer
ttctacgtggataaagatggttctctggccttctggtgcccagcatccgg

SEQ ID NO: 201 - DNA, 50 bp, Artificial Sequence
  Primer
tttcaaaaccacggcgaatactaagtatccgcgtagcgagctgcgtgaaa

SEQ ID NO: 202 - DNA, 50 bp, Artificial Sequence
  Primer
tgctggacccggataatcatgctgttaattggggctggcagggcacccac

SEQ ID NO: 203 - DNA, 50 bp, Artificial Sequence
  Primer
gaaatgaacctgcgcggtgcagttatgcacgtttccccgtccggtaaaac

SEQ ID NO: 204 - DNA, 50 bp, Artificial Sequence
  Primer
catcgtcatgcagatccacgcagttatgccggacggttccaatgcgccac

SEQ ID NO: 205 - DNA, 50 bp, Artificial Sequence
  Primer
cactggttaaaggccagttctacaaaaacacgctggacttcctggtgaaa

SEQ ID NO: 206 - DNA, 50 bp, Artificial Sequence
  Primer
aattctgcggctggtggtaaagatactcactacgtgttcgaaggcatcga

SEQ ID NO: 207 - DNA, 50 bp, Artificial Sequence
  Primer
actgggtaaaccatacgacgctcagatcaaagttgtagatggtgtcctgt

SEQ ID NO: 208 - DNA, 50 bp, Artificial Sequence
  Primer
ctatgaccgttaatggtcagactaaaactgttgacttcgtggctaaagat

SEQ ID NO: 209 - DNA, 50 bp, Artificial Sequence
  Primer
gcgggctggaaggatctgaaattctatttcaaggcaggtaactatctgca

SEQ ID NO: 210 - DNA, 50 bp, Artificial Sequence
  Primer
ggaccgccaggccgacggctccgatacctctgccctggtaaagctgtaca

SEQ ID NO: 211 - DNA, 48 bp, Artificial Sequence
  Primer
gctggaatgtttaacgtccagtttgtacagctttaccagggcagaggt

SEQ ID NO: 212 - DNA, 50 bp, Artificial Sequence
  Primer
atcggagccgtcggcctggcggtcctgcagatagttacctgccttgaaat

SEQ ID NO: 213 - DNA, 50 bp, Artificial Sequence
  Primer
agaatttcagatccttccagcccgcatctttagccacgaagtcaacagtt

SEQ ID NO: 214 - DNA, 50 bp, Artificial Sequence
  Primer
ttagtctgaccattaacggtcatagacaggacaccatctacaactttgat

SEQ ID NO: 215 - DNA, 50 bp, Artificial Sequence
  Primer
ctgagcgtcgtatggtttacccagttcgatgccttcgaacacgtagtgag

SEQ ID NO: 216 - DNA, 50 bp, Artificial Sequence
  Primer
tatctttaccaccagccgcagaatttttcaccaggaagtccagcgtgttt

SEQ ID NO: 217 - DNA, 50 bp, Artificial Sequence
  Primer
ttgtagaactggcctttaaccagtggtggcgcattggaaccgtccggcat

SEQ ID NO: 218 - DNA, 50 bp, Artificial Sequence
  Primer
aactgcgtggatctgcatgacgatggttttaccggacggggaaacgtgca

SEQ ID NO: 219 - DNA, 50 bp, Artificial Sequence
  Primer
taactgcaccgcgcaggttcatttcgtgggtgccctgccagccccaatta

SEQ ID NO: 220 - DNA, 50 bp, Artificial Sequence
  Primer
acagcatgattatccgggtccagcatttcacgcagctcgctacgcggata

SEQ ID NO: 221 - DNA, 50 bp, Artificial Sequence
  Primer
cttagtattcgccgtggttttgaaaccggatgctgggcaccagaaggcca

SEQ ID NO: 222 - DNA, 50 bp, Artificial Sequence
  Primer
gagaaccatctttatccacgtagaaatacttgtcctgataacccttcagc

SEQ ID NO: 223 - DNA, 50 bp, Artificial Sequence
  Primer
agatcgcgggtagcaacatcgatcgggtcaacagggatctgcagtttcca

SEQ ID NO: 224 - DNA, 50 bp, Artificial Sequence
  Primer
tttggacagatcgatggaagagtgtgctgccgccggagcggaaaccggcg

SEQ ID NO: 225 - DNA, 50 bp, Artificial Sequence
  Primer
cctgagcagccgcaccctgtggcttatacgccagcagagtgccagagccg

SEQ ID NO: 226 - DNA, 50 bp, Artificial Sequence
  Primer
ccggtgcgcggatcgaagattggcacggtcataaagcccagctcatctgc

SEQ ID NO: 227 - DNA, 50 bp, Artificial Sequence
  Primer
aaagccgaaacgcgcacgctgatattcgatccagttcagatcgccgcgac

SEQ ID NO: 228 - DNA, 50 bp, Artificial Sequence
  Primer
ccggtttaaaagcgcgcgtgtcctgcggttcgctcgcgtatttcttgatc

SEQ ID NO: 229 - DNA, 50 bp, Artificial Sequence
  Primer
agatccggattctttacggccgcgaatacgaatttacgagcagaatggat

SEQ ID NO: 230 - DNA, 50 bp, Artificial Sequence
  Primer
atcacgaccgttttccttgtaagcatacagatcgataccctgacgagagg

SEQ ID NO: 231 - DNA, 50 bp, Artificial Sequence
  Primer
cagtctcagcgatcatggtcagcggcagcatcgcatagttctgataatgc

SEQ ID NO: 232 - DNA, 50 bp, Artificial Sequence
  Primer
aggctctgttcgtgacgagtcatttcgtgaacgaaggaaccatcttcgtt

SEQ ID NO: 233 - DNA, 50 bp, Artificial Sequence
  Primer
gatcagacccatcgcctgtacataacgacccagaccccaacggaacagtt

SEQ ID NO: 234 - DNA, 50 bp, Artificial Sequence
  Primer
catcatccttggaaataacgccgatgatggtagcctcctgaccacgccag

SEQ ID NO: 235 - DNA, 50 bp, Artificial Sequence
  Primer
taagaatgattgttacagcagctagtgtcgccacccggaaaagaagtctg

SEQ ID NO: 236 - DNA, 50 bp, Artificial Sequence
  Primer
gtgacgtgctacacggttcagccatttcacaacacgctcacgctgcgcgg

SEQ ID NO: 237 - DNA, 50 bp, Artificial Sequence
  Primer
tgtccacgttcggctctgccatcatagtgctcagggcaaaggccgccgtg

SEQ ID NO: 238 - DNA, 50 bp, Artificial Sequence
  Primer
gctgcggaccattctacttgataccagctctgagatttcgggtcatagtt

SEQ ID NO: 239 - DNA, 50 bp, Artificial Sequence
  Primer
cagcagcgcgtctgctttagcccatttgtccagcatgttcagcagacaag

SEQ ID NO: 240 - DNA, 50 bp, Artificial Sequence
  Primer
ttgcgtacactggtttaccagtcgcaacgtacaggttacccagggtcgcg

SEQ ID NO: 241 - DNA, 50 bp, Artificial Sequence
  Primer
ctgattttttcgaagtcgcgatacagagtgacaaccggctcgtaatccgg

SEQ ID NO: 242 - DNA, 50 bp, Artificial Sequence
  Primer
attcaccgggccgtggttaccggacagatagcggcgtgggatcaccattt

SEQ ID NO: 243 - DNA, 50 bp, Artificial Sequence
  Primer
caccctgctgtgggttcggcgtcgcttcggtggtacagtcatattccttc

SEQ ID NO: 244 - DNA, 50 bp, Artificial Sequence
  Primer
ggcagcgctgctttcaggcgatcatccagttgaccgctttgcaggaaagt

SEQ ID NO: 245 - DNA, 50 bp, Artificial Sequence
  Primer
acgacgcgctttaacgtcaacataggacgcagtcggatctttcacaactg

SEQ ID NO: 246 - DNA, 50 bp, Artificial Sequence
  Primer
cttggtcgaacgggtgagcttgcagagaacctgcgaacgcgcaaccggcg

SEQ ID NO: 247 - DNA, 50 bp, Artificial Sequence
  Primer
aaggccagcgccgcaacggccagacgacgaccgtgacccggaaacgggat

SEQ ID NO: 248 - DNA, 50 bp, Artificial Sequence
  Primer
gtcgaacatagaagagtcgccgcttttctcacgagcagcaccaacacgag

SEQ ID NO: 249 - DNA, 32 bp, Artificial Sequence
  Primer
gtgtgtggatccatgcctctggcttgtctggc

SEQ ID NO: 250 - DNA, 41 bp, Artificial Sequence
  Primer
gggtttaagcttagctggaatgtttaacgtccagtttgtac

SEQ ID NO: 251 - DNA, 18 bp, Artificial Sequence
  Primer
gcgtgtcctgcggttcgc

SEQ ID NO: 252 - DNA, 23 bp, Artificial Sequence
  Primer
gctgagactgcctctcgtcaggg

SEQ ID NO: 253 - DNA, 29 bp, Artificial Sequence
  Primer
cgcggatccgataactcaaatggttcaac

SEQ ID NO: 254 - DNA, 34 bp, Artificial Sequence
  Primer
gtataaggttaaagagagcttacgcgttgctatg

SEQ ID NO: 255 - DNA, 34 bp, Artificial Sequence
  Primer
catagcaacgcgtaagctctctttaaccttatac

SEQ ID NO: 256 - DNA, 32 bp, Artificial Sequence
  Primer
cccaagcttttaattagtttcacgcgtataac

SEQ ID NO: 257 - DNA, 29 bp, Artificial Sequence
  Primer
cgcggatccgataactcaaatggttcaac

SEQ ID NO: 258 - DNA, 32 bp, Artificial Sequence; identical to SEQ ID NO: 256
  Primer

SEQ ID NO: 259 - DNA, 25 bp, Artificial Sequence
  Primer
cgggatccatgttcaggtttaaagg

SEQ ID NO: 260 - DNA, 50 bp, Artificial Sequence
  Primer
aataaggataatgattaaccataaaaaactgtttatttacagcgcaattg

SEQ ID NO: 261 - DNA, 50 bp, Artificial Sequence
  Primer
cgacaagttcagcgctatctcatgctgcaacaattaataatgcaggcttt

SEQ ID NO: 262 - DNA, 50 bp, Artificial Sequence
  Primer
gaaagtggctttagtaactggaacgaaaccgacccagccgctatttcttc

SEQ ID NO: 263 - DNA, 50 bp, Artificial Sequence
  Primer
agatgcttacagtggctcaaaatcgttaaaaattcagggcagtccagcac

SEQ ID NO: 264 - DNA, 50 bp, Artificial Sequence
  Primer
gggtttatcaagtggtagatatacagcctaacactgaatacaccctaagt

SEQ ID NO: 265 - DNA, 50 bp, Artificial Sequence
  Primer
gcttatgtgctgggtaaagggcaaattggtgtaaacgatttaaatggttt

SEQ ID NO: 266 - DNA, 50 bp, Artificial Sequence
  Primer
atttaaaaaccaaacctttaatgtttcttcgtggactaaagtaacaaaaa

SEQ ID NO: 267 - DNA, 50 bp, Artificial Sequence
  Primer
catttacctcagcaaacaccaattcacttcaggtttttgctaaacattac

SEQ ID NO: 268 - DNA, 50 bp, Artificial Sequence
  Primer
gacaacaccagcgatgtaaggtttgataatttttccttgattgagggcag

SEQ ID NO: 269 - DNA, 50 bp, Artificial Sequence
  Primer
cggtagtaatgatggtggctcagatggcggcagcgataactcaaatggtt

SEQ ID NO: 270 - DNA, 50 bp, Artificial Sequence
  Primer
caacaattcctagcagcataaccagtggtagcatttttgatttagaaggg

SEQ ID NO: 271 - DNA, 50 bp, Artificial Sequence
  Primer
gataacccaaatcctctcgttgacgatagcaccttagtgtttgtgccgtt

SEQ ID NO: 272 - DNA, 50 bp, Artificial Sequence
  Primer
aggggcacaacatattacgcctaatggtaatggctggcgtcatgagtata

SEQ ID NO: 273 - DNA, 50 bp, Artificial Sequence
  Primer
aggttaaagaaagtttacgcgttgctatgactcaaacctatgaagtgttc

SEQ ID NO: 274 - DNA, 50 bp, Artificial Sequence
  Primer
gaagctacggtaaaagttgagatgtctgatggcggaaaaacaattatatc

SEQ ID NO: 275 - DNA, 50 bp, Artificial Sequence
  Primer
gcagcaccatgctagtgataccggcactatatctaaagtgtatgtgtcgg

SEQ ID NO: 276 - DNA, 50 bp, Artificial Sequence
  Primer
atactgatgaatcgggctttaatgatagcgtagcgaacaacgggattttt

SEQ ID NO: 277 - DNA, 50 bp, Artificial Sequence
  Primer
gatgtgtacgtacgtttacgtaataccagcggtaatgaagaaaaatttgc

SEQ ID NO: 278 - DNA, 50 bp, Artificial Sequence
  Primer
tttgggtacaatgaccagcggtgagacatttaacttgcgggtagttaata

SEQ ID NO: 279 - DNA, 50 bp, Artificial Sequence
  Primer
actacggcgatgtagaggttacggcattcggtaactcgttcggtataccg

SEQ ID NO: 280 - DNA, 50 bp, Artificial Sequence
  Primer
gtagaggatgattcgcagtcatactttaagtttggtaactacctgcaatc

SEQ ID NO: 281 - DNA, 50 bp, Artificial Sequence
  Primer
gcaagacccgtacacattagataaatgtggtgaggccggaaactctaact

SEQ ID NO: 282 - DNA, 50 bp, Artificial Sequence
  Primer
cgtttaaaaactgttttgaggatttaggcattacagagtcaaaagtgacg

SEQ ID NO: 283 - DNA, 50 bp, Artificial Sequence
  Primer
atgaccaatgtgagttatacgcgtgaaactaattaagcttggtctagagc

SEQ ID NO: 284 - DNA, 50 bp, Artificial Sequence
  Primer
tttatggttaatcattatccttattcctttaaacctgaacatggatcccg

SEQ ID NO: 285 - DNA, 50 bp, Artificial Sequence
  Primer
gcatgagatagcgctgaacttgtcgcaattgcgctgtaaataaacagttt

SEQ ID NO: 286 - DNA, 50 bp, Artificial Sequence
  Primer
cgttccagttactaaagccactttcaaagcctgcattattaattgttgca

SEQ ID NO: 287 - DNA, 50 bp, Artificial Sequence
  Primer
cgattttgagccactgtaagcatctgaagaaatagcggctgggtcggttt

SEQ ID NO: 288 - DNA, 50 bp, Artificial Sequence
  Primer
tgtatatctaccacttgataaacccgtgctggactgccctgaatttttaa

SEQ ID NO: 289 - DNA, 50 bp, Artificial Sequence
  Primer
tttgccctttacccagcacataagcacttagggtgtattcagtgttaggc

SEQ ID NO: 290 - DNA, 50 bp, Artificial Sequence
  Primer
aacattaaaggtttggtttttaaataaaccatttaaatcgtttacaccaa

SEQ ID NO: 291 - DNA, 50 bp, Artificial Sequence
  Primer
gaattggtgtttgctgaggtaaatgtttttgttactttagtccacgaaga

SEQ ID NO: 292 - DNA, 50 bp, Artificial Sequence
  Primer
caaaccttacatcgctggtgttgtcgtaatgtttagcaaaaacctgaagt

SEQ ID NO: 293 - DNA, 50 bp, Artificial Sequence
  Primer
atctgagccaccatcattactaccgctgccctcaatcaaggaaaaattat

SEQ ID NO: 294 - DNA, 50 bp, Artificial Sequence
  Primer
ctggttatgctgctaggaattgttgaaccatttgagttatcgctgccgcc

SEQ ID NO: 295 - DNA, 50 bp, Artificial Sequence
  Primer
cgtcaacgagaggatttgggttatccccttctaaatcaaaaatgctacca

SEQ ID NO: 296 - DNA, 50 bp, Artificial Sequence
  Primer
attaggcgtaatatgttgtgcccctaacggcacaaacactaaggtgctat

SEQ ID NO: 297 - DNA, 50 bp, Artificial Sequence
  Primer
gcaacgcgtaaactttctttaaccttatactcatgacgccagccattacc

SEQ ID NO: 298 - DNA, 50 bp, Artificial Sequence
  Primer
acatctcaacttttaccgtagcttcgaacacttcataggtttgagtcata

SEQ ID NO: 299 - DNA, 50 bp, Artificial Sequence
  Primer
gccggtatcactagcatggtgctgcgatataattgtttttccgccatcag

SEQ ID NO: 300 - DNA, 50 bp, Artificial Sequence
  Primer
tcattaaagcccgattcatcagtatccgacacatacactttagatatagt

SEQ ID NO: 301 - DNA, 50 bp, Artificial Sequence
  Primer
tattacgtaaacgtacgtacacatcaaaaatcccgttgttcgctacgcta

SEQ ID NO: 302 - DNA, 50 bp, Artificial Sequence
  Primer
ctcaccgctggtcattgtacccaaagcaaatttttcttcattaccgctgg

SEQ ID NO: 303 - DNA, 50 bp, Artificial Sequence
  Primer
gccgtaacctctacatcgccgtagttattaactacccgcaagttaaatgt

SEQ ID NO: 304 - DNA, 50 bp, Artificial Sequence
  Primer
agtatgactgcgaatcatcctctaccggtataccgaacgagttaccgaat

SEQ ID NO: 305 - DNA, 50 bp, Artificial Sequence
  Primer
tttatctaatgtgtacgggtcttgcgattgcaggtagttaccaaacttaa

SEQ ID NO: 306 - DNA, 50 bp, Artificial Sequence
  Primer
aaatcctcaaaacagtttttaaacgagttagagtttccggcctcaccaca

SEQ ID NO: 307 - DNA, 50 bp, Artificial Sequence
  Primer
cacgcgtataactcacattggtcatcgtcacttttgactctgtaatgcct

SEQ ID NO: 308 - DNA, 25 bp, Artificial Sequence
  Primer
gctctagaccaagcttaattagttt

SEQ ID NO: 309 - DNA, 25 bp, Artificial Sequence
  Primer
cgggatccatgaatatcgacaaagc

SEQ ID NO: 310 - DNA, 50 bp, Artificial Sequence
  Primer
gttggtactgcgtacctgtgcaaataacatggccgatcattgcggcctta

SEQ ID NO: 311 - DNA, 50 bp, Artificial Sequence
  Primer
tatggcccgcctccggcacggtggaatccaaatactggcagtcaaccagg

SEQ ID NO: 312 - DNA, 50 bp, Artificial Sequence
  Primer
cggcatgagaatggtctggtcggtttactgtggggcgctggaaccagcgc

SEQ ID NO: 313 - DNA, 50 bp, Artificial Sequence
  Primer
ttttctaagcgtgcatgccgatgcgcgatggaaagtctgtgaagtcgccg

SEQ ID NO: 314 - DNA, 50 bp, Artificial Sequence
  Primer
ttgcagacatcatcggtctggaagagccggggatggtcaagtttccgcgg

SEQ ID NO: 315 - DNA, 50 bp, Artificial Sequence
  Primer
gccgaggtggttcatgtcggcgacaggatcagcgcatcacacttcatttc

SEQ ID NO: 316 - DNA, 50 bp, Artificial Sequence
  Primer
ggcacgtcaggccgaccctgcatcaacgccaacgccaacgccaacgccaa

SEQ ID NO: 317 - DNA, 50 bp, Artificial Sequence
  Primer
tggccacgcccacgcctgcggcagcaaatatcgcgttaccggtggtagaa

SEQ ID NO: 318 - DNA, 50 bp, Artificial Sequence
  Primer
cagcccagtcatgaagtgttcgatgtggcgttggtcagcgcagctgcccc

SEQ ID NO: 319 - DNA, 50 bp, Artificial Sequence
  Primer
ctcagtaaacaccctgccggtgacgacgccgcagaatttgcagaccgcta

SEQ ID NO: 320 - DNA, 50 bp, Artificial Sequence
  Primer
cttacggcagcacgttgagtggcgacaacaacagccggctcattgccggt

SEQ ID NO: 321 - DNA, 50 bp, Artificial Sequence
  Primer
tatggcagtaacgagaccgctggcaaccacagtgatctgattgccggtac

SEQ ID NO: 322 - DNA, 50 bp, Artificial Sequence
  Primer
aggcgggcatgactgcacgctgatggcgggagaccaaagcagattgaccg

SEQ ID NO: 323 - DNA, 50 bp, Artificial Sequence
  Primer
caggaaagaacagtatcttgacggcaggcgcgcgtagcaaacttattggc

SEQ ID NO: 324 - DNA, 50 bp, Artificial Sequence
  Primer
agtgaaggctcgacgctctcggctggagaagactcaacgcttattttcag

SEQ ID NO: 325 - DNA, 50 bp, Artificial Sequence
  Primer
gctctgggacgggaaaaggtacaggcaactggttgccagaacgggtgaga

SEQ ID NO: 326 - DNA, 50 bp, Artificial Sequence
  Primer
acggtgttgaagccgacataccgtattacgtgaacgaagatgacgatatt

SEQ ID NO: 327 - DNA, 50 bp, Artificial Sequence
  Primer
gtcgataaacccgacgaggacgatgactggatagaggtcgagtctagagc

SEQ ID NO: 328 - DNA, 50 bp, Artificial Sequence
  Primer
atttgcacaggtacgcagtaccaacgctttgtcgatattcatggatcccg

SEQ ID NO: 329 - DNA, 50 bp, Artificial Sequence
  Primer
tccaccgtgccggaggcgggccatataaggccgcaatgatcggccatgtt

SEQ ID NO: 330 - DNA, 50 bp, Artificial Sequence
  Primer
aaccgaccagaccattctcatgccgcctggttgactgccagtatttggat

SEQ ID NO: 331 - DNA, 50 bp, Artificial Sequence
  Primer
cgcatcggcatgcacgcttagaaaagcgctggttccagcgccccacagta

SEQ ID NO: 332 - DNA, 50 bp, Artificial Sequence
  Primer
tcttccagaccgatgatgtctgcaacggcgacttcacagactttccatcg

SEQ ID NO: 333 - DNA, 50 bp, Artificial Sequence
  Primer
tgtcgccgacatgaaccacctcggcccgcggaaacttgaccatccccggc

SEQ ID NO: 334 - DNA, 50 bp, Artificial Sequence
  Primer
tgatgcagggtcggcctgacgtgccgaaatgaagtgtgatgcgctgatcc

SEQ ID NO: 335 - DNA, 50 bp, Artificial Sequence
  Primer
gctgccgcaggcgtgggcgtggccattggcgttggcgttggcgttggcgt

SEQ ID NO: 336 - DNA, 50 bp, Artificial Sequence
  Primer
catcgaacacttcatgactgggctgttctaccaccggtaacgcgatattt

SEQ ID NO: 337 - DNA, 50 bp, Artificial Sequence
  Primer
cgtcaccggcagggtgtttactgagggggcagctgcgctgaccaacgcca

SEQ ID NO: 338 - DNA, 50 bp, Artificial Sequence
  Primer
tcgccactcaacgtgctgccgtaagtagcggtctgcaaattctgcggcgt

SEQ ID NO: 339 - DNA, 50 bp, Artificial Sequence
  Primer
tgccagcggtctcgttactgccataaccggcaatgagccggctgttgttg

SEQ ID NO: 340 - DNA, 50 bp, Artificial Sequence
  Primer
catcagcgtgcagtcatgcccgcctgtaccggcaatcagatcactgtggt

SEQ ID NO: 341 - DNA, 50 bp, Artificial Sequence
  Primer
gccgtcaagatactgttctttcctgcggtcaatctgctttggtctcccgc

SEQ ID NO: 342 - DNA, 50 bp, Artificial Sequence
  Primer
cagccgagagcgtcgagccttcactgccaataagtttgctacgcgcgcct

SEQ ID NO: 343 - DNA, 50 bp, Artificial Sequence
  Primer
cctgtaccttttcccgtcccagagcctgaaaataagcgttgagtcttctc

SEQ ID NO: 344 - DNA, 50 bp, Artificial Sequence
  Primer
tacggtatgtcggcttcaacaccgttctcacccgttctggcaaccagttg

SEQ ID NO: 345 - DNA, 50 bp, Artificial Sequence
  Primer
catcgtcctcgtcgggtttatcgacaatatcgtcatcttcgttcacgtaa

SEQ ID NO: 346 - DNA, 25 bp, Artificial Sequence
  Primer
gctctagactcgacctctatccagt

SEQ ID NO: 347 - DNA, 29 bp, Artificial Sequence
  Primer
gtgtgtggatcccctctggcttgtctggc

SEQ ID NO: 348 - DNA, 31 bp, Artificial Sequence
  Primer
gtgtgtggatcccacccgttcgaccaagcag

SEQ ID NO: 349 - DNA, 28 bp, Artificial Sequence
  Primer
gggtttggatccgctccggcggcagcac

SEQ ID NO: 350 - DNA, 31 bp, Artificial Sequence; identical to SEQ ID NO: 348
  Primer

SEQ ID NO: 351 - DNA, 41 bp, Artificial Sequence; identical to SEQ ID NO: 250
  Primer

SEQ ID NO: 352 - DNA, 35 bp, Artificial Sequence
  Primer
gggtttaagcttatggcttatacgccagcagagtg

SEQ ID NO: 353 - DNA, 29 bp, Artificial Sequence
  Primer
cgcggatccgataactcaaatggttcaac

SEQ ID NO: 354 - DNA, 42 bp, Artificial Sequence
  Primer
gggtttaagcttaattagtttcacgcgtataactcacattgg

SEQ ID NO: 355 - DNA, 40 bp, Artificial Sequence
  Primer
tttgggtctagagctggaatgtttaacgtccagtttgtac

SEQ ID NO: 356 - DNA, 34 bp, Artificial Sequence
  Primer
tttgggtctagatggcttatacgccagcagagtg

SEQ ID NO: 357 - DNA, 41 bp, Artificial Sequence
  Primer
tttgggtctagaattagtttcacgcgtataactcacattgg

SEQ ID NO: 358 - DNA, 55 bp, Artificial Sequence
  Primer
ggaaatttgcatgcgaattcttttaaaaaattcatttgctaaacgcttcaaattc

SEQ ID NO: 359 - DNA, 51 bp, Artificial Sequence
  Primer
caatttatgaagtatattatacgagaatttgaagcgtttagcaaatgaatt

SEQ ID NO: 360 - DNA, 49 bp, Artificial Sequence
  Primer
tcgtataatatacttcataaattgataaacaaaaatcacacagatatca

SEQ ID NO: 361 - DNA, 51 bp, Artificial Sequence
  Primer
atatatccatggtttctcctctttaatgatatctgtgtgatttttgtttat

SEQ ID NO: 362 - DNA, 54 bp, Artificial Sequence
  Primer
ggaaatttgcatgcgaattcaactgcaaaaatagtttgacaccctagccgatag

SEQ ID NO: 363 - DNA, 51 bp, Artificial Sequence
  Primer
gaactgggtacatcttaaagcctatcggctagggtgtcaaactatttttgc

SEQ ID NO: 364 - DNA, 50 bp, Artificial Sequence
  Primer
gctttaagatgtacccagttcgatgagagcgataactcacacagatatca

SEQ ID NO: 365 - DNA, 55 bp, Artificial Sequence
  Primer
atatatccatggtttctcctctttaatgatatctgtgtgagttatcgctctcatc

SEQ ID NO: 366 - DNA, 50 bp, Artificial Sequence
  Primer
ggaaatttgcatgcgaattcaagaatcataaaaaatttatttgctttcag

SEQ ID NO: 367 - DNA, 49 bp, Artificial Sequence
  Primer
gaatctattatacagaaaaattttcctgaaagcaaataaattttttatg

SEQ ID NO: 368 - DNA, 51 bp, Artificial Sequence
  Primer
gaaaatttttctgtataatagattcataaatttgagagaggagtttcacac

SEQ ID NO: 369 - DNA, 52 bp, Artificial Sequence
  Primer
atatatccatggtttctcctctttaatgatatctgtgtgaaactcctctctc

SEQ ID NO: 370 - DNA, 54 bp, Artificial Sequence
  Primer
ggaaatttgcatgcgaattcttatctctggcggtgttgacataaataccactgg

SEQ ID NO: 371 - DNA, 53 bp, Artificial Sequence
  Primer
cgtcctgctgatgtgctcagtatcaccgccagtggtatttatgtcaacaccgc

SEQ ID NO: 372 - DNA, 49 bp, Artificial Sequence
  Primer
cggtgatactgagcacatcagcaggacgcactgacctcacacagatatc

SEQ ID NO: 373 - DNA, 45 bp, Artificial Sequence
  Primer
atatatccatggtttctcctctttaatgatatctgtgtgaggtca

SEQ ID NO: 374 - DNA, 53 bp, Artificial Sequence
  Primer
ggaaatttgcatgcgaattcttatcaaaaagagtattgacttaaagtctaacc

SEQ ID NO: 375 - DNA, 51 bp, Artificial Sequence
  Primer
cgatggctgtaagtatcctataggttagactttaagtcaatactctttttg

SEQ ID NO: 376 - DNA, 50 bp, Artificial Sequence
  Primer
tataggatacttacagccatcgagagggacacggcgatcacacagatatc

SEQ ID NO: 377 - DNA, 55 bp, Artificial Sequence
  Primer
atatatccatggtttctcctctttaatgatatctgtgtgatcgccgtgtccctct

SEQ ID NO: 378 - DNA, 49 bp, Artificial Sequence
  Primer
ggaaatttgcatgcgaattcatcaaaaaaatattgacaacataaaaaac

SEQ ID NO: 379 - DNA, 50 bp, Artificial Sequence
  Primer
tgtagcgttacaagtataacacaaagttttttatgttgtcaatatttttt

SEQ ID NO: 380 - DNA, 45 bp, Artificial Sequence
  Primer
tttgtgttatacttgtaacgctacatggagattaactcaatctag

SEQ ID NO: 381 - DNA, 38 bp, Artificial Sequence
  Primer
atatatccatggtaccctctagattgagttaatctcca

SEQ ID NO: 382 - DNA, 51 bp, Artificial Sequence
  Primer
cccacctgaccccatgccgaactccatggaattcgagctcggtaccctttg

SEQ ID NO: 383 - DNA, 41 bp, Artificial Sequence
  Primer
tttaaaccatggttctccatatattcaaaacactatgtctg

SEQ ID NO: 384 - DNA, 40 bp, Artificial Sequence
  Primer
ggaaatttgcatgcgaattccacggaacaacggcaaacac

SEQ ID NO: 385 - DNA, 58 bp, Artificial Sequence
  Primer
atatatccatggtttctcctctttaatgatatctgtgtgagctttttctcagcggcgc

SEQ ID NO: 386 - DNA, 53 bp, Artificial Sequence
  Primer
tttaaaggatccctcgagatatgcatgccggaagcataaagtgtaaagcctgg

SEQ ID NO: 387 - DNA, 27 bp, Artificial Sequence
  Primer
atatatggatccccgggtaccgagctc

SEQ ID NO: 388 - DNA, 44 bp, Artificial Sequence
  Primer
gcgcgcccatgcgagagactcgagggttattgtctcatgagcgg

SEQ ID NO: 389 - DNA, 76 bp, Artificial Sequence
  Primer
aagtttctgtataattactttataaattgatgagaaggaaatcacacagatatcattaaagaggagaaacccatgg

SEQ ID NO: 390 - DNA, 76 bp, Artificial Sequence; identical to SEQ ID NO: 389
  Primer

SEQ ID NO: 391 - DNA, 64 bp, Artificial Sequence
  Primer
gcaatcggtaaaatatcgatttaggcagttcacacagatatcattaaagaggagaaacccatgg

SEQ ID NO: 392 - DNA, 70 bp, Artificial Sequence
  Primer
tgggctattgtcaacaattttttagtagtctgagtgaattcgccctatagtgagtcgtattacaattcac

SEQ ID NO: 393 - DNA, 71 bp, Artificial Sequence
  Primer
caatactattataatattgttattaaagaggagaaattaacatgaaacgacatctgaatacctgctacagg

SEQ ID NO: 394 - DNA, 71 bp, Artificial Sequence; identical to SEQ ID NO: 393
  Primer

SEQ ID NO: 395 - DNA, 76 bp, Artificial Sequence
  Primer
aatttagaatatactgttagtaaacctaatggatcgacctttcacacagatatcattaaagaggagaaacccatgg

SEQ ID NO: 396 - DNA, 76 bp, Artificial Sequence; identical to SEQ ID NO: 395
  Primer

SEQ ID NO: 397 - DNA, 34 bp, Artificial Sequence
  Primer
aaatctagagctgacatcgttgtgcacccgggag

SEQ ID NO: 398 - DNA, 34 bp, Artificial Sequence
  Primer
aaatctagatatgggccggataacgaggccaata

SEQ ID NO: 399 - DNA, 33 bp, Artificial Sequence
  Primer
aaatctagacagagagtgaaccccggtggaagt

SEQ ID NO: 400 - DNA, 33 bp, Artificial Sequence
  Primer
aaatctagactgaatggtggcgaacagtggatg

SEQ ID NO: 401 - DNA, 34 bp, Artificial Sequence
  Primer
aaatctagaacagtggcaacggataccgttgtta

SEQ ID NO: 402 - DNA, 37 bp, Artificial Sequence
  Primer
aaatctagagccgtacgcacaaccatcaataaaaacg

SEQ ID NO: 403 - DNA, 34 bp, Artificial Sequence
  Primer
aaatctagacagactgtacatggtcacgcactgg

SEQ ID NO: 404 - DNA, 34 bp, Artificial Sequence
  Primer
aaatctagaaacagtgacggctggcagattgtca

SEQ ID NO: 405 - DNA, 34 bp, Artificial Sequence
  Primer
aaatctagaggtacagccacgaatgtcaccctga

SEQ ID NO: 406 - DNA, 43 bp, Artificial Sequence
  Primer
aaatctagattctctgttgtggagggtaaagctgataatgtcg

SEQ ID NO: 407 - DNA, 34 bp, Artificial Sequence
  Primer
aaatctagagccaccaccgtatccatgggaaatg

SEQ ID NO: 408 - DNA, 32 bp, Artificial Sequence
  Primer
aaatctagaggcggtgtactgctggccgattc

SEQ ID NO: 409 - DNA, 81 bp, Artificial Sequence
  Primer
aaatctagaggcggtgtactgctggccgattccggtgccgctgtcagtggtaccaataacggcgccatacttaccctttcc

SEQ ID NO: 410 - DNA, 79 bp, Artificial Sequence
  Primer
aaatctagaggcggtgtactgctggccgattccggtgccgctgtcagtggtacctcaggaagtggcacactcactgtca

SEQ ID NO: 411 - DNA, 79 bp, Artificial Sequence
  Primer
aaatctagaggcggtgtactgctggccgattccggtgccgctgtcagtggtaccagcactgtgctgaacggtgccattg

SEQ ID NO: 412 - DNA, 34 bp, Artificial Sequence
  Primer
aggaagcttcagaaggtcacattcagtgtggcct

SEQ ID NO: 413 - DNA, 45 bp, Artificial Sequence
  Primer
agaactcgagaactgcaaaaatagtttgacaccctagccgatagg

SEQ ID NO: 414 - DNA, 46 bp, Artificial Sequence
  Primer
aaggatgcatggttattgtctcatgagcggatacatatttgaatgt

SEQ ID NO: 415 - DNA, 26 bp, Artificial Sequence
  Primer
catgccatggagaagctggaacagcc

SEQ ID NO: 416 - DNA, 26 bp, Artificial Sequence
  Primer
catgccatgggtccctctgccccggc

SEQ ID NO: 417 - DNA, 28 bp, Artificial Sequence
  Primer
cgggatccttagaacggtttgggcaacg

SEQ ID NO: 418 - DNA, 28 bp, Artificial Sequence
  Primer
cgggatccttagaactgcttgggaaggg

SEQ ID NO: 419 - DNA, 27 bp, Artificial Sequence
  Primer
cgggatccgtcgacctgcagttcgaag

SEQ ID NO: 420 - DNA, 50 bp, Artificial Sequence
  Primer
tgtcaaacatgagaattaattccggttgatgagcagctttaaggtttaat

SEQ ID NO: 421 - DNA, 50 bp, Artificial Sequence
  Primer
attaaaccttaaagctgctcatcaaccggaattaattctcatgtttgaca

SEQ ID NO: 422 - DNA, 33 bp, Artificial Sequence
  Primer
cgggatcccatacgcttaagcccaaccaacagc

SEQ ID NO: 423 - DNA, 26 bp, Artificial Sequence
  Primer
ttgatgagcagctttaaggtttaatg

SEQ ID NO: 424 - DNA, 60 bp, Artificial Sequence
  Primer
ctcactatagggcgaattcgagctcggtacccggggatccgtgtaggctggagctgcttc

SEQ ID NO: 425 - DNA, 54 bp, Artificial Sequence
  Primer
tgtcaaacatgagaattaattccggtctaatcgaataacacttaatattaaagg

SEQ ID NO: 426 - DNA, 54 bp, Artificial Sequence
  Primer
cctttaatattaagtgttattcgattagaccggaattaattctcatgtttgaca

SEQ ID NO: 427 - DNA, 25 bp, Artificial Sequence
  Primer
actccgtatcgagttgtcgtcctaa

SEQ ID NO: 428 - DNA, 29 bp, Artificial Sequence
  Primer
tctaatcgaataacacttaatattaaagg

SEQ ID NO: 429 - DNA, 42 bp, Artificial Sequence
  Primer
gaatcgttttccgggacgccggatgaagctaattctgattag

SEQ ID NO: 430 - DNA, 26 bp, Artificial Sequence
  Primer
cgggtaccgggccccccctcgaggtc

SEQ ID NO: 431 - DNA, 29 bp, Artificial Sequence
  Primer
cgggatcctcagcacagaaactacttttg

SEQ ID NO: 432 - DNA, 42 bp, Artificial Sequence
  Primer
ctaatcagaattagcttcatccggcgtcccggaaaacgattc

SEQ ID NO: 433 - DNA, 25 bp, Artificial Sequence
  Primer
ctgaccgttctgtccgtcacttccc

SEQ ID NO: 434 - DNA, 60 bp, Artificial Sequence
  Primer
tcttcaaccacaatcacctgttccgtagtgcctaaaccatcagcacagaaactacttttg

SEQ ID NO: 435 - DNA, 25 bp, Artificial Sequence
  Primer
ctgaccgttctgtccgtcacttccc

SEQ ID NO: 436 - DNA, 60 bp, Artificial Sequence
  Primer
tttaatcgttagattctaatagctagcctccaattaggcgatctaagataattactgtcc

SEQ ID NO: 437 - DNA, 46 bp, Artificial Sequence
  Primer
gaatcgttttccgggacgccttaagtactcggctcttatttaaatg

SEQ ID NO: 438 - DNA, 49 bp, Artificial Sequence
  Primer
gttttattcatggtattaattccattttttaatggacgaggggaaagtg

SEQ ID NO: 439 - DNA, 48 bp, Artificial Sequence
  Primer
gaaataattttaaaagccccaataggggatgaagctaattctgattag

SEQ ID NO: 440 - DNA, 26 bp, Artificial Sequence
  Primer
cgggtaccgggccccccctcgaggtc

SEQ ID NO: 441 - DNA, 49 bp, Artificial Sequence
  Primer
cactttcccctcgtccattaaaaaatggaattaataccatgaataaaac

SEQ ID NO: 442 - DNA, 48 bp, Artificial Sequence
  Primer
ctaatcagaattagcttcatcccctattggggcttttaaaattatttc

SEQ ID NO: 443 - DNA, 29 bp, Artificial Sequence
  Primer
cgggatcctcagcacagaaactacttttg

SEQ ID NO: 444 - DNA, 46 bp, Artificial Sequence
  Primer
catttaaataagagccgagtacttaaggcgtcccggaaaacgattc

SEQ ID NO: 445 - DNA, 60 bp, Artificial Sequence
  Primer
tttaatcgttagattctaatagctagcctccaattaggcgtaatcactcgtcgtacttgt

SEQ ID NO: 446 - DNA, 60 bp, Artificial Sequence
  Primer
tttaatcgttagattctaatagctagcctccaattaggcggttgttgatttagaaggaaa

SEQ ID NO: 447 - DNA, 60 bp, Artificial Sequence; identical to SEQ ID NO: 434
  Primer

SEQ ID NO: 448 - DNA, 25 bp, Artificial Sequence
  Primer
aatcgctcaagacgtgtaatgctgc

SEQ ID NO: 449 - DNA, 50 bp, Artificial Sequence
  Primer
tcagaaaaggtcatttgaagggatatgtaggctggagctgcttcgaagtt

SEQ ID NO: 450 - DNA, 50 bp, Artificial Sequence
  Primer
aacttcgaagcagctccagcctacatatcccttcaaatgaccttttctga

SEQ ID NO: 451 - DNA, 25 bp, Artificial Sequence
  Primer
ttcatctcacccttttaagttcaat

SEQ ID NO: 452 - DNA, 25 bp, Artificial Sequence
  Primer
aatcgctcaagacgtgtaatgctgc

SEQ ID NO: 453 - DNA, 25 bp, Artificial Sequence
  Primer
ttcatctcacccttttaagttcaat

SEQ ID NO: 454 - DNA, 90 bp, Artificial Sequence
  Primer
ccctgggccaacttttggcgaaaatgagacgttgaataacttcgtatagtacacattatacgaagttatatccgtcgacctgcagttcga

SEQ ID NO: 455 - DNA, 60 bp, Artificial Sequence
  Primer
ctttcaaatcaattcatttaaataagagccgagtacttaattcatctcacccttttaagt

SEQ ID NO: 456 - DNA, 26 bp, Artificial Sequence
  Primer
cgggtaccgggccccccctcgaggtc

SEQ ID NO: 457 - DNA, 49 bp, Artificial Sequence
  Primer
gctgtcaaacatgagaattggtcggtccatggagtcaaaccgccacgtc

SEQ ID NO: 458 - DNA, 49 bp, Artificial Sequence
  Primer
gacgtggcggtttgactccatggaccgaccaattctcatgtttgacagc

SEQ ID NO: 459 - DNA, 50 bp, Artificial Sequence
  Primer
gctctagaaagagccgagtacttaaggatcatcaggaaaacaggacgccg

SEQ ID NO: 460 - DNA, 26 bp, Artificial Sequence
  Primer
cgggtaccgggccccccctcgaggtc

SEQ ID NO: 461 - DNA, 50 bp, Artificial Sequence; identical to SEQ ID NO: 459
  Primer

SEQ ID NO: 462 - DNA, 60 bp, Artificial Sequence
  Primer
ctggcttttcttctttcaaatcaattcatttaaataagagccgagtacttaaggatcatc

SEQ ID NO: 463 - DNA, 27 bp, Artificial Sequence
  Primer
cgggatccgtcgacctgcagttcgaag

SEQ ID NO: 464 - DNA, 50 bp, Artificial Sequence; identical to SEQ ID NO: 420
  Primer

SEQ ID NO: 465 - DNA, 50 bp, Artificial Sequence; identical to SEQ ID NO: 421
  Primer

SEQ ID NO: 466 - DNA, 33 bp, Artificial Sequence; identical to SEQ ID NO: 422
  Primer

SEQ ID NO: 467 - DNA, 27 bp, Artificial Sequence
  Primer
cgggatccgtcgacctgcagttcgaag

SEQ ID NO: 468 - DNA, 33 bp, Artificial Sequence; identical to SEQ ID NO: 422
  Primer

SEQ ID NO: 469 - DNA, 26 bp, Artificial Sequence
  Primer
ttgatgagcagctttaaggtttaatg

SEQ ID NO: 470 - DNA, 60 bp, Artificial Sequence; identical to SEQ ID NO: 424
  Primer

SEQ ID NO: 471 - DNA, 26 bp, Artificial Sequence
  Primer
cgggtaccgggccccccctcgaggtc

SEQ ID NO: 472 - DNA, 45 bp, Artificial Sequence
  Primer
gaatcgttttccgggacgccaatacacctacccaatcgccaattg

SEQ ID NO: 473 - DNA, 50 bp, Artificial Sequence
  Primer
cggctcgtttcaatttctacactgttagctcctactcgagacaaactcag

SEQ ID NO: 474 - DNA, 50 bp, Artificial Sequence
  Primer
gtatcaaaataaaagagttaatacatatgctgctaagcttaaaaaacact

SEQ ID NO: 475 - DNA, 50 bp, Artificial Sequence
  Primer
ccatataccccataagcgttgcggctcactgacttgaacggatattgacg

SEQ ID NO: 476 - DNA, 45 bp, Artificial Sequence
  Primer
caattggcgattgggtaggtgtattggcgtcccggaaaacgattc

SEQ ID NO: 477 - DNA, 50 bp, Artificial Sequence
  Primer
ctgagtttgtctcgagtaggagctaacagtgtagaaattgaaacgagccg

SEQ ID NO: 478 - DNA, 50 bp, Artificial Sequence
  Primer
agtgttttttaagcttagcagcatatgtattaactcttttattttgatac

SEQ ID NO: 479 - DNA, 50 bp, Artificial Sequence
  Primer
cgtcaatatccgttcaagtcagtgagccgcaacgcttatggggtatatgg

SEQ ID NO: 480 - DNA, 30 bp, Artificial Sequence
  Primer
gctctagagttgccgccctccggcaattcg

SEQ ID NO: 481 - DNA, 26 bp, Artificial Sequence
  Primer
cgggtaccgggccccccctcgaggtc

SEQ ID NO: 482 - DNA, 30 bp, Artificial Sequence; identical to SEQ ID NO: 480
  Primer

SEQ ID NO: 483 - DNA, 25 bp, Artificial Sequence
  Primer
ctgaccgttctgtccgtcacttccc

SEQ ID NO: 484 - DNA, 22 bp, Artificial Sequence
  Primer
gttgccgccctccggcaattcg

SEQ ID NO: 485 - DNA, 26 bp, Artificial Sequence
  Primer
cgggtaccgggccccccctcgaggtc

SEQ ID NO: 486 - DNA, 49 bp, Artificial Sequence
  Primer
gctgtcaaacatgagaattggtcggcccaactgcagctgcgacaaaagc

SEQ ID NO: 487 - DNA, 50 bp, Artificial Sequence
  Primer
tgtattagtggcgccaaacccgtagtacactcgccgacggcaaattctaa

SEQ ID NO: 488 - DNA, 49 bp, Artificial Sequence
  Primer
gcttttgtcgcagctgcagttgggccgaccaattctcatgtttgacagc

SEQ ID NO: 489 - DNA, 50 bp, Artificial Sequence
  Primer
ttagaatttgccgtcggcgagtgtactacgggtttggcgccactaataca

SEQ ID NO: 490 - DNA, 33 bp, Artificial Sequence
  Primer
gctctagaaatgccttaaaacttgatgcatata

SEQ ID NO: 491 - DNA, 26 bp, Artificial Sequence
  Primer
cgggtaccgggccccccctcgaggtc

SEQ ID NO: 492 - DNA, 33 bp, Artificial Sequence; identical to SEQ ID NO: 490
  Primer

SEQ ID NO: 493 - DNA, 25 bp, Artificial Sequence
  Primer
ctgaccgttctgtccgtcacttccc

SEQ ID NO: 494 - DNA, 60 bp, Artificial Sequence
  Primer
gccgttaaagattcgcaattggcgattgggtaggtgtattaatgccttaaaacttgatgc

SEQ ID NO: 495 - DNA, 26 bp, Artificial Sequence
  Primer
cgggtaccgggccccccctcgaggtc

SEQ ID NO: 496 - DNA, 45 bp, Artificial Sequence
  Primer
gaatcgttttccgggacgccacgcaactactgcgtaacgctatgg

SEQ ID NO: 497 - DNA, 50 bp, Artificial Sequence
  Primer
tagcgcagctattaagtgtgactaacccttaaaactgccagccgctatta

SEQ ID NO: 498 - DNA, 50 bp, Artificial Sequence
  Primer
actgcgccaccgtgtaatatcattgttacttaactaaacagcttggcgtg

SEQ ID NO: 499 - DNA, 50 bp, Artificial Sequence
  Primer
ctagatagaaaatagaattgtaagcgaggcgatgagcttctattaagtat

SEQ ID NO: 500 - DNA, 45 bp, Artificial Sequence
  Primer
ccatagcgttacgcagtagttgcgtggcgtcccggaaaacgattc

SEQ ID NO: 501 - DNA, 50 bp, Artificial Sequence
  Primer
taatagcggctggcagttttaagggttagtcacacttaatagctgcgcta

SEQ ID NO: 502 - DNA, 50 bp, Artificial Sequence
  Primer
cacgccaagctgtttagttaagtaacaatgatattacacggtggcgcagt

SEQ ID NO: 503 - DNA, 50 bp, Artificial Sequence
  Primer
atacttaatagaagctcatcgcctcgcttacaattctattttctatctag

SEQ ID NO: 504 - DNA, 33 bp, Artificial Sequence
  Primer
gctctagacgaattgcagacttttgcgtgattg

SEQ ID NO: 505 - DNA, 26 bp, Artificial Sequence
  Primer
cgggtaccgggccccccctcgaggtc

SEQ ID NO: 506 - DNA, 33 bp, Artificial Sequence; identical to SEQ ID NO: 504
  Primer

SEQ ID NO: 507 - DNA, 25 bp, Artificial Sequence
  Primer
ctgaccgttctgtccgtcacttccc

SEQ ID NO: 508 - DNA, 60 bp, Artificial Sequence
  Primer
aattagacaaagtatgcttttgtcgcagctgcagttgggccgaattgcagacttttgcgt

SEQ ID NO: 509 - DNA, 26 bp, Artificial Sequence
  Primer
cgggtaccgggccccccctcgaggtc

SEQ ID NO: 510 - DNA, 49 bp, Artificial Sequence
  Primer
gctgtcaaacatgagaattggtcgccgccgagacgacagcaagctggac

SEQ ID NO: 511 - DNA, 50 bp, Artificial Sequence
  Primer
cgaatacatacaccacctaaaatacagaggaaaaaatcatgttggcttct

SEQ ID NO: 512 - DNA, 49 bp, Artificial Sequence
  Primer
gtccagcttgctgtcgtctcggcggcgaccaattctcatgtttgacagc

SEQ ID NO: 513 - DNA, 50 bp, Artificial Sequence
  Primer
agaagccaacatgattttttcctctgtattttaggtggtgtatgtattcg

SEQ ID NO: 514 - DNA, 33 bp, Artificial Sequence
  Primer
cgggatccatatggagtgtttttttaatgttgt

SEQ ID NO: 515 - DNA, 26 bp, Artificial Sequence
  Primer
cgggtaccgggccccccctcgaggtc

SEQ ID NO: 516 - DNA, 33 bp, Artificial Sequence; identical to SEQ ID NO: 514
  Primer

SEQ ID NO: 517 - DNA, 25 bp, Artificial Sequence
  Primer
ctgaccgttctgtccgtcacttccc

SEQ ID NO: 518 - DNA, 60 bp, Artificial Sequence
  Primer
accttcactttagtgccatagcgttacgcagtagttgcgtatatggagtgtttttttaat

SEQ ID NO: 519 - DNA, 26 bp, Artificial Sequence
  Primer
cgggtaccgggccccccctcgaggtc

SEQ ID NO: 520 - DNA, 45 bp, Artificial Sequence
  Primer
gaatcgttttccgggacgccgaactaataatgagcgagcaataac

SEQ ID NO: 521 - DNA, 50 bp, Artificial Sequence
  Primer
aagccgtaacagtaccaatcaacataatcgtctccttgtttgagcgtgat

SEQ ID NO: 522 - DNA, 50 bp, Artificial Sequence
  Primer
gtggagggaggcaatcgctaattgaaaaattagagtgtgtggcatttgtt

SEQ ID NO: 523 - DNA, 45 bp, Artificial Sequence
  Primer
gttattgctcgctcattattagttcggcgtcccggaaaacgattc

SEQ ID NO: 524 - DNA, 50 bp, Artificial Sequence
  Primer
atcacgctcaaacaaggagacgattatgttgattggtactgttacggctt

SEQ ID NO: 525 - DNA, 50 bp, Artificial Sequence
  Primer
aacaaatgccacacactctaatttttcaattagcgattgcctccctccac

SEQ ID NO: 526 - DNA, 33 bp, Artificial Sequence
  Primer
cgggatcccttagtgaacctctgattgacgacc

SEQ ID NO: 527 - DNA, 26 bp, Artificial Sequence
  Primer
cgggtaccgggccccccctcgaggtc

SEQ ID NO: 528 - DNA, 33 bp, Artificial Sequence; identical to SEQ ID NO: 526
  Primer

SEQ ID NO: 529 - DNA, 25 bp, Artificial Sequence
  Primer
ctgaccgttctgtccgtcacttccc

SEQ ID NO: 530 - DNA, 60 bp, Artificial Sequence
  Primer
aaagtcgtttatatagtccagcttgctgtcgtctcggcggcttagtgaacctctgattga

SEQ ID NO: 531 - DNA, 25 bp, Artificial Sequence
  Primer
ctgaccgttctgtccgtcacttccc

SEQ ID NO: 532 - DNA, 60 bp, Artificial Sequence
  Primer
gccgttaaagattcgcaattggcgattgggtaggtgtattgaattcaactgcaaaaatag

SEQ ID NO: 533 - DNA, 60 bp, Artificial Sequence
  Primer
gccgttaaagattcgcaattggcgattgggtaggtgtattgaattcttatcaaaaagagt

SEQ ID NO: 534 - DNA, 60 bp, Artificial Sequence
  Primer
gccgttaaagattcgcaattggcgattgggtaggtgtattgaattcttttaaaaaattca

SEQ ID NO: 535 - DNA, 60 bp, Artificial Sequence
  Primer
gccgttaaagattcgcaattggcgattgggtaggtgtattgaattcatcaaaaaaatatt

SEQ ID NO: 536 - DNA, 60 bp, Artificial Sequence
  Primer
agccgctgtaaaaagttatagttgttgatttagaaggaaagaattcttttaaaaaattca

SEQ ID NO: 537 - DNA, 60 bp, Artificial Sequence
  Primer
ctttcaaatcaattcatttaaataagagccgagtacttaagaattcttttaaaaaattca

SEQ ID NO: 538 - DNA, 25 bp, Artificial Sequence
  Primer
ctgaccgttctgtccgtcacttccc

SEQ ID NO: 539 - DNA, 60 bp, Artificial Sequence
  Primer
tttaatcgttagattctaatagctagcctccaattaggcggaattcttttaaaaaattca

SEQ ID NO: 540 - DNA, 60 bp, Artificial Sequence; identical to SEQ ID NO: 537
  Primer

SEQ ID NO: 541 - DNA, 60 bp, Artificial Sequence
  Primer
taatcactcgtcgtacttgtaaacgttcggaacatccaccgaattcttttaaaaaattca

SEQ ID NO: 542 - DNA, 60 bp, Artificial Sequence
  Primer
ggacgagccgtctggacaaacaaatgagcaatagtaagtgattccggggatccgtcgacc

SEQ ID NO: 543 - DNA, 58 bp, Artificial Sequence
  Primer
ctcactatagggcgaattcgagctcggtacccggggatccgtgtaggctggagctgct

SEQ ID NO: 544 - DNA, 29 bp, Artificial Sequence
  Primer
tctaatcgaataacacttaatattaaagg

SEQ ID NO: 545 - DNA, 60 bp, Artificial Sequence; identical to SEQ ID NO: 424
  Primer

SEQ ID NO: 546 - DNA, 60 bp, Artificial Sequence
  Primer
actggtcagagcttctgctgtcaggaatgcctggtgcccggtgtaggctggagctgcttc

SEQ ID NO: 547 - DNA, 60 bp, Artificial Sequence
  Primer
cgacaccaatcagcgtgacaactgtcaggatagcagccaggtgtaggctggagctgcttc

SEQ ID NO: 548 - DNA, 60 bp, Artificial Sequence
  Primer
ttacatagattgagtgaaggtacgagtaataacgtcctgcgtgtaggctggagctgcttc

SEQ ID NO: 549 - DNA, 60 bp, Artificial Sequence
  Primer
ttagaacattaccttatgaccgtactgctcaagaatgcctgtgtaggctggagctgcttc

SEQ ID NO: 550 - DNA, 60 bp, Artificial Sequence
  Primer
atggtcattaaggcgcaaagcccggcgggtttcgcggaaggtgtaggctggagctgcttc

SEQ ID NO: 551 - DNA, 60 bp, Artificial Sequence
  Primer
ccccaaaaagactttactattcaggcaatacatattggctgtgtaggctggagctgcttc

SEQ ID NO: 552 - DNA, 60 bp, Artificial Sequence
  Primer
ttacttagtgcagttcgcgcactgtttgttgacgatttgcgtgtaggctggagctgcttc

SEQ ID NO: 553 - DNA, 60 bp, Artificial Sequence
  Primer
ttacttattaacgaactcttcgcccagggcgatatctttcgtgtaggctggagctgcttc

SEQ ID NO: 554 - DNA, 60 bp, Artificial Sequence
  Primer
tccaggtaacagaaagttaacctctgtgcccgtagtccccgtgtaggctggagctgcttc

SEQ ID NO: 555 - DNA, 60 bp, Artificial Sequence
  Primer
tggcggtgctgttttgtaacccgccaaatcggcggtaacggtgtaggctggagctgcttc

SEQ ID NO: 556 - DNA, 60 bp, Artificial Sequence
  Primer
ttagccggtattacgcatacctgccgcaatcccggcaatagtgtaggctggagctgcttc

SEQ ID NO: 557 - DNA, 60 bp, Artificial Sequence
  Primer
tttggctttgagctggaattttttgactttctgctgacggattccggggatccgtcgacc

SEQ ID NO: 558 - DNA, 60 bp, Artificial Sequence
  Primer
tctcaaaagtatacccgatgcgtagccataccgttgctgcattccggggatccgtcgacc

SEQ ID NO: 559 - DNA, 60 bp, Artificial Sequence
  Primer
ctgctgcaatggccaaagtggccgaagaggcgggtgtctaattccggggatccgtcgacc

SEQ ID NO: 560 - DNA, 60 bp, Artificial Sequence; identical to SEQ ID NO: 559
  Primer

SEQ ID NO: 561 - DNA, 60 bp, Artificial Sequence
  Primer
ttatcgcccctgaatggctaaatcacccggcagatttttcattccggggatccgtcgacc

SEQ ID NO: 562 - DNA, 60 bp, Artificial Sequence
  Primer
ttacagtttcggaccagccgctaccagcgcggcacccgcaattccggggatccgtcgacc

SEQ ID NO: 563 - DNA, 60 bp, Artificial Sequence
  Primer
atgcactttgcgtgccgcccgtgactacgcggcacgccatattccggggatccgtcgacc

SEQ ID NO: 564 - DNA, 60 bp, Artificial Sequence
  Primer
cgcggcagcggagcaacatatcttagtttatcaatataatattccggggatccgtcgacc

SEQ ID NO: 565 - DNA, 60 bp, Artificial Sequence
  Primer
ttacgcattacgttgcaacaacatcgacttgatatggccgattccggggatccgtcgacc

SEQ ID NO: 566 - DNA, 60 bp, Artificial Sequence
  Primer
ttactgctgctgtgcagactgaatcgcagtcagcgcgatgattccggggatccgtcgacc

SEQ ID NO: 567 - DNA, 60 bp, Artificial Sequence
  Primer
ttgcgtagtaatgtcagtatgctcggcaaagtgctgggagattccggggatccgtcgacc

SEQ ID NO: 568 - DNA, 25 bp, Artificial Sequence
  Primer
ctctagagtcgacctgcaggcatgc

SEQ ID NO: 569 - DNA, 50 bp, Artificial Sequence
  Primer
aacaatttcacacaggaaacagaccatggctgttactaatgtcgctgaac

SEQ ID NO: 570 - DNA, 50 bp, Artificial Sequence
  Primer
gttcagcgacattagtaacagccatggtctgtttcctgtgtgaaattgtt

SEQ ID NO: 571 - DNA, 30 bp, Artificial Sequence
  Primer
ttaagcggattttttcgcttttttctcagc

SEQ ID NO: 572 - DNA, 25 bp, Artificial Sequence
  Primer
ctctagagtcgacctgcaggcatgc

SEQ ID NO: 573 - DNA, 30 bp, Artificial Sequence; identical to SEQ ID NO: 571
  Primer

SEQ ID NO: 574 - DNA, 25 bp, Artificial Sequence
  Primer
ttagaaagcgctcaggaagagttct

SEQ ID NO: 575 - DNA, 50 bp, Artificial Sequence
  Primer
tgaagaagccattatatatacctccttagaggagcttgttaacaggctta

SEQ ID NO: 576 - DNA, 50 bp, Artificial Sequence
  Primer
agtataactcattatatatacctcctgtaggctggagctgcttcgaagtt

SEQ ID NO: 577 - DNA, 50 bp, Artificial Sequence
  Primer
taagcctgttaacaagctcctctaaggaggtatatataatggcttcttca

SEQ ID NO: 578 - DNA, 50 bp, Artificial Sequence
  Primer
aacttcgaagcagctccagcctacaggaggtatatataatgagttatact

SEQ ID NO: 579 - DNA, 25 bp, Artificial Sequence
  Primer
aatcgctcaagacgtgtaatgctgc

SEQ ID NO: 580 - DNA, 25 bp, Artificial Sequence
  Primer
ttagaaagcgctcaggaagagttct

SEQ ID NO: 581 - DNA, 25 bp, Artificial Sequence
  Primer
aatcgctcaagacgtgtaatgctgc

SEQ ID NO: 582 - DNA, 60 bp, Artificial Sequence
  Primer
actggtcagagcttctgctgtcaggaatgcctggtgcccgttagaaagcgctcaggaaga

SEQ ID NO: 583 - DNA, 60 bp, Artificial Sequence
  Primer
cgacaccaatcagcgtgacaactgtcaggatagcagccagttagaaagcgctcaggaaga

SEQ ID NO: 584 - DNA, 60 bp, Artificial Sequence
  Primer
ttacatagattgagtgaaggtacgagtaataacgtcctgcttagaaagcgctcaggaaga

SEQ ID NO: 585 - DNA, 60 bp, Artificial Sequence; identical to SEQ ID NO: 557
  Primer

SEQ ID NO: 586 - DNA, 60 bp, Artificial Sequence; identical to SEQ ID NO: 558
  Primer

SEQ ID NO: 587 - DNA, 60 bp, Artificial Sequence; identical to SEQ ID NO: 559
  Primer

SEQ ID NO: 588 - DNA, 59 bp, Artificial Sequence
  Primer
gcctatcggctagggtgtcaaactattttttgcagttttttgtaggctggagctgcttc

SEQ ID NO: 589 - DNA, 59 bp, Artificial Sequence
  Primer
gaaacttaagcattttagcaaataaaacttttaattaaaatgtaggctggagctgcttc

SEQ ID NO: 590 - DNA, 59 bp, Artificial Sequence
  Primer
gattgctgggctattgtcaacaattttttagtagtctgagtgtaggctggagctgcttc

SEQ ID NO: 591 - DNA, 59 bp, Artificial Sequence
  Primer
gtattggaaaattttatcaagaaatttttatttttccatatgtaggctggagctgcttc

SEQ ID NO: 592 - DNA, 59 bp, Artificial Sequence
  Primer
ctaaatttccacctgtgtcaataacggtttttatatccgctgtaggctggagctgcttc

SEQ ID NO: 593 - DNA, 60 bp, Artificial Sequence
  Primer
tttaagatgtacccagttcgatgagagcgataactcacacaatcgctcaagacgtgtaat

SEQ ID NO: 594 - DNA, 60 bp, Artificial Sequence
  Primer
tgtataattactttataaattgatgagaaggaaatcacacaatcgctcaagacgtgtaat

SEQ ID NO: 595 - DNA, 60 bp, Artificial Sequence
  Primer
ggtaaaatatcgatttaggcagttcacacagatatcattaaatcgctcaagacgtgtaat

SEQ ID NO: 596 - DNA, 60 bp, Artificial Sequence
  Primer
tattataatattgttattaaagaggagaaattaaccacacaatcgctcaagacgtgtaat

SEQ ID NO: 597 - DNA, 60 bp, Artificial Sequence
  Primer
aatatactgttagtaaacctaatggatcgacctttcacacaatcgctcaagacgtgtaat

SEQ ID NO: 598 - DNA, 60 bp, Artificial Sequence
  Primer
aaataggtaccgacagtataactcattatatatacctcctgtgtgagttatcgctctcat

SEQ ID NO: 599 - DNA, 60 bp, Artificial Sequence
  Primer
aaataggtaccgacagtataactcattatatatacctcctgtgtgatttccttctcatca

SEQ ID NO: 600 - DNA, 60 bp, Artificial Sequence
  Primer
aaataggtaccgacagtataactcattatatatacctccttaatgatatctgtgtgaact

SEQ ID NO: 601 - DNA, 60 bp, Artificial Sequence
  Primer
aaataggtaccgacagtataactcattatatatacctcctgtgtggttaatttctcctct

SEQ ID NO: 602 - DNA, 60 bp, Artificial Sequence
  Primer
aaataggtaccgacagtataactcattatatatacctcctgtgtgaaaggtcgatccatt

SEQ ID NO: 603 - DNA, 60 bp, Artificial Sequence; identical to SEQ ID NO: 557
  Primer

SEQ ID NO: 604 - DNA, 60 bp, Artificial Sequence; identical to SEQ ID NO: 558
  Primer

SEQ ID NO: 605 - DNA, 60 bp, Artificial Sequence; identical to SEQ ID NO: 559
  Primer

SEQ ID NO: 606 - DNA, 39 bp, Artificial Sequence
  Primer
cacacaccatggatgaaagctactaaactggtactgggc

SEQ ID NO: 607 - DNA, 28 bp, Artificial Sequence
  Primer
ccctttggatccgtccggacgagtgccg